(19) United States
(12) Patent Application Publication
(10) Pub. No.: US 2005/0118594 A1
(43) Pub. Date: Jun. 2, 2005

(54) Enzymes

(76) Inventors: Narinder K. Chawla, Union City, CA (US); Soo Yuen Lee, Daly City, CA (US); Huijun Z. Ring, Los Altos, CA (US); Ernestine A. Lee, Castro Valley, CA (US); Ian J. Forsythe, Redwood City, CA (US); Reena Khare, Saratoga, CA (US); Uyen K. Tran, San Jose, CA (US); Amy E. Kable, San Francisco, CA (US); Thomas W. Richardson, Redwood City, CA (US); Brooke M. Emerling, Palo Alto, CA (US); Erika A. Lindquist, Alameda, CA (US); Mariah R. Baughn, San Leandro, CA (US); April J.A. Hafalia, Santa Clara, CA (US); Pei Jin, Palo Alto, CA (US); Anita Swarnakar, San Francisco, CA (US); Joana X. Li, San Francisco, CA (US); Joseph P. Marquis, San Jose, CA (US); Ann E. Gorvad, Livermore, CA (US); William W. Sprague, Sacramento, CA (US); Shanya D. Becha, Castro Valley, CA (US); Vicki S. Elliott, San Jose, CA (US)

Correspondence Address: FOLEY AND LARDNER;SUITE 500, 3000 K STREET NW, WASHINGTON, DC, 20007, US

(21) Appl. No.: 10/498,788

(22) PCT Filed: Dec. 12, 2002
(86) PCT No.: PCT/US02/40161

Related U.S. Application Data
(60) Provisional application No. 60/340,357, filed on Dec. 14, 2001.
(60) Provisional application No. 60/342,962, filed on Dec. 20, 2001.
(60) Provisional application No. 60/343,558, filed on Dec. 21, 2001.
(60) Provisional application No. 60/351,107, filed on Jan. 22, 2002.

(51) Int. Cl. C12Q 1/68; G01N 33/53; C07H 21/04; C12N 9/00

(52) U.S. Cl. 435/6; 435/7.1; 435/69.1; 435/320.1; 435/325; 435/183; 530/388.26; 536/23.2

(57) ABSTRACT

Various embodiments of the invention provide human enzymes (ENZM) and polynucleotides which identify and encode-ENZM. Embodiments of the invention also provide expression vectors, host cells, antibodies, agonists, and antagonists. Other embodiments provide methods for diagnosing, treating, or preventing disorders associated with aberrant expression of ENZM.

### TECHNICAL FIELD

The invention relates to novel nucleic acids, enzymes encoded by these nucleic acids, and to the use of these nucleic acids and proteins in the diagnosis, treatment, and prevention of autoimmune/inflammatory disorders, infectious disorders, immune deficiencies, disorders of metabolism, reproductive disorders, neurological disorders, cardiovascular disorders, eye disorders, and cell proliferative disorders, including cancer. The invention also relates to the assessment of the effects of exogenous compounds on the expression of nucleic acids and enzymes.

### BACKGROUND OF THE INVENTION

The cellular processes of biogenesis and biodegradation involve a number of key enzyme classes including oxidoreductases, transferases, hydrolases, lyases, isomerases, ligases, and others. Each class of enzyme comprises many substrate-specific enzymes having precise and well regulated functions. Enzymes facilitate metabolic processes such as glycolysis, the tricarboxylic cycle, and fatty acid metabolism; synthesis or degradation of amino acids, steroids, phospholipids, and alcohols; regulation of cell signaling, proliferation, inflamation, and apoptosis; and through catalyzing critical steps in DNA replication and repair and the process of translation.

Oxidoreductases

Many pathways of biogenesis and biodegradation require oxidoreductase (dehydrogenase or reductase) activity, coupled to reduction or oxidation of a cofactor. Potential cofactors include cytochromes, oxygen, disulfide, iron-sulfur proteins, flavin adenine dinucleotide (FAD), and the nicotinamide adenine dinucleotides NAD and NADP (Newsholme, E. A. and A. R. Leech (1983) Biochemistry for the Medical Sciences, John Wiley and Sons, Chichester, U. K. pp. 779-793). Reductase activity catalyzes transfer of electrons between substrate(s) and cofactor(s) with concurrent oxidation of the cofactor. Reverse dehydrogenase activity catalyzes the reduction of a cofactor and consequent oxidation of the substrate. Oxidoreductase enzymes are a broad superfamily that catalyze reactions in all cells of organisms, including metabolism of sugar, certain detoxification reactions, and synthesis or degradation of fatty acids, amino acids, glucocorticoids, estrogens, androgens, and prostaglandins. Different family members may be referred to as oxidoreductases, oxidases, reductases, or dehydrogenases, and they often have distinct cellular locations such as the cytosol, the plasma membrane, mitochondrial inner or outer membrane, and peroxisomes.

Short-chain alcohol dehydrogenases (SCADs) are a family of dehydrogenases that share only 15% to 30% sequence identity, with similarity predominantly in the coenzyme binding domain and the substrate binding domain. In addition to their role in detoxification of ethanol, SCADs are involved in synthesis and degradation of fatty acids, steroids, and some prostaglandins, and are therefore implicated in a variety of disorders such as lipid storage disease, myopathy, SCAD deficiency, and certain genetic disorders. For example, retinol dehydrogenase is a SCAD-family member (Simon, A. et al. (1995) J. Biol. Chem. 270:1107-1112) that converts retinol to retinal, the precursor of retinoic acid. Retinoic acid, a regulator of differentiation and apoptosis, has been shown to down-regulate genes involved in cell proliferation and inflammation (Chai, X. et al. (1995) J. Biol. Chem. 270:3900-3904). In addition, retinol dehydrogenase has been linked to hereditary eye diseases such as autosomal recessive childhood-onset severe retinal dystrophy (Simon, A. et al. (1996) Genomics 36:424-430).

Membrane-bound succinate dehydrogenases (succinate:quinone reductases, SQR) and fumarate reductases (quinol:fumarate reductases, QFR) couple the oxidation of succinate to fumarate with the reduction of quinone to quinol, and also catalyze the reverse reaction. QFR and SQR complexes are collectively known as succinate:quinone oxidoreductases (EC 1.3.5.1) and have similar compositions. The complexes consist of two hydrophilic and one or two hydrophobic, membrane-integrated subunits. The larger hydrophilic subunit A carries covalently bound flavin adenine dinucleotide; subunit B contains three iron-sulphur centers (Lancaster, C. R. and A. Kroger (2000) Biochim. Biophys. Acta 1459:422-431). The full-length cDNA sequence for the flavoprotein subunit of human heart succinate dehydrogenase (succinate: (acceptor) oxidoreductase; EC 1.3.99.1) is similar to the bovine succinate dehydrogenase in that it contains a cysteine triplet and in that the active site contains an additional cysteine that is not present in yeast or prokaryotic SQRs (Morris, A. A. et al. (1994) Biochim. Biophys. Acta 29:125-128).

Propagation of nerve impulses, modulation of cell proliferation and differentiation, induction of the immune response, and tissue homeostasis involve neurotransmitter metabolism (Weiss, B. (1991) Neurotoxicology 12:379-386; Collins, S. M. et al. (1992) Ann. N.Y. Acad. Sci. 664:415-424; Brown, J. K. and H. Imam (1991) J. Inherit. Metab. Dis. 14:436-458). Many pathways of neurotransmitter metabolism require oxidoreductase activity, coupled to reduction or oxidation of a cofactor, such as NAD⁺/NADH (Newsholme and Leech, supra, pp. 779-793). Degradation of catecholamines (epinephrine or norepinephrine) requires alcohol dehydrogenase (in the brain) or aldehyde dehydrogenase (in peripheral tissue). NAD⁺-dependent aldehyde dehydrogenase oxidizes 5-hydroxyindole-3-acetate (the product of 5-hydroxytryptamine (serotonin) metabolism) in the brain, blood platelets, liver and pulmonary endothelium (Newsholme and Leech, supra, p. 786). Other neurotranlsmitter degradation pathways that utilize NAD⁺/NADH-dependent oxidoreductase activity include those of L-DOPA (precursor of dopamine, a neuronal excitatory compound), glycine (an inhibitory neurotranlsmitter in the brain and spinal cord), histamine (liberated from mast cells during the inflammatory response), and taurine (an inhibitory neurotransmitter of the brain stem, spinal cord and retina) (Newsholme and Leech, supra, pp. 790, 792). Epigenetic or genetic defects in neurotransmitter metabolic pathways can result in diseases including Parkinson disease and inherited myoclonus (McCance, K. L. and S. E. Huether (1994) Pathophysiology, Mosby-Year Book, Inc., St. Louis, Mo. pp. 402-404; Gundlach, A. L. (1990) FASEB J. 4:2761-2766).

Tetrahydrofolate is a derivatized glutamate molecule that acts as a carrier, providing activated one-carbon units to a wide variety of biosynthetic reactions, including synthesis of purines, pyrimidines, and the amino acid methionine. Tetrahydrofolate is generated by the activity of a holoenzyme complex called tetrahydrofolate synthase, which includes three enzyme activities: tetrahydrofolate dehydrogenase, tetrahydrofolate cyclohydrolase, and tetrahydrofolate synthetase. Thus, tetrahydrofolate dehydrogenase plays an important role in generating building blocks for nucleic and amino acids, crucial to proliferating cells.

3-Hydroxyacyl-CoA dehydrogenase (3HACD) is involved in fatty acid metabolism. It catalyzes the reduction of 3-hydroxyacyl-CoA to 3-oxoacyl-CoA, with concomitant oxidation of NAD to NADH, in the mitochondria and peroxisomes of eukaryotic cells. In peroxisomes, 3HACD and enoyl-CoA hydratase form an enzyme complex called bifunctional enzyme, defects in which are associated with peroxisomal bifunctional enzyme deficiency. This interruption in fatty acid metabolism produces accumulation of very-long chain fatty acids, disrupting development of the brain, bone, and adrenal glands. Infants born with this deficiency typically die within 6 months (Watkins, P. et al. (1989) 3. Clin. Invest. 83:771-777; Online Mendelian Inheritance in Man (OMIM), #261515). The neurodegeneration characteristic of Alzheimer's disease involves development of extracellular plaques in certain brain regions. A major protein component of these plaques is the peptide amyloid-β (Aβ), which is one of several cleavage products of amyloid precursor protein (APP). 3HACD has been shown to bind the Aβ peptide, and is overexpressed in neurons affected in Alzheimer's disease. In addition, an antibody against 3HACD can block the toxic effects of Aβ in a cell culture model of Alzheimer's disease (Yan, S. et al. (1997) Nature 389:689-695; OMIM, #602057).

Steroids such as estrogen, testosterone, and corticosterone are generated from a common precursor, cholesterol, and interconverted. Enzymes acting upon cholesterol include dehydrogenases. Steroid dehydrogenases, such as the hydroxysteroid dehydrogenases, are involved in hypertension, fertility, and cancer (Duax, W. L. and D. Ghosh (1997) Steroids 62:95-100). One such dehydrogenase is 3-oxo-5-α-steroid dehydrogenase (OASD), a microsomal membrane protein highly expressed in prostate and other androgen-responsive tissues. OASD catalyzes the conversion of testosterone into dihydrotestosterone, which is the most potent androgen. Dihydrotestosterone is essential for the formation of the male phenotype during embryogenesis, as well as for proper androgen-mediated growth of tissues such as the prostate and male genitalia. A defect in OASD leads to defective formation of the external genitalia (Andersson, S. et al. (1991) Nature 354:159-161; Labrie, F. et al. (1992) Endocrinology 131:1571-1573; OMIM #264600).

17β-hydroxysteroid dehydrogenase (17βHSD6) plays an important role in the regulation of the male reproductive hormone, dihydrotestosterone (DHTT). 17βHSD6 acts to reduce levels of DHTT by oxidizing a precursor of DHTT, 3α-diol, to androsterone which is readily glucuronidated and removed. 17βHSD6 is active with both androgen and estrogen substrates in embryonic kidney 293 cells. Isozymes of 17βHSD catalyze oxidation and/or reduction reactions in various tissues with preferences for different steroid substrates (Biswas, M. G. and D. W. Russell (1997) J. Biol. Chem. 272:15959-15966). For example, 17βHSD1 preferentially reduces estradiol and is abundant in the ovary and placenta. 17βHSD2 catalyzes oxidation of androgens and is present in the endometrium and placenta. 17βHSD3 is exclusively a reductive enzyme in the testis (Geissler, W. M. et al. (1994) Nature Genet. 7:34-39). An excess of androgens such as DHTT can contribute to diseases such as benign prostatic hyperplasia and prostate cancer.

The oxidoreductase isocitrate dehydrogenase catalyzes the conversion of isocitrate to a-ketoglutarate, a substrate of the citric acid cycle. Isocitrate dehydrogenase can be either NAD or NADP dependent, and is found in the cytosol, mitochondria, and peroxisomes. Activity of isocitrate dehydrogenase is regulated developmentally, and by hormones, neurotransmitters, and growth factors.

Hydroxypyruvate reductase (HPR), a peroxisomal 2-hydroxyacid dehydrogenase in the glycolate pathway, catalyzes the conversion of hydroxypyruvate to glycerate with the oxidation of both NADH and NADPH. The reverse dehydrogenase reaction reduces NAD⁺ and NADP⁺. HPR recycles nucleotides and bases back into pathways leading to the synthesis of ATP and GTP, which are used to produce DNA and RNA and to control various aspects of signal transduction and energy metabolism. Purine nucleotide biosynthesis inhibitors are used as antiproliferative agents to treat cancer and viral diseases. HPR also regulates biochemical synthesis of serine and cellular serine levels available for protein synthesis.

The mitochondrial electron transport (or respiratory) chain is the series of oxidoreductase-type enzyme complexes in the mitochondrial membrane that is responsible for the transport of electrons from NADH to oxygen and the coupling of this oxidation to the synthesis of ATP (oxidative phosphorylation). ATP provides energy to drive energy-requiring reactions. The key respiratory chain complexes are NADH:ubiquinone oxidoreductase (complex I), succinate:ubiquinone oxidoreductase (complex II), cytochrome c₁-b oxidoreductase (complex III), cytochrome c oxidase (complex IV), and ATP synthase (complex V) (Alberts, B. et al. (1994) Molecular Biology of the Cell, Garland Publishing, Inc., New York, N.Y., pp. 677-678). All of these complexes are located on the inner matrix side of the mitochondrial membrane except complex II, which is on the cytosolic side where it transports electrons generated in the citric acid cycle to the respiratory chain. Electrons released in oxidation of succinate to fumarate in the citric acid cycle are transferred through electron carriers in complex II to membrane bound ubiquinone (Q). Transcriptional regulation of these nuclear-encoded genes controls the biogenesis of respiratory enzymes. Defects and altered expression of enzymes in the respiratory chain are associated with a variety of disease conditions.

Other dehydrogenase activities using NAD as a cofactor include 3-hydroxyisobutyrate dehydrogenase (3BBD), which catalyzes the NAD-dependent oxidation of 3-hydroxyisobutyrate to methylmalonate semialdehyde within mitochondria. 3-hydroxyisobutyrate levels are elevated in ketoacidosis, methylmalonic acidemia, and other disorders (Rougraff, P. M. et al. (1989) J. Biol. Chem. 264:5899-5903). Another mitochondrial dehydrogenase important in amino acid metabolism is the enzyme isovaleryl-CoA-dehydrogenase (IVD). IVD is involved in leucine metabolism and catalyzes the oxidation of isovaleryl-CoA to 3-methylcrotonyl-CoA. Human IVD is a tetrameric flavoprotein synthesized in the cytosol with a mitochondrial import signal sequence. A mutation in the gene encoding IVD results in isovaleric acidemia (Vockley, J. et al. (1992) J. Biol. Chem. 267:2494-2501).

The family of glutathione peroxidases encompass tetrameric glutathione peroxidases (GPx1-3) and the monomeric phospholipid hydroperoxide glutathione peroxidase (PHGPx/GPx4). Although the overall homology between the tetrameric enzymes and GPx4 is less than 30%, a pronounced similarity has been detected in clusters involved in the active site and a common catalytic triad has been defined by structural and kinetic data (Epp, O. et al. (1983) Eur. J. Biochem. 133:51-69). GPx1 is ubiquitously expressed in cells, whereas GPx2 is present in the liver and colon, and GPx3 is present in plasma. GPx4 is found at low levels in all tissues but is expressed at high levels in the testis (Ursini, F. et al (1995) Meth. Enzymol. 252:38-53). GPx4 is the only monomeric glutathione peroxidase found in mammals and the only mammalian glutathione peroxidase to show high affinity for and reactivity with phospholipid hydroperoxides, and to be membrane associated. A tandem mechanism for the antioxidant activities of GPx4 and vitamin E has been suggested. GPx4 has alternative transcription and translation start sites which determine its subcellular localization (Esworthy, R. S. et al. (1994) Gene 144:317-318; and Maiorino, M. et al. (1990) Meth. Enzymol. 186:448-450).

The glutathione S-transferases (GST) are a ubiquitous family of enzymes with dual substrate specificities that perform important biochemical functions of xenobiotic biotransformation and detoxification, drug metabolism, and protection of tissues against peroxidative damage. They catalyze the conjugation of an electrophile with reduced glutathione (GSH) which results in either activation or deactivation/detoxification. The absolute requirement for binding reduced GSH to a variety of chemicals necessitates a diversity in GST structures in various organisms and cell types. GSTs are homodimeric or heterodimeric proteins localized in the cytosol. The major isozymes share common structural and catalytic properties and include four major classes, Alpha, Mu, Pi, and Theta. Each GST possesses a common binding site for GSH, and a variable hydrophobic binding site specific for its particular electrophilic substrates. Specific amino acid residues within GSTs have been identified as important for these binding sites and for catalytic activity. Residues Q67, T68, D101, E104, and R131 are important for the binding of GSH (Lee, H.-C. et al. (1995) J. Biol. Chem. 270:99-109). Residues R13, R20, and R69 are important for the catalytic activity of GST (Stenberg, G. et al. (1991) Biochem. J. 274:549-555).

GSTs normally deactivate and detoxify potentially mutagenic and carcinogenic chemicals. Some forms of rat and human GSTs are reliable preneoplastic markers of carcinogenesis. Dihalomethanes, which produce liver tumors in mice, are believed to be activated by GST (Thier, R. et al. (1993) Proc. Natl. Acad. Sci. USA 90:8567-8580). The mutagenicity of ethylene dibromide and ethylene dichloride is increased in bacterial cells expressing the human Alpha GST, A1-1, while the mutagenicity of aflatoxin B1 is substantially reduced by enhancing the expression of GST (Simula, T. P. et al. (1993) Carcinogenesis 14:1371-1376). Thus, control of GST activity may be useful in the control of mutagenesis and carcinogenesis.

GST has been implicated in the acquired resistance of many cancers to drug treatment, the phenomenon known as multi-drug resistance (MDR). MDR occurs when a cancer patient is treated with a cytotoxic drug such as cyclophosphamide and subsequently becomes resistant to this drug and to a variety of other cytotoxic agents as well. Increased GST levels are associated with some drug resistant cancers, and it is believed that this increase occurs in response to the drug agent which is then deactivated by the GST catalyzed GSH conjugation reaction. The increased GST levels then protect the cancer cells from other cytotoxic agents for which GST has affinity. Increased levels of A1-1 in tumors has been linked to drug resistance induced by cyclophosphamide treatment (Dirven, H. A. et al. (1994) Cancer Res. 54:6215-6220). Thus control of GST activity in cancerous tissues may be useful in treating MDR in cancer patients.

The reduction of ribonucleotides to the corresponding deoxyribonucleotides, needed for DNA synthesis during cell proliferation, is catalyzed by the enzyme ribonucleotide diphosphate reductase. Glutaredoxin is a glutathione (GSH)-dependent hydrogen donor for ribonucleotide diphosphate reductase and contains the active site consensus sequence -C-P-Y-C-. This sequence is conserved in glutaredoxins from such different organisms as Escherichia coli, vaccinia virus, yeast, plants, and mammalian cells. Glutaredoxin has inherent GSH-disulfide oxidoreductase (thioltransferase) activity in a coupled system with GSH, NADPH, and GSH-reductase, catalyzing the reduction of low molecular weight disulfides as well as proteins. Glutaredoxin has been proposed to exert a general thiol redox control of protein activity by acting both as an effective protein disulfide reductase, similar to thioredoxin, and as a specific GSH-mixed disulfide reductase (Padilla, C. A. et al. (1996) FEBS Lett. 378:69-73).

In addition to their important role in DNA synthesis and cell division, glutaredoxin and other thioproteins provide effective antioxidant defense against oxygen radicals and hydrogen peroxide (Schallreuter, K. U. and J. M. Wood (1991) Melanoma Res. 1:159-167). Glutaredoxin is the principal agent responsible for protein dethiolation in vivo and reduces dehydroascorbic acid in normal human neutrophils (Jung, C. H. and J. A. Thomas (1996) Arch. Biochem. Biophys. 335:61-72; Park, J. B. and M. Levine (1996) Biochem. J. 315:931-938).

The thioredoxin system serves as a hydrogen donor for ribonucleotide reductase and as a regulator of enzymes by redox control. It also modulates the activity of transcription factors such as NF-κB, AP-1, and steroid receptors. Several cytokines or secreted cytokine-like factors such as adult T-cell leukemia-derived factor, 3B6-interleukin-1, T-hybridoma-derived (MP-6) B cell stimulatory factor, and early pregnancy factor have been reported to be identical to thioredoxin (Holmgren, A. (1985) Annu. Rev. Biochem. 54:237-271; Abate, C. et al. (1990) Science 249:1157-1161; Tagaya, Y. et al. (1989) EMBO J. 8:757-764; Wakasugi, H. (1987) Proc. Natl. Acad. Sci. USA 84:804-808; Rosen, A. et al. (1995) Int. Immunol. 7:625-633). Thus thioredoxin secreted by stimulated lymphocytes (Yodoi, J. and T. Tursz (1991) Adv. Cancer Res. 57:381-411; Tagaya, N. et al. (1990) Proc. Natl. Acad. Sci. USA 87:8282-8286) has extracellular activities including a role as a regulator of cell growth and a mediator in the immune system (Miranda-Vizuete, A. et al. (1996) J. Biol. Chem. 271:19099-19103; Yamauchi, A. et al. (1992) Mol. Immunol. 29:263-270). Thioredoxin and thioredoxin reductase protect against cytotoxicity mediated by reactive oxygen species in disorders such as Alzheimer's disease (Lovell, M. A. (2000) Free Radic. Biol. Med. 28:418-427).

The selenoprotein thioredoxin reductase is secreted by both normal and neoplastic cells and has been implicated as both a growth factor and as a polypeptide involved in apoptosis (Soderberg, A. et al. (2000) Cancer Res. 60:2281-2289). An extracellular plasmin reductase secreted by hamster ovary cells (HT-1080) has been shown to participate in the generation of angiostatin from plasmin. In this case, the reduction of the plasmin disulfide bonds triggers the proteolytic cleavage of plasmin which yields the angiogenesis inhibitor, angiostatin (Stathakis, P. et al. (1997) J. Biol. Chem. 272:20641-20645). Low levels of reduced sulfhydryl groups in plasma has been associated with rheumatoid arthritis. The failure of these sulfhydryl groups to scavenge active oxygen species (e.g., hydrogen peroxide produced by activated neutrophils) results in oxidative damage to surrounding tissues and the resulting inflammation (Hall, N. D. et al. (1994) Rheumatol. Int. 4:35-38).

Another example of the importance of redox reactions in cell metabolism is the degradation of saturated and unsaturated fatty acids by mitochondrial and peroxisomal beta-oxidation enzymes which sequentially remove two-carbon units from Coenzyme A (CoA)-activated fatty acids. The main beta-oxidation pathway degrades both saturated and unsaturated fatty acids while the auxiliary pathway performs additional steps required for the degradation of unsaturated fatty acids.

The pathways of mitchondrial and peroxisomal beta-oxidation use similar enzymes, but have different substrate specificities and functions. Mitochondria oxidize short-, medium-, and long-chain fatty acids to produce energy for cells. Mitochondrial beta-oxidation is a major energy source for cardiac and skeletal muscle. In liver, it provides ketone bodies to the peripheral circulation when glucose levels are low as in starvation, endurance exercise, and diabetes (Eaton, S. et al. (1996) Biochem. J. 320:345-357). Peroxisomes oxidize medium-, long-, and very-long-chain fatty acids, dicarboxylic fatty acids, branched fatty acids, prostaglandins, xenobiotics, and bile acid intermediates. The chief roles of peroxisomal beta-oxidation are to shorten toxic lipophilic carboxylic acids to facilitate their excretion and to shorten very-long-chain fatty acids prior to mitochondrial beta-oxidation (Mannaerts, G. P. and P. P. Van Veldhoven (1993) Biochimie 75:147-158).

The auxiliary beta-oxidation enzyme 2,4-dienoyl-CoA reductase catalyzes the following reaction:
trans-2, cis/trans-4-dienoyl-CoA+NADPH+H⁺--->trans-3-enoyl-CoA+NADP⁺
This reaction removes even-numbered double bonds from unsaturated fatty acids prior to their entry into the main beta-oxidation pathway (Koivuranta, K. T. et al. (1994) Biochem. J. 304:787-792). The enzyme may also remove odd-numbered double bonds from unsaturated fatty acids (Smeland, T. E. et al. (1992) Proc. Natl. Acad. Sci. USA 89:6673-6677).

Rat 2,4-dienoyl-CoA reductase is located in both mitochondria and peroxisomes (Dommes, V. et al. (1981) J. Biol. Chem. 256:8259-8262). Two immunologically different forms of rat mitochondrial enzyme exist with molecular masses of 60 kDa and 120 kDa (Hakkola, E. H. and J. K. Hiltunen (1993) Eur. J. Biochem. 215:199-204). The 120 kDa mitochondrial rat enzyme is synthesized as a 335 amino acid precursor with a 29 amino acid N-terminal leader peptide which is cleaved to form the mature enzyme (Hirose, A. et al. (1990) Biochim. Biophys. Acta 1049:346-349). A human mitochondrial enzyme 83% similar to rat enzyme is synthesized as a 335 amino acid residue precursor with a 19 amino acid N-terminal leader peptide (Koivuranta et al., supra). These cloned human and rat mitochondrial enzymes function as homotetramers (Koivuranta et al., supra). A Saccharomyces cerevisiae peroxisomal 2,4-dienoyl-CoA reductase is 295 amino acids long, contains a C-terminal peroxisomal targeting signal, and functions as a homodimer (Coe, J. G. S. et al. (1994) Mol. Gen. Genet. 244:661-672; and Gurvitz, A. et al. (1997) J. Biol. Chem. 272:22140-22147). All 2,4-dienoyl-CoA reductases have a fairly well conserved NADPH binding site motif (Koivuranta et al., supra).

The main pathway beta-oxidation enzyme enoyl-CoA hydratase catalyzes the reaction:
2-trans-enoyl-CoA+H₂O<--->3-hydroxyacyl-CoA

This reaction hydrates the double bond between C-2 and C-3 of 2-trans-enoyl-CoA, which is generated from saturated and unsaturated fatty acids (Engel, C. K. et al. (1996) EMBO J. 15:5135-5145). This step is downstream from the step catalyzed by 2,4-dienoyl-reductase. Different enoyl-CoA hydratases act on short-, medium-, and long-chain fatty acids (Eaton et al., supra). Mitochondrial and peroxisomal enoyl-CoA hydratases occur as both mono-functional enzymes and as part of multi-functional enzyme complexes. Human liver mitochondrial short-chain enoyl-CoA hydratase is synthesized as a 290 amino acid precursor with a 29 amino acid N-terminal leader peptide (Kanazawa, M. et al. (1993) Enzyme Protein 47:9-13; and Janssen, U. et al. (1997) Genomics 40:470-475). Rat short-chain enoyl-CoA hydratase is 87% identical to the human sequence in the mature region of the protein and functions as a homohexamer (Kanazawa et al., supra; and Engel et al., supra). A mitochondrial trifunctional protein exists that has long-chain enoyl-CoA hydratase, 3-hydroxyacyl-CoA dehydrogenase, and long-chain 3-oxothiolase activities (Eaton et al., supra). In human peroxisomes, enoyl-CoA hydratase activity is found in both a 327 amino acid residue mono-functional enzyme and as part of a multi-functional enzyme, also known as bifunctional enzyme, which possesses enoyl-CoA hydratase, enoyl-CoA isomerase, and 3-hydroxyacyl-CoA hydrogenase activities (FitzPatrick, D. R. et al. (1995) Genomics 27:457-466; and Hoefler, G. et al. (1994) Genomics 19:60-67). A 339 amino acid residue human protein with short-chain enoyl-CoA hydratase activity also acts as an AU-specific RNA binding protein (Nakagawa, J. et al. (1995) Proc. Natl. Acad. Sci. USA 92:2051-2055). All enoyl-CoA hydratases share homology near two active site glutamic acid residues, with 17 amino acid residues that are highly conserved (Wu, W.-J. et al. (1997) Biochemistry 36:2211-2220).

Inherited deficiencies in mitochondrial and peroxisomal beta-oxidation enzymes are associated with severe diseases, some of which manifest soon after birth and lead to death within a few years. Mitochondrial beta-oxidation associated deficiencies include, e.g., carnitine palmitoyl transferase and carnitine deficiency, very-long-chain acyl-CoA dehydrogenase deficiency, medium-chain acyl-CoA dehydrogenase deficiency, short-chain acyl-CoA dehydrogenase deficiency, electron transport flavoprotein and electron transport flavoprotein:ubiquinone oxidoreductase deficiency, trifunctional protein deficiency, and short-chain 3-hydroxyacyl-CoA dehydrogenase deficiency (Eaton et al., supra). Mitochondrial trifunctional protein (including enoyl-CoA hydratase) deficient patients have reduced long-chain enoyl-CoA hydratase activities and suffer from non-ketotic hypoglycemia, sudden infant death syndrome, cardiomyopathy, hepatic dysfunction, and muscle weakness, and may die at an early age (Eaton et al., supra).

Defects in mitochondrial beta-oxidation are associated with Reye's syndrome, a disease characterized by hepatic dysfunction and encephalopathy that sometimes follows viral infection in children. Reye's syndrome patients may have elevated serum levels of free fatty acids (Cotran, R. S. et al. (1994) Robbins Pathologic Basis of Disease, W. B. Saunders Co., Philadelphia Pa., p. 866). Patients with mitochondrial short-chain 3-hydroxyacyl-CoA dehydrogenase deficiency and medium-chain 3-hydroxyacyl-CoA dehydrogenase deficiency also exhibit Reye-like illnesses (Eaton et al., supra; and Egidio, R. J. et al. (1989) Am. Fam. Physician 39:221-226).

Inherited conditions associated with peroxisomal beta-oxidation include Zellweger syndrome, neonatal adrenoleukodystrophy, infantile Refsum's disease, acyl-CoA oxidase deficiency, peroxisomal thiolase deficiency, and bifunctional protein deficiency (Suzuki, Y. et al. (1994) Am. J. Hum. Genet. 54:36-43; Hoefler et al., supra). Patients with peroxisomal bifunctional enzyme deficiency, including that of enoyl-CoA hydratase, suffer from hypotonia, seizures, psychomotor defects, and defective neuronal migration; accumulate very-long-chain fatty acids; and typically die within a few years of birth (Watkins, P. A. et al. (1989) J. Clin. Invest. 83:771-777).

Peroxisomal beta-oxidation is impaired in cancerous tissue. Although neoplastic human breast epithelial cells have the same number of peroxisomes as do normal cells, fatty acyl-CoA oxidase activity is lower than in control tissue (el Bouhtoury, F. et al. (1992) J. Pathol. 166:27-35). Human colon carcinomas have fewer peroxisomes than normal colon tissue and have lower fatty-acyl-CoA oxidase and bifunctional enzyme (including enoyl-CoA hydratase) activities than normal tissue (Cable, S. et al. (1992) Virchows Arch. B Cell Pathol. Incl. Mol. Pathol. 62:221-226).

6-phosphogluconate dehydrogenase (6-PGDH) catalyses the NADP⁺-dependent oxidative decarboxylation of 6-phosphogluconate to ribulose 5-phosphate with the production of NADPH. The absence or inhibition of 6-PGDH results in the accumulation of 6-phosphogluconate to toxic levels in eukaryotic cells. 6-PGDH is the third enzyme of the pentose phosphate pathway (PPP) and is ubiquitous in nature. In some heterofermentatative species, NAD+ is used as a cofactor with the subsequent production of NADH.

The reaction proceeds through a 3-keto intermediate which is decarboxylated to give the enol of ribulose 5-phosphate, then converted to the keto product following tautomerization of the enol (Berdis A. J. and P. F. Cook (1993) Biochemistry 32:2041-2046). 6-PGDH activity is regulated by the inhibitory effect of NADPH, and the activating effect of 6-phosphogluconate (Rippa, M. et al. (1998) Biochim. Biophys. Acta 1429:83-92). Deficiencies in 6-PGDH activity have been linked to chronic hemolytic anemia.

The targeting of specific forms of 6-PGDH (e.g., enzymes found in trypanosomes) has been suggested as a means for controlling parasitic infections (Tetaud, E. et al. (1999) Biochem. J. 338:55-60). For example, the Trypanosoma brucei enzyme is markedly more sensitive to inhibition by the substrate analogue 6-phospho-2-deoxygluconate and the coenzyme analogue adenosine 2′,5′-bisphosphate, compared to the mammalian enzyme (Hanau, S. et al. (1996) Eur. J. Biochem. 240:592-599).

Ribonucleotide diphosphate reductase catalyzes the reduction of ribonucleotide diphosphates (i.e., ADP, GDP, CDP, and UDP) to their corresponding deoxyribonucleotide diphosphates (i.e., dADP, dGDP, dCDP, and dUDP) which are used for the synthesis of DNA. Ribonucleotide diphosphate reductase thereby performs a crucial role in the de novo synthesis of deoxynucleotide precursors. Deoxynucleotides are also produced from deoxynucleosides by nucleoside kinases via the salvage pathway.

Mammalian ribonucleotide diphosphate reductase comprises two components, an effector-binding component (E) and a non-heme iron component (F). Component E binds the nucleoside triphosphate effectors while component F contains the iron radical necessary for catalysis. Molecular weight determinations of the E and F components, as well as the holoenzyme, vary according to the methods used in purification of the proteins and the particular laboratory. Component E is approximately 90-100 kDa, component F is approximately 100-120 kDa, and the holoenzyme is 200-250 kDa.

Ribonucleotide diphosphate reductase activity is adversely effected by iron chelators, such as thiosemicarbazones, as well as EDTA. Deoxyribonucleotide diphosphates also appear to be negative allosteric effectors of ribonucleotide diphosphate reductase. Nucleotide triphosphates (both ribo- and deoxyribo-) appear to stimulate the activity of the enzyme. 3-methyl-4-nitrophenyl, a metabolite of widely used organophosphate pesticides, is a potent inhibitor of ribonucleotide diphosphate reductase in mammalian cells. Some evidence suggests that ribonucleotide diphosphate reductase activity in DNA virus (e.g., herpes virus)-infected cells and in cancer cells is less sensitive to regulation by allosteric regulators and a correlation exists between high ribonucleotide diphosphate reductase activity levels and high rates of cell proliferation (e.g., in hepatomas). This observation suggests that virus-encoded ribonucleotide diphosphate reductases, and those present in cancer cells, are capable of maintaining an increased supply deoxyribonucleotide pool for the production of virus genomes or for the increased DNA synthesis which characterizes cancers cells. Ribonucleotide diphosphate reductase is thus a target for therapeutic intervention (Nutter, L. M. and Y.-C. Cheng (1984) Pharmac. Ther. 26:191-207; and Wright, J. A. (1983) Pharmac. Ther. 22:81-102).

Dihydrodiol dehydrogenases (DD) are monomeric, NAD(P)⁺-dependent, 34-37 kDa enzymes responsible for the detoxification of trans-dihydrodiol and anti-diol epoxide metabolites of polycyclic aromatic hydrocarbons (PAH) such as benzo[α]yrene, benz[α]anthracene, 7-methyl-benz[α]anthracene, 7,12-dimethyl-benz[α]anthracene, chrysene, and 5-methyl-chrysene. In mammalian cells, an environmental PAH toxin such as benzo[α]yrene is initially epoxidated by a microsomal cytochrome P450 to yield 7R,8R-arene-oxide and subsequently (−)-7R,8R-dihydrodiol ((−)-trans-7,8-dihydroxy-7,8-dihydrobenzo[α]pyrene or (−)-trans-B[α]P-diol) This latter compound is further transformed to the anti-diol epoxide of benzo[α]pyrene (i.e., (±)-anti-7β,8α-dihydroxy-9α,10α-epoxy-7,8,9,10-tetrahydrobenzo[α]pyrene), by the same enzyme or a different enzyme, depending on the species. This resulting anti-diol epoxide of benzo[α]yrene, or the corresponding derivative from another PAH compound, is highly mutagenic.

DD efficiently oxidizes the precursor of the anti-diol epoxide (i.e., trans-dihydrodiol) to transient catechols which auto-oxidize to quinones, also producing hydrogen peroxide and semiquinone radicals. This reaction prevents the formation of the highly carcinogenic anti-diol. Anti-diols are not themselves substrates for DD yet the addition of DD to a sample comprising an anti-diol compound results in a significant decrease in the induced mutation rate observed in the Ames test. In this instance, DD is able to bind to and sequester the anti-diol, even though it is not oxidized. Whether through oxidation or sequestration, DD plays an important role in the detoxification of metabolites of xenobiotic polycyclic compounds (Penning, T. M. (1993) Chemico-Biological Interactions 89:1-34).

15-oxoprostaglandin 13-reductase (PGR) and 15-hydroxyprostaglandin dehydrogenase (15-PGDH) are enzymes present in the lung that are responsible for degrading circulating prostaglandins. Oxidative catabolism via passage through the pulmonary system is a common means of reducing the concentration of circulating prostaglandins. 15-PGDH oxidizes the 15-hydroxyl group of a variety of prostaglandins to produce the corresponding 15-oxo compounds. The 15-oxo derivatives usually have reduced biological activity compared to the 15-hydroxyl molecule. PGR further reduces the 13,14 double bond of the 15-oxo compound which typically leads to a further decrease in biological activity. PGR is a monomer with a molecular weight of approximately 36 kDa. The enzyme requires NADH or NADPH as a cofactor with a preference for NADH. The 15-oxo derivatives of prostaglandins PGE₁, PGE₂, and PGE_{2α} are all substrates for PGR; however, the non-derivatized prostaglandins (i.e., PGE₁, PGE₂, and PGE_{2α}) are not substrates (Ensor, C. M. et al. (1998) Biochem. J. 330:103-108).

15-PGDH and PGR also catalyze the metabolism of lipoxin A₄ (LXA₄). Lipoxins (LX) are autacoids, lipids produced at the sites of localized inflammation, which down-regulate polymorphonuclear leukocyte (PMN) function and promote resolution of localized trauma. Lipoxin production is stimulated by the administration of aspirin in that cells displaying cyclooxygenase II (COX II) that has been acetylated by aspirin and cells that possess 5-lipoxygenase (5-LO) interact and produce lipoxin. 15-PGDH generates 15-oxo-LXA₄ with PGR further converting the 15-oxo compound to 13,14-dihydro-15-oxo-LXA₄ (Clish, C. B. et al. (2000) J. Biol. Chem. 275:25372-25380). This finding suggests a broad substrate specificity of the prostaglandin dehydrogenases and has implications for these enzymes in drug metabolism and as targets for therapeutic intervention to regulate inflammation.

The GMC (glucose-methanol-choline) oxidoreductase family of enzymes was defined based on sequence alignments of Drosophila melanogaster glucose dehydrogenase, Escherichia coli choline dehydrogenase, Aspergillus niger glucose oxidase, and Hansenula polymorpha methanol oxidase. Despite their different sources and substrate specificities, these four flavoproteins are homologous, being characterized by the presence of several distinctive sequence and structural features. Each molecule contains a canonical ADP-binding, beta-alpha-beta mononucleotide-binding motif close to the amino terminus. This fold comprises a four-stranded parallel beta-sheet sandwiched between a three-stranded antiparallel beta-sheet and alpha-helices. Nucleotides bind in similar positions relative to this chain fold (Cavener, D. R. (1992) J. Mol. Biol. 223:811-814; Wierenga, R. K. et al. (1986) J. Mol. Biol. 187:101-107). Members of the GMC oxidoreductase family also share a consensus sequence near the central region of the polypeptide. Additional members of the GMC oxidoreductase family include cholesterol oxidases from Brevibacterium sterolicum and Streptomyces; and an alcohol dehydrogenase from Pseudomonas oleovorans (Cavener, supra; Henikoff, S. and J. G. Henikoff (1994) Genonics 19:97-107; van Beilen, J. B. et al. (1992) Mol. Microbiol. 6:3121-3136).

IMP dehydrogenase and GMP reductase are two oxidoreductases which share many regions of sequence similarity. IMP dehydrogenase (EC 1.1.1.205) catalyes the NAD-dependent reduction of IMP (inosine monophosphate) into XMP (xanthine monophosphate) as part of de novo GTP biosynthesis (Collart, F. R. and E. Huberman (1988) J. Biol. Chem. 263:15769-15772). GMP reductase catalyzes the NADPH-dependent reductive deamination of GMP into IMP, helping to maintain the intracellular balance of adenine and guanine nucleotides (Andrews, S. C. and J. R. Guest (1988) Biochem. J. 255:35-43).

Pyridine nucleotide-disulphide oxidoreductases are FAD flavoproteins involved in the transfer of reducing equivalents from FAD to a substrate. These flavoproteins contain a pair of redox-active cysteines contained within a consensus sequence which is characteristic of this protein family (Kurlyan, J. et al. (1991) Nature 352:172-174). Members of this family of oxidoreductases include glutathione reductase (EC 1.6.4.2); thioredoxin reductase of higher eukaryotes (EC 1.6.4.5); trypanothione reductase (EC 1.6.4.8); lipoamide dehydrogenase (EC 1.8.1.4), the E3 component of alpha-ketoacid dehydrogenase complexes; and mercuric reductase (EC 1.16.1.1).

Transferases

Transferases are enzymes that catalyze the transfer of molecular groups. The reaction may involve an oxidation, reduction, or cleavage of covalent bonds, and is often specific to a substrateor to particular sites on a type of substrate. Transferases participate in reactions essential to such functions as synthesis and degradation of cell components, and regulation of cell functions including cell signaling, cell proliferation, inflammation, apoptosis, secretion and excretion. Transferases are involved in key steps in disease processes involving these functions. Transferases are frequently classified according to the type of group transferred. For example, methyl transferases transfer one-carbon methyl groups, amino transferases transfer nitrogenous amino groups, and similarly denominated enzymes transfer aldehyde or ketone, acyl, glycosyl, alkyl or aryl, isoprenyl, saccharyl, phosphorous-containing, sulfur-containing, or selenium-containing groups, as well as small enzymatic groups such as Coenzyme A.

Acyl transferases include peroxisomal carnitine octanoyl transferase, which is involved in the fatty acid beta-oxidation pathway, and mitochondrial carnitine palmitoyl transferases, involved in fatty acid metabolism and transport. Choline O-acetyl transferase catalyzes the biosynthesis of the neurotransmitter acetylcholine. N-acyltransferase enzymes catalyze the transfer of an amino acid conjugate to an activated carboxylic group. Endogenous compounds and xenobiotics are activated by acyl-CoA synthetases in the cytosol, microsomes, and mitochondria. The acyl-CoA intermediates are then conjugated with an amino acid (typically glycine, glutamine, or taurine, but also ornithine, arginine, histidine, serine, aspartic acid, and several dipeptides) by N-acyltransferases in the cytosol or mitochondria to form a metabolite with an amide bond. One well-characterized enzyme of this class is the bile acid-CoA:amino acid N-acyltransferase (BAT) responsible for generating the bile acid conjugates which serve as detergents in the gastrointestinal tract (Falany, C. N. et al. (1994) J. Biol. Chem. 269:19375-19379; Johnson, M. R. et al. (1991) J. Biol. Chem. 266:10227-10233). BAT is also useful as a predictive indicator for prognosis of hepatocellular carcinoma patients after partial hepatectomy (Furutani, M. et al. (1996) Hepatology 24:1441-1445).

Acetyltransferases

Acetyltransferases have been extensively studied for their role in histone acetylation. Histone acetylation results in the relaxing of the chromatin structure in eukaryotic cells, allowing transcription factors to gain access to promoter elements of the DNA templates in the affected region of the genome (or the genome in general). In contrast, histone deacetylation results in a reduction in transcription by closing the chromatin structure and limiting access of transcription factors. To this end, a common means of stimulating cell transcription is the use of chemical agents that inhibit the deacetylation of histones (e.g., sodium butyrate), resulting in a global (albeit artifactual) increase in gene expression. The modulation of gene expression by acetylation also results from the acetylation of other proteins, including but not limited to, p53, GATA-1, MyoD, ACTR, TFIIE, TFIIF and the high mobility group proteins (HMG). In the case of p53, acetylation results in increased DNA binding, leading to the stimulation of transcription of genes regulated by p53. The prototypic histone acetylase (HAT) is Gen5 from Saccharomyces cerevisiae. Gen5 is a member of a family of acetylases that includes Tetrahymena p55, human Gen5, and human p300/CBP. Histone acetylation is reviewed in (Cheung, W. L. et al. (2000) Curr. Opin. Cell Biol. 12:326-333 and Berger, S. L (1999) Curr. Opin. Cell Biol. 11:336-341). Some acetyltransferase enzymes possess the alpha/beta hydrolase fold (Center of Applied Molecular Engineering Inst. of Chemistry and Biochemistry—University of Salzburg, http://predict.sanger.ac.uk/irbm-course97/Docs/ms/) common to several other major classes of enzymes, including but not limited to, acetylcholinesterases and carboxylesterases (Structural Classification of Proteins, http://scop.mnrc-lmb.cam.ac.uk/scop/index.html).

N-acetyltransferases are cytosolic enzymes which utilize the cofactor acetyl-coenzyme A (acetyl-CoA) to transfer the acetyl group to aromatic amines and hydrazine containing compounds. In humans, there are two highly similar N-acetyltransferase enzymes, NAT1 and NAT2; mice appear to have a third form of the enzyme, NAT3. The human forms of N-acetyltransferase have independent regulation (NAT1 is widely-expressed, whereas NAT2 is in liver and gut only) and overlapping substrate preferences. Both enzymes appear to accept most substrates to some extent, but NAT1 does prefer some substrates (para-aminobenzoic acid, para-aminosalicylic acid, sulfamethoxazole, and sulfanilamide), while NAT2 prefers others (isoniazid, hydralazine, procainamide, dapsone, aminoglutethimide, and sulfamethazine). A recently isolated human gene, tubedown-1, is homologous to the yeast NAT-1 N-acetyltransferases and encodes a protein associated with acetyltransferase activity. The expression patterns of tubedown-1 suggest that it may be involved in regulating vascular and hematopoietic development (Gendron, R. L. et al. (2000) Dev. Dyn. 218:300-315).

Amino transferases comprise a family of pyridoxal 5′-phosphate (PLP)-dependent enzymes that catalyze transformations of amino acids. Amino transferases play key roles in protein synthesis and degradation, and they contribute to other processes as well. For example, GABA aminotransferase (GABA-T) catalyzes the degradation of GABA, the major inhibitory amino acid neurotransmitter. The activity of GABA-T is correlated to neuropsychiatric disorders such as alcoholism, epilepsy, and Alzheimer's disease (Sherif, F. M. and S. S. Ahmed (1995) Clin. Biochem. 28:145-154). Other members of the family include pyruvate aminotransferase, branched-chain amino acid aminotransferase, tyrosine aminotransferase, aromatic aminotransferase, alanine:glyoxylate aminotransferase (AGT), and kynurenine aminotransferase (Vacca, R. A. et al. (1997) J. Biol. Chem. 272:21932-21937). Kynurenine aminotransferase catalyzes the irreversible transamination of the L-tryptophan metabolite L-kynurenine to form kynurenic acid. The enzyme may also catalyzes the reversible transamination reaction between L-2-aminoadipate and 2-oxoglutarate to produce 2-oxoadipate and L-glutamate. Kynurenic acid is a putative modulator of glutamatergic neurotransmission, thus a deficiency in kynurenine aminotransferase may be associated with pleiotropic effects (Buchli, R. et al. (1995) J. Biol. Chem. 270:29330-29335).

Glycosyl transferases include the mammalian UDP-glucouronosyl transferases, a family of membrane-bound microsomal enzymes catalyzing the transfer of glucouronic acid to lipophilic substrates in reactions that play important roles in detoxification and excretion of drugs, carcinogens, and other foreign substances. Another mammalian glycosyl transferase, mammalian UDP-galactose-ceramide galactosyl transferase, catalyzes the transfer of galactose to ceramide in the synthesis of galactocerebrosides in myelin membranes of the nervous system. The UDP-glycosyl transferases share a conserved signature domain of about 50 amino acid residues (PROSITE: PDOC00359, http://expasy.hcuge.ch/sprot/prosite.html).

Methyl transferases are involved in a variety of pharmacologically important processes. Nicotinamide N-methyl transferase catalyzes the N-methylation of nicotinamides and other pyridines, an important step in the cellular handling of drugs and other foreign compounds. Phenylethanolamine N-methyl transferase catalyzes the conversion of noradrenalin to adrenalin. 6-O-methylguanine-DNA methyl transferase reverses DNA methylation, an important step in carcinogenesis. Uroporphyrin-III C-methyl transferase, which catalyzes the transfer of two methyl groups from S-adenosyl-L-methionine to uroporphyrinogen III, is the first specific enzyme in the biosynthesis of cobalamin, a dietary enzyme whose uptake is deficient in pernicious anemia. Protein-arginine methyl transferases catalyze the posttranslational methylation of arginine residues in proteins, resulting in the mono- and dimethylation of arginine on the guanidino group. Substrates include histones, myelin basic protein, and heterogeneous nuclear ribonucleoproteins involved in mRNA processing, splicing, and transport. Protein-arginine methyl transferase interacts with proteins upregulated by mitogens, with proteins involved in chronic lymphocytic leukemia, and with interferon, suggesting an important role for methylation in cytokine receptor signaling (Lin, W.-J. et al. (1996) J. Biol. Chem. 271:15034-15044; Abramovich, C. et al. (1997) EMBO J. 16:260-266; and Scott, H. S. et al. (1998) Genomics 48:330-340).

Phospho transferases catalyze the transfer of high-energy phosphate groups and are important in energy-requiring and -releasing reactions. The metabolic enzyme creatine kinase catalyzes the reversible phosphate transfer between creatine/creatine phosphate and ATP/ADP. Glycocyamine kinase catalyzes phosphate transfer from ATP to guanidoacetate, and arginine kinase catalyzes phosphate transfer from ATP to arginine. A cysteine-containing active site is conserved in this family (PROSITE: PDOC00103).

Prenyl transferases are heterodimers, consisting of an alpha and a beta subunit, that catalyze the transfer of an isoprenyl group. The Ras farnesyltransferase (FTase) enzyme transfers a farnesyl moiety from cytosolic farnesylpyrophosphate to a cysteine residue at the carboxyl terminus of the Ras oncogene protein. This modification is required to anchor Ras to the cell membrane so that it can perform its role in signal transduction. FTase inhibitors block Ras function and demonstrate antitumor activity_(Buolamwini, J. K. (1999) Curr. Opin. Chem. Biol. 3:500-509). Ftase, which shares structural similarity with geranylgeranyl transferase, or Rab GG transferase, prenylates Rab proteins, allowing them to perform their roles in regulating vesicle transport (Seabra, M. C. (1996) J. Biol. Chem. 271:14398-14404).

Saccharyl transferases are glycating enzymes involved in a variety of metabolic processes. Oligosaccharyl transferase-48, for example, is a receptor for advanced glycation endproducts, which accumulate in vascular complications of diabetes, macrovascular disease, renal insufficiency, and Alzheimer's disease (Thornalley, P. J. (1998) Cell Mol. Biol. (Noisy-Le-Grand) 44:1013-1023).

Coenzyme A (CoA) transferase catalyzes the transfer of CoA between two carboxylic acids. Succinyl CoA:3-oxoacid CoA transferase, for example, transfers CoA from succinyl-CoA to a recipient such as acetoacetate. Acetoacetate is essential to the metabolism of ketone bodies, which accumulate in tissues affected by metabolic disorders such as diabetes (PROSITE: PDOC00980).

Transglutaminase transferases (Tgases) are Ca²⁺ dependent enzymes capable of forming isopeptide bonds by catalyzing the transfer of the γ-carboxy group from protein-bound glutamine to the ε-amino group of protein-bound lysine residues or other primary amines. Tgases are the enzymes responsible for the cross-linking of cornified envelope (CE), the highly insoluble protein structure on the surface of corneocytes, into a chemically and mechanically resistant protein polymer. Seven known human Tgases have been identified. Individual transglutaminase gene products are specialized in the cross-linking of specific proteins or tissue structures, such as factor XIIIa which stabilizes the fibrin clot in hemostasis, prostrate transglutaminase which functions in semen coagulation, and tissue transglutaminase which is involved in GTP-binding in receptor signaling. Four (Tgases 1, 2, 3, and X) are expressed in terminally differentiating epithelia such as the epidermis. Tgases are critical for the proper cross-linking of the CE as seen in the pathology of patients suffering from one form of the skin diseases referred to as congenital ichthyosis which has been linked to mutations in the keratinocyte transglutaminase (TG_{K}) gene (Nemes, Z. et al. (1999) Proc. Natl. Acad. Sci. U.S.A. 96:8402-8407, Aeschlimann, D. et al. (1998) J. Biol. Chem. 273:3452-3460.)

Hydrolases

Hydrolases are a class of enzymes that catalyze the cleavage of various covalent bonds in a substrate by the introduction of a molecule of water. The reaction involves a nucleophilic attack by the water molecule's oxygen atom on a target bond in the substrate. The water molecule is split across the target bond, breaking the bond and generating two product molecules. Hydrolases participate in reactions essential to such functions as synthesis and degradation of cell components, and for regulation of cell functions including cell signaling, cell proliferation, inflamation, apoptosis, secretion and excretion. Hydrolases are involved in key steps in disease processes involving these functions. Hydrolytic enzymes, or hydrolases, may be grouped by substrate specificity into classes including phosphatases, peptidases, lysophospholipases, phosphodiesterases, glycosidases, glyoxalases, aminohydrolases, carboxylesterases, sulfatases, phosphohydrolases, nucleotidases, lysozymes, and many others.

Phosphatases hydrolytically remove phosphate groups from proteins, an energy-providing step that regulates many cellular processes, including intracellular signaling pathways that in turn control cell growth and differentiation, cell-cell contact, the cell cycle, and oncogenesis.

Peptidases, also called proteases, cleave peptide bonds that form the backbone of peptide or protein chains. Proteolytic processing is essential to cell growth, differentiation, remodeling, and homeostasis as well as inflammation and the immune response. Since typical protein half-lives range from hours to a few days, peptidases are continually cleaving precursor proteins to their active form, removing signal sequences from targeted proteins, and degrading aged or defective proteins. Peptidases function in bacterial, parasitic, and viral invasion and replication within a host. Examples of peptidases include trypsin and chymotrypsin (components of the complement cascade and the blood-clotting cascade) lysosomal cathepsins, calpains, pepsin, renin, and chymosin (Beynon, R. J. and J. S. Bond (1994) Proteolytic Enzymes: A Practical Approach, Oxford University Press, New York, N.Y., pp. 1-5).

Lysophospholipases (LPLs) regulate intracellular lipids by catalyzing the hydrolysis of ester bonds to remove an acyl group, a key step in lipid degradation. Small LPL isoforms, approximately 15-30 kD, function as hydrolases; larger isoforms function both as hydrolases and transacylases. A particular substrate for LPLs, lysophosphatidylcholine, causes lysis of cell membranes. LPL activity is regulated by signaling molecules important in numerous pathways, including the inflammatory response.

The phosphodiesterases catalyze the hydrolysis of one of the two ester bonds in a phosphodiester compound. Phosphodiesterases are therefore crucial to a variety of cellular processes. Phosphodiesterases include DNA and RNA endo- and exo-nucleases, which are essential to cell growth and replication as well as protein synthesis. Endonuclease V (deoxyinosine 3′-endonuclease) is an example of a type II site-specific deoxyribonuclease, a putative DNA repair enzyme that cleaves DNAs containing hypoxanthine, uracil, or mismatched bases. Escherichia coli endonuclease V has been shown to cleave DNA containing deoxyxanthosine at the second phosphodiester bond 3′ to deoxyxanthosine, generating a 3′-hydroxyl and a 5′-phosphoryl group at the nick site (He, B. et al. (2000) Mutat. Res. 459:109-114). It has been suggested that Escherichia coli endonuclease V plays a role in the removal of deaminated guanine, i.e., xanthine, from DNA, thus helping to protect the cell against the mutagenic effects of nitrosative deamination (Schouten, K. A. and B. Weiss (1999) Mutat. Res. 435:245-254). In eukaryotes, the process of tRNA splicing requires the removal of small tRNA introns that interrupt the anticodon loop 1 base 3′ to the anticodon. This process requires the stepwise action of an endonuclease, a ligase, and a phosphotransferase (Hong, L. et al. (1998) Science 280:279-284). Ribonuclease P (RNase P) is a ubiquitous RNA processing endonuclease that is required for generating the mature tRNA 5′-end during the tRNA splicing process. This is accomplished through the catalysis of the cleavage of P-3′O bonds to produce 5′-phosphate and 3′-hydroxyl end groups at a specific site on pre-tRNA. Catalysis by RNase P is absolutely dependent on divalent cations such as Mg²⁺ or Mn²⁺ (Kurz, J. C. et al. (2000) Curr. Opin. Chem. Biol. 4:553-558). Substrate recognition mechanisms of RNase P are well conserved among eukaryotes and bacteria (Fabbri, S. et al. (1998) Science 280:284-286). In Saccharomyces cerevisiae, POP1 (‘processing of precursor RNAs’) encodes a protein component of both RNase P and RNase MRP, another RNA processing protein. Mutations in yeast POP1 are lethal (Lygerou, Z. et al. (1994) Genes Dev. 8:1423-1433). Another phosphodiesterase, acid sphingomyelinase, hydrolyzes the membrane phospholipid sphingomyelin to ceramide and phosphorylcholine. Phosphorylcholine functions in synthesis of phosphatidylcholine, which is involved in intracellular signaling pathways. Ceramide is an essential precursor for the generation of gangliosides, membrane lipids found in high concentration in neural tissue. Defective acid sphingomyelinase phosphodiesterase leads to Niemann-Pick disease.

Glycosidases catalyze the cleavage of hemiacetyl bonds of glycosides, which are compounds that contain one or more sugar. Mammalian lactase-phlorizin hydrolase, for example, is an intestinal enzyme that splits lactose. Mammalian beta-galactosidase removes the terminal galactose from gangliosides, glycoproteins, and glycosaminoglycans, and deficiency of this enzyme is associated with a gangliosidosis known as Morquio disease type B (PROSITE PCDOC00910). Vertebrate lysosomal alpha-glucosidase, which hydrolyzes glycogen, maltose, and isomaltose, and vertebrate intestinal sucrase-isomaltase, which hydrolyzes sucrose, maltose, and isomaltose, are widely distributed members of this family with highly conserved sequences at their active sites.

The glyoxylase system is involved in gluconeogenesis, the production of glucose from storage compounds in the body. It consists of glyoxylase I, which catalyzes the formation of S-D-lactoylglutathione from methyglyoxal, a side product of triose-phosphate energy metabolism, and glyoxylase II, which hydrolyzes S-D-lactoylglutathione to D-lactic acid and reduced glutathione. Glyoxylases are involved in hyperglycemia, non-insulin-dependent diabetes mellitus, the detoxification of bacterial toxins, and in the control of cell proliferation and microtubule assembly.

NG,NG-dimethylarginine dimethylaminohydrolase (DDAH) is an enzyme that hydrolyzes the endogenous nitric oxide synthase (NOS) inhibitors, NG-monomethyl-arginine and NG,NG-dimethyl-L-arginine, to L-citrulline. Inhibiting DDAH can cause increased intracellular concentration of NOS inhibitors to levels sufficient to inhibit NOS. Therefore, DDAH inhibition may provide a method of NOS inhibition, and changes in the activity of DDAH could play a role in pathophysiological alterations in nitric oxide generation (MacAllister, R. J. et al. (1996) Br. J. Pharmacol. 119:1533-1540). DDAH was found in neurons displaying cytoskeletal abnormalities and oxidative stress in Alzheimer's disease. In age-matched control cases, DDAH was not found in neurons. This suggests that oxidative stress- and nitric oxide-mediated events play a role in the pathogenesis of Alzheimer's disease (Smith, M. A. et al. (1998) Free Rad. Biol. Med. 25:898-902).

Acyl-CoA thioesterase is another member of the carboxylesterase family (Alexson, S. E. et al. (1993) Eur. J. Biochem. 214:719-727). Evidence suggests that acyl-CoA thioesterase has a regulatory role in steroidogenic tissues (Finkielstein, C. et al. (1998) Eur. J. Biochem. 256:60-66).

The alpha/beta hydrolase protein fold is common to several hydrolases of diverse phylogenetic origin and catalytic function. Enzymes with the alpha/beta hydrolase fold have a common core structure consisting of eight beta-sheets connected by alpha-helices. The most conserved structural feature of this fold is the loops of the nucleophile-histidine-acid catalytic triad. The histidine in the catalytic triad is completely conserved, while the nucleophile and acid loops accommodate more than one type of amino acid (Ollis, D. L. et al. (1992) Protein Eng. 5:197-211).

Sulfatases are members of a highly conserved gene family that share extensive sequence homology and a high degree of structural similarity. Sulfatases catalyze the cleavage of sulfate esters. To perform this function, sulfatases undergo a unique post-translational modification in the endoplasmic reticulum that involves the oxidation of a conserved cysteine residue. A human disorder called multiple sulfatase deficiency is due to a defect in this post-translational modification step, leading to inactive sulfatases (Recksiek, M. et al. (1998) J. Biol. Chem. 273:6096-6103).

Phosphohydrolases are enzymes that hydrolyze phosphate esters. Some phosphohydrolases contain a mutT domain signature sequence. MutT is a protein involved in the GO system responsible for removing an oxidatively damaged form of guanine from DNA. A region of about 40 amino acid residues, found in the N-terminus of mutT, is also found in other proteins, including some phosphohydrolases (PROSITE PDOC00695).

Serine hydrolases are a large functional class of hydrolytic enzymes that contain a serine residue in their active site. This class of enzymes contains proteinases, esterases, and lipases which hydrolyze a variety of substrates and, therefore, have different biological roles. Proteins in this superfamily can be further grouped into subfamilies based on substrate specificity or amino acid similarities (Puente, X. S. and C. Lopez-Otin (1995) J. Biol. Chem. 270:12926-12932).

Neuropathy target esterase (NTE) is an integral membrane protein present in all neurons and in some non-neural-cell types of vertebrates. NTE is involved in a cell-signaling pathway controlling interactions between neurons and accessory glial cells in the developing nervous system. NTE has serine esterase activity and efficiently catalyses the hydrolysis of phenyl valerate (PV) in vitro, but its physiological substrate is unknown. NTE is not related to either the major serine esterase family, which includes acetylcholinesterase, nor to any other known serine hydrolases. NTE contains at least which contains the esterase activity and is, in part, conserved in proteins found in bacteria, yeast, nematodes and insects. NTE's effector domain contains three predicted transmembrane segments, and the active-site serine residue lies at the center of one of these segments. The isolated recombinant domain shows PV hydrolase activity only when incorporated into phospholipid liposomes. NTE's esterase activity is largely redundant in adult vertebrates, but organophosphates which react with NTE in vivo initiate unknown events which lead to a neuropathy with degeneration of long axons. These neuropathic organophosphates leave a negatively charged group covalently attached to the active-site serine residue, which causes a toxic gain of function in NTE (Glynn, P. (1999) Biochem. J. 344:625-631). Further, the Drosophila neurodegeneration gene swiss-cheese encodes a neuronal protein involved in glia-neuron interaction and is homologous to the above human NTE (Moser, M. et al. (2000) Mech. Dev. 90:279-282).

Chitinases are chitin-degrading enzymes present in a variety of organisms and participate in processes including cell wall remodeling, defense and catabolism. Chitinase activity has been found in human serum, leukocytes, granulocytes, and in association with fertilized oocytes in mammals (Escott, G. M. (1995) Infect. Immunol. 63:4770-4773; DeSouza, M. M. (1995) Endrocrinology 136:2485-2496). Glycolytic and proteolytic molecules in humans are associated with tissue damage in lung diseases and with increased tumorigenicity and metastatic potential of cancers (Mulligan, M. S. (1993) Proc. Natl. Acad. Sci. 90:11523-11527; Matrisian, L. M. (1991) Am. J. Med. Sci. 302:157-162; Witty, J. P. (1994) Cancer Res. 54:4805-4812). The discovery of a human enzyme with chitinolytic activity is noteworthy given the lack of endogenous chitin in the human body (Raghavan, N. (1994) Infect. Immun. 62:1901-1908). However, there is a group of mammalian proteins that share homology with chitinases from various non-mammalian organisms, such as bacteria, fungi, plants, and insects. The members of this family differ in their ability to hydrolyze chitin or chitin-like substrates. Some of the mammalian members of the family, such as a bovine whey chitotriosidase and human cartilage proteins which do not demonstrate specific chitinolytic activity, are expressed in association with tissue remodeling events (Rejman, J. J. (1988) Biochem. Biophys. Res. Commun. 150:329-334, Nyirkos, P. (1990) Biochem. J. 268:265-268). Elevated levels of human cartilage proteins have been reported in the synovial fluid and cartilage of patients with rheumatoid arthritis, a disease which produces a severe degradation of the cartilage and a proliferation of the synovial membrane in the affected joints (Hakala, B. E. (1993) J. Biol. Chem. 268:25803-25810).

A small subclass of hydrolases acting on ether bonds includes the thioether hydrolases. S-adenosyl-L-homocysteine hydrolase, also known as AdoHcyase or SAHH (PROSITE PDOC00603; EC 3.3.1.1), is a thioether hydrolase first described in rat liver extracts as the activity responsible for the reversible hydrolysis of S-adenosyl-L-homocysteine (AdoHcy) to adenosine and homocysteine (Sganga, M. W. et al. (1992) PNAS 89:6328-6332). SAHH is a cytosolic enzyme that has been found in all cells that have been tested, with the exception of Escherichia coli and certain related bacteria (Walker, R. D. et al. (1975) Can. J. Biochem. 53:312-319; Shimizu, S. et al. (1988) FEMS Microbiol. Lett. 51:177-180; Shimizu, S. et al. (1984) Eur. J. Biochem. 141:385-392). SAHH activity is dependent on NAD⁺ as a cofactor. Deficiency of SAHH is associated with hypermethioninemia (Online Mendelian Inheritance in Man (OMIM) #180960 Hypermethioninemia), a pathologic condition characterized by neonatal cholestasis, failure to thrive, mental and motor retardation, facial dysmorphism with abnormal hair and teeth, and myocaridopathy (Labrune, P. et al. (1990) J. Pediat. 117:220-226).

Another subclass of hydrolases includes those enzymes which act on carbon-nitrogen (C—N) bonds other than peptide bonds. To this subclass belong those enzymes hydrolyzing amides, amidines, and other C—N bonds. This subclass is further subdivided on the basis of substrate specificity such as linear amides, cyclic amides, linear amidines, cyclic amidines, nitrites and other compounds. A hydrolase belonging to the sub-subclass of enzymes acting on the cyclic amidines is adenosine deaminase (ADA). ADA catalyzes the breakdown of adenosine to inosine. ADA is present in many mammalian tissues, including placenta, muscle, lung, stomach, digestive diverticulum, spleen, erythrocytes, thymus, seminal plasma, thyroid, T-cells, bone marrow stem cells, and liver. A subclass of ADAs, ADAR, act on RNA and are classified as RNA editases. An ADAR from Drosophila, dADAR, expressed in the developing nervous system, may act on para voltage-gated Na⁺ channel transcripts in the central nervous system (Palladino, M. J. et al. (2000) RNA 6:1004-1018). ADA deficiency causes profound lymphopenia with severe combined immunodeficiency (SCID). Cells from patients with ADA deficiency contain low, sometimes undetectable, amounts of ADA catalytic activity and ADA protein. ADA deficiency stems from genetic mutations in the ADA gene (Hershfield, M. S. (1998) Semin. Hematol. 4:291-298). Metabolic consequences of ADA deficiency are associated with defects in alveogenesis, pulmonary inflammation, and airway obstruction (Blackburn, M. R. et al. (2000) J. Exp. Med. 192:159-170).

Pancreatic ribonucleases (RNase) are pyrimidine-specific endonucleases found in high quantity in the pancreas of certain mammalian taxa and of some reptiles (Beintema, J. J. et al (1988) Prog. Biophys. Mol. Biol. 51:165-192). Proteins in the mammalian pancreatic RNase superfamily are noncytosolic endonucleases that degrade RNA through a two-step transphosphorolytic-hydrolytic reaction (Beintema, J. J. et al. (1986) Mol. Biol. Evol. 3:262-275). Specifically, the enzymes are involved in endonucleolytic cleavage of 3′-phosphomononucleotides and 3′-phosphooligonucleotides ending in C—P or U—P with 2;3′-cyclic phosphate intermediates. Ribonucleases can unwind the DNA helix by complexing with single-stranded DNA; the complex arises by an extended multi-site cation-anion interaction between lysine and arginine residues of the enzyme and phosphate groups of the nucleotides. Some of the enzymes belonging to this family appear to play a purely digestive role, whereas others exhibit potent and unusual biological activities (D'Alessio, G. (1993) Trends Cell Biol. 3:106-109). Proteins belonging to the pancreatic RNase family include: bovine seminal vesicle and brain ribonucleases; kidney non-secretory ribonucleases (Beintema, J. J. et al (1986) FEBS Lett. 194:338-343); liver-type ribonucleases (Rosenberg, H. F. et al. (1989) PNAS U.S.A. 86:4460-4464); angiogenin, which induces vascularisation of normal and malignant tissues; eosinophil cationic protein (Hofsteenge, J. et al. (1989) Biochemistry 28:9806-9813), a cytotoxin and helminthotoxin with ribonuclease activity; and frog liver ribonuclease and frog sialic acid-binding lectin. The sequences of pancreatic RNases contain 4 conserved disulfide bonds and 3 amino acid residues involved in the catalytic activity.

ADP-ribosylation is a reversible post-translational protein modification in which an ADP-ribose moiety is transferred from β-NAD to a target amino acid such as arginine or cysteine. ADP-ribosylarginine hydrolases regenerate arginine by removing ADP-ribose from the protein, completing the ADP-ribosylation cycle (Moss, J. et al. (1997) Adv. Exp. Med. Biol. 419:25-33). ADP-ribosylation is a well-known reaction among bacterial toxins. Cholera toxin, for example, disrupts the adenylyl cyclase system by ADP-ribosylating the α-subunit of the stimulatory G-protein, causing an increase in intracellular cAMP (Moss, J. and M. Vaughan (Eds) (1990) ADP-ribosylating Toxins and G-Proteins: Insights into Signal Transduction, American Society for Microbiology, Washington, D.C.). ADP-ribosylation may also have a regulatory function in eukaryotes, affecting such processes as cytoskeletal assembly (Zhou, H. et al. (1996) Arch. Biochem. Biophys. 334:214-222) and cell proliferation in cytotoxic T-cells (Wang, J. et al. (1996) J. Immunol. 156:2819-2827).

Nucleotidases catalyze the formation of free nucleosides from nucleotides. The cytosolic nucleotidase cN-I (5′ nucleotidase-I) cloned from pigeon heart catalyzes the formation of adenosine from AMP generated during ATP hydrolysis (Sala-Newby, G. B. et al. (1999) J. Biol. Chem. 274:17789-17793). Increased adenosine concentration is thought to be a signal of metabolic stress, and adenosine receptors mediate effects including vasodilation, decreased stimulatory neuron firing and ischemic preconditioning in the heart (Schrader, 3. (1990) Circulation 81:389-391; Rubino, A. et al. (1992) Eur. J. Pharmacol. 220:95-98; de Jong, J. W. et al. (2000) Pharmacol. Ther. 87:141-149). Deficiency of pyrimidine 5′-nucleotidase can result in hereditary hemolytic anemia (OMIM #266120).

The lysozyme c superfamily consists of conventional lysozymes c, calcium-binding lysozymes c, and α-lactalbumin (Prager, E. M. and P. Jolles (1996) EXS 75:9-31). The proteins in this superfamily have 35-40% sequence homology and share a common three-dimensional fold, but can have different functions. Lysozymes c are ubiquitous in a variety of tissues and secretions and can lyse the cell walls of certain bacteria (McKenzie, H. A. (1996) EXS 75:365-409). Alpha-lactalbumin is a metallo-protein that binds calcium and participates in the synthesis of lactose (Iyer, L. K and P. K. Qasba (1999) Protein Eng. 12:129-139). Alpha-lactalbumin occurs in mammalian milk and colostrum (McKenzie, supra).

Lysozymes catalyze the hydrolysis of certain mucopolysaccharides of bacterial cell walls, specifically, the beta (1-4) glycosidic linkages between N-acetylmuramic acid and N-acetylglucosamine, and cause bacterial lysis. Lysozymes occur in diverse organisms including viruses, birds, and mammals. In humans, lysozymes are found in spleen, lung, kidney, white blood cells, plasma, saliva, milk, tears, and cartilage (OMIM #153450 Lysozyme; Weaver, L. H. et al. (1985) J. Mol. Biol. 184:739-741). Lysozyme c functions in ruminants as a digestive enzyme, releasing proteins from ingested bacterial cells, and may perform the same function in human newborns (Braun, O. H. et al. (1995) Kiln. Pediatr. 207:4-7).

The two known forms of lysozymes, chicken-type and goose-type, were originally isolated from chicken and goose egg white, respectively. Chicken-type and goose-type lysozymes have similar three-dimensional structures, but different amino acid sequences (Nakano, T. and T. Graf (1991) Biochim. Biophys. Acta 1090:273-276). In chickens, both forms of lysozyme are found in neutrophil granulocytes (heterophils), but only chicken-type lysozyme is found in egg white. Generally, chicken-type lysozyme mRNA is found in both adherent monocytes and macrophages and nonadherent promyelocytes and granulocytes as well as in cells of the bone marrow, spleen, bursa, and oviduct. Goose-type lysozyme mRNA is found in non-adherent cells of the bone marrow and lung. Several isozymes have been found in rabbits, including leukocytic, gastrointestinal, and possibly lymphoepithelial forms (OMIM #153450, supra; Nakano and Graf, supra; and GenBank GI 1310929). A human lysozyme gene encoding a protein similar to chicken-type lysozyme has been cloned (Yoshimura, K. et al. (1988) Biochem. Biophys. Res. Commun. 150:794-801). A consensus motif featuring regularly spaced cysteine residues has been derived from the lysozyme C enzymes of various species (PROSITE PS00128). Lysozyme C shares about 40% amino acid sequence identity with α-lactalbumin.

Lysozymes have several disease associations. Lysozymuria is observed in diabetic nephropathy (Shima, M. et al. (1986) Clin. Chem. 32:1818-1822), endemic nephropathy (Bruckner, I. et al. (1978) Med. Interne. 16:117-125), urinary tract infections (Heidegger, H. (1990) Minerva Ginecol. 42:243-250), and acute monocytic leukemia (Shaw, M. T. (1978) Am. J. Hematol. 4:97-103). Nakano and Graf (supra) suggested a role for lysozyme in host defense systems. Older rabbits with an inherited lysozyme deficiency show increased susceptibility to infections, such as subcutaneous abscesses (OMIM #153450, supra). Human lysozyme gene mutations cause hereditary systemic amyloidosis, a rare autosomal dominant disease in which amyloid deposits form in the viscera, including the kidney, adrenal glands, spleen, and liver. This disease is usually fatal by the fifth decade. The amyloid deposits contain variant forms of lysozyme. Renal amyloidosis is the most common and potentially the most serious form of organ involvement (Pepys, M. B. et al. (1993) Nature 362:553-557; OMIM #105200 Familial Visceral Amyloidosis; Cotran, R. S. et al. (1994) Robbins Pathologic Basis of Disease, W. B. Saunders Company, Philadelphia Pa., pp. 231-238). Increased levels of lysozyme and lactate have been observed in the cerebrospinal fluid of patients with bacterial meningitis (Ponka, A. et al. (1983) Infection 11:129-131). Acute monocytic leukemia is characterized by massive lysozymuria (Den Tandt, W. R. (1988) Int. J. Biochem. 20:713-719).

Lyases

Lyases are a class of enzymes that catalyze the cleavage of C—C, C—O, C—N, C—S, C—(halide), P—O, or other bonds without hydrolysis or oxidation to form two molecules, at least one of which contains a double bond (Stryer, L. (1995) Biochemistry, W.H. Freeman and Co., New York N.Y., p. 620). Under the International Classification of Enzymes (Webb, E. C. (1992) Enzyme Nomenclature 1992: Recommendations of the Nomenclature Committee of the International Union of Biochemistry and Molecular Biology on the Nomenclature and Classification of Enzymes, Academic Press, San Diego Calif.), lyases form a distinct class designated by the numeral 4 in the first digit of the enzyme number (i.e., EC 4.x.x.x).

Further classification of lyases reflects the type of bond cleaved as well as the nature of the cleaved group. The group of C—C lyases includes carboxyl-lyases (decarboxylases), aldehyde-lyases (aldolases), oxo-acid-lyases, and other lyases. The C—O lyase group includes hydro-lyases, lyases acting on polysaccharides, and other lyases. The C—N lyase group includes ammonia-lyases, amidine-lyases, amine-lyases (deaminases), and other lyases. Lyases are critical components of cellular biochemistry, with roles in metabolic energy production, including fatty acid metabolism and the tricarboxylic acid cycle, as well as other diverse enzymatic processes.

One important family of lyases are the carbonic anhydrases (CA), also called carbonate dehydratases, which catalyze the hydration of carbon dioxide in the reaction H₂O+CO₂≈HCO₃⁻+H+. CA accelerates this reaction by a factor of over 10⁶ by virtue of a zinc ion located in a deep cleft about 15 Å below the protein's surface and co-ordinated to the imidazole groups of three His residues. Water bound to the zinc ion is rapidly converted to HCO₃—.

Eight enzymatic and evolutionarily related forms of carbonic anhydrase are currently known to exist in humans: three cytosolic isozymes (CAI, CAII, and CAIII), two membrane-bound forms (CAIV and CAVII), a mitochondrial form (CAV), a secreted salivary form (CAVI) and a yet uncharacterized isozyme (PROSITE PDOC00146 Eukaryotic-type carbonic anhydrases signature). Though the isoenzymes CAI, CAII, and bovine CAIII have similar secondary structures and polypeptide-chain folds, CAI has 6 tryptophans, CAII has 7 and CAM has 8 (Boren, K. et al. (1996) Protein Sci. 5:2479-2484). CAII is the predominant CA isoenzyme in the brain of mammals.

CAs participate in a variety of physiological processes that involve pH regulation, CO₂ and HCO₃⁻ transport, ion transport, and water and electrolyte balance. For example, CAII contributes to H³⁰ secretion by gastric parietal cells, by renal tubular cells, and by osteoclasts that secrete H⁺ to acidify the bone-resorbing compartment. In addition, CAII promotes HCO₃⁻ secretion by pancreatic duct cells, cilary body epithelium, choroid plexus, salivary gland acinar cells, and distal colonal epithelium, thus playing a role in the production of pancreatic juice, aqueous humor, cerebrospinal fluid, and saliva, and contributing to electrolyte and water balance. CAII also promotes CO₂ exchange in proximal tubules in the kidney, in erythrocytes, and in lung. CAIV has roles in several tissues: it facilitates HCO₃⁻ reabsorption in the kidney; promotes CO₂ flux in tissues including brain, skeletal muscle, and heart muscle; and promotes CO₂ exchange from the blood to the alveoli in the lung. CAVI probably plays a role in pH regulation in saliva, along with CAII, and may have a protective effect in the esophagus and stomach. Mitochondrial CAV appears to play important roles in gluconeogenesis and ureagenesis, based on the effects of CA inhibitors on these pathways. (Sly, W. S. and P. Y. Hu (1995) Ann. Rev. Biochem. 64:375-401.) A number of disease states are marked by variations in CA activity. Mutations in CAII which lead to CAII deficiency are the cause of osteopetrosis with renal tubular acidosis (OMIM #259730 Osteopetrosis with Renal Tubular Acidosis). The concentration of CAII in the cerebrospinal fluid (CSF) appears to mark disease activity in patients with brain damage. High CA concentrations have been observed in patients with brain infarction. Patients with transient ischemic attack, multiple sclerosis, or epilepsy usually have CAII concentrations in the normal range, but higher CAII levels have been observed in the CSF of those with central nervous system infection, dementia, or trigeminal neuralgia (Parkkila, A. K. et al. (1997) Eur. J. Clin. Invest. 27:392-397). Colonic adenomas and adenocarcinomas have been observed to fail to stain for CA, whereas non-neoplastic controls showed CAI and CAII in the cytoplasm of the columnar cells lining the upper half of colonic crypts. The neoplasms show staining patterns similar to less mature cells lining the base of normal crypts (Gramlich T. L. et al. (1990) Arch. Pathol. Lab. Med. 114:415-419).

Therapeutic interventions in a number of diseases involve altering CA activity. CA inhibitors such as acetazolamide are used in the treatment of glaucoma (Stewart, W. C. (1999) Curr. Opin. Opthamol. 10:99-108), essential tremor and Parkinson's disease (Uitti, R. J. (1998) Geriatrics 53:46-48, 53-57), intermittent ataxia (Singhvi, J. P. et al. (2000) Neurology India 48:78-80), and altitude related illnesses (Klocke, D. L. et al. (1998) Mayo Clin. Proc. 73:988-992).

CA activity can be particularly useful as an indicator of long-term disease conditions, since the enzyme reacts relatively slowly to physiological changes. CAI and zinc concentrations have been observed to decrease in hyperthyroid Graves' disease (Yoshida, K. (1996) Tohoku J. Exp. Med. 178:345-356) and glycosylated CAI is observed in diabetes mellitus (Kondo, T. et al. (1987) Clin. Chim. Acta 166:227-236). A positive correlation has been observed between CAI and CAII reactivity and endometriosis (Brinton, D. A. et al. (1996) Ann. Clin. Lab. Sci. 26:409-420; D'Cruz, O. J. et al. (1996) Fertil. Steril. 66:547-556).

Another important member of the lyase family is ornithine decarboxylase (ODC), the initial rate-limiting enzyme in polyamine biosynthesis. ODC catalyses the transformation of ornithine into putrescine in the reaction L-ornithine≈putrescine+CO₂. Polyamines, which include putrescine and the subsequent metabolic pathway products spermidine and spermine, are ubiquitous cell components essential for DNA synthesis, cell differentiation, and proliferation. Thus the polyamines play a key role in tumor proliferation (Medina, M. A. et al. (1999) Biochem. Pharmacol. 57:1341-1344).

ODC is a pyridoxal-5′-phosphate (PLP)-dependent enzyme which is active as a homodimer. Conserved residues include those at the PLP binding site and a stretch of glycine residues thought to be part of a substrate binding region (PROSITE PDOC00685 Orn/DAP/Arg decarboxylase family 2 signatures). Mammalian ODCs also contain PEST regions, sequence fragments enriched in proline, glutamic acid, serine, and threonine residues that act as signals for intracellular degradation (Medina et al., supra).

Many chemical carcinogens and tumor promoters increase ODC levels and activity. Several known oncogenes may increase ODC levels by enhancing transcription of the ODC gene, and ODC itself may act as an oncogene when expressed at very high levels. A high level of ODC is found in a number of precancerous conditions, and elevation of ODC levels has been used as part of a screen for tumor-promoting compounds (Pegg, A. E. et al. (1995) J. Cell. Biochem. Suppl. 22:132-138).

Inhibitors of ODC have been used to treat tumors in animal models and human clinical trials, and have been shown to reduce development of tumors of the bladder, brain, esophagus, gastrointestinal tract, lung, oral cavity, mammary gland, stomach, skin and trachea (Pegg et al., supra; McCann, P. P. and A. E. Pegg (1992) Pharmac. Ther. 54:195-215). ODC also shows promise as a target for chemoprevention (Pegg et al., supra). ODC inhibitors have also been used to treat infections by African trypanosomes, malaria, and Pneumocystis carinii, and are potentially useful for treatment of autoimmune diseases such as lupus and rheumatoid arthritis (McCann and Pegg, supra).

Another family of pyridoxal-dependent decarboxylases are the group II decarboxylases. This family includes glutamate decarboxylase (GAD) which catalyzes the decarboxylation of glutamate into the neurotransmitter GABA; histidine decarboxylase (HDC), which catalyzes the decarboxylation of histidine to histamine; aromatic-L-amino-acid decarboxylase (DDC), also known as L-dopa decarboxylase or tryptophan decarboxylase, which catalyzes the decarboxylation of tryptophan to tryptamine and also acts on 5-hydroxy-tryptophan and dihydroxyphenylalanine (L-dopa); and cysteine sulfinic acid decarboxylase (CSD), the rate-limiting enzyme in the synthesis of taurine from cysteine (PROSITE PDOC00329 DDC/GAD/HDC/TyrDC pyridoxal-phosphate attachment site). Taurine is an abundant sulfonic amino acid in brain and is thought to act as an osmoregulator in brain cells (Bitoun, M. and M. Tappaz (2000) J. Neurochem. 75:919-924).

Isomerases

Isomerases are a class of enzymes that catalyze geometric or structural changes within a molecule to form a single product. This class includes racemases and epimerases, cis-trans-isomerases, intramolecular oxidoreductases, intramolecular transferases (mutases) and intramolecular lyases. Isomerases are critical components of cellular biochemistry with roles in metabolic energy production including glycolysis, as well as other diverse enzymatic processes (Stryer, supra, pp. 483-507).

Racemases are a subset of isomerases that catalyze inversion of a molecule's configuration around the asymmetric carbon atom in a substrate having a single center of asymmetry, thereby interconverting two racemers. Epimerases are another subset of isomerases that catalyze inversion of configuration around an asymmetric carbon atom in a substrate with more than one center of symmetry, thereby interconverting two epimers. Racemases and epimerases can act on amino acids and derivatives, hydroxy acids and derivatives, and carbohydrates and derivatives. The interconversion of UDP-galactose and UDP-glucose is catalyzed by UDP-galactose-4′-epimerase. Proper regulation and function of this epimerase is essential to the synthesis of glycoproteins and glycolipids. Elevated blood galactose levels have been correlated with UDP-galactose-4′-epimerase deficiency in screening programs of infants (Gitzelmann, R. (1972) Helv. Paediat. Acta 27:125-130).

Correct folding of newly synthesized proteins is assisted by molecular chaperones and folding catalysts, two unrelated groups of helper molecules. Chaperones suppress non-productive side reactions by stoichiometric binding to folding intermediates, whereas folding enzymes catalyze some of the multiple folding steps that enable proteins to attain their final functional configurations (Kern, G. et al. (1994) FEBS Lett. 348:145-148). One class of folding enzymes, the peptidyl prolyl cis-trans isomerases (PPIases), isomerizes certain proline imidic bonds in what is considered to be a rate limiting step in protein maturation and export. PPIases catalyze the cis to trans isomerization of certain proline imidic bonds in proteins. There are three evolutionarily unrelated families of PPIases: the cyclophilins, the FK506 binding proteins, and the newly characterized parvulin family (Rahfeld, J. U. et al. (1994) FEBS Lett. 352:180-184).

The cyclophilins (CyP) were originally identified as major receptors for the immunosuppressive drug cyclosporin A (CsA), an inhibitor of T-cell activation (Handschumacher, R. E. et al. (1984) Science 226:544-547; Harding, M. W. et al. (1986) J. Biol. Chem. 261:8547-8555). Thus, the peptidyl-prolyl isomerase activity of CyP may be part of the signaling pathway that leads to T-cell activation. Subsequent work demonstrated that CyP's isomerase activity is essential for correct protein folding and/or protein trafficking, and may also be involved in assembly/disassembly of protein complexes and regulation of protein activity. For example, in Drosophila, the CyP NinaA is required for correct localization of rhodopsins, while a mammalian CyP (Cyp40) is part of the Hsp90/Hsp70 complex that binds steroid receptors. The mammalian CyP (CypA) has been shown to bind the gag protein from human immunodeficiency virus 1 (HIV-1), an interaction that can be inhibited by cyclosporin. Since cyclosporin has potent anti-HIV-1 activity, CypA may play an essential function in HIV-1 replication. Finally, Cyp40 has been shown to bind and inactivate the transcription factor c-Myb, an effect that is reversed by cyclosporin. This effect implicates CyP in the regulation of transcription, transformation, and differentiation (Bergsma, D. J. et al (1991) J. Biol. Chem. 266:23204-23214; Hunter, T. (1998) Cell 92:141-143; and Leverson, J. D. and S. A. Ness (1998) Mol. Cell. 1:203-211).

One of the major rate limiting steps in protein folding is the thiol:disulfide exchange that is necessary for correct protein assembly. Although incubation of reduced, unfolded proteins in buffers with defined ratios of oxidized and reduced thiols can lead to native conformation, the rate of folding is slow and the attainment of native conformation decreases proportionately with the size and number of cysteines in the protein. Certain cellular compartments such as the endoplasmic reticulum of eukaryotes and the periplasmic space of prokaryotes are maintained in a more oxidized state than the surrounding cytosol. Correct disulfide formation can occur in these compartments, but at a rate that is insufficient for normal cell processes and inadequate for synthesizing secreted proteins. The protein disulfide isomerases, thioredoxins and glutaredoxins are able to catalyze the formation of disulfide bonds and regulate the redox environment in cells to enable the necessary thiol:disuffide exchanges (Loferer, H. (1995) J. Biol. Chem. 270:26178-26183).

Each of these proteins has somewhat different functions, but all belong to a group of disulfide-containing redox proteins that contain a conserved active-site sequence and are ubiquitously distributed in eukaryotes and prokaryotes. Protein disulfide isomerases are found in the endoplasmic reticulum of eukaryotes and in the periplasmic space of prokaryotes. They function by exchanging their own disulfide for a thiol in a folding peptide chain. In contrast, the reduced thioredoxins and glutaredoxins are generally found in the cytoplasm and function by directly reducing disulfides in the substrate proteins.

Oxidoreductases can be isomerases as well. Oxidoreductases catalyze the reversible transfer of electrons from a substrate that becomes oxidized to a substrate that becomes reduced. This class of enzymes includes dehydrogenases, hydroxylases, oxidases, oxygenases, peroxidases, and reductases. Proper maintenance of oxidoreductase levels is physiologically important. For example, genetically-linked deficiencies in lipoamide dehydrogenase can result in lactic acidosis (Robinson, B. H. et al. (1977) Pediat. Res. 11:1198-1202).

Another subgroup of isomerases are the transferases (or mutases). Transferases transfer a chemical group from one compound (the donor) to another compound (the acceptor). The types of groups transferred by these enzymes include acyl groups, amino groups, phosphate groups (phosphotransferases or phosphomutases), and others. The transferase carnitine palmitoyltransferase is an important component of fatty acid metabolism. Genetically-linked deficiencies in this transferase can lead to myopathy (Scriver, C. et al. (1995) The Metabolic and Molecular Basis of Inherited Disease, McGraw-Hill, New York N.Y., pp. 1501-1533).

Yet another subgroup of isomerases are the topoisomersases. Topoisomerases are enzymes that affect the topological state of DNA. For example, defects in topoisomerases or their regulation can affect normal physiology. Reduced levels of topoisomerase II have been correlated with some of the DNA processing defects associated with the disorder ataxia-telangiectasia (Singh, S. P. et al. (1988) Nucleic Acids Res. 16:3919-3929).

Ligases

Ligases catalyze the formation of a bond between two substrate molecules. The process involves the hydrolysis of a pyrophosphate bond in ATP or a similar energy donor. Ligases are classified based on the nature of the type of bond they form, which can include carbon-oxygen, carbon-sulfur, carbon-nitrogen, carbon-carbon and phosphoric ester bonds.

Ligases forming carbon-oxygen bonds include the aminoacyl-transfer RNA (tRNA) synthetases which are important RNA-associated enzymes with roles in translation. Protein biosynthesis depends on each amino acid forming a linkage with the appropriate tRNA. The aminoacyl-tRNA synthetases are responsible for the activation and correct attachment of an amino acid with its cognate tRNA. The 20 aminoacyl-tRNA synthetase enzymes can be divided into two structural classes, and each class is characterized by a distinctive topology of the catalytic domain. Class I enzymes contain a catalytic domain based on the nucleotide-binding “Rossman fold”. Class II enzymes contain a central catalytic domain, which consists of a seven-stranded antiparallel β-sheet motif, as well as N- and C-terminal regulatory domains. Class II enzymes are separated into two groups based on the heterodimeric or homodimeric structure of the enzyme; the latter group is further subdivided by the structure of the N- and C-terminal regulatory domains (Hartlein, M. and S. Cusack, (1995) J. Mol. Evol. 40:519-530). Autoantibodies against aminoacyl-tRNAs are generated by patients with dermatomyositis and polymyositis, and correlate strongly with complicating interstitial lung disease (ILD). These antibodies appear to be generated in response to viral infection, and coxsackie virus has been used to induce experimental viral myositis in animals.

Ligases forming carbon-sulfur bonds (acid-thiol ligases) mediate a large number of cellular biosynthetic intermediary metabolism processes involving intermolecular transfer of carbon atom-containing substrates (carbon substrates). Examples of such reactions include the tricarboxylic acid cycle, synthesis of fatty acids and long-chain phospholipids, synthesis of alcohols and aldehydes, synthesis of intermediary metabolites, and reactions involved in the amino acid degradation pathways. Some of these reactions require input of energy, usually in the form of conversion of ATP to either ADP or AMP and pyrophosphate.

In many cases, a carbon substrate is derived from a small molecule containing at least two carbon atoms. The carbon substrate is often covalently bound to a larger molecule which acts as a carbon substrate carrier molecule within the cell. In the biosynthetic mechanisms described above, the carrier molecule is coenzyme A. Coenzyme A (CoA) is structurally related to derivatives of the nucleotide ADP and consists of 4′-phosphopantetheine linked via a phosphodiester bond to the alpha phosphate group of adenosine 3′,5′-bisphosphate. The terminal thiol group of 4′-phosphopantetheine acts as the site for carbon substrate bond formation. The predominant carbon substrates which utilize CoA as a carrier molecule during biosynthesis and intermediary metabolism in the cell are acetyl, succinyl, and propionyl moieties, collectively referred to as acyl groups. Other carbon substrates include enoyl lipid, which acts as a fatty acid oxidation intermediate, and carnitine, which acts as an acetyl-CoA flux regulator/mitochondrial acyl group transfer protein. Acyl-CoA and acetyl-CoA are synthesized in the cell by acyl-CoA synthetase and acetyl-CoA synthetase, respectively.

Activation of fatty acids is mediated by at least three forms of acyl-CoA synthetase activity: i) acetyl-CoA synthetase, which activates acetate and several other low molecular weight carboxylic acids and is found in muscle mitochondria and the cytosol of other tissues; ii) medium-chain acyl-CoA synthetase, which activates fatty acids containing between four and eleven carbon atoms (predominantly from dietary sources), and is present only in liver mitochondria; and iii) acyl CoA synthetase, which is specific for long chain fatty acids with between six and twenty carbon atoms, and is found in microsomes and the mitochondria. Proteins associated with acyl-CoA synthetase activity have been identified from many sources including bacteria, yeast, plants, mouse, and man. The activity of acyl-CoA synthetase may be modulated by phosphorylation of the enzyme by cAMP-dependent protein kinase.

Ligases forming carbon-nitrogen bonds include amide synthases such as glutamine synthetase (glutamate-ammonia ligase) that catalyzes the amination of glutamic acid to glutamine by ammonia using the energy of ATP hydrolysis. Glutamine is the primary source for the amino group in various amide transfer reactions involved in de novo pyrimidine nucleotide synthesis and in purine and pyrimidine ribonucleotide interconversions. Overexpression of glutamine synthetase has been observed in primary liver cancer (Christa, L. et al. (1994) Gastroent. 106:1312-1320).

Acid-amino-acid ligases (peptide synthases) are represented by the ubiquitin conjugating enzymes which are associated with the ubiquitin conjugation system (UCS), a major pathway for the degradation of cellular proteins in eukaryotic cells and some bacteria. The UCS mediates the elimination of abnormal proteins and regulates the half-lives of important regulatory proteins that control cellular processes such as gene transcription and cell cycle progression. In the UCS pathway, proteins targeted for degradation are conjugated to ubiquitin (Ub), a small heat stable protein. Ub is first activated by a ubiquitin-activating enzyme (E1), and then transferred to one of several Ub-conjugating enzymes (E2). E2 then links the Ub molecule through its C-terminal glycine to an internal lysine (acceptor lysine) of a target protein. The ubiquitinated protein is then recognized and degraded by proteasome, a large, multisubunit proteolytic enzyme complex, and ubiquitin is released for reutilization by ubiquitin protease. The UCS is implicated in the degradation of mitotic cyclic kinases, oncoproteins, tumor suppressor genes such as p53, viral proteins, cell surface receptors associated with signal transduction, transcriptional regulators, and mutated or damaged proteins (Ciechanover, A. (1994) Cell 79:13-21).

Cyclo-ligases and other carbon-nitrogen ligases comprise various enzymes and enzyme complexes that participate in the de novo pathways of purine and pyrimidine biosynthesis. Because these pathways are critical to the synthesis of nucleotides for replication of both RNA and DNA, many of these enzymes have been the targets of clinical agents for the treatment of cell proliferative disorders such as cancer and infectious diseases.

Purine biosynthesis occurs de novo from the amino acids glycine and glutamine, and other small molecules. Three of the key reactions in this process are catalyzed by a trifunctional enzyme composed of glycinamide-ribonucleotide synthetase (GARS), aminoimidazole ribonucleotide synthetase (AIRS), and glycinamide ribonucleotide transformylase (GART). Together these three enzymes combine ribosylamine phosphate with glycine to yield phosphoribosyl aminoimidazole, a precursor to both adenylate and guanylate nucleotides. This trifunctional protein has been implicated in the pathology of Downs syndrome (Aimi, J. et al. (1990) Nucleic Acid Res. 18:6665-6672). Adenylosuccinate synthetase catalyzes a later step in purine biosynthesis that converts inosinic acid to adenylosuccinate, a key step on the path to ATP synthesis. This enzyme is also similar to another carbon-nitrogen ligase, argininosuccinate synthetase, that catalyzes a similar reaction in the urea cycle (Powell, S. M. et al. (1992) FEBS Lett. 303:4-10).

Adenylosuccinate synthetase, adenylosuccinate lyase, and AMP deaminase may be considered as a functional unit, the purine nucleotide cycle. This cycle converts AMP to inosine monophosphate (IMP) and reconverts IMP to AMP via adenylosuccinate, thereby producing NH₃ and forming fumarate from aspartate. In muscle, the purine nucleotide cycle functions, during intense exercise, in the regeneration of ATP by pulling the adenylate kinase reaction in the direction of ATP formation and by providing Krebs cycle intermediates. In kidney, the purine nucleotide cycle accounts for the release of NH₃ under normal acid-base conditions. In brain, the purine nucleotide cycle may contribute to ATP recovery. Adenylosuccinate lyase deficiency provokes psychomotor retardation, often accompanied by autistic features (Van den Berghe, G. et al. (1992) Prog Neurobiol.: 39:547-561). A marked imbalance in the enzymic pattern of purine metabolism is linked with transformation and/or progression in cancer cells. In rat hepatomas the specific activities of the anabolic enzymes, IMP dehydrogenase, GMP synthetase, adenylosuccinate synthetase, adenylosuccinase, AMP deaminase and amidophosphoribosyltransferase, increased to 13.5-, 3.7-, 3.1-, 1.8-, 5.5- and 2.8-fold, respectively, of those in normal liver (Weber, G. (1983) Clin. Biochem. 16:57-63).

Like the de novo biosynthesis of purines, de novo synthesis of the pyrimidine nucleotides uridylate and cytidylate also arises from a common precursor, in this instance the nucleotide orotidylate derived from orotate and phosphoribosyl pyrophosphate (PPRP). Again a trifunctional enzyme comprising three carbon-nitrogen ligases plays a key role in the process. In this case the enzymes aspartate transcarbamylase (ATCase), carbamyl phosphate synthetase II, and dihydroorotase (DHOase) are encoded by a single gene called CAD. Together these three enzymes combine the initial reactants in pyrimidine biosynthesis, glutamine, CO₂, and ATP to form dihydroorotate, the precursor to orotate and orotidylate (Iwahana, H. et al. (1996) Biochem. Biophys. Res. Commun. 219:249-255). Further steps then lead to the synthesis of uridine nucleotides from orotidylate. Cytidine nucleotides are derived from uridine-5′-triphosphate (UTP) by the amidation of UTP using glutamine as the amino donor and the enzyme CTP synthetase. Regulatory mutations in the human CTP synthetase are believed to confer multi-drug resistance to agents widely used in cancer therapy (Yamauchi, M. et al. (1990) EMBO J. 9:2095-2099).

Ligases forming carbon-carbon bonds include the carboxylases acetyl-CoA carboxylase and pyruvate carboxylase. Acetyl-CoA carboxylase catalyzes the carboxylation of acetyl-CoA from CO₂ and H₂O using the energy of ATP hydrolysis. Acetyl-CoA carboxylase is the rate-limiting enzyme in the biogenesis of long-chain fatty acids. Two isoforms of acetyl-CoA carboxylase, types I and types II, are expressed in human in a tissue-specific manner (Ha, J. et al. (1994) Eur. J. Biochem. 219:297-306). Pyruvate carboxylase is a nuclear-encoded mitochondrial enzyme that catalyzes the conversion of pyruvate to oxaloacetate, a key intermediate in the citric acid cycle.

Ligases forming phosphoric ester bonds include the DNA ligases involved in both DNA replication and repair. DNA ligases seal phosphodiester bonds between two adjacent nucleotides in a DNA chain using the energy from ATP hydrolysis to first activate the free 5′-phosphate of one nucleotide and then react it with the 3′-OH group of the adjacent nucleotide. This resealing reaction is used in DNA replication to join small DNA fragments called “Okazaki” fragments that are transiently formed in the process of replicating new DNA, and in DNA repair. DNA repair is the process by which accidental base changes, such as those produced by oxidative damage, hydrolytic attack, or uncontrolled methylation of DNA, are corrected before replication or transcription of the DNA can occur. Bloom's syndrome is an inherited human disease in which individuals are partially deficient in DNA ligation and consequently have an increased incidence of cancer (Alberts et al., supra, p. 247).

Pantothenate synthetase (D-pantoate; beta-alanine ligase (AMP-forming); EC 6.3.2.1) is the last enzyme of the pathway of pantothenate (vitamin B(5)) synthesis. It catalyzes the condensation of pantoate with beta-alanine in an ATP-dependent reaction. The enzyme is dimeric, with two well-defined domains per protomer: the N-terminal domain, a Rossmann fold, contains the active site cavity, with the C-terminal domain forming a hinged lid. The N-terminal domain is structurally very similar to class I aminoacyl-tRNA synthetases and is thus a member of the cytidylyltransferase superfamily (von Delft, F. et al. (2000) Structure (Camb) 9:439-450).

Farnesyl diphosphate synthase (FPPS) is an essential enzyme that is required both for cholesterol synthesis and protein prenylation. The enzyme catalyzes the formation of farnesyl diphosphate from dimethylallyl diphosphate and isopentyl diphosphate. FPPS is inhibited by nitrogen-containing biphosphonates, which can lead to the inhibition of osteoclast-mediated bone resorption by preventing protein prenylation (Dunford, J. E. et al. (2001) J. Pharmacol. Exp. Ther. 296:235-242).

5-aminolevulinate synthase (ALAS; delta-aminolevulinate synthase; EC 2.3.1.37) catalyzes the rate-limiting step in heme biosynthesis in both erythroid and non-erythroid tissues. This enzyme is unique in the heme biosynthetic pathway in being encoded by two genes, the first encoding ALAS1, the non-erythroid specific enzyme which is ubiquitously expressed, and the second encoding ALAS2, which is expressed exclusively in erythroid cells. The genes for ALAS1 and ALAS2 are located, respectively, on chromosome 3 and on the X chromosome. Defects in the gene encoding ALAS2 result in X-linked sideroblastic anemia. Elevated levels of ALAS are seen in acute hepatic porphyrias and can be lowered by zinc mesoporphyrin.

Drug Metabolizing Enzymes (DMEs)

The metabolism of a drug and its movement through the body (pharmacokinetics) are important in determining its effects, toxicity, and interactions with other drugs. The three processes governing pharmacokinetics are the absorption of the drug, distribution to various tissues, and elimination of drug metabolites. These processes are intimately coupled to drug metabolism, since a variety of metabolic modifications alter most of the physicochemical and pharmacological properties of drugs, including solubility, binding to receptors, and excretion rates. The metabolic pathways which modify drugs also accept a variety of naturally occurring substrates such as steroids, fatty acids, prostaglandins, leukotrienes, and vitamins. The enzymes in these pathways are therefore important sites of biochemical and pharmacological interaction between natural compounds, drugs, carcinogens, mutagens, and xenobiotics. It has long been appreciated that inherited differences in drug metabolism lead to drastically different levels of drug efficacy and toxicity among individuals. Advances in pharmacogenomics research, of which DMEs constitute an important part, are promising to expand the tools and information that can be brought to bear on questions of drug efficacy and toxicity (See Evans, W. E. and R. V. Relling (1999) Science 286:487-491). DMEs have broad substrate specificities, unlike antibodies, for example, which are diverse and highly specific. Since DMEs metabolize a wide variety of molecules, drug interactions may occur at the level of metabolism so that, for example, one compound may induce a DME that affects the metabolism of another compound.

Drug metabolic reactions are categorized as Phase I, which prepare the drug molecule for functioning and further metabolism, and Phase II, which are conjugative. In general, Phase I reaction products are partially or fully inactive, and Phase II reaction products are the chief excreted species. However, Phase I reaction products are sometimes more active than the original administered drugs; this metabolic activation principle is exploited by pro-drugs (e.g. L-dopa). Additionally, some nontoxic compounds (e.g. aflatoxin, benzo[α]pyrene) are metabolized to toxic intermediates through these pathways. Phase I reactions are usually rate-limiting in drug metabolism. Prior exposure to the compound, or other compounds, can induce the expression of Phase I enzymes however, and thereby increase substrate flux through the metabolic pathways. (See Klaassen, C. D. et al. (1996) Casarett and Doull's Toxicology: The Basic Science of Poisons, McGraw-Hill, New York, N.Y., pp. 113-186; Katzung, B. G. (1995) Basic and Clinical Pharmacology, Appleton and Lange, Norwalk, Conn., pp. 48-59; Gibson, G. G. and P. Skett (1994) Introduction to Drug Metabolism, Blackie Academic and Professional, London.).

The major classes of Phase I enzymes include, but are not limited to, cytochrome P450 and flavin-containing monooxygenase. Other enzyme classes involved in Phase I-type catalytic cycles and reactions include, but are not limited to, NADPH cytochrome P450 reductase (CPR), the microsomal cytochrome b5/NADH cytochrome b5 reductase system, the ferredoxin/ferredoxin reductase redox pair, aldo/keto reductases, and alcohol dehydrogenases. The major classes of Phase II enzymes include, but are not limited to, UDP glucuronyltransferase, sulfotransferase, glutathione S-transferase, N-acyltransferase, and N-acetyl transferase.

Cytochrome P450 and P450 Catalytic Cycle-Associated Enzymes

Members of the cytochrome P450 superfamily of enzymes catalyze the oxidative metabolism of a variety of substrates, including natural compounds such as steroids, fatty acids, prostaglandins, leukotrienes, and vitamins, as well as drugs, carcinogens, mutagens, and xenobiotics. Cytochromes P450, also known as P450 heme-thiolate proteins, usually act as terminal oxidases in multi-component electron transfer chains, called P450-containing monooxygenase systems. Specific reactions catalyzed include hydroxylation, epoxidation, N-oxidation, sulfooxidation, N-, S-, and O-dealkylations, desulfation, deamination, and reduction of azo, nitro, and N-oxide groups. These reactions are involved in steroidogenesis of glucocorticoids, cortisols, estrogens, and androgens in animals; insecticide resistance in insects; herbicide resistance and flower coloring in plants; and environmental bioremediation by microorganisms. Cytochrome P450 actions on drugs, carcinogens, mutagens, and xenobiotics can result in detoxification or in conversion of the substance to a more toxic product. Cytochromes P450 are abundant in the liver, but also occur in other tissues; the enzymes are located in microsomes. (See ExPASY ENZYME EC 1.14.14.1; Prosite PDOC00081 Cytochrome P450 cysteine heme-iron ligand signature; PRINTS EP450I E-Class P450 Group I signature; Graham-Lorence, S. and J. A. Peterson (1996) FASEB J. 10:206-214.)

Four hundred cytochromes P450 have been identified in diverse organisms including bacteria, fungi, plants, and animals (Graham-Lorence and Peterson, supra). The B-class is found in prokaryotes and fungi, while the E-class is found in bacteria, plants, insects, vertebrates, and mammals. Five subclasses or groups are found within the larger family of E-class cytochromes P450 (PRINTS EP450I E-Class P450 Group I signature).

All cytochromes P450 use a heme cofactor and share structural attributes. Most cytochromes P450 are 400 to 530 amino acids in length. The secondary structure of the enzyme is about 70% alpha-helical and about 22% beta-sheet. The region around the heme-binding site in the C-terminal part of the protein is conserved among cytochromes P450. A ten amino acid signature sequence in this heme-iron ligand region has been identified which includes a conserved cysteine involved in binding the heme iron in the fifth coordination site. In eukaryotic cytochromes P450, a membrane-spanning region is usually found in the first 15-20 amino acids of the protein, generally consisting of approximately 15 hydrophobic residues followed by a positively charged residue. (See Prosite PDOC00081, supra; Graham-Lorence and Peterson, supra.)

Cytochrome P450 enzymes are involved in cell proliferation and development. The enzymes have roles in chemical mutagenesis and carcinogenesis by metabolizing chemicals to reactive intermediates that form adducts with DNA (Nebert, D. W. and F. J. Gonzalez (1987) Ann. Rev. Biochem. 56:945-993). These adducts can cause nucleotide changes and DNA rearrangements that lead to oncogenesis. Cytochrome P450 expression in liver and other tissues is induced by xenobiotics such as polycyclic aromatic hydrocarbons, peroxisomal proliferators, phenobarbital, and the glucocorticoid dexamethasone (Dogra, S. C. et al. (1998) Clin. Exp. Pharmacol. Physiol. 25:1-9). A cytochrome P450 protein may participate in eye development as mutations in the P450 gene CYP1B1 cause primary congenital glaucoma (OMIM #601771 Cytochrome P450, subfamily I (dioxin-inducible), polypeptide 1; CYP1B1).

Cytochromes P450 are associated with inflammation and infection. Hepatic cytochrome P450 activities are profoundly affected by various infections and inflammatory stimuli, some of which are suppressed and some induced (Morgan, E. T. (1997) Drug Metab. Rev. 29:1129-1188). Effects observed in vivo can be mimicked by proinflammatory cytokines and interferons. Autoantibodies to two cytochrome P450 proteins were found in patients with autoimmune polyenodocrinopathy-candidiasis-ectodermal dystrophy (APECED), a polyglandular autoimmune syndrome (OMIM #240300 Autoimmune polyenodocrinopathy-candidiasis-ectodermal dystrophy).

Mutations in cytochromes P450 have been linked to metabolic disorders, including congenital adrenal hyperplasia, the most common adrenal disorder of infancy and childhood; pseudovitamin D-deficiency rickets; cerebrotendinous xanthomatosis, a lipid storage disease characterized by progressive neurologic dysfunction, premature atherosclerosis, and cataracts; and an inherited resistance to the anticoagulant drugs coumarin and warfarin (Isselbacher, K. J. et al. (1994) Harrison's Principles of Internal Medicine, McGraw-Hill, Inc. New York, N.Y., pp. 1968-1970; Takeyama, K. et al. (1997) Science 277:1827-1830; Kitanaka, S. et al. (1998) N. Engl. J. Med. 338:653-661; OMIM #213700 Cerebrotendinous xanthomatosis; and OMIM #122700 Coumarin resistance). Extremely high levels of expression of the cytochrome P450 protein aromatase were found in a fibrolamellar hepatocellular carcinoma from a boy with severe gynecomastia (feminization) (Agarwal, V. R. (1998) J. Clin. Endocrinol. Metab. 83:1797-1800).

The cytochrome P450 catalytic cycle is completed through reduction of cytochrome P450 by NADPH cytochrome P450 reductase (CPR). Another microsomal electron transport system consisting of cytochrome b5 and NADPH cytochrome b5 reductase has been widely viewed as a minor contributor of electrons to the cytochrome P450 catalytic cycle. However, a recent report by Lamb, D. C. et al. (1999; FEBS Lett. 462:283-288) identifies a Candida albicans cytochrome P450 (CYP51) which can be efficiently reduced and supported by the microsomal cytochrome b5/NADPH cytochrome b5 reductase system. Therefore, there are likely many cytochromes P450 which are supported by this alternative electron donor system.

Cytochrome b5 reductase is also responsible for the reduction of oxidized hemoglobin (methemoglobin, or ferrihemoglobin, which is unable to carry oxygen) to the active hemoglobin (ferrohemoglobin) in red blood cells. Methemoglobinemia results when there is a high level of oxidant drugs or an abnormal hemoglobin (hemoglobin M) which is not efficiently reduced. Methemoglobinemia can also result from a hereditary deficiency in red cell cytochrome b5 reductase (Reviewed in Mansour, A. and A. A. Lurie (1993) Am. J. Hematol. 42:7-12).

Members of the cytochrome P450 family are also closely associated with vitamin D synthesis and catabolism. Vitamin D exists as two biologically equivalent prohormones, ergocalciferol (vitamin D₂), produced in plant tissues, and cholecalciferol (vitamin D₃), produced in animal tissues. The latter form, cholecalciferol, is formed upon the exposure of 7-dehydrocholesterol to near ultraviolet light (i.e., 290-310 nm), normally resulting from even minimal periods of skin exposure to sunlight (reviewed in Miller, W. L. and A. A. Portale (2000) Trends Endocrinol. Metab. 11:315-319).

Both prohormone forms are further metabolized in the liver to 25-hydroxyvitamin D (25(OH)D) by the enzyme 25-hydroxylase. 25(OH)D is the most abundant precursor form of vitamin D which must be further metabolized in the kidney to the active form, 1α,25-dihydroxyvitamin D (1α,25(OH)₂D), by the enzyme 25-hydroxyvitamin D 1α-hydroxylase (1α-hydroxylase). Regulation of 1α,25(OH)₂D production is primarily at this final step in the synthetic pathway. The activity of 1α-hydroxylase depends upon several physiological factors including the circulating level of the enzyme product (1α,25(OH)₂D) and the levels of parathyroid hormone (PTH), calcitonin, insulin, calcium, phosphorus, growth hormone, and prolactin. Furthermore, extrarenal 1α-hydroxylase activity has been reported, suggesting that tissue-specific, local regulation of 1α,25(OH)2D production may also be biologically important. The catalysis of 1α,25(OH)₂D to 24,25-dihydroxyvitamin D (24,25(OH)₂D), involving the enzyme 25-hydroxyvitamin D 24-hydroxylase (24-hydroxylase), also occurs in the kidney. 24-hydroxylase can also use 25(OH)D as a substrate (Shinki, T. et al. (1997) Proc. Natl. Acad. Sci. U.S.A. 94:12920-12925; Miller and Portale, supra; and references within).

Vitamin D 25-hydroxylase, 1-hydroxylase, and 24-hydroxylase are all NADPH-dependent, type I (mitochondrial) cytochrome P450 enzymes that show a high degree of homology with other members of the family. Vitamin D 25-hydroxylase also shows a broad substrate specificity and may also perform 26-hydroxylation of bile acid intermediates and 25, 26, and 27-hydroxylation of cholesterol (Dilworth, F. J. et al. (1995) J. Biol. Chem. 270:16766-16774; Miller and Portale, supra; and references within).

The active form of vitamin D (1α,25(OH)₂D) is involved in calcium and phosphate homeostasis and promotes the differentiation of myeloid and skin cells. Vitamin D deficiency resulting from deficiencies in the enzymes involved in vitamin D metabolism (e.g., 1α-hydroxylase) causes hypocalcemia, hypophosphatemia, and vitamin D-dependent (sensitive) rickets, a disease characterized by loss of bone density and distinctive clinical features, including bandy or bow leggedness accompanied by a waddling gait. Deficiencies in vitamin D 25-hydroxylase cause cerebrotendinous xanthomatosis, a lipid-storage disease characterized by the deposition of cholesterol and cholestanol in the Achilles' tendons, brain, lungs, and many other tissues. The disease presents with progressive neurologic dysfunction, including postpubescent cerebellar ataxia, atherosclerosis, and cataracts. Vitamin D 25-hydroxylase deficiency does not result in rickets, suggesting the existence of alternative pathways for the synthesis of 25(OH)D (Griffin, J. E. and J. E. Zerwekh (1983) J. Clin. Invest. 72:1190-1199; Gamblin, G. T. et al. (1985) J. Clin. Invest. 75:954-960; and Miller and Portale, supra).

Ferredoxin and ferredoxin reductase are electron transport accessory proteins which support at least one human cytochrome P450 species, cytochrome P450c27 encoded by the CYP27 gene (Dilworth, F. J. et al. (1996) Biochem. J. 320:267-71). A Streptomyces griseus cytochrome P450, CYP104D1, was heterologously expressed in Escherichia coli and found to be reduced by the endogenous ferredoxin and ferredoxin reductase enzymes (Taylor, M. et al. (1999) Biochem. Biophys. Res. Commun. 263:838-842), suggesting that many cytochrome P450 species may be supported by the ferredoxin/ferredoxin reductase pair. Ferredoxin reductase has also been found in a model drug metabolism system to reduce actinomycin D, an antitumor antibiotic, to a reactive free radical species (Flitter, W. D. and R. P. Mason (1988) Arch. Biochem. Biophys. 267:632-639).

Flavin-Containing Monooxygenase (FMO)

Flavin-containing monooxygenases oxidize the nucleophilic nitrogen, sulfur, and phosphorus heteroatom of an exceptional range of substrates. Like cytochromes P450, FMOs are microsomal and use NADPH and O₂; there is also a great deal of substrate overlap with cytochromes P450. The tissue distribution of FMOs includes liver, kidney, and lung.

Isoforms of FMO in mammals include FMO1, FMO2, FMO3, FMO4, and FMO5, which are expressed in a tissue-specific manner. The isoforms differ in their substrate specificities and properties such as inhibition by various compounds and stereospecificity of reaction. FMOs have a 13 amino acid signature sequence, the components of which span the N-terminal two-thirds of the sequences and include the FAD binding region and the FATGY motif found in many N-hydroxylating enzymes (Stehr, M. et al. (1998) Trends Biochem. Sci. 23:56-57; PRINTS FMOXYGENASE Flavin-containing monooxygenase signature). Specific reactions include oxidation of nucleophilic tertiary amines to N-oxides, secondary amines to hydroxylamines and nitrones, primary amines to hydroxylamines and oximes, and sulfur-containing compounds and phosphines to S- and P-oxides. Hydrazines, iodides, selenides, and boron-containing compounds are also substrates. FMOs are more heat labile and less detergent-sensitive than cytochromes P450 in vitro though FMO isoforms vary in thermal stability and detergent sensitivity.

FMOs play important roles in the metabolism of several drugs and xenobiotics. FMO (FMO3 in liver) is predominantly responsible for metabolizing (S)-nicotine to (S)-nicotine N-1′-oxide, which is excreted in urine. FMO is also involved in S-oxygenation of cimetidine, an H₂-antagonist widely used for the treatment of gastric ulcers. Liver-expressed forms of FMO are not under the same regulatory control as cytochrome P450. In rats, for example, phenobarbital treatment leads to the induction of cytochrome P450, but the repression of FMO1.

Lysyl Oxidase

Lysyl oxidase (lysine 6-oxidase, LO) is a copper-dependent amine oxidase involved in the formation of connective tissue matrices by crosslinking collagen and elastin. LO is secreted as an N-glycosylated precursor protein of approximately 50 kDa and cleaved to the mature form of the enzyme by a metalloprotease, although the precursor form is also active. The copper atom in LO is involved in the transport of electrons to and from oxygen to facilitate the oxidative deamination of lysine residues in these extracellular matrix proteins. While the coordination of copper is essential to LO activity, insufficient dietary intake of copper does not influence the expression of the apoenzyme. However, the absence of the functional LO is linked to the skeletal and vascular tissue disorders that are associated with dietary copper deficiency. LO is also inhibited by a variety of semicarbazides, hydrazines, and amino nitrites, as well as heparin. Beta-aminopropionitrile is a commonly used inhibitor. LO activity is increased in response to ozone, cadmium, and elevated levels of hormones released in response to local tissue trauma, such as transforming growth factor-beta, platelet-derived growth factor, angiotensin II, and fibroblast growth factor. Abnormalities in LO activity have been linked to Menkes syndrome and occipital horn syndrome. Cytosolic forms of the enzyme have been implicated in abnormal cell proliferation (reviewed in Rucker, R. B. et al. (1998) Am. J. Clin. Nutr. 67:996S-1002S and Smith-Mungo, L. I. and H M. Kagan (1998) Matrix Biol. 16:387-398).

Dihydrofolate Reductases

Dihydrofolate reductases (DHFR) are ubiquitous enzymes that catalyze the NADPH-dependent reduction of dihydrofolate to tetrahydrofolate, an essential step in the de novo synthesis of glycine and purines as well as the conversion of deoxyuridine monophosphate (dUMP) to deoxythymidine monophosphate (dTMP). The basic reaction is as follows:
7,8-dihydrofolate+NADPH→5,6,7,8-tetrahydrofolate+NADP⁺

The enzymes can be inhibited by a number of dihydrofolate analogs, including trimethroprim and methotrexate. Since an abundance of dTMP is required for DNA synthesis, rapidly dividing cells require the activity of DHFR. The replication of DNA viruses (i.e., herpesvirus) also requires high levels of DHFR activity. As a result, drugs that target DHFR have been used for cancer chemotherapy and to inhibit DNA virus replication. (For similar reasons, thymidylate synthetases are also target enzymes.) Drugs that inhibit DHFR are preferentially cytotoxic for rapidly dividing cells (or DNA virus-infected cells) but have no specificity, resulting in the indiscriminate destruction of dividing cells. Furthermore, cancer cells may become resistant to drugs such as methotrexate as a result of acquired transport defects or the duplication of one or more DHFR genes (Stryer, L. (1988) Biochemistry. W.H Freeman and Co., Inc. New York. pp. 511-519).

Aldo/Keto Reductases

Aldo/keto reductases are monomeric NADPH-dependent oxidoreductases with broad substrate specificities (Bohren, K. M. et al. (1989) J. Biol. Chem. 264:9547-9551). These enzymes catalyze the reduction of carbonyl-containing compounds, including carbonyl-containing sugars and aromatic compounds, to the corresponding alcohols. Therefore, a variety of carbonyl-containing drugs and xenobiotics are likely metabolized by enzymes of this class.

One known reaction catalyzed by a family member, aldose reductase, is the reduction of glucose to sorbitol, which is then further metabolized to fructose by sorbitol dehydrogenase. Under normal conditions, the reduction of glucose to sorbitol is a minor pathway. In hyperglycemic states, however, the accumulation of sorbitol is implicated in the development of diabetic complications (OMIM #103880 Aldo-keto reductase family 1, member B1). Members of this enzyme family are also highly expressed in some liver cancers (Cao, D. et al. (1998) J. Biol. Chem. 273:11429-11435).

Alcohol Dehydrogenases

Alcohol dehydrogenases (ADHs) oxidize simple alcohols to the corresponding aldehydes. ADH is a cytosolic enzyme, prefers the cofactor NAD⁺, and also binds zinc ion. Liver contains the highest levels of ADH, with lower levels in kidney, lung, and the gastric mucosa.

Known ADH isoforms are dimeric proteins composed of 40 kDa subunits. There are five known gene loci which encode these subunits (a, b, g, p, c), and some of the loci have characterized allelic variants (b₁, b₂, b₃, g₁, g₂). The subunits can form homodimers and heterodimers; the subunit composition determines the specific properties of the active enzyme. The holoenzymes have therefore been categorized as Class I (subunit compositions aa, ab, ag, bg, gg), Class II (pp), and Class III (cc). Class I ADH isozymes oxidize ethanol and other small aliphatic alcohols, and are inhibited by pyrazole. Class II isozymes prefer longer chain aliphatic and aromatic alcohols, are unable to oxidize methanol, and are not inhibited by pyrazole. Class III isozymes prefer even longer chain aliphatic alcohols (five carbons and longer) and aromatic alcohols, and are not inhibited by pyrazole.

The short-chain alcohol dehydrogenases include a number of related enzymes with a variety of substrate specificities. Included in this group are the mammalian enzymes D-beta-hydroxybutyrate dehydrogenase, (R)-3-hydroxybutyrate dehydrogenase, 15-hydroxyprostaglandin dehydrogenase, NADPH-dependent carbonyl reductase, corticosteroid 11-beta-dehydrogenase, and estradiol 17-beta-dehydrogenase, as well as the bacterial enzymes acetoacetyl-CoA reductase, glucose 1-dehydrogenase, 3-beta-hydroxysteroid dehydrogenase, 20-beta-hydroxysteroid dehydrogenase, ribitol dehydrogenase, 3-oxoacyl reductase, 2,3-dihydro-2,3-dihydroxybenzoate dehydrogenase, sorbitol-6-phosphate 2-dehydrogenase, 7-alpha-hydroxysteroid dehydrogenase, cis-1,2-dihydroxy-3,4 cyclohexadiene-1-carboxylate dehydrogenase, cis-toluene dihydrodiol dehydrogenase, cis-benzene glycol dehydrogenase, biphenyl-2,3-dihydro-2,3-diol dehydrogenase, N-acylmannosamine 1-dehydrogenase, and 2-deoxy-D-gluconate 3-dehydrogenase (Krozowski, Z. (1994) J. Steroid Biochem. Mol. Biol. 51:125-130; Krozowski, Z. (1992) Mol. Cell Endocrinol. 84:C25-31; and Marks, A. R. et al. (1992) J. Biol. Chem. 267:15459-15463).

Sulfotransferases

Sulfate conjugation occurs on many of the same substrates which undergo O-glucuronidation to produce a highly water-soluble sulfuric acid ester. Sulfotransferases (ST) catalyze this reaction by transferring So₃⁻ from the cofactor 3′-phosphoadenosine-5′-phosphosulfate (PAPS) to the substrate. ST substrates are predominantly phenyls and aliphatic alcohols, but also include aromatic amines and aliphatic amines, which are conjugated to produce the corresponding sulfamates. The products of these reactions are excreted mainly in urine.

STs are found in a wide range of tissues, including liver, kidney, intestinal tract, lung, platelets, and brain. The enzymes are generally cytosolic, and multiple forms are often co-expressed. For example, there are more than a dozen forms of ST in rat liver cytosol. These biochemically characterized STs fall into five classes based on their substrate preference: arylsulfotransferase, alcohol sulfotransferase, estrogen sulfotransferase, tyrosine ester sulfotransferase, and bile salt sulfotransferase.

ST enzyme activity varies greatly with sex and age in rats. The combined effects of developmental cues and sex-related hormones are thought to lead to these differences in ST expression profiles, as well as the profiles of other DMEs such as cytochromes P450. Notably, the high expression of STs in cats partially compensates for their low level of UDP glucuronyltransferase activity.

Several forms of ST have been purified from human liver cytosol and cloned. There are two phenyl sulfotransferases with different thermal stabilities and substrate preferences. The thermostable enzyme catalyzes the sulfation of phenyls such as para-nitrophenyl, minoxidil, and acetaminophen; the thermolabile enzyme prefers monoamine substrates such as dopamine, epinephrine, and levadopa. Other cloned STs include an estrogen sulfotransferase and an N-acetylglucosamine-6-O-sulfotransferase. This last enzyme is illustrative of the other major role of STs in cellular biochemistry, the modification of carbohydrate structures that may be important in cellular differentiation and maturation of proteoglycans. Indeed, an inherited defect in a sulfotransferase has been implicated in macular corneal dystrophy, a disorder characterized by a failure to synthesize mature keratan sulfate proteoglycans (Nakazawa, K. et al. (1984) J. Biol. Chem. 259:13751-13757; OMIM #217800 Macular dystrophy, corneal).

Galactosyltransferases

Galactosyltransferases are a subset of glycosyltransferases that transfer galactose (Gal) to the terminal N-acetylglucosamine (GlcNAc) oligosaccharide chains that are part of glycoproteins or glycolipids that are free in solution (Kolbinger, F. et al. (1998) J. Biol. Chem. 273:433-440; Amado, M. et al. (1999) Biochim. Biophys. Acta 1473:35-53). Galactosyltransferases have been detected on the cell surface and as soluble extracellular proteins, in addition to being present in the Golgi. β1,3-galactosyltransferases form Type I carbohydrate chains with Gal (β1-3)GlcNAc linkages. Known human and mouse β1,3-galactosyltransferases appear to have a short cytosolic domain, a single transmembrane domain, and a catalytic domain with eight conserved regions. (Kolbinger et al., supra; and Hennet, T. et al. (1998) J. Biol. Chem. 273:58-65). In mouse UDP-galactose:β-N-acetylglucosamine β1,3-galactosyltransferase-I region 1 is located at amino acid residues 78-83, region 2 is located at amino acid residues 93-102, region 3 is located at amino acid residues 116-119, region 4 is located at amino acid residues 147-158, region 5 is located at amino acid residues 172-183, region 6 is located at amino acid residues 203-206, region 7 is located at amino acid residues 236-246, and region 8 is located at amino acid residues 264-275. A variant of a sequence found within mouse UDP-galactose:β-N-acetylglucosamine β1,3-galactosyltransferase-I region 8 is also found in bacterial galactosyltransferases, suggesting that this sequence defines a galactosyltransferase sequence motif (Hennet et al., supra). Recent work suggests that brainiac protein is a β1,3-galactosyltransferase (Yuan, Y. et al. (1997) Cell 88:9-11; and Hennet et al., supra).

UDP-Gal:GlcNAc-1,4-galactosyltransferase (−1,4-GalT) (Sato, T. et al., (1997) EMBO J. 16:1850-1857) catalyzes the formation of Type II carbohydrate chains with Gal (β1-4)GlcNAc linkages. As is the case with the β1,3-galactosyltransferase, a soluble form of the enzyme is formed by cleavage of the membrane-bound form. Amino acids conserved among β1,4-galactosyltransferases include two cysteines linked through a disulfide-bond and a putative UDP-galactose-binding site in the catalytic domain (Yadav, S. and K. Brew (1990) J. Biol. Chem. 265:14163-14169; Yadav, S. P. and K. Brew (1991) J. Biol. Chem. 266:698-703; and Shaper, N. L. et al. (1997) J. Biol. Chem. 272:31389-31399). β1,4-galactosyltransferases have several specialized roles in addition to synthesizing carbohydrate chains on glycoproteins or glycolipids. In mammals a β31,4-galactosyltransferase, as part of a heterodimer with α-lactalbumin, functions in lactating mammary gland lactose production. A β1,4-galactosyltransferase on the surface of sperm functions as a receptor that specifically recognizes the egg. Cell surface β1,4-galactosyltransferases also function in cell adhesion, cell/basal lamina interaction, and normal and metastatic cell migration. (Shur, B. (1993) Curr. Opin. Cell Biol. 5:854-863; and Shaper, J. (1995) Adv. Exp. Med. Biol. 376:95-104).

Gamma-Glutamyl Transpeptidase

Gamma-glutamyl transpeptidases are ubiquitously expressed enzymes that initiate extracellular glutathione (GSH) breakdown by cleaving gamma-glutamyl amide bonds. The breakdown of GSH provides cells with a regional cysteine pool for biosynthetic pathways. Gamma-glutamyl transpeptidases also contribute to cellular antioxidant defenses and expression is induced by oxidative stress. The cell surface-localized glycoproteins are expressed at high levels in cancer cells. Studies have suggested that the high level of gamma-glutamyl transpeptidase activity present on the surface of cancer cells could be exploited to activate precursor drugs, resulting in high local concentrations of anti-cancer therapeutic agents (Hanigan, M. H. (1998) Chem. Biol. Interact. 111-112:333-342; Taniguchi, N. and Y. Ikeda (1998) Adv. Enzymol. Relat. Areas Mol. Biol. 72:239-278; Chikhi, N. et al. (1999) Comp. Biochem. Physiol. B. Biochem. Mol. Biol. 122:367-380).

Aminotransferases

Aminotransferases comprise a family of pyridoxal 5′-phosphate (PLP)-dependent enzymes that catalyze transformations of amino acids. Aspartate aminotransferase (AspAT) is the most extensively studied PLP-containing enzyme. It catalyzes the reversible transamination of dicarboxylic L-amino acids, aspartate and glutamate, and the corresponding 2-oxo acids, oxalacetate and 2-oxoglutarate. Other members of the family include pyruvate aminotransferase, branched-chain amino acid aminotransferase, tyrosine aminotransferase, aromatic aminotransferase, alanine:glyoxylate aminotransferase (AGT), and kynurenine aminotransferase (Vacca, R. A. et al. (1997) J. Biol. Chem. 272:21932-21937).

Primary hyperoxaluria type-I is an autosomal recessive disorder resulting in a deficiency in the liver-specific peroxisomal enzyme, alanine:glyoxylate aminotransferase-1. The phenotype of the disorder is a deficiency in glyoxylate metabolism. In the absence of AGT, glyoxylate is oxidized to oxalate rather than being transaminated to glycine. The result is the deposition of insoluble calcium oxalate in the kidneys and urinary tract, ultimately causing renal failure (Lumb, M. J. et al. (1999) J. Biol. Chem. 274:20587-20596).

Kynurenine aminotransferase catalyzes the irreversible transamination of the L-tryptophan metabolite L-kynurenine to form kynurenic acid. The enzyme may also catalyze the reversible transamination reaction between L-2-aminoadipate and 2-oxoglutarate to produce 2-oxoadipate and L-glutamate. Kynurenic acid is a putative modulator of glutamatergic neurotransmission; thus a deficiency in kynurenine aminotransferase may be associated with pleotrophic effects (Buchli, R. et al. (1995) J. Biol. Chem. 270:29330-29335).

Catechol-O-methyltransferase

Catechol-O-methyltransferase (COMT) catalyzes the transfer of the methyl group of S-adenosyl-L-methionine (AdoMet; SAM) donor to one of the hydroxyl groups of the catechol substrate (e.g., L-dopa, dopamine, or DBA). Methylation of the 3′-hydroxyl group is favored over methylation of the 4′-hydroxyl group and the membrane bound isoform of COMT is more regiospecific than the soluble form. Translation of the soluble form of the enzyme results from utilization of an internal start codon in a full-length mRNA (1.5 kb) or from the translation of a shorter mRNA (1.3 kb), transcribed from an internal promoter. The proposed S_{N}2-like methylation reaction requires Mg⁺⁺ and is inhibited by Ca⁺⁺. The binding of the donor and substrate to COMT occurs sequentially. AdoMet first binds COMT in a Mg⁺⁺-independent manner, followed by the binding of Mg⁺⁺ and the binding of the catechol substrate.

The amount of COMT in tissues is relatively high compared to the amount of activity normally required, thus inhibition is problematic. Nonetheless, inhibitors have been developed for in vitro use (e.g., gallates, tropolone, U-0521, and 3,4′-dihydroxy-2-methyl-propiophetropolone) and for clinical use (e.g., nitrocatechol-based compounds and tolcapone). Administration of these inhibitors results in the increased half-life of L-dopa and the consequent formation of dopamine. Inhibition of COMT is also likely to increase the half-life of various other catechol-structure compounds, including but not limited to epinephrine/norepinephrine, isoprenaline, rimiterol, dobutamine, fenoldopam, apomorphine, and α-methyldopa. A deficiency in norepinephrine has been linked to clinical depression, hence the use of COMT inhibitors could be usefull in the treatment of depression. COMT inhibitors are generally well tolerated with minimal side effects and are ultimately metabolized in the liver with only minor accumulation of metabolites in the body (Männistö, P. T. and S. Kaakkola (1999) Pharmacol. Rev. 51:593-628).

Copper-Zinc Superoxide Dismutases

Copper-zinc superoxide dismutases are compact homodimeric metalloenzymes involved in cellular defenses against oxidative damage. The enzymes contain one atom of zinc and one atom of copper per subunit and catalyze the dismutation of superoxide anions into O₂ and H₂O₂. The rate of dismutation is diffusion-limited and consequently enhanced by the presence of favorable electrostatic interactions between the substrate and enzyme active site. Examples of this class of enzyme have been identified in the cytoplasm of all the eukaryotic cells as well as in the periplasm of several bacterial species. Copper-zinc superoxide dismutases are robust enzymes that are highly resistant to proteolytic digestion and denaturing by urea and SDS. In addition to the compact structure of the enzymes, the presence of the metal ions and intrasubunit disulfide bonds is believed to be responsible for enzyme stability. The enzymes undergo reversible denaturation at temperatures as high as 70° C. (Battistoni, A. et al. (1998) J. Biol. Chem. 273:5655-5661).

Overexpression of superoxide dismutase has been implicated in enhancing freezing tolerance of transgenic alfalfa as well as providing resistance to environmental toxins such as the diphenyl ether herbicide, acifluorfen (McKersie, B. D. et al. (1993) Plant Physiol. 103:1155-1163). In addition, yeast cells become more resistant to freeze-thaw damage following exposure to hydrogen peroxide which causes the yeast cells to adapt to further peroxide stress by upregulating expression of superoxide dismutases. In this study, mutations to yeast superoxide dismutase genes had a more detrimental effect on freeze-thaw resistance than mutations which affected the regulation of glutathione metabolism, long suspected of being important in determining an organism's survival through the process of cryopreservation (Jong-In Park, J.-I. et al. (1998) J. Biol. Chem. 273:22921-22928).

Expression of superoxide dismutase is also associated with Mycobacterium tuberculosis, the organism that causes tuberculosis. Superoxide dismutase is one of the ten major proteins secreted by M. tuberculosis and its expression is upregulated approximately 5-fold in response to oxidative stress. M. tuberculosis expresses almost two orders of magnitude more superoxide dismutase than the nonpathogenic mycobacterium M. smegmatis, and secretes a much higher proportion of the expressed enzyme. The result is the secretion of 350-fold more enzyme by M. tuberculosis than M. smegmatis, providing substantial resistance to oxidative stress (Harth, G. and M. A. Horwitz (1999) J. Biol. Chem. 274:4281-4292).

The reduced expression of copper-zinc superoxide dismutases, as well as other enzymes with anti-oxidant capabilities, has been implicated in the early stages of cancer. The expression of copper-zinc superoxide dismutases is reduced in prostatic intraepithelial neoplasia and prostate carcinomas, (Bostwick, D. G. (2000) Cancer 89:123-134).

Phosphoesterases

Phosphotriesterases (PTE, paraoxonases) are enzymes that hydrolyze toxic organophosphorus compounds and have been isolated from a variety of tissues. Phosphotriesterases play a central role in the detoxification of insecticides by mammals. Birds and insects lack PTE, and as a result have reduced tolerance for organophosphorus compounds (Vilanova, E. and M. A. Sogorb (1999) Crit. Rev. Toxicol. 29:21-57). Phosphotriesterase activity varies among individuals and is lower in infants than adults. PTE knockout mice are markedly more sensitive to the organophosphate-based toxins diazoxon and chlorpyrifos oxon (Furlong, C. E., et al. (2000) Neurotoxicology 21:91-100). Phosphotriesterase is also implicated in atherosclerosis and diseases involving lipoprotein metabolism.

Glycerophosphoryl diester phosphodiesterase (also known as glycerophosphodiester phosphodiesterase) is a phosphodiesterase which hydrolyzes deacetylated phospholipid glycerophosphodiesters to produce sn-glycerol-3-phosphate and an alcohol. Glycerophosphocholine, glycerophosphoethanolamine, glycerophosphoglycerol, and glycerophosphoinositol are examples of substrates for glycerophosphoryl diester phosphodiesterases. A glycerophosphoryl diester phosphodiesterase from E. coli has broad specificity for glycerophosphodiester substrates (Larson, T. J. et al. (1983) J. Biol. Chem. 248:5428-5432).

Cyclic nucleotide phosphodiesterases (PDEs) are crucial enzymes in the regulation of the cyclic nucleotides cAMP and cGMP. cAMP and cGMP function as intracellular second messengers to transduce a variety of extracellular signals including hormones, light, and neurotransmitters. PDEs degrade cyclic nucleotides to their corresponding monophosphates, thereby regulating the intracellular concentrations of cyclic nucleotides and their effects on signal transduction. Due to their roles as regulators of signal transduction, PDEs have been extensively studied as chemotherapeutic targets (Perry, M. J. and G. A. Higgs (1998) Curr. Opin. Chem. Biol. 2:472-481; Torphy, J. T. (1998) Am. J. Resp. Crit. Care Med. 157:351-370).

Families of mammalian PDEs have been classified based on their substrate specificity and affinity, sensitivity to cofactors, and sensitivity to inhibitory agents (Beavo, J. A. (1995) Physiol. Rev. 75:725-748; Conti, M. et al. (1995) Endocrine Rev. 16:370-389). Several of these families contain distinct genes, many of which are expressed in different tissues as splice variants. Within PDE families, there are multiple isozymes and multiple splice variants of these isozymes (Conti, M. and S.-L. C. Jin (1999) Prog. Nucleic Acid Res. Mol. Biol. 63:1-38). The existence of multiple PDE families, isozymes, and splice variants is an indication of the variety and complexity of the regulatory pathways involving cyclic nucleotides (Houslay, M. D. and G. Milligan (1997) Trends Biochem. Sci. 22:217-224).

Type 1 PDEs (PDE1s) are Ca²⁺/calmodulin-dependent and appear to be encoded by at least three different genes, each having at least two different splice variants (Kakkar, R. et al. (1999) Cell Mol. Life Sci. 55:1164-1186). PDE1s have been found in the lung, heart, and brain. Some PDE1 isozymes are regulated in vitro by phosphorylation/dephosphorylation. Phosphorylation of these PDE1 isozymes decreases the affinity of the enzyme for calmodulin, decreases PDE activity, and increases steady state levels of cAMP (Kakkar et al., supra). PDE1s may provide useful therapeutic targets for disorders of the central nervous system and the cardiovascular and immune systems, due to the involvement of PDE1s in both cyclic nucleotide and calcium signaling (Perry and Higgs, supra).

PDE2s are cGMP-stimulated PDEs that have been found in the cerebellum, neocortex, heart, kidney, lung, pulmonary artery, and skeletal muscle (Sadhu, K. et al. (1999) J. Histochem. Cytochem. 47:895-906). PDE2s are thought to mediate the effects of cAMP on catecholamine secretion, participate in the regulation of aldosterone (Beavo, supra), and play a role in olfactory signal transduction (Juilfs, D. M. et al. (1997) Proc. Natl. Acad. Sci. USA 94:3388-3395).

PDE3s have high affinity for both cGMP and cAMP, and so these cyclic nucleotides act as competitive substrates for PDE3s. PDE3s play roles in stimulating myocardial contractility, inhibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. The PDE3 family of phosphodiesterases are sensitive to specific inhibitors such as cilostamide, enoximone, and lixazinone. Isozymes of PDE3 can be regulated by cAMP-dependent protein kinase, or by insulin-dependent kinases (Degerman, E. et al. (1997) J. Biol. Chem. 272:6823-6826).

PDE4s are specific for cAMP; are localized to airway smooth muscle, the vascular endothelium, and all inflammatory cells; and can be activated by cAMP-dependent phosphorylation. Since elevation of cAMP levels can lead to suppression of inflammatory cell activation and to relaxation of bronchial smooth muscle, PDE4s have been studied extensively as possible targets for novel anti-inflammatory agents, with special emphasis placed on the discovery of asthma treatments. PDE4 inhibitors are currently undergoing clinical trials as treatments for asthma, chronic obstructive pulmonary disease, and atopic eczema. All four known isozymes of PDE4 are susceptible to the inhibitor rolipram, a compound which has been shown to improve behavioral memory in mice (Barad, M. et al. (1998) Proc. Natl. Acad. Sci. USA 95:15020-15025). PDE4 inhibitors have also been studied as possible therapeutic agents against acute lung injury, endotoxemia, rheumatoid arthritis, multiple sclerosis, and various neurological and gastrointestinal indications (Doherty, A. M. (1999) Curr. Opin. Chem. Biol. 3:466-473).

PDE5 is highly selective for cGMP as a substrate (Turko, I. V. et al. (1998) Biochemistry 37:4200-4205), and has two allosteric cGMP-specific binding sites (McAllister-Lucas, L. M. et al. (1995) J. Biol. Chem. 270:30671-30679). Binding of cGMP to these allosteric binding sites seems to be important for phosphorylation of PDE5 by cGMP-dependent protein kinase rather than for direct regulation of catalytic activity. High levels of PDE5 are found in vascular smooth muscle, platelets, lung, and kidney. The inhibitor zaprinast is effective against PDE5 and PDE1s. Modification of zaprinast to provide specificity against PDE5 has resulted in sildenafil (VIAGRA; Pfizer, Inc., New York N.Y.), a treatment for male erectile dysfunction (Terrett, N. et al. (1996) Bioorg. Med. Chem. Lett. 6:1819-1824). Inhibitors of PDE5 are currently being studied as agents for cardiovascular therapy (Perry and Higgs, supra).

PDE6s, the photoreceptor cyclic nucleotide phosphodiesterases, are crucial components of the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which are thought to play a regulatory role in PDE6 function (Artemyev, N. O. et al. (1998) Methods 14:93-104). Defects in PDE6s have been associated with retinal disease. Retinal degeneration in the rd mouse (Yan, W. et al. (1998) Invest. Opthalmol. Vis. Sci. 39:2529-2536), autosomal recessive retinitis pigmentosa in humans (Danciger, M. et al. (1995) Genomics 30:1-7), and rod/cone dysplasia 1 in Irish Setter dogs (Suber, M. L. et al. (1993) Proc. Natl. Acad. Sci. USA 90:3968-3972) have been attributed to mutations in the PDE6B gene.

The PDE7 family of PDEs consists of only one known member having multiple splice variants (Bloom, T. J. and J. A. Beavo (1996) Proc. Natl. Acad. Sci. USA 93:14188-14192). PDE7s are cAMP specific, but little else is known about their physiological function. Although mRNAs encoding PDE7s are found in skeletal muscle, heart, brain, lung, kidney, and pancreas, expression of PDE7 proteins is restricted to specific tissue types (Han, P. et al. (1997) J. Biol. Chem. 272:16152-16157; Perry and Higgs, supra). PDE7s are very closely related to the PDE4 family; however, PDE7s are not inhibited by rolipram, a specific inhibitor of PDE4s (Beavo, supra).

PDE8s are cAMP specific, and are closely related to the PDE4 family. PDE8s are expressed in thyroid gland, testis, eye, liver, skeletal muscle, heart, kidney, ovary, and brain. The cAMP-hydrolyzing activity of PDE8s is not inhibited by the PDE inhibitors rolipram, vinpocetine, milrinone, IBMX (3-isobutyl-1-methylxanthine), or zaprinast, but PDE8s are inhibited by dipyridamole (Fisher, D. A. et al. (1998) Biochem. Biophys. Res. Commun. 246:570-577; Hayashi, M. et al. (1998) Biochem. Biophys. Res. Commun. 250:751-756; Soderling, S. H. et al. (1998) Proc. Natl. Acad. Sci. USA 95:8991-8996).

PDE9s are cGMP specific and most closely resemble the PDE8 family of PDEs. PDE9s are expressed in kidney, liver, lung, brain, spleen, and small intestine. PDE9s are not inhibited by sildenafil (VIAGRA; Pfizer, Inc., New York N.Y.), rolipram, vinpocetine, dipyridamole, or IBMX (3-isobutyl-1-methylxanthine), but they are sensitive to the PDE5 inhibitor zaprinast (Fisher, D. A. et al. (1998) J. Biol. Chem. 273:15559-15564; Soderling, S. H. et al. (1998) J. Biol. Chem. 273:15553-15558).

PDE10s are dual-substrate PDEs, hydrolyzing both cAMP and cGMP. PDE10s are expressed in brain, thyroid, and testis. (Soderling, S. H. et al. (1999) Proc. Natl. Acad. Sci. USA 96:7071-7076; Fujishige, K. et al. (1999) J. Biol. Chem. 274:18438-18445; Loughney, K. et al (1999) Gene 234:109-117).

PDEs are composed of a catalytic domain of about 270-300 amino acids, an N-terminal regulatory domain responsible for binding cofactors, and, in some cases, a hydrophilic C-terminal domain of unknown function (Conti and Jin, supra). A conserved, putative zinc-binding motif has been identified in the catalytic domain of all PDEs. N-terminal regulatory domains include non-catalytic cGMP-binding domains in PDE2s, PDE5s, and PDE6s; calmodulin-binding domains in PDE1s; and domains containing phosphorylation sites in PDE3s and PDE4s. In PDE5, the N-terminal cGMP-binding domain spans about 380 amino acid residues and comprises tandem repeats of a conserved sequence motif (McAllister-Lucas, L. M. et al. (1993) J. Biol. Chem. 268:22863-22873). The NKXnD motif has been shown by mutagenesis to be important for cGMP binding (Turko, I. V. et al. (1996) J. Biol. Chem. 271:22240-22244). PDE families display approximately 30% amino acid identity within the catalytic domain; however, isozymes within the same family typically display about 85-95% identity in this region (e.g. PDE4A vs PDE4B). Furthermore, within a family there is extensive similarity (>60%) outside the catalytic domain; while across families, there is little or no sequence similarity outside this domain.

Many of the constituent functions of immune and inflammatory responses are inhibited by agents that increase intracellular levels of cAMP (Verghese, M. W. et al. (1995) Mol. Pharmacol. 47:1164-1171). A variety of diseases have been attributed to increased PDE activity and associated with decreased levels of cyclic nucleotides. For example, a form of diabetes insipidus in mice has been associated with increased PDE4 activity, an increase in low-Kₘ cAMP PDE activity has been reported in leukocytes of atopic patients, and PDE3 has been associated with cardiac disease.

Many inhibitors of PDEs have undergone clinical evaluation (Perry and Higgs, supra; Torphy, T. J. (1998) Am. J. Respir. Crit. Care Med. 157:351-370). PDE3 inhibitors are being developed as antithrombotic agents, antihypertensive agents, and as cardiotonic agents useful in the treatment of congestive heart failure. Rolipram, a PDE4 inhibitor, has been used in the treatment of depression, and other PDE4 inhibitors have an anti-inflammatory effect. Rolipram may inhibit HIV-1 replication (Angel, J. B. et al. (1995) AIDS 9:1137-1144). Additionally, rolipram suppresses the production of cytokines such as TNF-a and b and interferon g, and thus is effective against encephalomyelitis. Rolipram may also be effective in treating tardive dyskinesia and multiple sclerosis (Sommer, N. et al. (1995) Nat. Med. 1:244-248; Sasaki, H. et al. (1995) Eur. J. Pharmacol. 282:71-76). Theophylline is a nonspecific PDE inhibitor used in treatment of bronchial asthma and other respiratory diseases. Theophylline is believed to act on airway smooth muscle function and in an anti-inflammatory or immunomodulatory capacity (Banner, K. H. and C. P. Page (1995) Eur. Respir. J. 8:996-1000). Pentoxifylline is another nonspecific PDE inhibitor used in the treatment of intermittent claudication and diabetes-induced peripheral vascular disease. Pentoxifylline is also known to block TNF-a production and may inhibit HIV-1 replication (Angel et al., supra).

PDEs have been reported to affect cellular proliferation of a variety of cell types (Conti et al. (1995) Endocrine Rev. 16:370-389) and have been implicated in various cancers. Growth of prostate carcinoma cell lines DU145 and LNCaP was inhibited by delivery of cAMP derivatives and PDE inhibitors (Bang, Y. J. et al. (1994) Proc. Natl. Acad. Sci. USA 91:5330-5334). These cells also showed a permanent conversion in phenotype from epithelial to neuronal morphology. It has also been suggested that PDE inhibitors can regulate mesangial cell proliferation (Matousovic, K. et al. (1995) J. Clin. Invest. 96:401-410) and lymphocyte proliferation (Joulain, C. et al. (1995) J. Lipid Mediat. Cell Signal. 11:63-79). One cancer treatment involves intracellular delivery of PDEs to particular cellular compartments of tumors, resulting in cell death (Deonarain, M. P. and A. A. Epenetos (1994) Br. J. Cancer 70:786-794).

Members of the UDP glucuronyltransferase family (UGTs) catalyze the transfer of a glucuronic acid group from the cofactor uridine diphosphate-glucuronic acid (UDP-glucuronic acid) to a substrate. The transfer is generally to a nucleophilic heteroatom (O, N, or S). Substrates include xenobiotics which have been functionalized by Phase I reactions, as well as endogenous compounds such as bilirubin, steroid hormones, and thyroid hormones. Products of glucuronidation are excreted in urine if the molecular weight of the substrate is less than about 250 g/mol, whereas larger glucuronidated substrates are excreted in bile.

UGTs are located in the microsomes of liver, kidney, intestine, skin, brain, spleen, and nasal mucosa, where they are on the same side of the endoplasmic reticulum membrane as cytochrome P450 enzymes and flavin-containing monooxygenases. UGTs have a C-terminal membrane-spanning domain which anchors them in the endoplasmic reticulum membrane, and a conserved signature domain of about 50 amino acid residues in their C terminal section (PROSITE PDOC00359 UDP-glycosyltransferase signature).

UGTs involved in drug metabolism are encoded by two gene families, UGT1 and UGT2. Members of the UGT1 family result from alternative splicing of a single gene locus, which has a variable substrate binding domain and constant region involved in cofactor binding and membrane insertion. Members of the UGT2 family are encoded by separate gene loci, and are divided into two families, UGT2A and UGT2B. The 2A subfamily is expressed in olfactory epithelium, and the 2B subfamily is expressed in liver microsomes. Mutations in UGT genes are associated with hyperbilirubinemia (OMIM #143500 Hyperbilirubinemia 1); Crigler-Najjar syndrome, characterized by intense hyperbilirubinemia from birth (OMIM #218800 Crigler-Najjar syndrome); and a milder form of hyperbilirubinemia termed Gilbert's disease (OMIM #191740 UGT1).

Thioesterases

Two soluble thioesterases involved in fatty acid biosynthesis have been isolated from mammalian tissues, one which is active only toward long-chain fatty-acyl thioesters and one which is active toward thioesters with a wide range of fatty-acyl chain-lengths. These thioesterases catalyze the chain-terminating step in the de novo biosynthesis of fatty acids. Chain termination involves the hydrolysis of the thioester bond which links the fatty acyl chain to the 4′-phosphopantetheine prosthetic group of the acyl carrier protein (ACP) subunit of the fatty acid synthase (Smith, S. (1981a) Methods Enzymol. 71:181-188; Smith, S. (1981b) Methods Enzymol. 71:188-200).

E. coli contains two soluble thioesterases, thioesterase I which is active only toward long-chain acyl thioesters, and thioesterase II (TEII) which has a broad chain-length specificity (Naggert, J. et al. (1991) J. Biol. Chem. 266:11044-11050). E. coli TEII does not exhibit sequence similarity with either of the two types of mammalian thioesterases which function as chain-terminating enzymes in de novo fatty acid biosynthesis. Unlike the mammalian thioesterases, E. coli TEII lacks the characteristic serine active site gly-X-ser-X-gly sequence motif and is not inactivated by the serine modifying agent diisopropyl fluorophosphate. However, modification of histidine 58 by iodoacetamide and diethylpyrocarbonate abolished TEII activity. Overexpression of TEII did not alter fatty acid content in E. coli, which suggests that it does not function as a chain-terminating enzyme in fatty acid biosynthesis (Naggert et al., supra). For that reason, Naggert et al. (supra) proposed that the physiological substrates for E. coli TEII may be coenzyme A (CoA)-fatty acid esters instead of ACP-phosphopanthetheine-fatty acid esters.

Carboxylesterases

Mammalian carboxylesterases are a multigene family expressed in a variety of tissues and cell types. Acetylcholinesterase, butyrylcholinesterase, and carboxylesterase are grouped into the serine superfamily of esterases (B-esterases). Other carboxylesterases include thyroglobulin, thrombin, Factor IX, gliotactin, and plasminogen. Carboxylesterases catalyze the hydrolysis of ester- and amide-groups from molecules and are involved in detoxification of drugs, environmental toxins, and carcinogens. Substrates for carboxylesterases include short- and long-chain acyl-glycerols, acylcarnitine, carbonates, dipivefrin hydrochloride, cocaine, salicylates, capsaicin, palmitoyl-coenzyme A, imidapril, haloperidol, pyrrolizidine alkaloids, steroids, p-nitrophenyl acetate, malathion, butanilicaine, and isocarboxazide. Carboxylesterases are also important for the conversion of prodrugs to free acids, which may be the active form of the drug (e.g., lovastatin, used to lower blood cholesterol) (reviewed in Satoh, T. and Hosokawa, M. (1998) Annu. Rev. Pharmacol. Toxicol. 38:257-288). Neuroligins are a class of molecules that (i) have N-terminal signal sequences, (ii) resemble cell-surface receptors, (iii) contain carboxylesterase domains, (iv) are highly expressed in the brain, and (v) bind to neurexins in a calcium-dependent manner. Despite the homology to carboxylesterases, neuroligins lack the active site serine residue, implying a role in substrate binding rather than catalysis (Ichtchenko, K. et al. (1996) J. Biol. Chem. 271:2676-2682).

Squalene Epoxidase

Squalene epoxidase (squalene monooxygenase, SE) is a microsomal membrane-bound, FAD-dependent oxidoreductase that catalyzes the first oxygenation step in the sterol biosynthetic pathway of eukaryotic cells. Cholesterol is an essential structural component of cytoplasmic membranes acquired via the LDL receptor-mediated pathway or the biosynthetic pathway. SE converts squalene to 2,3(S)-oxidosqualene, which is then converted to lanosterol and then cholesterol.

High serum cholesterol levels result in the formation of atherosclerotic plaques in the arteries of higher organisms. This deposition of highly insoluble lipid material onto the walls of essential blood vessels results in decreased blood flow and potential necrosis. HMG-CoA reductase is responsible for the first committed step in cholesterol biosynthesis, conversion of 3-hydroxyl-3-methyl-glutaryl CoA (HMG-CoA) to mevalonate. HMG-CoA is the target of a number of pharmaceutical compounds designed to lower plasma cholesterol levels, but inhibition of MHG-CoA also results in the reduced synthesis of non-sterol intermediates required for other biochemical pathways. Since SE catalyzes a rate-limiting reaction that occurs later in the sterol synthesis pathway with cholesterol as the only end product, SE is a better ideal target for the design of anti-hyperlipidemic drugs (Nakamura, Y. et al. (1996) 271:8053-8056).

Epoxide Hydrolases

Epoxide hydrolases catalyze the addition of water to epoxide-containing compounds, thereby hydrolyzing epoxides to their corresponding 1,2-diols. They are related to bacterial haloalkane dehalogenases and show sequence similarity to other members of the α/β hydrolase fold family of enzymes. This family of enzymes is important for the detoxification of xenobiotic epoxide compounds which are often highly electrophilic and destructive when introduced. Examples of epoxide hydrolase reactions include the hydrolysis of some leukotoxin to leukotoxin diol, and isoleukotoxin to isoleukotoxin diol. Leukotoxins alter membrane permeability and ion transport and cause inflammatory responses. In addition, epoxide carcinogens are produced by cytochrome P450 as intermediates in the detoxification of drugs and environmental toxins. Epoxide hydrolases possess a catalytic triad composed of Asp, Asp, and His (Arand, M. et al. (1996) J. Biol. Chem. 271:4223-4229; Rink, R. et al. (1997) J. Biol. Chem. 272:14650-14657; Argiriadi, M. A. et al. (2000) J. Biol. Chem. 275:15265-15270).

Enzymes Involved in Tyrosine Catalysis

The degradation of the amino acid tyrosine, to either succinate and pyruvate or fumarate and acetoacetate, requires a large number of enzymes and generates a large number of intermediate compounds. In addition, many xenobiotic compounds may be metabolized using one or more reactions that are part of the tyrosine catabolic pathway. Enzymes involved in the degradation of tyrosine to succinate and pyruvate (e.g., in Arthrobacter species) include 4-hydroxyphenylpyruvate oxidase, 4-hydroxyphenylacetate 3-hydroxylase, 3,4-dihydroxyphenylacetate 2,3-dioxygenase, 5-carboxymethyl-2-hydroxymuconic semialdehyde dehydrogenase, trans,cis-5-carboxymethyl-2-hydroxymuconate isomerase, homoprotocatechuate isomerase/decarboxylase, cis-2-oxohept-3-ene-1,7-dioate hydratase, 2,4-dihydroxyhept-trans-2-ene-1,7-dioate aldolase, and succinic semialdehyde dehydrogenase. Enzymes involved in the degradation of tyrosine to fumarate and acetoacetate (e.g., in Pseudomonas species) include 4-hydroxyphenylpyruvate dioxygenase, homogentisate 1,2-dioxygenase, maleylacetoacetate isomerase, fumarylacetoacetase and 4-hydroxyphenylacetate. Additional enzymes associated with tyrosine metabolism in different organisms include 4-chlorophenylacetate-3,4-dioxygenase, aromatic aminotransferase, 5-oxopent-3-ene-1,2,5-tricarboxylate decarboxylase, 2-oxo-hept-3-ene-1,7-dioate hydratase, and 5-carboxymethyl-2-hydroxymuconate isomerase (Ellis, L. B. M. et al., (1999) Nucleic Acids Res. 27:373-376; Wackett, L. P. and Ellis, L. B. M. (1996) J. Microbiol. Meth. 25:91-93; and Schmidt, M. (1996) Amer. Soc. Microbiol. News 62:102).

In humans, acquired or inherited genetic defects in enzymes of the tyrosine degradation pathway may result in hereditary tyrosinemia. One form of this disease, hereditary tyrosinemia 1 (H1) is caused by a deficiency in the enzyme fumarylacetoacetate hydrolase, the last enzyme in the pathway in organisms that metabolize tyrosine to fumarate and acetoacetate. HT1 is characterized by progressive liver damage beginning at infancy, and increased risk for liver cancer (Endo, F. et al. (1997) J. Biol. Chem. 272:24426-24432).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Genes Expressed in Breast Cancer

The potential application of gene expression profiling is particularly relevant to improving diagnosis, prognosis, and treatment of disease. For example, both the levels and sequences expressed in tissues from subjects with breast cancer may be compared with the levels and sequences expressed in normal tissue.

There are more than 180,000 new cases of breast cancer diagnosed each year, and the mortality rate for breast cancer approaches 10% of all deaths in females between the ages of 45-54 (K. Gish (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou C M et al. (2000) Nature 406:747-752).

Breast cancer is a genetic disease commonly caused by mutations in breast tissues. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to noninherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie et al., supra, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S S et al. (1994) Am J Clin Pathol 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou Z et al. (1998) Int J Cancer 78:95-99; Chen, L et al. (1990) Oncogene 5:1391-1395; Ulrix W et al. (1999) FEBS Lett 455:23-26; Sager, R et al. (1996) Curr Top Microbiol Immunol 213:51-64; and Lee, S W et al. (1992) Proc Natl Acad Sci USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba I I et al. (1998) Clin Cancer Res 4:2931-2938). Such a model is particularly useful for comparing phenotypic and molecular characteristics of human mammary epithelial cells at various stages of malignant transformation.

Genes Expressed in Colon Cancer

The potential application of gene expression profiling is particularly relevant to improving diagnosis, prognosis, and treatment of disease. For example, both the levels and sequences expressed in tissues from subjects with colon cancer may be compared with the levels and sequences expressed in normal tissue.

Colorectal cancer is the fourth most common cancer and the second most common cause of cancer death in the United States with approximately 130,000 new cases and 55,000 deaths per year. Colon and rectal cancers share many environmental risk factors and both are found in individuals with specific genetic syndromes. (See Potter, J D (1999) J Natl Cancer Institute 91:916-932 for a review of colorectal cancer.) Colon cancer is the only cancer that occurs with approximately equal frequency in men and women, and the five-year survival rate following diagnosis of colon cancer is around 55% in the United States (Ries et al. (1990) National Institutes of Health, DHHS Publ No. (NIH)90-2789).

Colon cancer is causally related to both genes and the environment. Several molecular pathways have been linked to the development of colon cancer, and the expression of key genes in any of these pathways may be lost by inherited or acquired mutation or by hypermethylation. There is a particular need to identify genes for which changes in expression may provide an early indicator of colon cancer or a predisposition for the development of colon cancer.

For example, it is well known that abnormal patterns of DNA methylation occur consistently in human tumors and include, simultaneously, widespread genomic hypomethylation and localized areas of increased methylation. In colon cancer in particular, it has been found that these changes occur early in tumor progression such as in premalignant polyps that precede colon cancer. Indeed, DNA methyltransferase, the enzyme that performs DNA methylation, is significantly increased in histologically normal mucosa from patients with colon cancer or the benign polyps that precede cancer, and this increase continues during the progression of colonic neoplasms (Wafik, S et al. (1991) Proc Natl Acad Sci USA 88:3470-3474). Increased DNA methylation occurs in G+C rich areas of genomic DNA termed “CpG islands” that are important for maintenance of an “open” transcriptional conformation around genes, and that hypermethylation of these regions results in a “closed” conformation that silences gene transcription. It has been suggested that the silencing or downregulation of differentiation genes by such abnormal methylation of CpG islands may prevent differentiation in immortalized cells (Anteguera, F. et al. (1990) Cell 62:503-514).

Familial Adenomatous Polyposis (FAP) is a rare autosomal dominant syndrome that precedes colon cancer and is caused by an inherited mutation in the adenomatous polyposis coli (APC) gene. FAP is characterized by the early development of multiple colorectal adenomas that progress to cancer at a mean age of 44 years. The APC gene is a part of the APC-β-catenin-Tcf (T-cell factor) pathway. Impairment of this pathway results in the loss of orderly replication, adhesion, and migration of colonic epithelial cells that results in the growth of polyps. A series of other genetic changes follow activation of the APC-β-catenin-Tcf pathway and accompanies the transition from normal colonic mucosa to metastatic carcinoma. These changes include mutation of the K-Ras proto-oncogene, changes in methylation patterns, and mutation or loss of the tumor suppressor genes p53 and Smad4/DPC4. While the inheritance of a mutated APC gene is a rare event, the loss or mutation of APC and the consequent effects on the APC-β-catenin-Tcf pathway is believed to be central to the majority of colon cancers in the general population.

Hereditary nonpolyposis Colorectal Cancer (HNPCC) is another inherited autosomal dominant syndrome with a less well defined phenotype than FAP. HNPCC, which accounts for about 2% of colorectal cancer cases, is distinguished by the tendency to early onset of cancer and the development of other cancers, particularly those involving the endometrium, urinary tract, stomach and biliary system. HNPCC results from the mutation of one or more genes in the DNA mis-match repair (MMR) pathway. Mutations in two human MMR genes, MSH2 and MLH1, are found in a large majority of HNPCC families identified to date. The DNA MMR pathway identifies and repairs errors that result from the activity of DNA polymerase during replication. Furthermore, loss of My activity contributes to cancer progression through accumulation of other gene mutations and deletions, such as loss of the BAX gene which controls apoptosis, and the TGFβ receptor II gene which controls cell growth. Because of the potential for irreparable damage to DNA in an individual with a DNA MMR defect, progression to carcinoma is more rapid than usual.

Although ulcerative colitis is a minor contributor to colon cancer, affected individuals have about a 20-fold increase in risk for developing cancer. Progression is characterized by loss of the p53 gene which may occur early, appearing even in histologically normal tissue. The progression of the disease from ulcerative colitis to dysplasia/carcinoma without an intermediate polyp state suggests a high degree of mutagenic activity resulting from the exposure of proliferating cells in the colonic mucosa to the colonic contents.

Almost all colon cancers arise from cells in which the estrogen receptor (ER) gene has been silenced. The silencing of ER gene transcription is age related and linked to hypermethylation of the ER gene (Issa, J-P J et al. (1994) Nature Genetics 7:536-540). Introduction of an exogenous ER gene into cultured colon carcinoma cells results in marked growth suppression. The connection between loss of the ER protein in colonic epithelial cells and the consequent development of cancer has not been established.

Clearly there are a number of genetic alterations associated with colon cancer and with the development and progression of the disease, particularly the downregulation or deletion of genes, that potentially provide early indicators of cancer development, and which may also be used to monitor disease progression or provide possible therapeutic targets. The specific genes affected in a given case of colon cancer depend on the molecular progression of the disease. Identification of additional genes associated with colon cancer and the precancerous state would provide more reliable diagnostic patterns associated with the development and progression of the disease.

Genes Regulated in Dendritic Cell Differentiation

The potential application of gene expression profiling is particularly relevant to characterizing lineage differences during cellular development that will improve diagnosis, prognosis, and treatment of disease. For example, both the levels and sequences expressed in dendritic cells from subjects with autoimmunity may be compared with the levels and sequences expressed in dendritic cells from normal subjects.

Dendritic cells (DC) are antigen presenting cells (APC) that play a key role in the primary immune response because of their unique ability to present antigens to naive T cells. In addition, DC differentiate into separate subsets that sustain and regulate immune responses following initial contact with antigen. DC subsets include those that preferentially induce particular T helper 1 (Th1) or T helper 2 (Th2) responses and those that regulate B cell responses. Moreover, DC are increasingly being used to manipulate immune responses, either to downregulate an aberrant autoimmune response or to enhance vaccination or a tumor-specific response.

DC are functionally specialized in correlation with their particular differentiation state. CD34+ myeloid cells found in the bone marrow mature in response to as yet unclear signals into CD 14+ CD11c+ monocytes. An innate or antigen non-specific response takes place initially when monocytes circulate to nonlymphoid tissues and respond to lipopolysaccharide (LPS), a bacterially-derived mitogen, and viruses. Such direct encounter with antigen causes secretion of pro-inflammatory cytokines that attract and regulate natural killer cells, macrophages, and eosinophils in the first line of defense against invading pathogens. Monocytes then mature into DC, which capture antigen highly efficiently through endocytosis and antigen receptor uptake. Antigen processing and presentation trigger activation and differentiation into mature DC that express MHC class II molecules on the cell surface and efficiently activate T cells, initiating antigen-specific T cell and B cell responses. In turn, T cells activate DC through CD40 ligand-CD40 interactions, which stimulate expression of the costimulatory molecules CD80 and CD86, the latter most potent in amplifying T cell responses. DC interaction via CD40 with T cells also stimulates the production of inflammatory cytokines such as TNF alpha and IL-1. Engagement of RANK, a member of the TNF receptor family by its ligand, TRANCE, which is expressed on activated T cells, enhances the survival of DC through inhibition of apoptosis, thereby enhancing T cell activation. The maturation and differentiation of monocytes into mature DC links the antigen non-specific innate immune response to the antigen-specific adaptive immune response.

The process by which monocytes differentiate into immature dendritic cells in vivo has not been fully elucidated. Incubation of monocytes with granulocyte-macrophage colony stimulating factor (GM-CSF) and interleukin (IL)-4 in vitro yields cells that exhibit functional and morphological characteristics equivalent to immature dendritic cells found in vivo. Moreover, incubation in vitro of immature dendritic cells with tumor necrosis factor alpha (TNF-a), CD40 ligand, LPS, or monocyte-conditioned medium yields mature dendritic cells that are potent activators of naive T cells.

The ability to manipulate DC in vitro and their capacity to mount an effective immune response with small numbers of DC and little antigen has led to potential immunotherapies for diseases such as cancer, AIDS, and infectious diseases; and enhancing vaccine efficacy. Spontaneous remissions of particular cancers such as renal cell carcinomas and melanomas indicate that the immune system can respond to tumor antigens and eliminate tumors. However, tumors escape immune surveillance through a number of means including secretion of IL-10, macrophage colony stimulating factor, IL-6, and vascular endothelial growth factor, all of which inhibit DC activity and promote tolerance of tumor tissue. Delivery of tumor antigen-loaded DC to tumors can induce tumor-specific rejection in animal models. Similarly, pathogens can escape immune surveillance by altering antigen processing and presentation pathways or interfering with maturation of antigen presenting cells. Rather than providing resistance, DC can complicate infection by hosting latent viruses such as Kaposi's virus and cytomegalovirus, complicating infection. HIV-1 and measles virus particles are efficiently produced in DC. Vaccines against tumors or infectious pathogens could be improved by systemic or local administration of DC loaded with tumor antigens or attenuated viral particles or components, respectively.

The expression of killer-inhibitor regulatory molecules, chemokines, chemokine receptors, and proteinases have been identified in DC through sequencing of ESTs. Continuing this search may reveal new lymphocyte-binding and antigen-processing molecules, transmembrane and secretory products, and transcription factors that may help to explain the specialized features of DC and allow manipulation of the immune system.

Genes Expressed in Foam Cell Differentiation

Atherosclerosis and the associated coronary artery disease and cerebral stroke represent the most common cause of death in industrialized nations. Although certain key risk factors have been identified, a full molecular characterization that elucidates the causes and provide care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus.

An early step in the development of atherosclerosis is formation of the “fatty streak”. Lipoproteins, such as the cholesterol-rich low-density lipoprotein (LDL), accumulate in the extracellular space of the vascular intima, and undergo modification. Oxidation of LDL occurs most avidly in the sub-endothelial space where circulating antioxidant defenses are less effective. The degree of LDL oxidation affects its interaction with target cells. “Minimally oxidized” LDL (MM-LDL) is able to bind to LDL receptor but not to the oxidized LDL (Ox-LDL) or “scavenger” receptors that have been identified, including scavenger receptor types A and B, CD36, CD68/macrosialin and LOX-1 (Navab et al. (1994) Arterioscler Thromb Vasc Biol 16:831-842; Kodama et al. (1990) Nature 343:531-535; Acton et al. (1994) J Biol Chem 269:21003-21009; Endemann et al. (1993) J Biol Chem 268:11811-11816; Ramprasad et al. (1996) Proc Natl Acad Sci 92:14833-14838; Kataoka et al. (1999) Circulation 99:3110-3117). MM-LDL can increase the adherence and penetration of monocytes, stimulate the release of monocyte chemotactic protein 1 (MCP-1) by endothelial cells, and induce scavenger receptor A (SRA) and CD36 expression in macrophages (Cushing et al. (1990) Proc Natl Acad Sci 87:5134-5138; Yoshida et al. (1998) Arterioscler Thromb Vasc Biol 18:794-802; Steinberg (1997) J Biol Chem 272:20963-20966). SRA and the other scavenger receptors can bind Ox-LDL and enhance uptake of lipoprotein particles.

Mononuclear phagocytes enter the intima, differentiate into macrophages, and ingest modified lipids including Ox-LDL. In most cell types, cholesterol content is tightly controlled by feedback regulation of LDL receptors and biosynthetic enzymes (Brown and Goldstein (1986) Science 232:34-47). In macrophages, however, the additional scavenger receptors lead to unregulated uptake of cholesterol (Brown and Goldstein (1983) Annu Rev Biochem 52:223-261) and accumulation of multiple intracellular lipid droplets producing a “foam cell” phenotype. Cholesterol-engorged and dead macrophages contribute most of the mass of early “fatty streak” plaques and typical “advanced” lesions of diseased arteries. Numerous studies have described a variety of foam cell responses that contribute to growth and rupture of atherosclerotic vessel wall plaques. These responses include production of multiple growth factors and cytokines, which promote proliferation and adherence of neighboring cells; chemokines, which further attract circulating monocytes into the growing plaque; proteins, which cause remodeling of the extracellular matrix; and tissue factor, which can trigger thrombosis (Ross (1993) Nature 362:801-809; Quin et al. (1987) Proc Natl Acad Sci 84:2995-2998). Thus, cholesterol-loaded macrophages which occur in abundance in most stages of the atherosclerotic plaque formation contribute to inception of the atheroscerotic process and to eventual plaque rupture and occlusive thrombus.

During Ox-LDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase. Thus, genes differentially expressed during foam cell formation may reasonably be expected to be markers of the atherosclerotic process.

Alzheimer's Disease

The potential application of gene expression profiling is also relevant to improving diagnosis, prognosis, and treatment of diseases such as Alzheimer's disease. For example, both the levels and sequences expressed in tissues from subjects with Alzheimer's disease may be compared with the levels and sequences expressed in normal brain tissue.

Akzheimer's disease is a progressive neurodegenerative disorder that is characterized by the formation of senile plaques and neurofibrillary tangles containing amyloid beta peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

Approximately twenty million people worldwide suffer with dementia that results from Alzheimer's disease. The disease can be early onset affecting individuals as young as 30 years of age, or it can be familial or sporadic. Familial Alzheimer's disease was once thought to be inherited strictly as an autosomal dominant trait; however, this view is changing as more genetic determinants are isolated. For example, some normal allelic variants of apolipoprotein E (ApoE), which is found in senile plaques, can either protect against or increase the risk of developing the disease (Strittmatter et al. (1993) Proc Natl Acad Sci 90:1977-1981).

Mutations in four genes are known to predispose an individual to Alzheimer's disease: ApoE, amyloid precursor protein (APP), presenilin-1, and presenilin-2 (Selkoe (1999) Nature 399:A23-A31). The e4 allele of the ApoE gene confers increased risk for late onset Alzheimer's disease. β-amyloid protein (Aβ) is the major component of senile plaques, and it is normally formed when β- and γ-secretases cleave APP. In Alzheimer's disease patients, large quantities of Aβ are generated and accumulate extracellularly in these neuropathological plaques. Efforts to understand the mechanism underlying Aβ deposition have recently focused on the APP-cleaving secretases. In fact, two yeast aspartyl proteases have been shown to process APP in vitro (Zhang et al. (1997) Biochim Biophys Acta 1359:110-122). Evidence using peptidomimetic probes further confirms that the secretases are intramembrane-cleaving aspartyl proteases (Wolfe et al. (1999) Biochemistry 38:4720-4727). The presenilin-1 gene is a candidate for the γ-secretase that cleaves the APP carboxyl terminus. Several lines of evidence support the involvement of preosenilins in the disease process. Presenilin can be coimmunoprecipitated with APP, and mutations in the presenilin genes increase production of the 42-amino acid peptide form of Aβ. These missense point mutations result in a particularly aggressive, early onset form of the disease (Haas and DeStrooper (1999) Science 286:916-919).

The proteases, BACE1 and BACE2 (β-site APP cleaving enzymes 1 and 2) which appear to be β-secretases, are potential therapeutic targets because of their ability to cleave APP. Vasser et al. (1999; Science 286:735-741) have found that BACE1 is an aspartyl protease with O-secretase activity which cleaves APP to produce Aβ peptide in vitro. It is expressed at moderate levels across all brain regions and is concentrated in neurons but not in glia. BACE2, which has 52% amino acid identity with BACE1, has been described by Saunders et al (1999; Science ²86:1255a). Whereas BACE1 maps to the long arm of chromosome 11, BACE2 maps to the Down syndrome region of chromosome 21 (Acquati et al. (2000) 468: 59-64; Saunders et al. supra). This location is significant because middle-aged Down syndrome patients have enhanced β-amyloid deposits. Other members of the BACE family may also participate in this APP cleavage: the amino terminals of Aβ peptides appear to be cleaved heterogeneously indicating that there may be several β-secretases involved in APP processing (Vasser (1999) Science 286:735-741).

Associations between Alzheimer's disease and many other genes and proteins have been reported. Fetal Alzheimer antigen (FALZ) and synuclein α (SNCA) are found in brain plaques and tangles. Inheritance of some gene polymorphisms is also linked to increased risk of developing the disease. For example, a polymorphism in the gene encoding β2-macroglobulin, a protein that can act as a protease inhibitor, is associated with increased risk for developing a late-onset form of Alzheimer's disease.

C3A Cell Line

The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) convertion of ammonia to urea and glutamine; iv) metabolize aromatic amino acids; and v) are able to proliferate in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-416).

Clofibrate is an hypolidemic drug which lowers elevated levels of serum triglycerides. In rodents, chronic treatment produces hepatomegaly and an increase in hepatic peroxisomes (peroxisome proliferation). Peroxisome proliferators (PPs) are a class of drugs which activate the PP-activated receptor in rodent liver, leading to enzyme induction, stimulation of S-phase, and a suppression of apoptosis (Hasmall and Roberts (1999) Pharmacol. Ther. 82:63-70). PPs include the fibrate class of hypolideric drugs, phenobarbitone, thiazolidinediones, certain non-steroidal anti-inflammatory drugs, and naturally-occuring fatty acid-derived molecules (Gelman et al. (1999) Cell. Mol. Life Sci. 55:932-943). Clofibrate has been shown to increase levels of cytochrome P450 4A. It is also involved in transcription of O-oxidation genes as well as induction of PP-activated receptors (Kawashima et al. (1997) Arch. Biochem. Biophys. 347:148-154). Peroxisome proliferation that is induced by both clofibrate and the chemically-related compound fenofibrate is mediated by a common inhibitory effect on mitochondrial membrane depolarization (Zhou and Wallace (1999) Toxicol. Sci. 48:82-89).

Elevated triglyceride and cholesterol levels may be dramatically lowered by treatment with fibric acid agents. Improvement in angina and intermittent claudication also occurs. Gemfibrozil treatment for mild hypertriglyceridemia (e.g., triglycerides <400 mg/dl) usually produces a decrease in triglyceride levels of 50% or more, an increase in HDL cholesterol concentrations of 15-25%, and either no change or an increase in LDL cholesterol levels, particularly in subjects with familial combined hyperlipidemia. Second generation agents, such as fenofibrate, bezafibrate, and ciprofibrate, lower VLDL levels to a degree similar to that produced by gemfibrozil, but they also decrease LDL levels by 15-20%. Patients with more marked hypertriglyceridemia, for example with triglyceride levels of 400 to 1000 mg/dl, experience a similar reduction in triglycerides, but increases in LDL of 10-30% frequently occur. In contrast, treatment of patients with heterozygous familial hypercholesterolemia usually produces a decrease in LDL levels of 10% with gemfibrozil, and of 20-30% with the other agents. Clofibrate is available for oral administration and it may be useful in patients with dysbetalipoproteinemia who do not respond to gemfibrozil. Fenofibrate is not available in the United States but is widely used in Europe and may be helpful as an adjunct to gemfibrozil, to lower LDL levels.

Human Peripheral Blood Mononuclear Cells

Human peripheral blood mononuclear cells (PBMCs) represent the major cellular components of the immune system. PBMCs contain B lymphocytes, T lymphocytes, NK cells, dendritic and progenitor cells.

Staphylococcal exotoxins specifically activate human T cells, expressing an appropriate TCR-Vbeta chain. Although polyclonal in nature, T cells activated by Staphylococcal exotoxins require antigen presenting cells (APCs) to present the exotoxin molecules to the T cells and deliver the costimulatory signals required for optimum T cell activation. Although, Staphylococcal exotoxins must be presented to T cells by APCs, these molecules are not required to be processed by APC. Indeed, Staphylococcal exotoxins directly bind to a non-polymorphic portion of the human MHC class II molecules, bypassing the need for capture, cleavage, and binding of the peptides to the polymorphic antigenic groove of the MHC class II molecules.

There is a need in the art for new compositions, including nucleic acids and proteins, for the diagnosis, prevention, and treatment of autoimmune/inflammatory disorders, infectious disorders, immune deficiencies, disorders of metabolism, reproductive disorders, neurological disorders, cardiovascular disorders, eye disorders, and cell proliferative disorders, including cancer.

### SUMMARY OF THE INVENTION

Various embodiments of the invention provide purified polypeptides, enzymes, referred to collectively as ‘ENZM’ and individually as ‘ENZM-1,’ ‘ENZM-2,’ ‘ENZM-3,’ ‘ENZM-4,’ ‘ENZM-5,’ ‘ENZM-6,’ ‘ENZM-7,’ ‘ENZM-8,’ ‘ENZM-9,’ ‘ENZM-10,’ ‘ENZM-11,’ ‘ENZM-12,’ ‘ENZM-13,’ ‘ENZM-14,’ ‘ENZM-15,’ ‘ENZM-16,’ ‘ENZM-17,’ ‘ENZM-18,’ ‘ENZM-19,’ ‘ENZM-20,’ ‘ENZM-21,’ ‘ENZM-22,’ ‘ENZM-23,’ ‘ENZM-24,’ ‘ENZM-25,’ ‘ENZM-26,’ ‘ENZM-27,’ ‘ENZM-28,’ ‘ENZM-29,’ ‘ENZM-30,’ ‘ENZM-31,’ ‘ENZM-32,’ ‘ENZM-33,’ ‘ENZM-34,’ ‘ENZM-35,’ ‘ENZM-36,’ ‘ENZM-37,’ ‘ENZM-38,’ ‘ENZM-39,’ ‘ENZM-40,’ ‘ENZM-41,’ and ‘ENZM-42’ and methods for using these proteins and their encoding polynucleotides for the detection, diagnosis, and treatment of diseases and medical conditions. Embodiments also provide methods for utilizing the purified enzymes and/or their encoding polynucleotides for facilitating the drug discovery process, including determination of efficacy, dosage, toxicity, and pharmacology. Related embodiments provide methods for utilizing the purified enzymes and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO:1-42, c) a biologically active fragment of a polypeptide having an amino acid sequence selected from the group consisting of SEQ ID NO:1-42, and d) an immunogenic fragment of a polypeptide having an amino acid sequence selected from the group consisting of SEQ ID NO:1-42. Another embodiment provides an isolated polypeptide comprising an amino acid sequence of SEQ ID NO:1-42.

Still another embodiment provides an isolated polynucleotide encoding a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO: 1-42, c) a biologically active fragment of a polypeptide having an amino acid sequence selected from the group consisting of SEQ ID NO:1-42, and d) an immunogenic fragment of a polypeptide having an amino acid sequence selected from the group consisting of SEQ ID NO:1-42. In another embodiment, the polynucleotide encodes a polypeptide selected from the group consisting of SEQ ID NO:1-42. In an alternative embodiment, the polynucleotide is selected from the group consisting of SEQ ID NO:43-84.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ BD NO:1-42, b) a polypeptide comprising a naturally occurring amino acid sequence at least 90% identical or at least about 90% identical to an amino acid sequence selected from the group consisting of SEQ ID NO:1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO: 1-42, b) a polypeptide comprising a naturally occurring amino acid sequence at least 90% identical or at least about 90% identical to an amino acid sequence selected from the group consisting of SEQ ID NO: 1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO: 1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42.

Still yet another embodiment provides an isolated polynucleotide selected from the group consisting of a) a polynucleotide comprising a polynucleotide sequence selected from the group consisting of SEQ ID NO:43-84, b) a polynucleotide comprising a naturally occurring polynucleotide sequence at least 90% identical or at least about 90% identical to a polynucleotide sequence selected from the group consisting of SEQ ID NO:43-84, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:43-84, b) a polynucleotide comprising a naturally occurring polynucleotide sequence at least 90% identical or at least about 90% identical to a polynucleotide sequence selected from the group consisting of SEQ ID NO:43-84, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:43-84, b) a polynucleotide comprising a naturally occurring polynucleotide sequence at least 90% identical or at least about 90% identical to a polynucleotide sequence selected from the group consisting of SEQ ID NO:43-84, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO:1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42, and a pharmaceutically acceptable excipient. In one embodiment, the composition can comprise an amino acid sequence selected from the group consisting of SEQ ID NO:1-42. Other embodiments provide a method of treating a disease or condition associated with decreased or abnormal expression of functional ENZM, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO:1-42, c) a biologically active fragment of a polypeptide having an amino acid sequence selected from the group consisting of SEQ ID NO:1-42, and d) an immunogenic fragment of a polypeptide having an amino acid sequence selected from the group consisting of SEQ ID NO:1-42. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional ENZM, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO:1-42, c) a biologically active fragment of a polypeptide having an amino acid sequence selected from the group consisting of SEQ ID NO:1-42, and d) an immunogenic fragment of a polypeptide having an amino acid sequence selected from the group consisting of SEQ ID NO:1-42. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional ENZM, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-42, b) a polypeptide comprising a naturally occurring amino acid sequence at least 90% identical or at least about 90% identical to an amino acid sequence selected from the group consisting of SEQ ID NO: 1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO: 1-42, b) a polypeptide comprising a naturally occurring amino acid sequence at least 90% identical or at least about 90% identical to an amino acid sequence selected from the group consisting of SEQ ID NO: 1-42, c) a biologically active fragment of a polypeptide having an amino acid sequence selected from the group consisting of SEQ ID NO: 1-42, and d) an immunogenic fragment of a polypeptide having an amino acid sequence selected from the group consisting of SEQ ID NO:1-42. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:43-84, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:43-84, ii) a polynucleotide comprising a naturally occurring polynucleotide sequence at least 90% identical or at least about 90% identical to a polynucleotide sequence selected from the group consisting of SEQ ID NO:43-84, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:43-84, ii) a polynucleotide comprising a naturally occurring polynucleotide sequence at least 90% identical or at least about 90% identical to a polynucleotide sequence selected from the group consisting of SEQ ID NO:43-84, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“ENZM” refers to the amino acid sequences of substantially purified ENZM obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of ENZM. Agonists may include proteins, nucleic acids, carbohydrates, small molecules, or any other compound or composition which modulates the activity of ENZM either by directly interacting with ENZM or by acting on components of the biological pathway in which ENZM participates.

An “allelic variant” is an alternative form of the gene encoding ENZM. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding ENZM include those sequences with deletions, insertions, or substitutions of different nucleotides, resulting in a polypeptide the same as ENZM or a polypeptide with at least one functional characteristic of ENZM. Included within this definition are polymorphisms which may or may not be readily detectable using a particular oligonucleotide probe of the polynucleotide encoding ENZM, and improper or unexpected hybridization to allelic variants, with a locus other than the normal chromosomal locus for the polynucleotide encoding ENZM. The encoded protein may also be “altered,” and may contain deletions, insertions, or substitutions of amino acid residues which produce a silent change and result in a functionally equivalent ENZM. Deliberate amino acid substitutions may be made on the basis of one or more similarities in polarity, charge, solubility, hydrophobicity, hydrophilicity, and/or the amphipathic nature of the residues, as long as the biological or immunological activity of ENZM is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of ENZM. Antagonists may include proteins such as antibodies, anticalins, nucleic acids, carbohydrates, small molecules, or any other compound or composition which modulates the activity of ENZM either by directly interacting with ENZM or by acting on components of the biological pathway in which ENZM participates.

The term “antibody” refers to intact immunoglobulin molecules as well as to fragments thereof, such as Fab, F(ab′)₂, and Fv fragments, which are capable of binding an epitopic determinant. Antibodies that bind ENZM polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic ENZM, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding ENZM or fragments of ENZM may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| | |
|---|---|
| Original Residue | Conservative Substitution |

| | |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Gln | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| The | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of ENZM or a polynucleotide encoding ENZM which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ D NO:43-84 can comprise a region of unique polynucleotide sequence that specifically identifies SEQ ID NO:43-84, for example, as distinct from any other sequence in the genome from which the fragment was obtained. A fragment of SEQ ID NO:43-84 can be employed in one or more embodiments of methods of the invention, for example, in hybridization and amplification technologies and in analogous methods that distinguish SEQ ID NO:43-84 from related polynucleotides. The precise length of a fragment of SEQ ID NO:43-84 and the region of SEQ ID NO:43-84 to which the fragment corresponds are routinely determinable by one of ordinary skill in the art based on the intended purpose for the fragment.

A fragment of SEQ ID NO:1-42 is encoded by a fragment of SEQ ID NO:43-84. A fragment of SEQ ID NO:1-42 can comprise a region of unique amino acid sequence that specifically identifies SEQ ID NO:1-42. For example, a fragment of SEQ ID NO:1-42 can be used as an immunogenic peptide for the development of antibodies that specifically recognize SEQ ID NO:1-42. The precise length of a fragment of SEQ ID NO:1-42 and the region of SEQ ID NO: 1-42 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nh.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6× SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2× SSC and about 0.1% SDS, for 1 hour. Alternatively, temperatures of about 65° C., 60° C., 55° C., or 42° C. may be used. SSC concentration may be varied from about 0.1 to 2× SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., Cot or Rot analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of ENZM which is capable of eliciting an immune response when introduced into a living organism, for example, a mammal. The term “immunogenic fragment” also includes any polypeptide or oligopeptide fragment of ENZM which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of ENZM. For example, modulation may cause an increase or a decrease in protein activity, binding characteristics, or any other biological, functional, or immunological properties of ENZM.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an ENZM may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of ENZM.

“Probe” refers to nucleic acids encoding ENZM, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4th ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing ENZM, nucleic acids encoding ENZM, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human enzymes (ENZM), the polynucleotides encoding ENZM, and the use of these compositions for the diagnosis, treatment, or prevention of autoimmune/inflammatory disorders, infectious disorders, immune deficiencies, disorders of metabolism, reproductive disorders, neurological disorders, cardiovascular disorders, eye disorders, and cell proliferative disorders, including cancer.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are enzymes. For example, SEQ ID NO: 12 is 64% identical, from residue L6 to residue K468 and is 43% identical, from residue E343 to residue E469, to hexokinase I from common carp, [Cyprinus carpio] (GenBank ID g6840980) as determined by the Basic Local Alignment Search Tool (BLAST). (See Table 2.) The BLAST probability scores are 5.5e-165 and 4.4e-21 respectively, which indicate the probabilities of obtaining the observed polypeptide sequence alignments by chance. As determined by BLAST analysis using the PROTEOME database, SEQ ID NO:12 also has homology to rat hexokinase Type I (ATP:D-hexose 6-phosphotransferase) which catalyzes ATP-dependent conversion of glucose to glucose 6 phosphate in glycolysis. Deficiency of human hexokinase Type I may lead to non-spherocytic hemolyticanemia SEQ ID NO: 12 also contains a hexokinase domain as determined by searching for statistically significant matches in the hidden Markov model (HMM)-based PFAM database of conserved protein family domains. (See Table 3.) Data from BLIMPS, PROFILESCAN and additonal BLAST analyses provide further corroborative evidence that SEQ ID NO:12 is a hexokinase. In an alternative example, SEQ ID NO:17 is 96% identical, from residue M1 to residue K420, to human muscle-specific enolase (GenBank ID g34789) as determined by the Basic Local Alignment Search Tool (BLAST). (See Table 2.) The BLAST probability score is 9.1e-220, which indicates the probability of obtaining the observed polypeptide sequence alignment by chance. As determined by BLAST analysis using the PROTEOME database, SEQ ID NO: 17 also has homology to human and murine muscle-specific enolases (beta enolases) which convert 2-phospho-D-glycerate to phosphoenolpyruvate in glycolysis (PROTEOME IDs 335214|ENO3 and 582749|Eno3). SEQ ID NO: 17 also contains an enolase (enol-ase) domain as determined by searching for statistically significant matches in the hidden Markov model (HMM)-based PFAM database of conserved protein family domains. (See Table 3.) Data from BLIMPS, MOTIFS, PROFILESCAN, and additional BLAST analyses provide further corroborative evidence that SEQ ID NO:17 is an enolase. In an alternative example, SEQ ID NO:28 is 95% identical, from residue Ml to residue A283, to human peroxisomal D3,D2-enoyl-CoA isomerase (GenBank ID g12803665) as determined by the Basic Local Alignment Search Tool (BLAST). (See Table 2.) The BLAST probability score is 7.4e-192, which indicates the probability of obtaining the observed polypeptide sequence alignment by chance. SEQ ID NO:28 also has homology to peroxisomal D3,D2-enoyl-CoA isomerase, as determined by BLAST analysis using the PROTEOME database. SEQ ID NO:28 also contains an acyl CoA binding protein domain and an enoyl-CoA hydratase/isomerase family domain as determined by searching for statistically significant matches in the hidden Markov model (HMM)-based PFAM database of conserved protein family domains. (See Table 3.) Data from BUMPS, MOTIFS, and PROFILESCAN analyses provide further corroborative evidence that SEQ ID NO:28 is a peroxisomal D3,D2-enoyl-CoA isomerase. In an alternative example, SEQ ID NO:38 is 99% identical, from residue Ml to residue Q439, to human UDP-glucuronosyltransferase 2 family polypeptide B (GenBank ID g184475) as determined by the Basic Local Alignment Search Tool (BLAST). (See Table 2.) The BLAST probability score is 1.4E-236, which indicates the probability of obtaining the observed polypeptide sequence alignment by chance. As determined by BLAST analysis using the PROTEOME database, SEQ ID NO:38 also has homology to human UDP glycosyltransferase 2 family polypeptide B, a UDP-glucuronosyltransferase that glucuronidates bilirubin IX alpha. SEQ ID NO:38 also contains a UDP-glucoronosyl and UDP-glucosyl transferase domain as determined by searching for statistically significant matches in the hidden Markov model (HMM)-based PFAM database of conserved protein family domains. (See Table 3.) Data from BUMPS, PROFILESCAN, MOTIFS, and additional BLAST analyses provide further corroborative evidence that SEQ ID NO:38 is a member of the UDP-glucuronosyltransferase family. SEQ ID NO:1-11, SEQ ID NO:13-16, SEQ ID NO:18-27, SEQ ID NO:29-37, and SEQ ID NO:39-42 were analyzed and annotated in a similar manner. The algorithms and parameters for the analysis of SEQ ID NO:1-42 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:43-84 or that distinguish between SEQ ID NO:43-84 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or ‘NT’) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}NYYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, |
| GFG, | for example, GENSCAN (Stanford University, CA, USA) |
| ENST | or FGENES (Computer Genomics Group, The Sanger Centre, |
| | Cambridge, UK) |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses ENZM variants. Various embodiments of ENZM variants can have at least about 80%, at least about 90%, or at least about 95% amino acid sequence identity to the ENZM amino acid sequence, and can contain at least one functional or structural characteristic of ENZM.

Various embodiments also encompass polynucleotides which encode ENZM. In a particular embodiment, the invention encompasses a polynucleotide sequence comprising a sequence selected from the group consisting of SEQ ID NO:43-84, which encodes ENZM. The polynucleotide sequences of SEQ ID NO:43-84, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding ENZM. In particular, such a variant polynucleotide will have at least about 70%, or alternatively at least about 85%, or even at least about 95% polynucleotide sequence identity to a polynucleotide encoding ENZM. A particular aspect of the invention encompasses a variant of a polynucleotide comprising a sequence selected from the group consisting of SEQ ID NO:43-84 which has at least about 70%, or alternatively at least about 85%, or even at least about 95% polynucleotide sequence identity to a nucleic acid sequence selected from the group consisting of SEQ ID NO:43-84. Any one of the polynucleotide variants described above can encode a polypeptide which contains at least one functional or structural characteristic of ENZM.

In addition, or in the alternative, a polynucleotide variant of the invention is a splice variant of a polynucleotide encoding ENZM. A splice variant may have portions which have significant sequence identity to a polynucleotide encoding ENZM, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding ENZM over its entire length; however, portions of the splice variant will have at least about 70%, or alternatively at least about 85%, or alternatively at least about 95%, or alternatively 100% polynucleotide sequence identity to portions of the polynucleotide encoding ENZM. For example, a polynucleotide comprising a sequence of SEQ ID NO:47, a polynucleotide comprising a sequence of SEQ ID NO:50, a polynucleotide comprising a sequence of SEQ ID NO:51, a polynucleotide comprising a sequence of SEQ ID NO:61, and a polynucleotide comprising a sequence of SEQ ID NO:73 are splice variants of each other; a polynucleotide comprising a sequence of SEQ ID NO:57 and a polynucleotide comprising a sequence of SEQ ID NO:67 are splice variants of each other; a polynucleotide comprising a sequence of SEQ ID NO:58 and a polynucleotide comprising a sequence of SEQ ID NO:72 are splice variants of each other; and a polynucleotide comprising a sequence of SEQ ID NO:62, a polynucleotide comprising a sequence of SEQ ID NO:63, and a polynucleotide comprising a sequence of SEQ ID NO:64 are splice variants of each other. Any one of the splice variants described above can encode a polypeptide which contains at least one functional or structural characteristic of ENZM.

It will be appreciated by those skilled in the art that as a result of the degeneracy of the genetic code, a multitude of polynucleotide sequences encoding ENZM, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring ENZM, and all such variations are to be considered as being specifically disclosed.

Although polynucleotides which encode ENZM and its variants are generally capable of hybridizing to polynucleotides encoding naturally occurring ENZM under appropriately selected conditions of stringency, it may be advantageous to produce polynucleotides encoding ENZM or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding ENZM and its derivatives without altering the encoded amino acid sequences include the production of RNA transcripts having more desirable properties, such as a greater half-life, than transcripts produced from the naturally occurring sequence.

The invention also encompasses production of polynucleotides which encode ENZM and ENZM derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding ENZM or any fragment thereof.

Embodiments of the invention can also include polynucleotides that are capable of hybridizing to the claimed polynucleotides, and, in particular, to those having the sequences shown in SEQ ID NO:43-84 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding ENZM may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode ENZM may be cloned in recombinant DNA molecules that direct expression of ENZM, or fragments or functional equivalents thereof, in appropriate host cells. Due to the inherent degeneracy of the genetic code, other polynucleotides which encode substantially the same or a functionally equivalent polypeptides may be produced and used to express ENZM.

The polynucleotides of the invention can be engineered using methods generally known in the art in order to alter ENZM-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of ENZM, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding ENZM may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, ENZM itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of ENZM, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active ENZM, the polynucleotides encoding ENZM or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′untranslated regions in the vector and in polynucleotides encoding ENZM. Such elements may vary in their strength and specificity. Specific initiation signals may also be used to achieve more efficient translation of polynucleotides encoding ENZM. Such signals include the ATG initiation codon and adjacent sequences, e.g. the Kozak sequence. In cases where a polynucleotide sequence encoding ENZM and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding ENZM and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding ENZM. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding ENZM. For example, routine cloning, subcloning, and propagation of polynucleotides encoding ENZM can be achieved using a multifunctional E. coli vector such as PBLUESCRIPT (Stratagene, La Jolla Calif.) or PSPORT1 plasmid (Invitrogen). Ligation of polynucleotides encoding ENZM into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of ENZM are needed, e.g. for the production of antibodies, vectors which direct high level expression of ENZM may be used. For example, vectors containing the strong, inducible SP6 or T7 bacteriophage promoter may be used.

Yeast expression systems may be used for production of ENZM. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of ENZM. Transcription of polynucleotides encoding ENZM may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding ENZM may be ligated into an adenovirus transcription/translation complex consisting of the late promoter and tripartite leader sequence. Insertion in a non-essential E1 or E3 region of the viral genome may be used to obtain infective virus which expresses ENZM in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of ENZM in cell lines is preferred. For example, polynucleotides encoding ENZM can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding ENZM is inserted within a marker gene sequence, transformed cells containing polynucleotides encoding ENZM can be identified by the absence of marker gene function. Alternatively, a marker gene can be placed in tandem with a sequence encoding ENZM under the control of a single promoter. Expression of the marker gene in response to induction or selection usually indicates expression of the tandem gene as well.

In general, host cells that contain the polynucleotide encoding ENZM and that express ENZM may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of ENZM using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on ENZM is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding ENZM include oligolabeling, nick translation, end-labeling, or PCR amplification using a labeled nucleotide. Alternatively, polynucleotides encoding ENZM, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding ENZM may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode ENZM may be designed to contain signal sequences which direct secretion of ENZM through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding ENZM may be ligated to a heterologous sequence resulting in translation of a fusion protein in any of the aforementioned host systems. For example, a chimeric ENZM protein containing a heterologous moiety that can be recognized by a commercially available antibody may facilitate the screening of peptide libraries for inhibitors of ENZM activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the ENZM encoding sequence and the heterologous protein sequence, so that ENZM may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled ENZM may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

ENZM, fragments of ENZM, or variants of ENZM may be used to screen for compounds that specifically bind to ENZM. One or more test compounds may be screened for specific binding to ENZM. In various embodiments, 1, 2, 3, 4, 5, 10, 20, 50, 100, or 200 test compounds can be screened for specific binding to ENZM. Examples of test compounds can include antibodies, anticalins, oligonucleotides, proteins (e.g., ligands or receptors), or small molecules.

In related embodiments, variants of ENZM can be used to screen for binding of test compounds, such as antibodies, to ENZM, a variant of ENZM, or a combination of ENZM and/or one or more variants ENZM. In an embodiment, a variant of ENZM can be used to screen for compounds that bind to a variant of ENZM, but not to ENZM having the exact sequence of a sequence of SEQ ID NO:1-42. ENZM variants used to perform such screening can have a range of about 50% to about 99% sequence identity to ENZM, with various embodiments having 60%, 70%, 75%, 80%, 85%,90%, and 95% sequence identity.

In an embodiment, a compound identified in a screen for specific binding to ENZM can be closely related to the natural ligand of ENZM, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor ENZM (Howard, A. D. et al. (2001) Trends Pharmacol. Sci. 22:132-140; Wise, A. et al. (2002) Drug Discovery Today 7:235-246).

In other embodiments, a compound identified in a screen for specific binding to ENZM can be closely related to the natural receptor to which ENZM binds, at least a fragment of the receptor, or a fragment of the receptor including all or a portion of the ligand binding site or binding pocket. For example, the compound may be a receptor for ENZM which is capable of propagating a signal, or a decoy receptor for ENZM which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG, (Taylor, P. C. et al. (2001) Curr. Opin. Immunol. 13:611-616).

In one embodiment, two or more antibodies having similar or, alternatively, different specificities can be screened for specific binding to ENZM, fragments of ENZM, or variants of ENZM. The binding specificity of the antibodies thus screened can thereby be selected to identify particular fragments or variants of ENZM. In one embodiment, an antibody can be selected such that its binding specificity allows for preferential identification of specific fragments or variants of ENZM. In another embodiment, an antibody can be selected such that its binding specificity allows for preferential diagnosis of a specific disease or condition having increased, decreased, or otherwise abnormal production of ENZM.

In an embodiment, anticalins can be screened for specific binding to ENZM, fragments of ENZM, or variants of ENZM. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit ENZM involves producing appropriate cells which express ENZM, either as a secreted protein or on the cell membrane. Preferred cells can include cells from mammals, yeast, Drosophila, or E. coli. Cells expressing ENZM or cell membrane fractions which contain ENZM are then contacted with a test compound and binding, stimulation, or inhibition of activity of either ENZM or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with ENZM, either in solution or affixed to a solid support, and detecting the binding of ENZM to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

ENZM, fragments of ENZM, or variants of ENZM may be used to screen for compounds that modulate the activity of ENZM. Such compounds may include agonists, antagonists, or partial or inverse agonists. In one embodiment, an assay is performed under conditions permissive for ENZM activity, wherein ENZM is combined with at least one test compound, and the activity of ENZM in the presence of a test compound is compared with the activity of ENZM in the absence of the test compound. A change in the activity of ENZM in the presence of the test compound is indicative of a compound that modulates the activity of ENZM. Alternatively, a test compound is combined with an in vitro or cell-free system comprising ENZM under conditions suitable for ENZM activity, and the assay is performed. In either of these assays, a test compound which modulates the activity of ENZM may do so indirectly and need not come in direct contact with the test compound. At least one and up to a plurality of test compounds may be screened.

In another embodiment, polynucleotides encoding ENZM or their mammalian homologs may be “Knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding ENZM may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding ENZM can also be used to create “knockin” humanized animals (pigs) or transgenic animals (mice or rats) to model human disease. With knockin technology, a region of a polynucleotide encoding ENZM is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress ENZM, e.g., by secreting ENZM in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of ENZM and enzymes. In addition, examples of tissues expressing ENZM can be found in Table 6 and can also be found in Example XI. Therefore, ENZM appears to play a role in autoimmune/inflammatory disorders, infectious disorders, immune deficiencies, disorders of metabolism, reproductive disorders, neurological disorders, cardiovascular disorders, eye disorders, and cell proliferative disorders, including cancer. In the treatment of disorders associated with increased ENZM expression or activity, it is desirable to decrease the expression or activity of ENZM. In the treatment of disorders associated with decreased ENZM expression or activity, it is desirable to increase the expression or activity of ENZM.

Therefore, in one embodiment, ENZM or a fragment or derivative thereof may be administered to a subject to treat or prevent a disorder associated with decreased expression or activity of ENZM. Examples of such disorders include, but are not limited to, an autoimmune/inflammatory disorder such as acquired immunodeficiency syndrome (ADS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, and trauma; an infectious disorder such as a viral infection, e.g., caused by an adenovirus (acute respiratory disease, pneumonia), an arenavirus (lymphocytic choriomeningitis), a bunyavirus (Hantavirus), a coronavirus (pneumonia, chronic bronchitis), a hepadnavirus (hepatitis), a herpesvirus (herpes simplex virus, varicella-zoster virus, Epstein-Barr virus, cytomegalovirus), a flavivirus (yellow fever), an orthomyxovirus (influenza), a papillomavirus (cancer), a paramyxovirus (measles, mumps), a picornovirus (rhinovirus, poliovirus, coxsackie-virus), a polyomavirus (BK virus, JC virus), a poxvirus (smallpox), a reovirus (Colorado tick fever), a retrovirus (human immunodeficiency virus, human T lymphotropic virus), a rhabdovirus (rabies), a rotavirus (gastroenteritis), and a togavirus (encephalitis, rubella), and a bacterial infection, a fungal infection, a parasitic infection, a protozoal infection, and a helminthic infection; an immune deficiency, such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a disorder of metabolism such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, a lipid myopathy, a lipodystrophy, a lysosomal storage disease, mannosidosis, neuramimidase deficiency, obesity, pentosuria phenylketonuria, pseudovitamin D-deficiency rickets; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis, cancer of the breast, fibrocystic breast disease, and galactorrhea, disruptions of spermatogenesis, abnormal sperm physiology, cancer of the testis, cancer of the prostate, benign prostatic hyperplasia, prostatitis, Peyronie's disease, impotence, carcinoma of the male breast, and gynecomastia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, amnesia, catatonia, diabetic neuropathy, tardive dyskinesia, dystonias, paranoid psychoses, postherpetic neuralgia, and Tourette's disorder; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an eye disorder such as ocular hypertension and glaucoma; a disorder of cell proliferation such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and a cancer, including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing ENZM or a fragment or derivative thereof may be administered to a subject to treat or prevent a disorder associated with decreased expression or activity of ENZM including, but not limited to, those described above.

In a further embodiment, a composition comprising a substantially purified ENZM in conjunction with a suitable pharmaceutical carrier may be administered to a subject to treat or prevent a disorder associated with decreased expression or activity of ENZM including, but not limited to, those provided above.

In still another embodiment, an agonist which modulates the activity of ENZM may be administered to a subject to treat or prevent a disorder associated with decreased expression or activity of ENZM including, but not limited to, those listed above.

In a further embodiment, an antagonist of ENZM may be administered to a subject to treat or prevent a disorder associated with increased expression or activity of ENZM. Examples of such disorders include, but are not limited to, those autoimmune/inflammatory disorders, infectious disorders, immune deficiencies, disorders of metabolism, reproductive disorders, neurological disorders, cardiovascular disorders, eye disorders, and cell proliferative disorders, including cancer described above. In one aspect, an antibody which specifically binds ENZM may be used directly as an antagonist or indirectly as a targeting or delivery mechanism for bringing a pharmaceutical agent to cells or tissues which express ENZM.

In an additional embodiment, a vector expressing the complement of the polynucleotide encoding ENZM may be administered to a subject to treat or prevent a disorder associated with increased expression or activity of ENZM including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of ENZM may be produced using methods which are generally known in the art. In particular, purified ENZM may be used to produce antibodies or to screen libraries of pharmaceutical agents to identify those which specifically bind ENZM. Antibodies to ENZM may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with ENZM or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenyl. Among adjuvants used in humans, BCG (bacilli Calmette-Guerin) and Corynebacterium parvum are especially preferable.

It is preferred that the oligopeptides, peptides, or fragments used to induce antibodies to ENZM have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of ENZM amino acids may be fused with those of another protein, such as KLH, and antibodies to the chimeric molecule may be produced.

Monoclonal antibodies to ENZM may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce ENZM-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for ENZM may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between ENZM and its specific antibody. A two-site, monoclonal-based immunoassay utilizing monoclonal antibodies reactive to two non-interfering ENZM epitopes is generally used, but a competitive binding assay may also be employed (Pound, supra).

Various methods such as Scatchard analysis in conjunction with radioimmunoassay techniques may be used to assess the affinity of antibodies for ENZM. Affinity is expressed as an association constant, Kₐ, which is defined as the molar concentration of ENZM-antibody complex divided by the molar concentrations of free antigen and free antibody under equilibrium conditions. The Kₐ determined for a preparation of polyclonal antibodies, which are heterogeneous in their affinities for multiple ENZM epitopes, represents the average affinity, or avidity, of the antibodies for ENZM. The Kₐ determined for a preparation of monoclonal antibodies, which are monospecific for a particular ENZM epitope, represents a true measure of affinity. High-affinity antibody preparations with Kₐ ranging from about 10⁹ to 10¹² L/mole are preferred for use in immunoassays in which the ENZM-antibody complex must withstand rigorous manipulations. Low-affinity antibody preparations with Kₐ ranging from about 10⁶ to 10⁷ L/mole are preferred for use in immunopurification and similar procedures which ultimately require dissociation of ENZM, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of ENZM-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding ENZM, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding ENZM. Such technology is well known in the art, and antisense oligonucleotides or larger fragments can be designed from various locations along the coding or control regions of sequences encoding ENZM (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding ENZM may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in ENZM expression or regulation causes disease, the expression of ENZM from an appropriate population of transduced cells may alleviate the clinical manifestations caused by the genetic deficiency.

In a further embodiment of the invention, diseases or disorders caused by deficiencies in ENZM are treated by constructing mammalian expression vectors encoding ENZM and introducing these vectors by mechanical means into ENZM-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of ENZM include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). ENZM may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PINE; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding ENZM from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to ENZM expression are treated by constructing a retrovirus vector consisting of (i) the polynucleotide encoding ENZM under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding ENZM to cells which have one or more genetic abnormalities with respect to the expression of ENZM. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding ENZM to target cells which have one or more genetic abnormalities with respect to the expression of ENZM. The use of herpes simplex virus (HSV)-based vectors may be especially valuable for introducing ENZM to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding ENZM to target cells. The biology of the prototypic alphavirus, Semliki Forest Virus (SFV), has been studied extensively and gene transfer vectors have been based on the SFV genome (Garoff, H. and K.-J. L1 (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for ENZM into the alphavirus genome in place of the capsid-coding region results in the production of a large number of ENZM-coding RNAs and the synthesis of high levels of ENZM in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of ENZM into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding ENZM.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding ENZM. Such DNA sequences may be incorporated into a wide variety of vectors with suitable RNA polymerase promoters such as 17 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding ENZM. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased ENZM expression or activity, a compound which specifically inhibits expression of the polynucleotide encoding ENZM may be therapeutically useful, and in the treatment of disorders associated with decreased ENZM expression or activity, a compound which specifically promotes expression of the polynucleotide encoding ENZM may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding ENZM is exposed to at least one test compound thus obtained. The sample may comprise, for example, an intact or permeabilized cell, or an in vitro cell-free or reconstituted biochemical system. Alterations in the expression of a polynucleotide encoding ENZM are assayed by any method commonly known in the art. Typically, the expression of a specific nucleotide is detected by hybridization with a probe having a nucleotide sequence complementary to the sequence of the polynucleotide encoding ENZM. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of ENZM, antibodies to ENZM, and mimetics, agonists, antagonists, or inhibitors of ENZM.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising ENZM or fragments thereof. For example, liposome preparations containing a cell-impermeable macromolecule may promote cell fusion and intracellular delivery of the macromolecule. Alternatively, ENZM or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example ENZM or fragments thereof, antibodies of ENZM, and agonists, antagonists or inhibitors of ENZM, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅s/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind ENZM may be used for the diagnosis of disorders characterized by expression of ENZM, or in assays to monitor patients being treated with ENZM or agonists, antagonists, or inhibitors of ENZM. Antibodies useful for diagnostic purposes may be prepared in the same manner as described above for therapeutics. Diagnostic assays for ENZM include methods which utilize the antibody and a label to detect ENZM in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring ENZM, including ELISAs, RIAs, and FACS, are known in the art and provide a basis for diagnosing altered or abnormal levels of ENZM expression. Normal or standard values for ENZM expression are established by combining body fluids or cell extracts taken from normal mammalian subjects, for example, human subjects, with antibodies to ENZM under conditions suitable for complex formation. The amount of standard complex formation may be quantitated by various methods, such as photometric means. Quantities of ENZM expressed in subject, control, and disease samples from biopsied tissues are compared with the standard values. Deviation between standard and subject values establishes the parameters for diagnosing disease.

In another embodiment of the invention, polynucleotides encoding ENZM may be used for diagnostic purposes. The polynucleotides which may be used include oligonucleotides, complementary RNA and DNA molecules, and PNAs. The polynucleotides may be used to detect and quantify gene expression in biopsied tissues in which expression of ENZM may be correlated with disease. The diagnostic assay may be used to determine absence, presence, and excess expression of ENZM, and to monitor regulation of ENZM levels during therapeutic intervention.

In one aspect, hybridization with PCR probes which are capable of detecting polynucleotides, including genomic sequences, encoding ENZM or closely related molecules may be used to identify nucleic acid sequences which encode ENZM. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding ENZM, allelic variants, or related sequences.

Probes may also be used for the detection of related sequences, and may have at least 50% sequence identity to any of the ENZM encoding sequences. The hybridization probes of the subject invention may be DNA or RNA and may be derived from the sequence of SEQ ID NO:43-84 or from genomic sequences including promoters, enhancers, and introns of the ENZM gene.

Means for producing specific hybridization probes for polynucleotides encoding ENZM include the cloning of polynucleotides encoding ENZM or ENZM derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding ENZM may be used for the diagnosis of disorders associated with expression of ENZM. Examples of such disorders include, but are not limited to, an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, and trauma; an infectious disorder such as a viral infection, e.g., caused by an adenovirus (acute respiratory disease, pneumonia), an arenavirus (lymphocytic choriomeningitis), a bunyavirus (Hantavirus), a coronavirus (pneumonia, chronic bronchitis), a hepadnavirus (hepatitis), a herpesvirus (herpes simplex virus, varicella-zoster virus, Epstein-Barr virus, cytomegalovirus), a flavivirus (yellow fever), an orthomyxovirus (influenza), a papillomavirus (cancer), a paramyxovirus (measles, mumps), a picornovirus (rhinovirus, poliovirus, coxsackie-virus), a polyomavirus (BK virus, JC virus), a poxvirus (smallpox), a reovirus (Colorado tick fever), a retrovirus (human immunodeficiency virus, human T lymphotropic virus), a rhabdovirus (rabies), a rotavirus (gastroenteritis), and a togavirus (encephalitis, rubella), and a bacterial infection, a fungal infection, a parasitic infection, a protozoal infection, and a helminthic infection; an immune deficiency, such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a disorder of metabolism such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, a lipid myopathy, a lipodystrophy, a lysosomal storage disease, mannosidosis, neuramimidase deficiency, obesity, pentosuria phenylketonuria, pseudovitamin D-deficiency rickets; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis, cancer of the breast, fibrocystic breast disease, and galactorrhea, disruptions of spermatogenesis, abnormal sperm physiology, cancer of the testis, cancer of the prostate, benign prostatic hyperplasia, prostatitis, Peyronie's disease, impotence, carcinoma of the male breast, and gynecomastia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, amnesia, catatonia, diabetic neuropathy, tardive dyskinesia, dystonias, paranoid psychoses, postherpetic neuralgia, and Tourette's disorder; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an eye disorder such as ocular hypertension and glaucoma; a disorder of cell proliferation such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and a cancer, including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Polynucleotides encoding ENZM may be used in Southern or northern analysis, dot blot, or other membrane-based technologies; in PCR technologies; in dipstick, pin, and multiformat ELISA-like assays; and in microarrays utilizing fluids or tissues from patients to detect altered ENZM expression. Such qualitative or quantitative methods are well known in the art.

In a particular embodiment, polynucleotides encoding ENZM may be used in assays that detect the presence of associated disorders, particularly those mentioned above. Polynucleotides complementary to sequences encoding ENZM may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding ENZM in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of ENZM, a normal or standard profile for expression is established. This may be accomplished by combining body fluids or cell extracts taken from normal subjects, either animal or human, with a sequence, or a fragment thereof, encoding ENZM, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding ENZM may involve the use of PCR. These oligomers may be chemically synthesized, generated enzymatically, or produced in vitro. Oligomers will preferably contain a fragment of a polynucleotide encoding ENZM, or a fragment of a polynucleotide complementary to the polynucleotide encoding ENZM, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding ENZM may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding ENZM are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of ENZM include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, ENZM, fragments of ENZM, or antibodies specific for ENZM may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or ill vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for ENZM to quantify the levels of ENZM expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding ENZM may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding ENZM on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, ENZM, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between ENZM and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with ENZM, or fragments thereof, and washed. Bound ENZM is then detected by methods well known in the art. Purified ENZM can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding ENZM specifically compete with a test compound for binding ENZM. In this manner, antibodies can be used to detect the presence of any peptide which shares one or more antigenic determinants with ENZM.

In additional embodiments, the nucleotide sequences which encode ENZM may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/340,357, U.S. Ser. No. 60/342,962, U.S. Ser. No. 60/343,558, and U.S. Ser. No. 60/351,107, are hereby expressly incorporated by reference.

### EXAMPLES

1. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenyl or in a suitable mixture of denaturants, such as TRIZOL (Invitrogen), a monophasic solution of phenyl and guanidine isothiocyanate. The resulting lysates were centrifuged over CsCl cushions or extracted with chloroform. RNA was precipitated from the lysates with either isopropanol or sodium acetate and ethanol, or by other routine methods.

Phenyl extraction and precipitation of RNA were repeated as necessary to increase RNA purity. In some cases, RNA was treated with DNase. For most libraries, poly(A)⁺ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g. the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example 1 were recovered from host cells by ill vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus noivegicus, Mus musculus, Caenorhabditis elegans, Saccharomyces cerevisiae, Schizosaccharomyces pombe, and Candida albicans (Incyte Genomics, Palo Alto Calif.); hidden Markov model (HIM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS. DOMO, PRODOM, Prosite, hidden Markov model (HMM)-based protein family databases such as PFAM, ICY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:43-84. Fragments from about 20 to about 4000 nucleotides which are useful in hybridization and amplification technologies are described in Table 4, column 2.

IV. Identification and Editing of Coding Sequences from Genomic DNA

Putative enzymes were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode enzymes, the encoded polypeptides were analyzed by querying against PFAM models for enzymes. Potential enzymes were also identified by homology to Incyte cDNA sequences that had been annotated as enzymes. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example In were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of ENZM Encoding Polynucleotides

The sequences which were used to assemble SEQ ID NO:43-84 were compared with sequences from the Incyte LIFESEQ database and public domain databases using BLAST and other implementations of the Smith-Waterman algorithm. Sequences from these databases that matched SEQ ID NO:43-84 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosomes p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding ENZM are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding ENZM. cDNA sequences and cDNA library/tissue information are found in the LIFESEQ GOLD database (Incyte Genomics, Palo Alto Calif.).

VIII. Extension of ENZM Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in ENZM Encoding Polynucleotides

Common DNA sequence variants known as single nucleotide polymorphisms (SNPs) were identified in SEQ ID NO:43-84 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:43-84 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 107 counts per minute of the labeled probe is used in a typical membrane-based hybridization analysis of human genomic DNA digested with one of the following endonucleases: Ase I, Bgl II, Eco RI, Pst I, Xba 1, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 μg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5× SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5× SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5× SSC in a corner of the chamber. The chamber containing the arrays is incubated for about 6.5 hours at 60° C. The arrays are washed for 10 min at 45° C. in a first wash buffer (1× SSC, 0.1% SDS), three times for 10 minutes each at 45° C. in a second wash buffer (0.1× SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

For example, the expression of SEQ ID NO:49, as determined by microarray analysis, was decreased by at least two fold in breast tumor tissues relative to normal breast tissues. The breast tumor tissues were harvested from a 43 year old female donor diagnosed with invasive lobular carcinoma. The tumor was well differentiated and metastatic in 2 out of 13 lymph nodes. The normal breast tissues were harvested from grossly uninvolved breast tissue of the same donor. Therefore, SEQ ID NO:49 is useful in diagnosis of, and monitoring treatment for breast cancer.

In an alternative example, the expression of SEQ ID NO:49, as determined by microarray analysis, was decreased by at least two fold in colon cancer tissues relative to normal colon tissues. The colon cancer tissues were harvested from an 83 year old donor diagnosed with colon cancer. The normal colon tissues were harvested from grossly uninvolved colon tissue of the same donor. Therefore, SEQ ID NO:49 is useful in diagnosis of and monitoring treatment for colon cancer.

In an alternative example, SEQ ID NO:51 showed differential expression, as determined by microarray analysis, in dendritic cells treated with anti-CD40 antibodies. CD40 is a type I integral membrane glycoprotein belonging to the TNF-receptor family. It is expressed on all mature B lymphocytes, dendritic cells, and some epithelial cells. Antibodies specific for CD40 molecules can induce proliferation of B cells when presented with IL-4 or antibodies specific for CD20 molecules. Dendritic cells (DCs), as professional antigen presenting cells, play a crucial role in the initiation of the immune response. DCs therefore represent one of the most promising cell targets for the induction of a specific immune response in vaccination protocols. In this experiment, human monocytic DCs were derived in vitro from the adherent cellular fraction of the peripheral blood of 4 healthy donors. The adherent leukocytes, mostly monocytes, were incubated for 13 days in the presence of recombinant IL-4 (10 ng/ml) and GM-CSF (10 ng/ml). The differentiated DCs were collected after 13 days from the non-adherent cellular fraction and activated in the presence of soluble mouse anti-human CD40 antibodies for 2, 8, and 24 hours. The anti-CD40 treated DCs were compared to untreated DCs. The experiment showed that SEQ ID NO:51 showed at least a two-fold decrease in expression treated with anti-CD40 for 24 hours compared to untreated DCs. Therefore, SEQ ID NO:51 is useful as potential a biological marker in diagnosis of and monitoring treatment for autoimmune/inflammatory disorders.

In an alternative example, the expression of SEQ ID NO:57, as determined by microarray analysis, was decreased by at least two fold in colon adenocarcinoma tissues relative to normal colon tissues. The colon adenocarcinoma tissues were harvested from a 38 year old male donor with invasive, poorly differentiated adenocarcinoma with metastases in 2 out of 13 lymph nodes surveyed. The normal colon tissues were harvested from grossly uninvolved colon tissue of the same donor. Therefore, SEQ ID NO:57 is useful in diagnostic assays for and monitoring treatment of colon cancer.

In an alternative example, SEQ ID NO:59 showed differential expression, as determined by microarray analysis, in Alzheimer's Disease (AD). In a comparison of various brain tissues (amygdala, cingulate cortex, cerebellum, and striatum) from donors with moderate or severe AD to matched brain tissues from matched donors, the expression of SEQ ID NO:59 was decreased at least two fold in Alzheimer's brain tissues. Therefore, SEQ ID NO:59 is useful in diagnostic assays for AD and as a potential biological marker and therapeutic agent in the treatment of AD.

In an alternative example, expression of SEQ ID NO:66 is decreased in colon cancer versus normal colon tissue. In one experiment, matched normal and tumor samples from a 64-year-old female diagnosed with moderately differentiated colon adenocarcinoma (Huntsman Cancer Institute, Salt Lake City, Utah) were compared by competitive hybridization. In another experiment, matched normal and tumor samples from an 85-year-old male diagnosed with colon cancer (Huntsman Cancer Institute, Salt Lake City, Utah) were compared by competitive hybridization. In another experiment, gene expression profiles were obtained by comparing normal colon tissue from a 56-year-old female diagnosed with poorly differentiated metastatic adenocarcinoma of possible ovarian origin and a clinical history of recurrent cecal mass, to colon tumor tissue from the same donor (Huntsman Cancer Institute, Salt Lake City, Utah) by competitive hybridization. In another experiment, matched normal and tumor samples from a 58-year-old female diagnosed with mucinous adenocarcinoma (Huntsman Cancer Institute, Salt Lake City, Utah) were compared by competitive hybridization. Therefore, SEQ ID NO:66 is useful in diagnosis of, and monitoring treatment for, colon cancer and other cell proliferative disorders.

In an alternative example, expression of SEQ ID NO:70 is decreased in C3A cells treated with clofibrate versus untreated C3A cells. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein; iii) conversion of ammonia to urea and glutamine; iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416). C3A cells were treated with clofibrate and compared with untreated cells. In one experiment, early confluent C3A cells were treated with clofibrate at 50, 200, 1600, and 2000 μM for 1, 3, and 6 hours. The treated cells were compared to untreated early-confluent C3A cells. Therefore, SEQ ID NO:70 is useful in diagnosis of, and monitoring treatment for, atherosclerosis and other autoimmune/inflammatory disorders.

As another example, expression of SEQ ID NO:70 is decreased in C3A cells treated with fenofibrate versus untreated C3A cells. Early confluent C3A cells were treated with fenofibrate at 50, 200, 1600, and 2000 μM for 1, 3, and 6 hours. The treated cells were compared to untreated early-confluent C3A cells. Therefore, SEQ ID NO:70 is useful in diagnosis of, and monitoring treatment for, atherosclerosis and other autoimmune/inflammatory disorders.

In an alternative example, expression of SEQ ID NO:72 is decreased in colon tumor versus normal colon tissue. In one experiment, matched normal and tumor samples from an 81-year-old male diagnosed with colon cancer (Huntsman Cancer Institute, Salt Lake City, Utah) were compared by competitive hybridization. In another experiment, the gene expression profile of 6 colon cancer tissue samples were compared by competitive hybridization. Tissues were: a) Poorly differentiated metastatic adenocarcinoma from a 56 year old female, b) Colon cancer from an 85 year old male, c) Colon cancer from an 81 year old male, d) Colon cancer from a 73 year old Female, e) Colon cancer from an 83 year old female, and f) Mucinous adenocarcinoma from a 58 year old female. In another experiment, gene expression profiles were obtained by comparing normal sigmoid colon tissue from a 48-year-old female to a sigmoid colon tumor originating from a metastatic gastric sarcoma (stromal tumor) from the same donor (Huntsman Cancer Institute, Salt Lake City, Utah). Therefore, SEQ ID NO:72 is useful in diagnosis of, and monitoring treatment for, colon cancer and other cell proliferative disorders.

In an alternative example, expression of SEQ ID NO:74 is increased in peripheral blood mononuclear cells (PBMCs) treated with staphlococcal exotoxin B (SEB) versus untreated PBMCs. In one experiment, peripheral blood mononuclear cells (PBMCs) of healthy volunteer donors were stimulated in vitro with SEB for various time periods. The SEB treated PBMCs from each donor were compared to PBMCs from the same donor, kept in culture for 24 hours in the absence of SEB. Therefore, SEQ ID NO:74 is useful in diagnosis of, and monitoring treatment for, autoimmune/inflammatory disorders.

In an alternative example, the expression of SEQ ID NO:75, as determined by microarray analysis, was decreased by at least four fold in tumorous colon tissues relative to normal colon tissues. Both the tumor and the normal colon tissues were isolated from a 38 year old male with invasive, poorly differentiated adenocarcinoma with metastases in 2 out of 13 lymph nodes surveyed. Gene expression profiles were obtained by comparing normal colon tissue to tumorous rectal tissue from this donor. Individual pieces of normal tissue were compared against a pool of normal tissue from the same donor to determine gene expression variation in normal colon tissue. Therefore, SEQ ID NO:75 is useful as a diagnostic marker or as a potential therapeutic target for colon cancer.

In an alternative example, SEQ ID NO:77 showed differential expression in inflammatory responses as determined by microarray analysis. The expression of SEQ ID NO:77 was increased by at least two-fold in the Jurkat T-cell leukemia cell line that had been stimulated for one hour with PMA (phorbol 12-myristate 13-acetate) concentrations varying from 5 nM to 1 μM and with ionomycin concentrations varying between 50 ng/ml and 1 μg/ml when compared to untreated Jurkat cells. Jurkat is an acute T-cell leukemia cell line that grows in the absence of external stimuli and has been used extensively to study signaling in human T-cells. PMA is a broad activator of protein kinase C-dependent pathways. Ionomycin is a calcium ionophore that permits the entry of calcium in the cell, hence increasing the cytosolic calcium concentration. The combination of PMA and ionomycin activates two of the major signaling pathways used by mammalian cells to interact with their environment. In T-cells, the combination of PMA and ionomycin mimics the type of secondary signaling events elicited during optimal B-cell activation. Therefore, SEQ ID NO:77 is useful in diagnostic assays for inflammatory responses.

In an alternative example, SEQ ID NO:79 also showed differential expression in inflammatory responses as determined by microarray analysis. The expression of SEQ ID NO: 18 was decreased by at least two-fold in ECV304 cell line that had been stimulated for 2, 4, and 8 hours with 0.1 μM PMA (phorbol 12-myristate 13-acetate) and with 1 μg/ml ionomycin when compared to untreated ECV304 cells in the absence of stimuli. ECV304 is a cell line derived from the endothelium of the human umbilical vein. This cell model has been extensively used to study the functional biology of human endothelial cells in vitro. PMA is a broad activator of the protein kinase C-dependent pathways. Ionomycin is a calcium ionophore that permits the entry of calcium in the cell, hence increasing the cytosolic calcium concentration. The combination of PMA and ionomycin activates two of the major signaling pathways used by mammalian cells to interact with their environment. Therefore, SEQ ID NO:79 is useful in diagnostic assays for inflammatory responses.

XII. Complementary Polynucleotides

Sequences complementary to the ENZM-encoding sequences, or any parts thereof, are used to detect, decrease, or inhibit expression of naturally occurring ENZM. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of ENZM. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the ENZM-encoding transcript.

XIII. Expression of ENZM

Expression and purification of ENZM is achieved using bacterial or virus-based expression systems. For expression of ENZM in bacteria, cDNA is subcloned into an appropriate vector containing an antibiotic resistance gene and an inducible promoter that directs high levels of cDNA transcription. Examples of such promoters include, but are not limited to, the trp-tac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express ENZM upon induction with isopropyl beta-D-thiogalactopyranoside (IPTG). Expression of ENZM in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding ENZM by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, ENZM is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from ENZM at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified ENZM obtained by these methods can be used directly in the assays shown in Examples XVII, XVIII, and XIX, where applicable.

XIV. Functional Assays

ENZM function is assessed by expressing the sequences encoding ENZM at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of ENZM on gene expression can be assessed using highly purified populations of cells transfected with sequences encoding ENZM and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding ENZM and other genes of interest can be analyzed by northern analysis or microarray techniques.

XV. Production of ENZM Specific Antibodies

ENZM substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the ENZM amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-ENZM activity by, for example, binding the peptide or ENZM to a substrate, blocking with 1% BSA, reacting with rabbit antisera, washing, and reacting with radio-iodinated goat anti-rabbit IgG.

XVI. Purification of Naturally Occurring ENZM Using Specific Antibodies

Naturally occurring or recombinant ENZM is substantially purified by immunoaffinity chromatography using antibodies specific for ENZM. An immunoaffinity column is constructed by covalently coupling anti-ENZM antibody to an activated chromatographic resin, such as CNBr-activated SEPHAROSE (Amersham Biosciences). After the coupling, the resin is blocked and washed according to the manufacturer's instructions.

Media containing ENZM are passed over the immunoaffinity column, and the column is washed under conditions that allow the preferential absorbance of ENZM (e.g., high ionic strength buffers in the presence of detergent). The column is eluted under conditions that disrupt antibody/ENZM binding (e.g., a buffer of pH 2 to pH 3, or a high concentration of a chaotrope, such as urea or thiocyanate ion), and ENZM is collected.

XVII. Identification of Molecules Which Interact with ENZM

ENZM, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled ENZM, washed, and any wells with labeled ENZM complex are assayed. Data obtained using different concentrations of ENZM are used to calculate values for the number, affinity, and association of ENZM with the candidate molecules.

Alternatively, molecules interacting with ENZM are analyzed using the yeast two-hybrid system as described in Fields, S. and O. Song (1989; Nature 340:245-246), or using commercially available kits based on the two-hybrid system, such as the MATCHMAKER system (Clontech).

ENZM may also be used in the PATHCALLING process (CuraGen Corp., New Haven Conn.) which employs the yeast two-hybrid system in a high-throughput manner to determine all interactions between the proteins encoded by two large libraries of genes (Nandabalan. K. et al. (2000) U.S. Pat. No. 6,057,101).

XVIII. Demonstration of ENZM Activity

ENZM activity is demonstrated through a variety of specific enzyme assays; some of which are outlined below.

ENZM oxidoreductase activity is measured by the increase in extinction coefficient of NAD(P)H coenzyme at 340 nm for the measurement of oxidation activity, or the decrease in extinction coefficient of NAD(P)H coenzyme at 340 nm for the measurement of reduction activity (Dalziel, K. (1963) J. Biol. Chem. 238:2850-2858). One of three substrates may be used: Asn-βGal, biocytidine, or ubiquinone-10. The respective subunits of the enzyme reaction, for example, cytochrome c₁-b oxidoreductase and cytochrome c, are reconstituted. The reaction mixture contains a) 1-2 mg/ml ENZM; and b) 15 mM substrate, 2.4 mM NAD(P)⁺ in 0.1 M phosphate buffer, pH 7.1 (oxidation reaction), or 2.0 mM NAD(P)H, in 0.1 M Na₂HPO₄ buffer, pH 7.4 (reduction reaction); in a total volume of 0.1 ml. Changes in absorbance at 340 nm (A₃₄₀) are measured at 23.5° C. using a recording spectrophotometer (Shimadzu Scientific Instruments, Inc., Pleasanton, Calif.). The amount of NAD(P)H is stoichiometrically equivalent to the amount of substrate initially present, and the change in A₃₄₀ is a direct measure of the amount of NAD(P)H produced; ΔA₃₄₀=6620[NADH]. ENZM activity is proportional to the amount of NAD(P)H present in the assay.

Aldo/keto reductase activity of ENZM is proportional to the decrease in absorbance at 340 nm as NADPH is consumed (or increased absorbance if NADPH is produced, i.e., if the reverse reaction is monitored). A standard reaction mixture is 135 mM sodium phosphate buffer (pH 6.2-7.2 depending on enzyme), 0.2 mM NADPH, 0.3 M lithium sulfate, 0.5-2.5 mg ENZM and an appropriate level of substrate. The reaction is incubated at 30° C. and the reaction is monitored continuously with a spectrophotometer. ENZM activity is calculated as mol NADPH consumed/mg of ENZM.

Acyl-CoA dehydrogenase activity of ENZM is measured using an anaerobic electron transferring flavoprotein (ETF) assay. The reaction mixture comprises 50 mM Tris-HCl (pH 8.0), 0.5% glucose, and 50 μM acyl-CoA substrate (i.e., isovaleryl-CoA) that is pre-warmed to 32° C. The mixture is depleted of oxygen by repeated exposure to vacuum followed by layering with argon. Trace amounts of oxygen are removed by the addition of glucose oxidase and catalase followed by the addition of ETF to a final concentration of 1 μM. The reaction is initiated by addition of purified ENZM or a sample containing ENZM and exciting the reaction at 342 nm. Quenching of fluorescence caused by the transfer of electrons from the substrate to ETF is monitored at 496 nm. 1 unit of acyl-CoA dehydrogenase activity is defined as the amount of ENZM required to reduce 1 μmol of ETF per minute (Reinard, T. et al. (2000) J. Biol. Chem. 275:33738-33743).

Alcohol dehydrogenase activity of ENZM is measured by following the conversion of NAD⁺ to NADH at 340 nm (ε₃₄₀=6.22 mM⁻¹ cm⁻¹) at 25° C. in 0.1 M potassium phosphate (pH 7.5), 0.1 M glycine (pH 10.0), and 2.4 mM NAD⁺. Substrate (e.g., ethanol) and ENZM are then added to the reaction. The production of NADH results in an increase in absorbance at 340 nm and correlates with the oxidation of the alcohol substrate and the amount of alcohol dehydrogenase activity in the ENZM sample (Svensson, S. (1999) J. Biol. Chem. 274:29712-29719).

Aldehyde dehydrogenase activity of ENZM is measured by determining the total hydrolase+dehydrogenase activity of ENZM and subtracting the hydrolase activity. Hydrolase activity is first determined in a reaction mixture containing 0.05 M Tris-HCl (pH 7.8), 100 nM 2-mercaptoethanol, and 0.5-18 μM substrate, e.g., 10-HCO-HPteGlu (10-formyltetrahydrofolate; HPteGlu, tetrahydrofolate) or 10-FDDF (10-formyl-5,8-dideazafolate). Approximately lug of ENZM is added in a final volume of 1.0 ml. The reaction is monitored and read against a blank cuvette, containing all components except enzyme. The appearance of product is measured at either 295 nm for 5,8-dideazafolate or 300 nm for HPteGlu using molar extinction coefficients of 1.89×10⁴ and 2.17×10⁴ for 5,8-dideazafolate and HPteGlu, respectively. The addition of NADP⁺ to the reaction mixture allows the measurement of both dehydrogenase and hydrolase activity (assays are performed as before). Based on the production of product in the presence of NADP⁺ and the production of product in the absence of the cofactor, aldehyde dehydrogenase activity is calculated for ENZM. In the alternative, aldehyde dehydrogenase activity is assayed using propanal as substrate. The reaction mixture contains 60 mM sodium pyrophosphate buffer (pH 8.5), 5 mM propanal, 1 mM NADP⁺, and ENZM in a total volume of 1 ml. Activity is determined by the increase in absorbance at 340 nm, resulting from the generation of NADPH, and is proportional to the aldehyde dehydrogenase activity in the sample (Krupenko, S. A. et al. (1995) J. Biol. Chem. 270:519-522).

6-phosphogluconate dehydrogenase activity of ENZM is measured by incubating purified ENZM, or a composition comprising ENZM, in 120 mM triethanolamine (pH 7.5), 0.1 mM EDTA, 0.5 mM NADP⁺, and 10-150 μM 6-phosphogluconate as substrate at 20-25° C. The production of NADPH is measured fluorimetrically (340 nm excitation, 450 nm emission) and is indicative of 6-phosphogluconate dehydrogenase activity. Alternatively, the production of NADPH is measured photometrically, based on absorbance at 340 nm. The molar amount of NADPH produced in the reaction is proportional to the 6-phosphogluconate dehydrogenase activity in the sample (Tetaud et al., supra).

Ribonucleotide diphosphate reductase activity of ENZM is determined by incubating purified ENZM, or a composition comprising ENZM, along with dithiothreitol, Mg⁺⁺, and ADP, GDP, CDP, or UDP substrate. The product of the reaction, the corresponding deoxyribonucleotide, is separated from the substrate by thin-layer chromatography. The reaction products can be distinguished from the reactants based on rates of migration. The use of radiolabeled substrates is an alternative for increasing the sensitivity of the assay. The amount of deoxyribonucleotides produced in the reaction is proportional to the amount of ribonucleotide diphosphate reductase activity in the sample (note that this is true only for pre-steady state kinetic analysis of ribonucleotide diphosphate reductase activity, as the enzyme is subject to negative feedback inhibition by products) (Nutter and Cheng, supra).

Dihydrodiol dehydrogenase activity of ENZM is measured by incubating purified ENZM, or a composition comprising ENZM, in a reaction mixture comprising 50 nM glycine (pH 9.0), 2.3 mM NADP⁺, 8% DMSO, and a trans-dihydrodiol substrate, selected from the group including but not limited to, (±)-trans-naphthalene-1,2-dihydrodiol, (±)-trans-phenanthrene-1,2-dihydrodiol, and (±)-trans-chrysene-1,2-dihydrodiol. The oxidation reaction is monitored at 340 nm to detect the formation of NADPH, which is indicative of the oxidation of the substrate. The reaction mixture can also be analyzed before and after the addition of ENZM by circular dichroism to determine the stereochemistry of the reaction components and determine which enantiomers of a racemic substrate composition are oxidized by the ENZM (Penning, supra).

Glutathione S-transferase (GST) activity of ENZM is determined by measuring the ENZM catalyzed conjugation of GSH with 1-chloro-2,4-dinitrobenzene (CDNB), a common substrate for most GSTs. ENZM is incubated with 1 nM CDNB and 2.5 mM GSH together in 0.1M potassium phosphate buffer, pH 6.5, at 25° C. The conjugation reaction is measured by the change in absorbance at 340 nm using an ultraviolet spectrophometer. ENZM activity is proportional to the change in absorbance at 340 nm.

15-oxoprostaglandin 13-reductase (PGR) activity of ENZM is measured following the separation of contaminating 15-hydroxyprostaglandin dehydrogenase (15-PGDH) activity by DEAE chromatography. Following isolation of PGR containing fractions (or using the purified ENZM), activity is assayed in a reaction comprising 0.1 M sodium phosphate (pH 7.4), 1 mM 2-mercaptoethanol, 20 μg substrate (e.g., 15-oxo derivatives of prostaglandins PGE₁, PGE₂, and PGE_{2α}), and 1 mM NADH (or a higher concentration of NADPH). ENZM is added to the reaction which is then incubated for 10 min at 37° C. before termination by the addition of 0.25 ml 2 N NaOH. The amount of 15-oxo compound remaining in the sample is determined by measuring the maximum absorption at 500 nm of the terminated reaction and comparing this value to that of a terminated control reaction that received no ENZM. 1 unit of enzyme is defined as the amount required to catalyze the oxidation of 1 μmol substrate per minute and is proportional to the amount of PGR activity in the sample.

Choline dehydrogenase activity of ENZM is identified by the ability of E. coli, transformed with an ENZM expression vector, to grow on media containing choline as the sole carbon and nitrogen source. The ability of the transformed bacteria to thrive is indicative of choline dehydrogenase activity (Magne Østeråfs, M. (1998) Proc. Natl. Acad. Sci. USA 95:11394-11399).

ENZM thioredoxin activity is assayed as described (Luthman, M. (1982) Biochemistry 21:6628-6633). Thioredoxins catalyze the formation of disulfide bonds and regulate the redox environment in cells to enable the necessary thiol:disulfide exchanges. One way to measure the thiol:disulfide exchange is by measuring the reduction of insulin in a mixture containing 0.1 M potassium phosphate, pH 7.0, 2 mM EDTA, 0.16 μM insulin, 0.33 mM DTT, and 0.48 mM NADPH. Different concentrations of ENZM are added to the mixture, and the reaction rate is followed by monitoring the oxidation of NADPH at 340 nM.

ENZM transferase activity is measured through assays such as a methyl transferase assay in which the transfer of radiolabeled methyl groups between a donor substrate and an acceptor substrate is measured (Bokar, J. A. et al. (1994) J. Biol. Chem. 269:17697-17704). Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg ENZM, and acceptor substrate (0.4 μg [³⁵S]RNA or 6-mercaptopurine (6-MP) to 1 mM final concentration). Reaction mixtures are incubated at 30° C. for 30 minutes, then at 65° C. for 5 minutes. The products are separated by chromatography or electrophoresis and the level of methyl transferase activity is determined by quantification of methyl-3H recovery.

Aminotransferase activity of ENZM is assayed by incubating samples containing ENZM for 1 hour at 37° C. in the presence of 1 mM L-kynurenine and 1 mM 2-oxoglutarate in a final volume of 200 μl of 150 mM Tris acetate buffer (pH 8.0) containing 70 μM PLP. The formation of kynurenic acid is quantified by HPLC with spectrophotometric detection at 330 nm using the appropriate standards and controls well known to those skilled in the art. In the alternative, L-3-hydroxykynurenine is used as substrate and the production of xanthurenic acid is determined by HPLC analysis of the products with UV detection at 340 nm. The production of kynurenic acid and xanthurenic acid, respectively, is indicative of aminotransferase activity (Buchli et al., supra).

In another alternative, aminotransferase activity of ENZM is measured by determining the activity of purified ENZM or crude samples containing ENZM toward various amino and oxo acid substrates under single turnover conditions by monitoring the changes in the UV/VIS absorption spectrum of the enzyme-bound cofactor, pyridoxal 5′-phosphate (PLP). The reactions are performed at 25° C. in 50 mM 4-methylmorpholine (pH 7.5) containing 9 μM purified ENZM or ENZM containing samples and substrate to be tested (amino and oxo acid substrates). The half-reaction from amino acid to oxo acid is followed by measuring the decrease in absorbance at 360 nm and the increase in absorbance at 330 nm due to the conversion of enzyme-bound PLP to pyridoxamine 5′ phosphate (PMP). The specificity and relative activity of ENZM is determined by the activity of the enzyme preparation against specific substrates (Vacca, supra).

ENZM chitinase activity is determined with the fluorogenic substrates 4-methylumbelliferyl chitotriose, methylumbelliferyl chitobiose, or methylumbelliferyl N-acetylglucosamine. Purified ENZM is incubated with 0.5 uM substrate at pH 4.0 (0.1M citrate buffer), pH 5.0 (0.1M phosphate buffer), or 1.57 pH 6.0 (0.1M Tris-HCL). After various times of incubation, the reaction is stopped by the addition of 0.1M glycine buffer, pH 10.4, and the concentration of free methylumbelliferone is determined fluorometrically. Chitinase B from Serratia marcescens may be used as a positive control (Hakala, supra).

ENZM isomerase activity is determined by measuring 2-hydroxyhepta-2,4-diene,1,7 dioate isomerase (HHDD isomerase) activity, as described by Garrido-Peritierra, A. and R. A. Cooper (1981; Eur. J. Biochem. 17:581-584). The sample is combined with 5-carboxymethyl-2-oxo-hex-3-ene-1,5, dioate (CMHD), which is the substrate for HHDD isomerase. CMHD concentration is monitored by measuring its absorbance at 246 nm. Decrease in absorbance at 246 nm is proportional to HHDD isomerase activity of ENZM.

ENZM isomerase activity such as peptidyl prolyl cis/trans isomerase activity can be assayed by an enzyme assay described by Rahfeld (supra). The assay is performed at 10° C. in 35 mM HEPES buffer, pH 7.8, containing chymotrypsin (0.5 mg/ml) and ENZM at a variety of concentrations. Under these assay conditions, the substrate, Suc-Ala-Xaa-Pro-Phe-4-NA, is in equilibrium with respect to the prolyl bond, with 80-95% in trans and 5-20% in cis conformation. An aliquot (2 μl) of the substrate dissolved in dimethyl sulfoxide (10 mg/ml) is added to the reaction mixture described above. Only the cis isomer is a substrate for cleavage by chymotrypsin. Thus, as the substrate is isomerized by ENZM, the product is cleaved by chymotrypsin to produce 4-nitroanilide, which is detected by its absorbance at 390 nm. 4-Nitroanilide appears in a time-dependent and a ENZM concentration-dependent manner.

Alternatively, peptidyl prolyl cis-trans isomerase activity of ENZM can be assayed using a chromogenic peptide in a coupled assay with chymotrypsin (Fischer, G. et al. (1984) Biomed. Biochim. Acta 43:1101-1111).

UDP glucuronyltransferase activity of ENZM is measured using a calorimetric determination of free amine groups (Gibson, G. G. and P. Skett (1994) Introduction to Drug Metabolism, Blackie Academic and Professional, London). An amine-containing substrate, such as 2-aminophenyl, is incubated at 37° C. with an aliquot of the enzyme in a reaction buffer containing the necessary cofactors (40 mM Tris pH 8.0, 7.5 mM MgCl₂, 0.025% Triton X-100, 1 mM ascorbic acid, 0.75 mM UDP-glucuronic acid). After sufficient time, the reaction is stopped by addition of ice-cold 20% trichloroacetic acid in 0.1 M phosphate buffer pH 2.7, incubated on ice, and centrifuged to clarify the supernatant. Any unreacted 2-aminophenyl is destroyed in this step. Sufficient freshly-prepared sodium nitrite is then added; this step allows formation of the diazonium salt of the glucuronidated product. Excess nitrite is removed by addition of sufficient ammonium sulfamate, and the diazonium salt is reacted with an aromatic amine (for example, N-naphthylethylene diamine) to produce a colored azo compound which can be assayed spectrophotometrically (at 540 nm, for example). A standard curve can be constructed using known concentrations of aniline, which will form a chromophore with similar properties to 2-aminophenyl glucuronide.

Adenylosuccinate synthetase activity of ENZM is measured by synthesis of AMP from IMP. The sample is combined with AMP. IMP concentration is monitored spectrophotometrically at 248 nm at 23° C. (Wang, W. et al. (1995) J. Biol. Chem. 270:13160-13163). The increase in IMP concentration is proportional to ENZM activity.

Alternatively, AMP binding activity of ENZM is measured by combining the sample with ³²P-labeled AMP. The reaction is incubated at 37° C. and terminated by addition of trichloroacetic acid. The acid extract is neutralized and subjected to gel electrophoresis to remove unbound label. The radioactivity retained in the gel is proportional to ENZM activity.

In another alternative, xenobiotic carboxylic acid:CoA ligase activity of ENZM is measured by combining the sample with γ-³³P-ATP and measuring the formation of γ-³³P— pyrophosphate with time (Vessey, D. A. et al. (1998) J. Biochem. Mol. Toxicol. 12:151-155).

Protein phosphatase (PP) activity can be measured by the hydrolysis of P-nitrophenyl phosphate (PNPP). ENZM is incubated together with PNPP in HEPES buffer pH 7.5, in the presence of 0.1% β-mercaptoethanol at 37° C. for 60 min. The reaction is stopped by the addition of 6 ml of 10 N NaOH (Diamond, R. H. et al. (1994) Mol. Cell. Biol. 14:3752-62).

Alternatively, acid phosphatase activity of ENZM is demonstrated by incubating ENZM containing extract with 100 μl of 10 mM PNPP in 0.1 M sodium citrate, pH 4.5, and 50 μl of 40 mM NaCl at 37° C. for 20 min. The reaction is stopped by the addition of 0.5 ml of 0.4 M glycine/NaOH, pH 10.4 (Saftig, P. et al. (1997) J. Biol. Chem. 272:18628-18635). The increase in light absorbance at 410 nm resulting from the hydrolysis of PNPP is measured using a spectrophotometer. The increase in light absorbance is proportional to the activity of ENZM in the assay.

In the alternative, ENZM activity is determined by measuring the amount of phosphate removed from a phosphorylated protein substrate. Reactions are performed with 2 or 4 nM ENZM in a final volume of 30 μl containing 60 mM Tris, pH 7.6, 1 mM EDTA, 1 mM EGTA, 0.1% 2-mercaptoethanol and 10 μM substrate, ³²P-labeled on serine/threonine or tyrosine, as appropriate. Reactions are initiated with substrate and incubated at 30° C. for 10-15 nin. Reactions are quenched with 450 μl of 4% (w/v) activated charcoal in 0.6 M HCl, 90 mM Na₄P₂O₇, and 2 mM NaH₂PO₄, then centrifuged at 12,000×g for 5 min. Acid-soluble ³²Pi is quantified by liquid scintillation counting (Sinclair, C. et al. (1999) J. Biol. Chem. 274:23666-23672).

The adenosine deaminase activity of ENZM is determined by measuring the rate of deamination that occurs when adenosine substrate is incubated with ENZM. Reactions are performed with a predetermined amount of ENZM in a final volume of 3.0 ml containing 53.3 mM potassium phosphate and 0.045 mM adenosine. Assay reagents excluding ENZM are mixed in a quartz cuvette and equilibrated to 25° C. Reactions are initiated by the addition of ENZM and are mixed immediately by inversion. The decrease in light absorbance at 265 nm resulting from the hydrolysis of adenosine to inosine is measured using a spectrophotometer. The decrease in the A_{265 nm} is recorded for approximately 5 minutes. The decrease in light absorbance is proportional to the activity of ENZM in the assay.

ENZM hydrolase activity is measured by the hydrolysis of appropriate synthetic peptide substrates conjugated with various chromogenic molecules in which the degree of hydrolysis is quantified by spectrophotometric (or fluorometric) absorption of the released chromophore (Beynon and Bond, supra, pp. 25-55). Peptide substrates are designed according to the category of protease activity as endopeptidase (serine, cysteine, aspartic proteases), aminopeptidase (leucine aminopeptidase), or carboxypeptidase (Carboxypeptidase A and B, procollagen C-proteinase).

An assay for carbonic anhydrase activity of ENZM uses the fluorescent pH indicator 8-hydroxypyrene-1,3,6-trisulfonate (pyranine) in combination with stopped-flow fluorometry to measure carbonic anhydrase activity (Shingles, et al. 1997, Anal. Biochem. 252:190-197). A pH 6.0 solution is mixed with a pH 8.0 solution and the initial rate of bicarbonate dehydration is measured. Addition of carbonic anhydrase to the pH 6.0 solution enables the measurement of the initial rate of activity at physiological temperatures with resolution times of 2 ms. Shingles et al. (supra) used this assay to resolve differences in activity and sensitivity to sulfonamides by comparing mammalian carbonic anhydrase isoforms. The fluorescent technique's sensitivity allows the determination of initial rates with a protein concentration as little as 65 ng/ml.

Decarboxylase activity of ENZM is measured as the release of CO₂ from labeled substrate. For example, ornithine decarboxylase activity of ENZM is assayed by measuring the release of CO₂ from L-[1-¹⁴C]-ornithine (Reddy, S. G et al. (1996) J. Biol. Chem. 271:24945-24953). Activity is measured in 200 μl assay buffer (50 mM Tris/HCl, pH 7.5, 0.1 mM EDTA. 2 nM dithiothreitol, 5 mM NaF, 0.1% Brij35, 1 mM PMSF, 60 μM pyridoxal-5-phosphate) containing 0.5 mM L-ornithine plus 0.5 μCi L-[1-¹⁴C]ornithine. The reactions are stopped after 15-30 minutes by addition of 1 M citric acid, and the ¹⁴CO₂ evolved is trapped on a paper disk filter saturated with 20 μl of 2 N NaOH. The radioactivity on the disks is determined by liquid scintillation spectography. The amount of ¹⁴CO₂ released is proportional to ornithine decarboxylase activity of ENZM.

AdoHCYase activity of ENZM in the hydrolytic direction is performed spectroscopically by measuring the rate of the product (homocysteine) formed by reaction with 5,5′-Dithiobis(2-nitrobenzoic acid) (DTNB). To 800 μl of an enzyme solution containing 4.7 μg of ENZM and 4 units of adenosine deaminase in 50 mM potassium phosphate buffer, pH 7.2, containing 1 mM EDTA (buffer A), is added 200 μl of S-Adenosyl-L-homocysteine (500 μM) containing 250 μM DTNB in buffer A. The reaction mixture is incubated at 37° C. for 2 minutes. Hydrolytic activity is monitored at 412 nm continuously using a diode array UV spectrophotometer. Enzyme activity is defined as the amount of enzyme that can hydrolyze 1 μmol of S-Adenosyl-L-homocysteine/minute (Yuan, C-S et al. (1996) J. Biol. Chem. 271:28009-28015).

AdoHCYase activity of ENZM can be measured in the synthetic direction as the production of S-adenosyl homocysteine using 3-deazaadenosine as a substrate (Sganga et al. supra). Briefly, ENZM is incubated in a 100 μl volume containing 0.1 mM 3-deazaadenosine, 5 mM homocysteine, 20 mM HEPES (pH 7.2). The assay mixture is incubated at 37° C. for 15 minutes. The reaction is terminated by the addition of 10 μl of 3 M perchloric acid. After incubation on ice for 15 minutes, the mixture is centrifuged for 5 minutes at 18,000×g in a microcentrifuge at 4° C. The supernatant is removed, neutralized by the addition of 1 M potassium carbonate, and centrifuged again. A 50/μl aliquot of supernatant is then chromatographed on an Altex Ultrasphere ODS column (5 μm particles, 4.6×250 mm) by isocratic elution with 0.2 M ammonium dihydrogen phosphate (Aldrich) at a flow rate of 1 ml/min. Protein is determined by the bicinchoninic acid assay (Pierce).

Alternatively, AdoHCYase activity of ENZM can be measured in the synthetic direction by a TLC method (Hershfield, M. S. et al. (1979) J. Biol. Chem. 254:22-25). In a preincubation step, 50 μM [8⁻¹⁴C]adenosine is incubated with 5 molar equivalents of NAD⁺ for 15 minutes at 22° C. Assay samples containing ENZM in a 50 μl final volume of 50 mM potassium phosphate buffer, pH 7.4, 1 mM DTT, and 5 mM homocysteine, are mixed with the preincubated [8⁻¹⁴C]adenosine/NAD⁺ to initiate the reaction. The reaction is incubated at 37° C., and 1 μl samples are spotted on TLC plates at 5 minute intervals for 30 minutes. The chromatograms are developed in butanol-1/glacial acetic acid/water (12:3:5, v/v) and dried. Standards are used to identify substrate and products under ultraviolet light. The complete spots containing [¹⁴C]adenosine and [¹⁴C]SAH are then detected by exposing x-ray film to the TLC plate. The radiolabeled substrate and product are then cut from the chromatograms and counted by liquid scintillation spectrometry. Specific activity of the enzyme is determined from the linear least squares slopes of the product vs time plots and the milligrams of protein in the sample (Bethin, K. E. et al. (1995) J. Biol. Chem. 270:20698-20702).

Asparaginase activity of ENZM can be measured in the hydrolytic direction by determining the amount of radiolabeled L-aspartate released from 0.6 mM N⁴-β′-N-acetylglucosaminyl-L-asparagine substrate when it is incubated at 25° C. with ENZM in 50 mM phosphate buffer, pH 7.5 (Kaartinen, V. et al. (1991) J. Biol. Chem. 266:5860-5869).

Acyl CoA Acid Hydrolase activity of ENZM in the hydrolytic direction is performed spectroscopically by monitoring the appearance of the product (CoASH) formed by reaction of substrate (acyl-CoA) and ENZM with 5,5′-Dithiobis(2-nitrobenzoic acid) (DTNB). The final reaction volume is 1 ml of 0.05 M potassium phosphate buffer, pH 8, containing 0.11 mM DTNB, 20 μg/ml bovine serum albumin, 10 μM of acyl-CoA of different lengths (C6-CoA, C10-CoA, C14-CoA and C18-CoA, Sigma), and ENZM. The reaction mixture is incubated at 22° C. for 7 minutes. Hydrolytic activity is monitored spectrophotometrically by measuring absorbance at 412 nm (Poupon, V. et al. (1999) J. Biol. Chem. 274:19188-19194).

ENZM activity of ENZM can be measured spectrophotometrically by determining the amount of solubilized RNA that is produced as a result of incubation of RNA substrate with ENZM. 5 μl (20 μg) of a 4 mg/ml solution of yeast tRNA (Sigma) is added to 0.8 ml of 40 mM sodium phosphate, pH 7.5, containing ENZM. The reaction is incubated at 25° C. for 15 minutes. The reaction is stopped by addition of 0.5 ml of an ice-cold fresh solution of 20 nM lanthanum nitrate plus 3% perchloric acid. The stopped reaction is incubated on ice for at least 15 min, and the insoluble tRNA is removed by centrifugation for 5 min at 10,000 g. Solubilized tRNA is determined as UV absorbance (260 nm) of the remaining supernatant, with A₂₆₀ of 1.0 corresponding to 40 μg of solubilized RNA (Rosenberg, H. F. et al. (1996) Nucleic Acids Research 24:3507-3513).

ENZM activity can be determined as the ability of ENZM to cleave ³²P internally labeled T. thermophila pre-tRNA^{Gln}. ENZM and substrate are added to reaction vessels and reactions are carried out in MBB buffer (50 mM Tris-HCl (pH 7.5), 10 mM MgCl₂) for 1 hour at 37° C. Reactions are terminated with the addition of an equal volume of sample loading buffer (SLB: 40 mM EDTA, 8 M urea, 0.2% xylene cyanol, and 0.2% bromophenyl blue). The reaction products are separated by electrophoresis on 8 M urea, 6% polyacrylamide gels and analyzed using detection instruments and software capable of quantification of the products. One unit of ENZM activity is defined as the amount of enzyme required to cleave 10% of 28 fmol of T. thermophila pre-tRNA^{Gln} to mature products in 1 hour at 37° C. (True, H. L. et al. (1996) J. Biol. Chem. 271:16559-16566).

Alternatively, cleavage of ³²P internally labeled substrate tRNA by ENZM can be determined in a 20 μl reaction mixture containing 30 mM HEPES-KOH (pH 7.6), 6 mM MgCl₂, 30 mM KCl, 2 mM DTT, 25 μg/ml bovine serum albumin, 1 unit/μl rRNasin, and 5,000-50,000 cpm of gel-purified substrate RNA. 3.0 μl of ENZM is added to the reaction mixture, which is then incubated at 37° C. for 30 minutes. The reaction is stopped by guanidinium/phenyl extraction, precipitated with ethanol in the presence of glycogen, and subjected to denaturing polyacrylamide gel electrophoresis (6 or 8% polyacrylamide, 7 M urea) and autoradiography (Rossmanith, W. et al. (1995) J. Biol. Chem. 270:12885-12891). The ENZM activity is proportional to the amount of cleavage products detected.

ENZM activity can be measured by determining the amount of free adenosine produced by the hydrolysis of AMP, as described by Sala-Newby et al., supra. Briefly, ENZM is incubated with AMP in a suitable buffer for 10 minutes at 37° C. Free adenosine is separated from AMP and measured by reverse phase HPLC.

Alternatively, ENZM activity is measured by the hydrolysis of ADP-ribosylarginine (Konczalik, P. and J. Moss (1999) J. Biol. Chem. 274:16736-16740). 50 ng of ENZM is incubated with 100 μM ADP-ribosyl-[¹⁴C]arginine (78,000 cpm) in 50 mM potassium phosphate, pH 7.5, 5 mM dithiothreitol, 10 mM MgCl₂ in a final volume of 100 μl. After 1 h at 37° C., 90 μl of the sample is applied to a column (0.5×4 cm) of Affi-Gel 601 (boronate) equilibrated and eluted with five 1-mil portions of 0.1 M glycine, pH 9.0, 0.1 M NaCl, and 10 mM MgCl₂. Free ¹⁴C-Arg in the total eluate is measured by liquid scintillation counting.

Epoxide hydrolase activity of ENZM can be determined with a radiometric assay utilizing [H³]-labeled trans-stilbene oxide (TSO) as substrate. Briefly, ENZM is preincubated in Tris-HCl pH 7.4 buffer in a total volume of 100 μl for 1 minute at 37° C. 1 μl of [H]-labeled TSO (0.5 μM in EtOH) is added and the reaction mixture is incubated at 37° C. for 10 minutes. The reaction mixture is extracted with 200 μl n-dodecane. 50 μl of the aqueous phase is removed for quantification of diol product in a liquid scintillation counter (LSC). ENZM activity is calculated as nmol diol product/min/mg protein (Gill, S. S. et al. (1983) Analytical Biochemistry 131:273-282).

Lysophosphatidic acid acyltransferase activity of ENZM is measured by incubating samples containing ENZM with 1 mM of the thiol reagent 5,5′-dithiobis(2-nitrobenzoic acid) (DTNB), 50 μm LPA, and 50 μm acyl-CoA in 100 mM Tris-HCl, pH 7.4. The reaction is initiated by addition of acyl-CoA, and allowed to reach equilibrium. Transfer of the acyl group from acyl-CoA to LPA releases free CoA, which reacts with DTNB. The product of the reaction between DTNB and free CoA absorbs at 413 nm. The change in absorbance at 413 nm is measured using a spectrophotometer, and is proportional to the lysophosphatidic acid acyltransferase activity of ENZM in the sample.

N-acyltransferase activity of ENZM is measured using radiolabeled amino acid substrates and measuring radiolabel incorporation into conjugated products. ENZM is incubated in a reaction buffer containing an unlabeled acyl-CoA compound and radiolabeled amino acid, and the radiolabeled acyl-conjugates are separated from the unreacted amino acid by extraction into n-butanol or other appropriate organic solvent. For example, Johnson, M. R. et al. (1990; J. Biol. Chem. 266:10227-10233) measured bile acid-CoA:amino acid N-acyltransferase activity by incubating the enzyme with cholyl-CoA and ³H-glycine or ³H-taurine, separating the tritiated cholate conjugate by extraction into n-butanol, and measuring the radioactivity in the extracted product by scintillation. Alternatively, N-acyltransferase activity is measured using the spectrophotometric determination of reduced CoA (CoASH) described below.

N-acetyltransferase activity of ENZM is measured using the transfer of radiolabel from [¹⁴C]acetyl-CoA to a substrate molecule (for example, see Deguchi, T. (1975) J. Neurochem. 24:1083-5). Alternatively, a newer spectrophotometric assay based on DTNB reaction with CoASH may be used. Free thiol-containing CoASH is formed during N-acetyltransferase catalyzed transfer of an acetyl group to a substrate. CoASH is detected using the absorbance of DTNB conjugate at 412 nm (De Angelis, J. et al. (1997) J. Biol. Chem. 273:3045-3050). ENZM activity is proportional to the rate of radioactivity incorporation into substrate, or the rate of absorbance increase in the spectrophotometric assay.

Galactosyltransferase activity of ENZM is determined by measuring the transfer of galactose from UDP-galactose to a GlcNAc-terminated oligosaccharide chain in a radioactive assay. (Kolbinger, F. et al. (1998) J. Biol. Chem. 273:58-65.) The ENZM sample is incubated with 14 μl of assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcβO—(CH₂), —CO₂Me (37 mg/ml in dimethyl sulfoxide) for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GlcNAcβO—(CH₂), —CO₂Me remains bound to the column during the water washes and is eluted with 5 ml of methanol. Radioactivity in the eluted material is measured by liquid scintillation counting and is proportional to galactosyltransferase activity of ENZM in the starting sample.

Phosphoribosyltransferase activity of ENZM is measured as the transfer of a phosphoribosyl group from phosphoribosylpyrophosphate (PRPP) to a purine or pyrimidine base. Assay mixture (20 μl) containing 50 mM Tris acetate, pH 9.0, 20 nM 2-mercaptoethanol, 12.5 mM MgCl₂, and 0.1 mM labeled substrate, for example, [¹⁴C]uracil, is mixed with 20 μl of ENZM diluted in 0.1 M Tris acetate, pH 9.7, and 1 mg/ml bovine serum albumin. Reactions are preheated for 1 min at 37° C., initiated with 10 μl of 6 mM PRPP, and incubated for 5 min at 37° C. The reaction is stopped by heating at 100° C. for 1 min. The product [¹⁴C]UMP is separated from [¹⁴C]uracil on DEAE-cellulose paper (Turner, R. J. et al. (1998) J. Biol. Chem. 273:5932-5938). The amount of [¹⁴C]UMP produced is proportional to the phosphoribosyltransferase activity of ENZM.

ADP-ribosyltransferase activity of ENZM is measured as the transfer of radiolabel from adenine-NAD to agmatine (Weng, B. et al. (1999) J. Biol. Chem. 274:31797-31803). Purified ENZM is incubated at 30° C. for 1 hr in a total volume of 300 μl containing 50 mM potassium phosphate (pH. 7.5), 20 mM agmatine, and 0.1 mM [adenine-U-¹⁴C]NAD (0.05 mCi). Samples (100 μl) are applied to Dowex columns and [¹⁴C]ADP-ribosylagmatine eluted with 5 ml of water for liquid scintillation counting. The amount of radioactivity recovered is proportional to ADP-ribosyltransferase activity of ENZM.

An ENZM activity assay measures aminoacylation of tRNA in the presence of a radiolabeled substrate. SYNT is incubated with [¹⁴C]-labeled amino acid and the appropriate cognate tRNA (for example, [¹⁴C]alanine and tRNA^{ala}) in a buffered solution. ¹⁴C-labeled product is separated from free [¹⁴C]amino acid by chromatography, and the incorporated ¹⁴C is quantified using a scintillation counter. The amount of ¹⁴C-labeled product detected is proportional to the activity of ENZM in this assay (Ibba, M. et al. (1997) Science 278:1119-1122).

Alternatively, argininosuccinate synthase activity of ENZM is measured based on the conversion of [³H]aspartate to [³H]argininosuccinate. ENZM is incubated with a mixture of [³H]aspartate, citrulline, Tris-HCl (pH 7.5), ATP, MgCl₂, KCl, phosphoenolpyruvate, pyruvate kinase, myokinase, and pyrophosphatase, and allowed to proceed for 60 minutes at 37° C. Enzyme activity was terminated with addition of acetic acid and heating for 30 minutes at 90° C. [³H]argininosuccinate is separated from un-catalyzed [³H]aspartate by chromatography and quantified by liquid scintillation spectrometry. The amount of [³H]argininosuccinate detected is proportional to the activity of ENZM in this assay (O'Brien, W. E. (1979) Biochemistry 18:5353-5356).

Alternatively, the esterase activity of ENZM is assayed by the hydrolysis of p-nitrophenylacetate (NPA). ENZM is incubated together with 0.1 μM NPA in 0.1 M potassium phosphate buffer (pH 7.25) containing 150 nM NaCl. The hydrolysis of NPA is measured by the increase of absorbance at 400 nm with a spectrophotometer. The increase in light absorbance is proportional to the activity of ENZM (Probst, M. R. et al. (1994) J. Biol. Chem. 269:21650-21656).

XIX. Identification of ENZM Agonists and Antagonists

Agonists or antagonists of ENZM activation or inhibition may be tested using the assays described in section XVIII. Agonists cause an increase in ENZM activity and antagonists cause a decrease in ENZM activity.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | Incyte Full |
| Incyte Project ID | SEQ ID NO: | Polypeptide ID | SEQ ID NO: | ID | Length Clones |
| 70612021 | 1 | 70612021CD1 | 43 | 70612021CB1 | 2289282CA2 |
| 71847235 | 2 | 71847235CD1 | 44 | 71847235CB1 | 1529076CA2 |
| 7505230 | 3 | 7505230CD1 | 45 | 7505230CB1 | |
| 7505235 | 4 | 7505235CD1 | 46 | 7505235CB1 | 90029889CA2 |
| 7505793 | 5 | 7505793CD1 | 47 | 7505793CB1 | 90117405CA2 |
| 7505861 | 6 | 7505861CD1 | 48 | 7505861CB1 | |
| 7505864 | 7 | 7505864CD1 | 49 | 7505864CB1 | 90179680CA2, |
| | | | | | 90179696CA2 |
| 7506427 | 8 | 7506427CD1 | 50 | 7506427CB1 | 90117620CA2 |
| 7506429 | 9 | 7506429CD1 | 51 | 7506429CB1 | 90117484CA2, |
| | | | | | 90117627CA2, |
| | | | | | 90117651CA2 |
| 7505799 | 10 | 7505799CD1 | 52 | 7505799CB1 | 1364806CA2 |
| 7505843 | 11 | 7505843CD1 | 53 | 7505843CB1 | 2482380CA2 |
| 90001378 | 12 | 90001378CD1 | 54 | 90001378CB1 | 90001378CA2 |
| 7504923 | 13 | 7504923CD1 | 55 | 7504923CB1 | 90055941CA2 |
| 7506151 | 14 | 7506151CD1 | 56 | 7506151CB1 | |
| 7506450 | 15 | 7506450CD1 | 57 | 7506450CB1 | 90117309CA2, |
| | | | | | 90167712CA2, |
| | | | | | 90167752CA2 |
| 71380031 | 16 | 71380031CD1 | 58 | 71380031CB1 | 1457779CA2 |
| 7506054 | 17 | 7506054CD1 | 59 | 7506054CB1 | |
| 7506139 | 18 | 7506139CD1 | 60 | 7506139CB1 | |
| 7506426 | 19 | 7506426CD1 | 61 | 7506426CB1 | 90117392CA2 |
| 7506741 | 20 | 7506741CD1 | 62 | 7506741CB1 | 90111740CA2 |
| 7506743 | 21 | 7506743CD1 | 63 | 7506743CB1 | 90111896CA2 |
| 7506746 | 22 | 7506746CD1 | 64 | 7506746CB1 | 90222101CA2 |
| 7506748 | 23 | 7506748CD1 | 65 | 7506748CB1 | 90111972CA2 |
| 1419966 | 24 | 1419966CD1 | 66 | 1419966CB1 | |
| 7506451 | 25 | 7506451CD1 | 67 | 7506451CB1 | 90117633CA2 |
| 90015249 | 26 | 90015249CD1 | 68 | 90015249CB1 | 90015133CA2, |
| | | | | | 90015241CA2 |
| 7487231 | 27 | 7487231CD1 | 69 | 7487231CB1 | 90196175CA2 |
| 7506260 | 28 | 7506260CD1 | 70 | 7506260CB1 | |
| 7506270 | 29 | 7506270CD1 | 71 | 7506270CB1 | 3689418CA2 |
| 7506306 | 30 | 7506306CD1 | 72 | 7506306CB1 | 1457687CA2, |
| | | | | | 1457779CA2 |
| 7506428 | 31 | 7506428CD1 | 73 | 7506428CB1 | 90117492CA2, |
| | | | | | 95004171CA2 |
| 7678032 | 32 | 7678032CD1 | 74 | 7678032CB1 | |
| 7508332 | 33 | 7508332CD1 | 75 | 7508332CB1 | 1945315CA2, |
| | | | | | 95140542CA2 |
| 1288969 | 34 | 1288969CD1 | 76 | 1288969CB1 | 1288969CA2 |
| 72069135 | 35 | 72069135CD1 | 77 | 72069135CB1 | |
| 7506247 | 36 | 7506247CD1 | 78 | 7506247CB1 | |
| 7506363 | 37 | 7506363CD1 | 79 | 7506363CB1 | |
| 7509068 | 38 | 7509068CD1 | 80 | 7509068CB1 | 2517260CA2 |
| | | | | | 90068365CA2, |
| | | | | | 90068509CA2, |
| | | | | | 90068517CA2, |
| | | | | | 90068525CA2, |
| 7505897 | 39 | 7505897CD1 | 81 | 7505897CB1 | 90068533CA2, |
| | | | | | 90068541CA2, |
| | | | | | 90068609CA2 |
| 7505898 | 40 | 7505898CD1 | 82 | 7505898CB1 | |
| 7505907 | 41 | 7505907CD1 | 83 | 7505907CB1 | |
| 7505925 | 42 | 7505925CD1 | 84 | 7505925CB1 | |

TABLE 2

| Poly- | | | | |
|---|---|---|---|---|
| peptide | | GenBank ID NO: | | |
| SEQ | Incyte | or PROTEOME | Probability | |
| ID NO: | Polypeptide ID | ID NO: | Score | Annotation |
| 1 | 70612021CD1 | g1226246 | 6.5E−194 | [Homo sapiens] mono-ADP-ribosyltransferase |
| | | | | (Levy, I. et al. (1996) FEBS Lett. 382: 276-280.) |
| | | 343974\|ART3 | 1.9E−194 | [Homo sapiens][Transferase][Plasma membrane] |
| | | | | ADP-ribosyltransferase 3, member of the mono (ADP-ribosyl) |
| | | | | transferase family, may have ADP-ribosyltransferase activity, |
| | | | | may be involved in signaling during immune responses within the |
| | | | | airway |
| | | | | (Levy, I. et al. (supra); Balducci, E. et al. (1999) Am. |
| | | | | J. Respir. Cell. Mol. Biol. 21: 337-346.) |
| | | 326576\|4930569 | 1.2E−112 | [Mus musculus] Protein with high similarity to human ART3, |
| | | O04Rik | | which is a member of the mono (ADP-ribosyl) transferase family |
| | | | | that is specifically expressed in spleen and testis |
| 2 | 71847235CD1 | g182146 | 0.0 | [Homo sapiens] eosinophil peroxidase |
| | | | | (Sakamaki, K. et al. (1989) J. Biol. Chem. 264: 16828-16836.) |
| | | 344170\|EPX | 0.0 | [Homo sapiens][Oxidoreductase] Eosinophil peroxidase, |
| | | | | participates in host defense against extracellular pathogens |
| | | | | through the generation of reactive oxidants; may play a role in |
| | | | | tissue damage in asthma and other chronic inflammatory conditions |
| | | | | (Sakamaki, K. et al. (supra); Henderson, J. P. (2001) Proc. Natl. |
| | | | | Acad. Sci. USA 98: 1631-1636.) |
| | | 319456\|Epx | 0.0 | [Mus musculus][Oxidoreductase] Eosinophil peroxidase, |
| | | | | has role in host defense and is expressed in tissues containing |
| | | | | eosinophil progenitor cells |
| | | | | (Horton, M. A. et al. (1996) J. Leukoc. Biol. 60: 285-294; |
| | | | | Duguet, A. et al. (2001) Am. J. Respir. Crit. Care Med. 164: 1119-1126.) |
| 3 | 7505230CD1 | g14195001 | 0.0 | [Homo sapiens] glycosylphosphatidylinositol-specific |
| | | | | phospholipase D precursor |
| | | 583097\|Gpld1 | 0.0 | [Mus musculus][Hydrolase][Extracellular (excluding |
| | | | | cell wall)] Glycosylphosphatidylinositol specific |
| | | | | phospholipase D, hydrolyzes the inositol phosphate |
| | | | | linkage in phosphatidylinositol-glycan anchored proteins |
| | | | | (LeBoeuf, R. C. et al. (1998) Mamm. Genome 9: 710-714.) |
| | | 335584\|GPLD1 | 0.0 | [Homo sapiens][Hydrolase] Phosphatidylinositol-glycan |
| | | | | specific phospholipase D, hydrolyzes the inositol phosphate |
| | | | | linkage in phosphatidylinositol-glycan anchored proteins, |
| | | | | may affect localization and expression of phosphatidylinositol- |
| | | | | glycan anchored proteins |
| | | | | (Scallon, B. J. et al. (1991) Science 252: 446-448). |
| 4 | 7505235CD1 | g179423 | 5.5E−144 | [Homo sapiens] beta-galactosidase precursor (EC 3.2.1.23) |
| | | | | (Yamamoto, Y. et al. (1990) DNA 9: 119-127.) |
| | | 618314\|GLB1 | 4.8E−145 | [Homo sapiens][Transferase; Hydrolase][Lysosome/vacuole; |
| | | | | Nuclear; Cytoplasmic[ Beta-galactosidase, catalyzes cleavage of |
| | | | | the terminal galactose from gangliosides, glycoproteins, and |
| | | | | glycosaminoglycans; deficiency is associated with the lysosomal |
| | | | | storage disorders GM1 gangliosidosis and Morquio syndrome type B |
| | | | | (Oshima, A. et al. (1998) Biochem. Biophys. Res. Commun. 157: 238-244; |
| | | | | Wilkening, G. et al. (2000) J. Biol. Chem. 275: 35814-35819.) |
| | | 584331\|Glb1 | 1.9E−143 | [Mus musculus][Hydrolase][Lysosome/vacuole; |
| | | | | Cytoplasmic] Acid beta-galactosidase, catalyzes cleavage of |
| | | | | the terminal galactose from gangliosides, glycoproteins, and |
| | | | | glycosaminoglycans |
| | | | | (Nanba, E., and Suzuki, K. (1990) Biochem. Biophys. Res. Commun. |
| | | | | 173: 141-148; Hahn, C. N. et al. (1997) Hum. Mol. Genet. 6: 205-211.) |
| 5 | 7505793CD1 | g178077 | 7.4E−123 | [Homo sapiens] adenosine deaminase |
| | | | | (Wiginton, D. A. et al. (1986) Biochemistry 25: 8234-8244) |
| | | 339048\|ADA | 6.5E−124 | [Homo sapiens][Hydrolase][Plasma membrane] |
| | | | | Adenosine deaminase, has roles in immune response and may be |
| | | | | associated with autism; deficiency causes a form of severe |
| | | | | combined immunodeficiency |
| | | | | (Persico, A. M. et al. (2000) Am. J. Med. Genet. 96: 784-790.) |
| | | 583545\|Ada | 5.9E−102 | [Mus musculus][Hydrolase] Adenosine deaminase, has |
| | | | | roles in immune response and various developmental processes; |
| | | | | mutants die pernatally and show liver-cell degeneration, |
| | | | | atelectasis and small intestinal cell death, but retain an |
| | | | | apparently normal thymus |
| | | | | (Migchielsen, A. A. et al. (1995) Nat. Genet. 10: 279-287; |
| | | | | Wakamiya, M. et al. (1995) Proc. Natl. Acad. Sci. USA 92: 3673-3677.) |
| 6 | 7505861CD1 | g4679012 | 7.2E−21 | [Homo sapiens] lysophospholipase isoform |
| | | | | (Zhang, Q. H. et al. (2000) Genome Res. 10: 1546-1560.) |
| | | 343508\|LYPLA1 | 5.1E−20 | [Homo sapiens][Hydrolase] Lysophospholipid-specific |
| | | | | lysophospholipase, hydrolyzes lysophosphatidyl choline and |
| | | | | regulates multifunctional lysophospholipids on membranes |
| | | | | (Wang, A.(1999) Biochim. Biophys. Acta 1437: 157-169.) |
| | | 328450\|Lypla1 | 8.3E−20 | [Rattus norvegicus][Hydrolase] Lysophospholipase |
| | | | | (calcium-independent phospholipase_a2), becomes activated in |
| | | | | proximal tubule cells upon hypoxic injury |
| | | | | (Sugimoto, H. et al. (1998) J. Biol. Chem. 273: 12536-12542.) |
| 7 | 7505864CD1 | g1763011 | 4.1E−150 | [Homo sapiens] lysophospholipase homolog |
| | | | | (Wall, E. M. et al. (1997) Virus Res. 52: 157-167.) |
| | | 428408\|HU-K5 | 3.6E−151 | [Homo sapiens][Hydrolase] Monoglyceride lipase, may |
| | | | | catalyze the hydrolysis of monoglyceride to fatty acid and |
| | | | | glycerol, may play a role in inflammation |
| | | | | (Karlsson, M. et al. (2001) Gene 272: 11-18.) |
| | | 585299\|Mgl1 | 2.1E−127 | [Mus musculus][Hydrolase] Monoglyceride lipase, may |
| | | | | transport and hydrolyze 2-monoglycerides in adipocytes, activity |
| | | | | is inhibited by p-chloromercuribenzoic acid and mercury chloride |
| | | | | (Karlsson, M. et al. (1997) J. Biol. Chem. 272: 27218-27223.) |
| 8 | 7506427CD1 | g14043373 | 4.9E−182 | [Homo sapiens] adenosine deaminase |
| | | 339048\|ADA | 1.4E−182 | [Homo sapiens][Hydrolase][Plasma membrane] |
| | | | | Adenosine deaminase, has roles in immune response and may be |
| | | | | associated with autism; deficiency causes a form of severe |
| | | | | combined immunodeficiency |
| | | | | (Persico, A. M. et al. (supra)) |
| | | 583545\|Ada | 3.1E−152 | [Mus musculus][Hydrolase] Adenosine deaminase, has |
| | | | | roles inimmune response and various developmental processes; |
| | | | | mutants die perinatally and show liver-cell degeneration, |
| | | | | atelectasis and small intestinal cell death, but retain an |
| | | | | apparently normal thymus |
| | | | | (Migchielsen, A. A. et al. (supra); Wakamiya, M. et al. (supra)) |
| 9 | 7506429CD1 | g178077 | 1.2E−61 | [Homo sapiens] adenosine deaminase |
| | | | | (Wiginton, D. A. et al. (1986) Biochemistry 25: 8234-8244) |
| | | 339048\|ADA | 1.0E−62 | [Homo sapiens][Hydrolase][Plasma membrane] |
| | | | | Adenosine deaminase, has roles in immune response and may be |
| | | | | associated with autism; deficiency causes a form of severe |
| | | | | combined immunodeficiency (Persico, A. M. et al. (supra)) |
| | | 583545\|Ada | 1.3E−55 | [Mus musculus][Hydrolase] Adenosine deaminase, has |
| | | | | roles inimmune response and various developmental processes; |
| | | | | mutants die perinatally and show liver-cell degeneration, |
| | | | | atelectasis and small intestinal cell death, but retain an |
| | | | | apparently normal thymus |
| | | | | (Migchielsen, A. A. et al. (supra); Wakamiya, M. et al. (supra)) |
| 10 | 7505799CD1 | g13925310 | 6.2E−67 | [Homo sapiens] cytochrome c oxidase subunit IV isoform |
| | | | | 2 precursor |
| | | | | (Huttemann, M. et al. (2001) Gene 267: 111-123.) |
| | | 731465\|COXIV-2 | 5.4E−68 | [Homo sapiens] Isoform of subunit IV of cytochrome c |
| | | | | oxidase, highly expressed in adult lung (Huttemann, M. et al. (supra)) |
| | | 732943\|CoxIV-2 | 1.6E−42 | [Rattus norvegicus] Isoform of subunit IV of cytochrome c oxidase, |
| | | | | highly expressed in adult lung (Huttemann, M. et al. (supra)) |
| 11 | 7505843CD1 | g1695155 | 2.5E−150 | [Homo sapiens] NADH-cytochrome-b5 reductase |
| | | 568342\|DIA1 | 1.0E−140 | [Homo sapiens][Oxidoreductase; Small molecule-binding |
| | | | | protein][Cytoplasmic; Mitochondrial] NADH-cytochrome b5 |
| | | | | reductase(diaphorase); mutations in the corresponding gene are |
| | | | | associated with hereditary methemoglobinemia types I (red cell |
| | | | | type) and II (generalized type) |
| | | | | (Du, M. et al. (1997) Biochem. Biophys. Res. Commun. 235: |
| | | | | 779-783; Dekker, J. et al. (2001) Blood 97: 1106-1114.) |
| | | 333516\|Rn.37520 | 4.1E−135 | [Rattus norvegicus][Oxidoreductase; Small molecule-binding |
| | | | | protein][Cytoplasmic; Plasma membrane; Mitochondrial] |
| | | | | NADH-cytochrome b5 reductase, may exist as either a soluble |
| | | | | or membrane-bound isoform, has strong similarity to human DIA1 |
| | | | | (diaphorase), which is associated with type I and type II |
| | | | | methemoglobinemia (Wetts, R. et al. (1995) Dev. Dyn. 202: 215-228.) |
| 12 | 90001378CD1 | g6840980 | 5.5E−165 | [Cyprinus carpio] hexokinase I |
| | | 331490\|Hk1 | 9.0E−165 | [Rattus norvegicus][Transferase; Other kinase; Small |
| | | | | molecule-binding protein][Cytoplasmic] Hexokinase Type I |
| | | | | (ATP: D-hexose 6-phosphotransferase), catalyzes ATP-dependent |
| | | | | conversion of glucose to glucose 6 phosphate in glycolysis; |
| | | | | deficiency of human HK1 may lead to non-spherocytic |
| | | | | hemolyticanemia (Schwab, D. A., and Wilson, J. E. (1988) J. Biol. |
| | | | | Chem. 263: 3220-3224; McNay, E. C. et al. (2000) Proc. Natl. |
| | | | | Acad. Sci. USA 97: 2881-2885.) |
| 13 | 7504923CD1 | g15073505 | 3.7E−40 | [Sinorhizobium meliloti] Ribitol type dehydrogenase protein |
| | | | | (Capela, D. et al. (2001) Proc. Natl. Acad. Sci. USA 98: 9877-9882.) |
| | | 343288\|FVT1 | 4.6E−151 | [Homo sapiens][Extracellular (excluding cell wall)] |
| | | | | Follicular lymphoma variant translocation 1, a protein that may |
| | | | | be secreted; correseponding gene has a high rate of transcription |
| | | | | in T-cell malignancies and in phytohemagglutinin-stimulated |
| | | | | lymphocytes |
| | | | | (Rimokh, R. et al. (1993) Blood 81: 136-142.) |
| 14 | 7506151CD1 | g28420 | 1.6E−174 | [Homo sapiens] aldolase B |
| | | | | (Paolella, G. et al. (1984) Nucleic Acids Res. 12: 7401-7410.) |
| | | 339086\|ALDOB | 6.0E−175 | [Homo sapiens][Lyase][Cytoplasmic] Aldolase B |
| | | | | (fructose-bisphosphate aldolase B, liver-type aldolase B), |
| | | | | reversibly cleaves fructose-1,6-bisphosphate or fructose-1- |
| | | | | phosphate into trioses in glycolysis and gluconeogenesis, |
| | | | | deficiency causes hereditary fructose intolerance |
| | | | | (Cross, N. C. et al. (1988) Cell 53: 881-885; Rellos, P. et al. |
| | | | | (2000) J. Biol. Chem. 275: 1145-1151.) |
| | | 330786\|Aldob | 3.1E−169 | [Rattus norvegicus][Lyase] Aldolase B (fructose- |
| | | | | bisphosphate aldolase B), reversibly cleaves fructose-1,6- |
| | | | | bisphosphate or fructose-1-phosphate into trioses in glycolysis |
| | | | | and gluconeogenesis; deficiency of human ALDOB causes fructose |
| | | | | intolerance |
| | | | | (Skala, H. et al. (1987) Eur. J. Biochem. 163: |
| | | | | 513-518; Cross, N. C. et al. (supra)) |
| 15 | 7506450CD1 | g3153832 | 2.8E−24 | [Homo sapiens] UDP-glucuronosyltransferase 2B4 precursor |
| | | 348401\|UGT2B4 | 2.5E−25 | [Homo sapiens][Transferase][Endoplasmic reticulum; |
| | | | | Cytoplasmic] UDP-glucuronosyltransferase 2B member 4, enzyme |
| | | | | that catalyzes the glucuronidation of bile acids and steroids |
| | | | | which can inactivate and increase the solubility of these |
| | | | | substrates and lead to their excretion |
| | | | | (Turgeon, D. et al. (2001) Endocrinology 142: 778-787.) |
| | | 332512\|Rn.24945 | 5.7E−20 | [Rattus norvegicus][Transferase][Endoplasmic |
| | | | | reticulum; Cytoplasmic] Member of a UDP-glucuronosyltransferase |
| | | | | family of endoplasmic reticulum glycoproteins that conjugate |
| | | | | lipophilic aglycon substrates with glucuronic acid, |
| | | | | glucuronidates endogenous steroid substrates but not foreign |
| | | | | compounds |
| | | | | (Li, Y. Q. et al. (1999) Pharm. Res. 16: 191-197.) |
| 16 | 71380031CD1 | g3320415 | 5.8E−123 | [Gallus gallus] ecto-ATP-diphosphohydrolase |
| | | | | (Nagy, A. K. et al. (1998) J. Biol. Chem. 273: 16043-16049.) |
| | | 584459\|Cd39l1 | 1.4E−102 | [Mus musculus][Hydrolase; ATPase][Extracellular |
| | | | | (excluding cell wall)] Ectonucleoside triphosphate |
| | | | | diphosphohydrolase 2, a member of the CD39-like family that |
| | | | | functions as a ecto-ATPase |
| | | | | (Gao, L. et al. (1998) J. Biol. Chem. 273: 15358-15365; Chadwick, |
| | | | | B. P., and Frischauf, A. M. (1997) Mamm. Genome 8: 668-672.) |
| | | 334536\|ENTPD1 | 4.3E−97 | [Homo sapiens][Adhesin/agglutinin; Hydrolase; ATPase] |
| | | | | [Plasma membrane] Vascular ATP diphosphohydrolase, an |
| | | | | integral membrane ectoapyrase that hydrolyzes extracellular ATP |
| | | | | and ADP to AMP, expressed primarily on activated lymphoid cells, |
| | | | | mediates B cell homotypic adhesion, platelet aggregation and |
| | | | | immunological escape of tumor |
| | | | | (Dzhandzhugazyan, K. N. et al. (1998) FEBS Lett. 430: 227-230.) |
| 17 | 7506054CD1 | g34789 | 9.1E−220 | [Homo sapiens] muscle-specific enolase |
| | | | | (Cali, L. et al. (1990) Nucleic Acids Res. 18: 1893.) |
| | | 335214\|ENO3 | 7.9E−221 | [Homo sapiens][Lyase] Muscle-specific enolase (beta |
| | | | | enolase), catalyzes conversion of 2-phospho-D-glycerate to |
| | | | | phosphoenolpyruvate in glycolysis |
| | | 582749\|Eno3 | 6.6E−217 | [Mus musculus][Lyase][Cytoplasmic] Muscle-specific |
| | | | | enolase (beta enolase), converts 2-phospho-D-glycerate to |
| | | | | phosphoenolpyruvate in glycolysis, expressed post-natally |
| | | | | (Merkulova, T. et al. (2000) Eur. J. Biochem. 267: 3735-3743; |
| | | | | Lamande, N. et al. (1989) Proc. Natl. Acad. Sci. USA 86: 4445-4449.) |
| 18 | 7506139CD1 | g2342486 | 2.0E−275 | [Homo sapiens] dihydropyrimidinase related protein 4 |
| | | | | (Hamajima, N. et al. (1996) Gene 180: 157-163.) |
| | | 623866\|DPYSL4 | 1.8E−276 | [Homo sapiens][Hydrolase] Member of the |
| | | | | dihydropyrimidinase family that may function in neuronal |
| | | | | differentiation |
| | | | | (Byk, T. et al. (1998) Eur. J. Biochem. 254: 14-24; Honnorat, J. |
| | | | | et al. (1999) Eur. J. Neurosci. 11: 4226-4232.) |
| | | 323008\|Dpysl4 | 3.8E−263 | [Mus musculus][Hydrolase] Member of the |
| | | | | dihydropyrimidinase family that may function in neuronal |
| | | | | differentiation |
| | | | | (Quach, T. T. et al. (2000) Gene 242: 175-182; Byk, T. et al. (supra)) |
| 19 | 7506426CD1 | g14043373 | 7.6E−175 | [Homo sapiens] adenosine deaminase |
| | | 339048\|ADA | 2.3E−175 | [Homo sapiens][Hydrolase][Plasma membrane] |
| | | | | Adenosine deaminase, has roles in immune response and may be |
| | | | | associated with autism; deficiency causes a form of severe |
| | | | | combined immunodeficiency |
| | | | | (Persico, A. M. et al. (2000) Am. J. Med. Genet. 96: 784-790.) |
| | | 583545\|Ada | 3.7E−149 | [Mus musculus][Hydrolase] Adenosine deaminase, has |
| | | | | roles in immune response and various developmental processes; |
| | | | | mutants die pernatally and show liver-cell degeneration, |
| | | | | atelectasis and small intestinal cell death, but retain an |
| | | | | apparently normal thymus |
| | | | | (Migchielsen, A. A. et al. (1995) Nat. Genet. 10: 279-287; |
| | | | | Wakamiya, M. et al. (1995) Proc. Natl. Acad. Sci. USA 92: 3673-3677.) |
| 20 | 7506741CD1 | g456998 | 1.4E−188 | [Homo sapiens] fatty acid omega-hydroxylase; CYP4A11v |
| | | | | (Imaoka, S. et al. (1993) DNA Cell Biol. 12: 893-899.) |
| | | 334968\|CYP4A11 | 2.2E−201 | [Homo sapiens][Oxidoreductase; Transporter; Small |
| | | | | molecule-binding protein][Endoplasmic reticulum; |
| | | | | Cytoplasmic] Cytochrome P450, subfamily 4A, polypeptide 11, |
| | | | | fatty acid omega-hydroxylase, hydroxylates lauric acid, palmitic |
| | | | | acid, arachidonic acid, and prostaglandins, metabolites of which |
| | | | | regulate kidney function as well as arterial blood pressure |
| | | | | (Oliw, E. H. et al. (2001) Biochem. Pharmacol. 62: 407-415.) |
| | | 665657\|Cyp4a10 | 3.1E−154 | [Mus musculus][Oxidoreductase; Transporter; Small molecule- |
| | | | | binding protein] Cytochrome P450 (subfamily CYP4A polypeptide |
| | | | | 10), lauric acid omega-hydroxylase, hydroxylates fatty acids and |
| | | | | catalyzes peroxidation of endogenous lipids in the liver; |
| | | | | transcriptionally regulated by PPAR alpha (Ppara) |
| | | | | (Leclercq, I. A. et al. (2000) J. Clin. Invest. 105: 1067-1075.) |
| 21 | 7506743CD1 | g456998 | 9.4E−115 | [Homo sapiens] fatty acid omega-hydroxylase; CYP4A11v |
| | | | | (Imaoka, S. et al. (supra)) |
| | | 334968\|CYP4A11 | 8.2E−116 | [Homo sapiens][0xidoreductase; Transporter; Small molecule- |
| | | | | binding protein][Endoplasmic reticulum; Cytoplasmic] |
| | | | | Cytochrome P450, subfamily 4A, polypeptide 11, fatty acid |
| | | | | omega-hydroxylase, hydroxylates lauric acid, palmitic acid, |
| | | | | arachidonic acid, and prostaglandins, metabolites of which |
| | | | | regulate kidney function as well as arterial blood pressure |
| | | | | (Oliw, E. H. et al. (supra)) |
| | | 329852\|Rn.10034 | 1.3E−85 | [Rattus norvegicus][Oxidoreductase; Transporter; Small |
| | | | | molecule-binding protein] Cytochrome P450 (subfamily CYP4A), |
| | | | | expression is induced by androgen treatment, highly expressed in |
| | | | | kidney proximal tubules |
| | | | | (Stromstedt, M. et al. (1990) DNA Cell Biol. 9: 569-577; Kocarek, |
| | | | | T. A. et al. (1998) Mol. Pharmacol. 54: 474-484.) |
| 22 | 7506746CD1 | g456998 | 3.6E−138 | [Homo sapiens] fatty acid omega-hydroxylase; CYP4A11v |
| | | | | (Imaoka, S. et al. (supra)) |
| | | 334968\|CYP4A11 | 3.1E−139 | [Homo sapiens][Oxidoreductase; Transporter; Small |
| | | | | molecule-binding protein][Endoplasmic reticulum; |
| | | | | Cytoplasmic] Cytochrome P450, subfamily 4A, polypeptide 11, |
| | | | | fatty acid omega-hydroxylase, hydroxylates lauric acid, palmitic |
| | | | | acid, arachidonic acid, and prostaglandins, metabolites of which |
| | | | | regulate kidney function as well as arterial blood pressure |
| | | | | (Oliw, E. H. et al. (supra)) |
| | | 665657\|Cyp4a10 | 3.6E−106 | [Mus musculus][Oxidoreductase; Transporter; Small |
| | | | | molecule-binding protein] Cytochrome P450 (subfamily CYP4A |
| | | | | polypeptide 10), lauric acid omega-hydroxylase, hydroxylates |
| | | | | fatty acids and catalyzes peroxidation of endogenous lipids in |
| | | | | the liver; transcriptionally regulated by PPAR alpha (Ppara) |
| | | | | (Leclercq, I. A. et al. (supra)) |
| 23 | 7506748CD1 | g183301 | 4.1E−80 | [Homo sapiens] glutathione transferase |
| | | | | (Vorachek, W. R. et al. (1991) Proc. Natl. Acad. Sci. U.S.A. |
| | | | | 88: 4443-4447.) |
| | | 335658\|GSTM2 | 3.6E−81 | [Homo sapiens][Transferase] Member of the mu class of |
| | | | | glutathione S-transferases, a family of detoxification enzymes |
| | | | | that catalyzes the conjugation of glutathione to electrophilic |
| | | | | compounds, inhibited by chloride ions |
| | | | | (Vorachek, W. R. et al. (supra); Patskovsky, Y. V. et al. (2000) |
| | | | | J. Biol. Chem. 275: 3296-3304.) |
| | | 586691\|Gstm6 | 2.6E−66 | [Mus musculus][Transferase] Member of the mu class of |
| | | | | glutathione S-transferases, a family of detoxification enzymes |
| | | | | that catalyze the conjugation of glutathione to electrophilic |
| | | | | compounds |
| | | | | (De Bruin, W. C. et al. (1998) Biochem. J. 330: 623-626; Fulcher, |
| | | | | K. D. et al. (1995) Mol. Reprod. Dev. 42: 415-424.) |
| 24 | 1419966CD1 | g619877 | 0.0 | [Homo sapiens] hydroxymethylglutaryl-CoA synthase |
| | | | | Mascaro, C. et al. (1995) Arch. Biochem. Biophys. 317: 385-390 |
| | | | | Molecular cloning and tissue expression of human mitochondrial |
| | | | | 3-hydroxy-3-methylglutaryl-CoA synthase. |
| | | HMGCS2 | 0.0 | [Homo sapiens] HMGCS2; mHS 3-hydroxy-3-methylglutaryl- |
| | | | | Coenzyme A (HMG-CoA) synthase 2, mitochondrial enzyme that |
| | | | | catalyzes the synthesis of HMG-CoA, functions in the regulation |
| | | | | of ketogenesis regulates its own gene expression by associating |
| | | | | with the nuclear hormone receptor PPARA |
| | | | | Serra, D. et al. (1996) J. Biol. Chem. 271: 7529-7534 |
| | | | | Tissue-specific expression and dietary regulation of chimeric |
| | | | | mitochondrial 3-hydroxy-3-methylglutaryl coenzyme A synthase/human |
| | | | | growth hormone gene in transgenic mice. |
| | | RN29594 | 0.0 | [Rattus norvegicus] 3-hydroxy-3-methylglutaryl-Coenzyme A |
| | | | | synthase 2, mitochondrial enzyme that appears to regulate |
| | | | | ketogenesis in both normal and regenerating liver; human |
| | | | | HMGCS2 regulates its gene expression by associating with the |
| | | | | nuclear hormone receptor PPARA |
| 25 | 7506451CD1 | g37589 | 3.8E−149 | [Homo sapiens] precursor |
| | | | | Jackson, M. R. et al. (1987) Biochem. J. 242: 581-588 |
| | | | | Cloning of a human liver microsomal UDP-glucuronosyltransferase cDNA. |
| | | 348401\|UGT2B4 | 3.3E−150 | [Homo sapiens] [Transferase] [Endoplasmic reticulum; Cytoplasmic] UDP- |
| | | | | glucuronosyliransferase 2B member 4, enzyme that catalyzes the |
| | | | | glucuronidation of bile acids and steroids which can inactivate |
| | | | | and increase the solubility of these substrates and lead to their |
| | | | | excretion |
| | | | | Turgeon, D. et al. (2001) Endocrinol. 142: 778-787 |
| | | | | Relative Enzymatic Activity, Protein Stability, and Tissue |
| | | | | Distribution of Human Steroid-Metabolizing UGT2B Subfamily Members. |
| | | 338816\|UGT2B7 | 3.5E−127 | [Homo sapiens] [Transferase] [Endoplasmic reticulum; Cytoplasmic; Unspecified |
| | | | | membrane] UDP glycosyltransferase 2 family (polypeptide B7), a |
| | | | | member of the UGT2B subfamily, glucuronidates androgens, |
| | | | | retinoids, lipids, and specific drugs, and may act on |
| | | | | other steroids in the liver and small intestine |
| 26 | 90015249CD1 | g16923707 | 2.1E−25 | [Homo sapiens] (AF435971) glutathione transferase T1-1 |
| | | | | Pemble, S. et al. (1994) Biochem. J. 300: 271-276 |
| | | | | Human glutathione S-transferase theta (GSTT1): cDNA cloning and |
| | | | | the characterization of a genetic polymorphism. |
| | | 335668\|GSTT1 | 1.8E−26 | [Homo sapiens] [Transferase] Member of the theta class |
| | | | | of glutathione S-transferases, conjugates glutathione to |
| | | | | dichloromethane and is localized in erythrocytes; null alleles |
| | | | | may confer a greater risk of myelodystrophic syndromes |
| | | | | Chen, H. et al. (1996) Lancet 347: 295-297 |
| | | | | Increased risk for myelodysplastic syndromes in individuals with |
| | | | | glutathione transferase theta 1 (GSTT1) gene defect. |
| | | 331668\|Rn.11122 | 3.7E−26 | [Rattus norvegicus] [Transferase] [Cytoplasmic; |
| | | | | Mitochondrial; Cell junction] Member of the theta class of |
| | | | | glutathione S-transferases; activity of murine ortholog Gstt1 |
| | | | | activity on dichloromethane and methylene chloride may be |
| | | | | responsible for the carcinogenicity of these compounds in mice |
| 27 | 7487231CD1 | g1872525 | 2.8E−204 | [Homo sapiens] 15-lipoxygenase |
| | | | | Kritzik, M. R. et al. (1997) Biochim. Biophys. Acta 1352: 267-281 |
| | | | | Characterization and sequence of an additional 15-lipoxygenase |
| | | | | transcript and of the human gene. |
| | | 334144\|ALOX15 | 2.4E−205 | [Homo sapiens] [Oxidoreductase] [Plasma membrane] |
| | | | | Arachidonate 15-lipoxygenase, converts arachidonic acid to 15 |
| | | | | hydro(pero)xyeicosatetraenoic acid (15 HPETE) and lipoxin A4, |
| | | | | involved in inflammatory responses, membrane remodeling, may |
| | | | | play a role in atherosclerosis |
| | | | | Kelavkar, U. P., and Badr, K. F. (1999) Proc. Natl. Acad. Sci. USA 96, 4378-4383 |
| | | | | Effects of mutant p53 expression on human 15-lipoxygenase-promoter |
| | | | | activity and murine 12/15-lipoxygenase gene expression: evidence |
| | | | | that 15-lipoxygenase is a mutator gene. |
| | | 704858\|Alox12 | 9.7E−154 | [Rattus norvegicus] [Oxidoreductase] Arachidonate |
| | | | | 12-lipoxygenase, converts arachidonic acid to 12 |
| | | | | hydroperoxyeicosatetraenoic (12 HPETE) and has some 15 |
| | | | | lipoxygenase activity; human ALOX15 may play a role in atherosclerosis |
| 28 | 7506260CD1 | g12803665 | 7.4E−192 | [Homo sapiens] peroxisomal D3,D2-enoyl-CoA isomerase |
| | | 343146\|PECI | 6.9E−174 | [Homo sapiens] [Isomerase][Cytoplasmic; |
| | | | | Peroxisome] Peroxisomal D3,D2-enoyl-CoA isomerase, catalyzes |
| | | | | the isomerization of 3-cis-octenoyl-CoA to 2-trans-octenoyl-CoAm, |
| | | | | a step in the beta oxidation of fatty acids in peroxisomes |
| | | | | Suk, K., et al. (1999) Biochem. Biophys. Acta 1454: 126-131 |
| | | | | Molecular cloning and expression of a novel human cDNA related |
| | | | | to the diazepam binding inhibitor. |
| | | 430126\|Peci | 3.0E−130 | [Mus musculus] [Isomerase] [Cytoplasmic; |
| | | | | Peroxisome] Peroxisomal delta3, delta2-enoyl-Coenzyme A |
| | | | | isomerase, may play a role in the beta oxidation of fatty acids |
| | | | | in peroxisomes |
| 29 | 7506270CD1 | g546603 | 9.6E−179 | [Homo sapiens] glutamine synthetase; GS |
| | | | | Christa, L. et al. (1994) Gastroenterology 106: 1312-1320 |
| | | | | Overexpression of glutamine synthetase in human primary liver cancer. |
| | | 335542\|GLUL | 2.2E−179 | [Homo sapiens] [Ligase] Glutamine synthase, catalyzes |
| | | | | the condensation of glutamate and ammonia to form glutamine, may |
| | | | | clear L-glutamate from neuronal synapses and remove ammonia from |
| | | | | the circulation |
| | | | | Toki, H. et al. (1998) J. Neurochem. 71: 913-919 |
| | | | | Enhancement of extracellular glutamate scavenge system in |
| | | | | injured motoneurons. |
| | | 437801\|Glul | 1.0E−170 | [Rattus norvegicus] [Ligase] Glutamine synthase, |
| | | | | catalyzes the condensation of glutamate and ammonia to form |
| | | | | glutamine, may clear L-glutamate from neuronal synapses |
| 30 | 7506306CD1 | g16518970 | 3.2E−124 | [Gallus gallus] (AF426405) ecto-ATP-diphosphohydrolase |
| | | 329080\|Rn.8276 | 3.3E−101 | [Rattus norvegicus] [Hydrolase; ATPase] |
| | | | | [Extracellular (excluding cell wall); Plasma membrane] |
| | | | | Ecto-ATPase, hydrolyzes extracellular ATP acting as a signaling |
| | | | | molecule |
| | | | | Okada, T. et al. Cell Tissue Res. (1999) 298: 511-518 |
| | | | | Dynamics of rat liver ecto-ATPase during development suggests its |
| | | | | involvement in bile acid efflux. A cytochemical view. |
| | | 334536\|ENTPD1 | 4.3E−97 | [Homo sapiens] [Adhesin/agglutinin; Hydrolase; ATPase] |
| | | | | [Plasma membrane] Vascular ATP diphosphohydrolase, an |
| | | | | integral membrane ectoapyrase that hydrolyzes extracellular |
| | | | | ATP and ADP to AMP, expressed primarily on activated lymphoid |
| | | | | cells, mediates B cell homotypic adhesion, platelet aggregation |
| | | | | and immunological escape of tumor |
| 31 | 7506428CD1 | g178077 | 5.6E−159 | [Homo sapiens] adenosine deaminase |
| | | | | Berkvens, T. M. (1990) Genomics 7: 486-490 |
| | | | | Identical 3250-bp deletion between two Alul repeats in the ADA |
| | | | | genes of unrelated ADA-SCID[adenosine deaminase-severe |
| | | | | combined immunodeficiency] patients. |
| | | 339048\|ADA | 1.6E−159 | [Homo sapiens] [Hydrolase] [Plasma membrane] |
| | | | | Adenosine deaminase, has roles in immune response and may be |
| | | | | associated with autism; deficiency causes a form of severe |
| | | | | combined immunodeficiency |
| | | | | Persico, A. M. (2000) Am. J. Med. Genet. 96: 784-790 |
| | | | | Adenosine deaminase alleles and autistic disorder: case-control |
| | | | | and family-based association studies. |
| | | 583545\|Ada | 1.1E−132 | [Mus musculus] [Hydrolase] Adenosine deaminase, has |
| | | | | roles in immune response and various developmental processes; |
| | | | | mutants die pernatally and show liver-cell degeneration, |
| | | | | atelectasis and small intestinal cell death, but retain an |
| | | | | apparently normal thymus |
| 32 | 7678032CD1 | g15375068 | 2.0E−68 | [Arabidopsis thaliana] gamma hydroxybutyrate dehydrogenase |
| | | 327644\|HIBADH | 4.3E−25 | [Rattus norvegicus] [Oxidoreductase] 3-Hydroxyisobutyrate |
| | | | | dehydrogenase, catalyzes the NAD-dependent oxidation of |
| | | | | 3-hydroxbutyrate to methylmalonate in valine catabolism |
| | | | | Hawes, J. W. et al. (1995) Biochemistry 34: 4231-4237 |
| | | | | Chemical modification and site-directed mutagenesis studies of |
| | | | | rat 3-hydroxyisobutyrate dehydrogenase. |
| 33 | 7508332CD1 | g4753766 | 4.2E−266 | [Homo sapiens] UDP glucuronosyltransferase |
| | | | | (Jedlitschky, G. et al. (1999) Biochem. J. 340: 837-843.) |
| | | 428234\|UGT2A1 | 3.6E−267 | [Homo sapiens][Transferase][Endoplasmic reticulum; |
| | | | | Cytoplasmic] Olfactory UDP-glucuronosyltansferase, catalyzes |
| | | | | the glucuronidation of substrates including odorants, steroids, |
| | | | | drugs, and carcinogens, may play a role in olfaction and in |
| | | | | protecting the neural system from airborne toxins |
| | | | | (Jedlitschky, G. et al. (1999) supra; Strassburg, C. P. et al. |
| | | | | (2000) J. Biol. Chem. 275: 36164-36171.) |
| | | 626952\|Ugt2alp | 9.1E−240 | [Rattus norvegicus][Transferase] Olfactory |
| | | | | UDP-glucuronosyltransferase, catalyzes the glucuronidation of |
| | | | | odorants, may play a role in olfaction and in protecting the |
| | | | | neural system from airborne toxins |
| | | | | (Lazard, D. et al. (1991) Nature 349: 790-793; Heydel, J. et |
| | | | | al. (2001) Drain Res. Mol. Brain Res. 90: 83-92.) |
| 34 | 1288969CD1 | g12803655 | 1.2E−173 | [Homo sapiens] carbonic anhydrase XI |
| | | 610758\|CA11 | 1.0E−174 | [Homo sapiens][Lyase] Carbonic anhydrase-related |
| | | | | protein 11, a member of the carbonic anhydrase family, may not |
| | | | | have catalytic activity due to the lack of active site residues, |
| | | | | may interact with proteins involved in intracellular signal |
| | | | | transduction |
| | | | | (Fujikawa-Adachi, K. et al. (1999) Biochim. Biophys. |
| | | | | Acta 1431: 518-524; Okamoto. N. et al. (2001) Biochim. Biophys. |
| | | | | Acta 1518: 311-316.) |
| | | 418781\|Car11 | 9.8E−106 | [Mus musculus][Lyase] Carbonic anhydrase-related |
| | | | | protein 11, a member of the carbonic anhydrase family, may not |
| | | | | have catalytic activity due to the lack of active-site residues, |
| | | | | may interact with other proteins involved in intracellular signal |
| | | | | transduction |
| | | | | (Lovejoy, D. A. et al. (1998) Genomics 54: 484-493.) |
| 35 | 72069135CD1 | g13925759 | 3.8E−81 | [Homo sapiens] isopentenyl diphosphate dimethylallyl diphosphate isomerase 1 |
| | | 340750\|IDI1 | 3.3E−82 | [Homo sapiens][Isomerase][Cytoplasmic; Peroxisome] |
| | | | | Isopentenyl-diphosphate delta isomerase (IPP isomerase), |
| | | | | catalyzes the interconversion of isopentenyl diphosphate and |
| | | | | dimethylallyl diphosphate in isoprenoid synthesis |
| | | | | (Krisans, S. K. et al. (1994) J. Biol. Chem. 269: 14165-14169; |
| | | | | Hahn. F. M. et al. (1996) Arch. Biochem. Biophys. 332: 30-34.) |
| | | 757016\|Idi1 | 4.9E−72 | [Rattus norvegicus][Isomerase][Cytoplasmic; |
| | | | | Peroxisome] Isopentenyl-diphosphate delta isomerase (IPP |
| | | | | isomerase), involved in the biosynthesis of cholesterol from |
| | | | | mevalonate |
| | | | | (Paton, V. G. et al. (1997) J. Biol. Chem. 272: 18945-18950; |
| | | | | Morihara, T. et al. (1999) Brain Res. Mol. Brain Res. 67: 231-238.) |
| 36 | 7506247CD1 | g13183088 | 2.9E−90 | [Homo sapiens] steroid dehydrogenase-like protein |
| | | 709509\|LOC83693 | 2.5E−91 | [Homo sapiens] Member of the short-chain dehydrogenase-reductase |
| | | | | family, has moderate similarity to rat Rn.10895, which is a 17 |
| | | | | beta-hydroxysteroid dehydrogenase that preferentially converts |
| | | | | androstenedione to testosterone and is regulated by glucose and |
| | | | | gonadotropin |
| 37 | 7506363CD1 | g3005057 | 4.0E−182 | [Homo sapiens] GDP-mannose 4,6 dehydratase; GDPMD |
| | | | | (Sullivan, F. X. et al. (1998) J. Biol. Chem. 273: 8193-8202.) |
| | | 335546\|GMDS | 3.4E−183 | [Homo sapiens][Lyase; Isomerase] GDP-mannose-4,6- |
| | | | | dehydratase, an epimerase that converts GDP-mannose to |
| | | | | GDP-mannose-4-keto-6-D-deoxymannose, plays a role in the synthesis |
| | | | | of fucosylated oligosaccharides |
| | | | | (Ohyama, C. et al. (1998) J. Biol. Chem. 273: 14582-14587; |
| | | | | Sullivan, F. X. et al. (1998) J. Biol. Chem. 273: 8193-8202.) |
| | | 713202\|C53B4.7 | 3.2E−109 | [Caenorhabditis elegans][Lyase][Cytoplasmic] |
| | | | | Putative GDP-mannose 4,6-dehydratase |
| 38 | 7509068CD1 | g184475 | 1.4E−236 | [Homo sapiens] UDP-glucuronosyltransferase 2 family |
| | | | | polypeptide B |
| | | | | (Ritter, J. K. et al. (1991) J. Biol. Chem. 266: 1043-1047.) |
| | | 432576\|UGT2B | 1.1E−237 | [Homo sapiens][Transferase][Endoplasmic reticulum; |
| | | | | Cytoplasmic] UDP glycosyltransferase 2 family polypeptide B, |
| | | | | a UDP-glucuronosyltransferase that glucuronidates bilirubin IX alpha |
| | | 333552\|Rn.37852 | 4.1E−171 | [Rattus norvegicus][Transferase][Endoplasmic |
| | | | | reticulum; Cytoplasmic] Bilirubin UDP-glucuronosyltransfcrase, |
| | | | | a putative UDP-glucuronosyltransferase and member of a UDP- |
| | | | | glucuronosyltransferase family of endoplasmic reticulum |
| | | | | glycoproteins that conjugate lipophilic aglycon substrates with |
| | | | | glucuronic acid |
| 39 | 7505897CD1 | g17225224 | 1.3E−88 | [Homo sapiens] (AF323990) cytosolic beta-glucosidase |
| | | 690772\|GLUC | 1.1E−89 | [Homo sapiens] Protein with moderate similarity to murine |
| | | | | Kl (klotho), which is a membrane protein that is related to |
| | | | | beta-glucosidases and associated with an aging-related syndrome |
| | | 336238\|LCT | 5.1E−43 | [Homo sapiens][Hydrolase][Plasma membrane] |
| | | | | Lactase-phlorizin hydrolase (brush border membrane beta- |
| | | | | glycosidase complexes), has both lactase and phloridzin hydrolase |
| | | | | activity; decreased activity in adults leads to adult type hypolactasia |
| 40 | 7505898CD1 | g12654243 | 5.9E−170 | [Homo sapiens] SELENOPHOSPHATE SYNTHETASE; Human selenium |
| | | | | donor protein |
| | | 428998\|SPS | 3.7E−162 | [Homo sapiens][Transferase] Selenophosphate synthetase |
| | | | | 1, a selenide, water dikinase that produces monoselenophosphate |
| | | | | from ATP and selenide; monoselenophosphate is the biologically |
| | | | | active selenium donor in the synthesis of selenocysteine for |
| | | | | incorporation into selenoproteins |
| | | 746641\|Sps2 | 9.6E−122 | [Mus musculus][Transferase] Selenophosphate synthetase |
| | | | | 2, a selenoprotein that catalyzes the formation of |
| | | | | monoselenophosphate from selenide and ATP, functions in the |
| | | | | synthesis of selenocysteine for incorporation into proteins |
| 41 | 7505907CD1 | g1150421 | 2.9E−218 | [Homo sapiens] rTSbeta |
| | | | | (Dolnick, B. J. and Black, A. R. (1996) Cancer Res. 56: 3207-3210.) |
| | | 599754\|HSRTSB | 2.5E−219 | [Homo sapiens] rTs beta protein, expression is upregulated |
| | | ETA | | in tumor cell lines resistant to thymidylate synthase inhibitors, |
| | | | | may function in a catabolic pathway to downregulate |
| | | | | thymidylate biosynthesis, may help detect starvation and play a |
| | | | | role in saturated cell growth |
| | | | | (Dolnick, B. J., and Black, A. R. (supra); Dolnick, B. J. et al. |
| | | | | (1997) Adv. Enzyme Regul. 37: 95-109.) |
| 42 | 7505925CD1 | g2342862 | 1.3E−172 | [Homo sapiens] branched chain aminotransferase precursor |
| | | | | (Bledsoe, R. K. et al. (1997) Biochim. Biophys. Acta 1339: 9-13.) |
| | | 334322\|BCAT2 | 1.1E−173 | [Homo sapiens][Transferase][Cytoplasmic; |
| | | | | Mitochondrial] Mitochondrial branched chain aminotransferase, |
| | | | | converts branched-chain alpha-keto acids to L-amino acids |
| | | | | (Eden, A. et al. (1996) J. Biol. Chem. 271: 20242-20245; |
| | | | | Bledsoe, R. K. et al. (1997) Biochim. Biophys. Acta 1339: 9-13.) |
| | | 628649\|Bcat2 | 1.7E−144 | [Rattus norvegicus][Transferase][Cytoplasmic; |
| | | | | Mitochondrial] Mitochondrial branched chain aminotransferase, |
| | | | | converts branched-chain alpha-keto acids to L-amino acids |
| | | | | (Drown, P. M. et al. (2000) Biochim. Biophys. Acta 1468: |
| | | | | 273-284; Hutson, S. M. et al. (1998) J. Neurochem. 71: 863-874.) |

TABLE 3

| | | Amino | | | | Analytical |
|---|---|---|---|---|---|---|
| SEQ | Incyte | Acid | Potential | Potential | | Methods |
| ID | Polypeptide | Resi- | Phosphorylation | Glycosylation | Signature Sequences, Domains | and |
| NO: | ID | dues | Sites | Sites | and Motifs | Databases |
| 1 | 70612021CD1 | 378 | S125 S156 S188 | N248 | signal_cleavage: M1-A26 | SPSCAN |
| | | | S219 S252 S293 | | NAD: arginine ADP-ribosyltransferase: | HMMER_PFAM |
| | | | T44 T68 | | M1-K326 | TMHMMER |
| | | | | | Cytosolic domain: L378—L378 | |
| | | | | | Transmembrane domain: L355-A377 | |
| | | | | | Non-cytosolic domain: M1-K354 | |
| | | | | | NAD: arginine ADP-ribosyltransferases | BLIMPS_BLOCKS |
| | | | | | proteins | |
| | | | | | BL01291: F115-L148, V161-F170, | |
| | | | | | F180-Q232, I243-G261, L29-E58, | |
| | | | | | V69-T82, P87-I102 | |
| | | | | | Arginine ADP-ribosyltransferase | BLIMPS_PRINTS |
| | | | | | signature PR00970: D30-Y51, | |
| | | | | | E59-W77, F90-E104, F133-L149, | |
| | | | | | A178-A193, T199-L215, E220-Q235 | |
| | | | | | TRANSFERASE ADP | BLAST_PRODOM |
| | | | | | RIBOSYLTRANSFERASE | |
| | | | | | NADP + ARGININE PRECURSOR | |
| | | | | | GLYCOSYLTRANSFERASE | |
| | | | | | SIGNAL NAD | |
| | | | | | MONOADPRIBOSYLTRANSFERASE | |
| | | | | | MONO ADP | |
| | | | | | RIBOSYLTRANSFERASE | |
| | | | | | PD004385: K25-P323 | |
| | | | | | TESTIS ECTO-ADP | BLAST_PRODOM |
| | | | | | RIBOSYLTRANSFERASE | |
| | | | | | PRECURSOR EC 2.4.2.31 | |
| | | | | | NADP + ARGININE ADP | |
| | | | | | RIBOSYLTRANSFERASE MONO-ADP | |
| | | | | | RIBOSYLTRANSFERASE | |
| | | | | | TRANSFERASE | |
| | | | | | GLYCOSYLTRANSFERASE | |
| | | | | | GLYCOPROTEIN | |
| | | | | | NAD SIGNAL G PD039596: P336-L378 | |
| | | | | | RIBOSYLTRANSFERASE; | BLAST_DOMO |
| | | | | | NAD; ADP; ARGININE; | |
| | | | | | DM02464\|P17982\|1-274: V28-I268 | |
| | | | | | DM02464\|JC4367\|1-300: | |
| | | | | | V24-E269, P330-H347 | |
| | | | | | DM02464\|A55461\|1-312: V28-L259 | |
| | | | | | DM02464\|P17981\|1-286: V28-N266 | |
| | | | | | NAD: arginine | MOTIFS |
| | | | | | ADP-ribosyltransferases | |
| | | | | | signature: F133-A145 | |
| 2 | 71847235CD1 | 715 | S57 S68 S142 S188 | N52 N113 N327 | signal_cleavage: M1-G22 | SPSCAN |
| | | | S216 S307 S322 | N363 N700 N708 | Signal Peptide: M1-P19, M1-G22, | HMMER |
| | | | S365 S456 S506 | | M1-D24, M1-A17, M1-P25 | |
| | | | T54 T149 T362 | | Animal haem peroxidase: K144-T683 | HMMER_PFAM |
| | | | T381 T430 T659 | | Animal haem peroxidase signature | BLIMPS_PRINTS |
| | | | Y51 Y415 | | PR00457: R169-S180, M223-S238, | |
| | | | | | F371-T389, T389-W409, L414-G440, | |
| | | | | | T467-L477, N595-W615, L666-G680 | |
| | | | | | PEROXIDASE OXIDOREDUCTASE | BLAST_PRODOM |
| | | | | | PRECURSOR SIGNAL HEME | |
| | | | | | GLYCOPROTEIN PROTEIN | |
| | | | | | SIMILAR MYELOPEROXIDASE | |
| | | | | | EOSINOPHIL PD001354: Q582-R690 | |
| | | | | | PEROXIDASE OXIDOREDUCTASE | BLAST_PRODOM |
| | | | | | PRECURSOR SIGNAL | |
| | | | | | MYELOPEROXIDASE HEME | |
| | | | | | GLYCOPROTEIN ASCORBATE | |
| | | | | | CATALASE L-ASCORBATE | |
| | | | | | PD000217: Y145-D234, N494-S581 | |
| | | | | | PEROXIDASE PRECURSOR | BLAST_PRODOM |
| | | | | | OXIDOREDUCTASE | |
| | | | | | HEME GLYCOPROTEIN SIGNAL | |
| | | | | | THYROID TPO TRANSMEMBRANE | |
| | | | | | EOSINOPHIL PD008832: Q18-K144 | |
| | | | | | MYELOPEROXIDASE | BLAST_DOMO |
| | | | | | DM01034\|P11678\|282-714: I282-T715 | |
| | | | | | DM01034\|P05164\|310-743: I282-R713 | |
| | | | | | DM01034\|B28894\|395-828: I282-R713 | |
| | | | | | DM01034\|C28894\|390-823: I282-R713 | |
| | | | | | Peroxidases proximal heme-ligand | MOTIFS |
| | | | | | signature: E380-L390 | |
| 3 | 7505230CD1 | 811 | S153 S159 S169 | N94 N271 N292 | Signal Peptide: M1-C18, M1-R20, | HMMER |
| | | | S233 S311 S361 | N307 N321 N501 | M1-P23, M1-C24 | |
| | | | S447 S505 S526 | N568 N591 N604 | FG-GAP repeat: Y510-T570, F686-K757, | HMMER_PFAM |
| | | | S555 S557 S687 | | F577-Y644, S447-D508, Y378-A437 | |
| | | | S696 S768 T28 | | Phospholipase D signature PR00718: | BLIMPS_PRINTS |
| | | | T112 T323 T386 | | G25-L40, F80-R101, H151-G164, | |
| | | | T480 T570 T601 | | G164-Y177, M385-P408, Y625-V647, | |
| | | | T643 T738 T743 | | M708-N733, D741-Y760, V761-K781 | |
| | | | T777 | | PHOSPHOLIPASE D PRECURSOR | BLAST_PRODOM |
| | | | | | SIGNAL PHOSPHATIDYL- | |
| | | | | | INOSITOL-GLYCAN-SPECIFIC | |
| | | | | | PIG PLD GLYCOPROTEIN | |
| | | | | | GLYCOSYL | |
| | | | | | PHOSPHATIDYLINOSITOL | |
| | | | | | PD014524: M1-G356 | |
| | | | | | PD151292: F536-P630 | |
| | | | | | PD018501: G740-D811 | |
| | | | | | INTEGRIN PRECURSOR | BLAST_PRODOM |
| | | | | | SIGNAL GLYCOPROTEIN | |
| | | | | | ALPHA CELL ADHESION | |
| | | | | | TRANSMEMBRANE EXTRACELLULAR | |
| | | | | | MATRIX PD001221: T310-A537 | |
| | | | | | INTEGRINS ALPHA CHAIN | BLAST_DOMO |
| | | | | | DM00147\|P80109\|335-421: T335-D423 | |
| | | | | | DM00147\|P80109\|423-491: L424-Q493 | |
| | | | | | DM00147\|P80109\|690-769: A662-M742 | |
| | | | | | Prokaryotic Lipoprotein M496-C606 | MOTIFS |
| 4 | 7505235CD1 | 564 | S7 S188 S277 S379 | N97 N153 N186 | signal_cleavage: M1-T27 | SPSCAN |
| | | | S460 S554 T237 | N396 N535 | Signal Peptide: M1-T27, M1-S29, M1-A25 | HMMER |
| | | | T252 T333 T374 | | Glycosyl hydrolases family: M1-K531 | HMMER_PFAM |
| | | | T513 | | Glycosyl hydrolases family 35 proteins | BLIMPS_BLOCKS |
| | | | | | BL01182: L62-A104, F105-L137, | |
| | | | | | N210-F220, T237-G249, K473-W487 | |
| | | | | | Glycosyl hydrolase family 35 signature | BLIMPS_PRINTS |
| | | | | | PR00742: D42-P59, W63-Y81, | |
| | | | | | L118-L137, V171-N186, A207-Y222, | |
| | | | | | L233-G249, S446-S460, K473-K489 | |
| | | | | | BETA-GALACTOSIDASE | BLAST_PRODOM |
| | | | | | LACTASE HYDROLASE | |
| | | | | | PRECURSOR SIGNAL | |
| | | | | | GLYCOSIDASE GLYCOPROTEIN | |
| | | | | | PROTEIN ACID BETA | |
| | | | | | PD003386: L13-P166, M154-M354, | |
| | | | | | F450-L496, L371-L436 | |
| | | | | | BETA-GALACTOSIDASE | BLAST_PRODOM |
| | | | | | PROTEIN LACTASE | |
| | | | | | HYDROLASE H22K11.2 T19B10.3 | |
| | | | | | PD142409: D463-L534 | |
| | | | | | BETA-GALACTOSIDASE CHAIN | BLAST_DOMO |
| | | | | | DM03771\|P16279\|5-510: | |
| | | | | | N153-P521, L9-N186 | |
| | | | | | DM03771\|P16278\|160-641: N153-P521 | |
| | | | | | DM03771\|P23780\|161-643: D152-P521 | |
| | | | | | DM03771\|P48982\|156-597: | |
| | | | | | F158-V498, A25-L53 | |
| 5 | 7505793CD1 | 233 | S21 S172 T57 T103 | N159 | Adenosine/AMP deaminase: M1-L216 | HMMER_PFAM |
| | | | T112 T181 T188 | | Adenosine and AMP deaminase proteins | BLIMPS_BLOCKS |
| | | | Y160 Y178 Y218 | | BL00485: A2-R32, D60-A71, | |
| | | | | | S77-L118, A141-L195 | |
| | | | | | Chromo domain proteins BL00598: L28-V49 | BLIMPS_BLOCKS |
| | | | | | Adenosine and AMP deaminase | PROFILESCAN |
| | | | | | signature: A141-F187 | |
| | | | | | ADENOSINE DEAMINASE | BLAST_PRODOM |
| | | | | | AMINOHYDROLASE HYDROLASE | |
| | | | | | NUCLEOTIDE METABOLISM | |
| | | | | | POLYMORPHISM ACETYLATION | |
| | | | | | PHARMACEUTICAL 3D STRUCTURE | |
| | | | | | PD008716: D8-L58 | |
| | | | | | DEAMINASE HYDROLASE | BLAST_PRODOM |
| | | | | | NUCLEOTIDE METABOLISM | |
| | | | | | ADENOSINE AMP | |
| | | | | | AMINOHYDROLASE ISOFORM | |
| | | | | | MULTIGENE FAMILY | |
| | | | | | PD005960: V75-L214, F61-R81 | |
| | | | | | Adenosine and AMP deaminase | MOTIFS |
| | | | | | signature: S161-P167 | |
| 6 | 7505861CD1 | 221 | S169 S198 T161 | | Phospholipase/Carboxylesterase: | HMMER_PFAM |
| | | | T184 T200 | | L9-L213 | |
| | | | | | PROTEIN LYSOPHOSPHOLIPASE | BLAST_PRODOM |
| | | | | | ESTERASE SERINE I HYDROLASE | |
| | | | | | CARBOXYL-ESTERASE | |
| | | | | | PHOSPHOLIPASE B INTERGENIC | |
| | | | | | PD004631: A22-I209 | |
| | | | | | CARBOXYLESTERASE; YLR118C; | BLAST_DOMO |
| | | | | | ESTERASE; MEMBRANE; | |
| | | | | | DM04666\|JU0277\|1-218: I13-L213 | |
| | | | | | Prokaryotic Lipoprotein I102-C112 | MOTIFS |
| 7 | 7505864CD1 | 283 | S10 S16 S65 S93 | | Lipases, serine active site: V107-P157 | PROFILESCAN |
| | | | S198 S271 T3 T50 | | Alpha/beta hydrolase fold | BLIMPS_PRINTS |
| | | | T141 T168 T240 | | signature PR00111: | |
| | | | Y248 | | A142-I155, D79-E94, L128-T141 | |
| | | | | | Prolyl aminopeptidase (S33) | BLIMPS_PRINTS |
| | | | | | family signature PR00793: | |
| | | | | | L54-G62, V82-S93, L128-A142 | |
| | | | | | H14E PROTEIN K6 MONOGLYCERIDE | BLAST_PRODOM |
| | | | | | LIPASE LYSOPHOSPHOLIPASE | |
| | | | | | HOMOLOG PD007213: M11-L81 | |
| | | | | | LYSOPHOSPHOLIPASE PUTATIVE | BLAST_PRODOM |
| | | | | | HOMOLOG ISOLOG PROTEIN | |
| | | | | | CWP1MBR1 INTERGENIC | |
| | | | | | REGION MONOGLYCERIDE | |
| | | | | | LIPASE PD008235: K206-R273 | |
| | | | | | Lipases, serine active site: V126-G135 | MOTIFS |
| 8 | 7506427CD1 | 339 | S21 S110 S162 | N265 | Adenosine/AMP deaminase: M1-L322 | HMMER_PFAM |
| | | | S278 T57 T125 | | Adenosine and AMP deaminase | BLIMPS_BLOCKS |
| | | | T209 T218 T287 | | proteins BL00485: A2-R32, D60-A71, | |
| | | | T294 Y97 Y266 | | A183-L224, A247-L301 | |
| | | | Y284 Y324 | | Adenosine and AMP deaminase | PROFILESCAN |
| | | | | | signature: A247-F293 | |
| | | | | | ADENOSINE DEAMINASE | BLAST_PRODOM |
| | | | | | AMINO-HYDROLASE | |
| | | | | | HYDROLASE NUCLEOTIDE | |
| | | | | | METABOLISM POLYMORPHISM | |
| | | | | | ACETYLATION | |
| | | | | | PHARMACEUTICAL 3D | |
| | | | | | STRUCTURE PD008716: D8-L58 | |
| | | | | | DEAMINASE HYDROLASE | BLAST_PRODOM |
| | | | | | NUCLEOTIDE METABOLISM | |
| | | | | | ADENOSINE AMP | |
| | | | | | AMINOHYDROLASE ISOFORM | |
| | | | | | MULTIGENE FAMILY | |
| | | | | | PD005960: P195-L320, F61-H105 | |
| | | | | | Adenosine and AMP deaminase | MOTIFS |
| | | | | | signature: S267-P273 | |
| 9 | 7506429CD1 | 130 | S21 S110 T57 Y97 | | Adenosine and AMP deaminase | BLIMPS_BLOCKS |
| | | | | | proteins BL00485: A2-R32, D60-A71 | |
| | | | | | Chromo domain proteins | BLIMPS_BLOCKS |
| | | | | | BL00598: L28-V49 | |
| | | | | | ADENOSINE DEAMINASE | BLAST_PRODOM |
| | | | | | AMINO-HYDROLASE | |
| | | | | | HYDROLASE NUCLEOTIDE | |
| | | | | | METABOLISM POLYMORPHISM | |
| | | | | | ACETYLATION | |
| | | | | | PHARMACEUTICAL 3D | |
| | | | | | STRUCTURE PD008716: D8-L58 | |
| 10 | 7505799CD1 | 127 | S116 T27 T76 T90 | | signal_cleavage: M1-G17 | SPSCAN |
| | | | | | Cytochrome c oxidase subunit IV: | HMMER_PFAM |
| | | | | | P85-K127, T55-L83 | |
| | | | | | CYTOCHROME C OXIDASE IV | BLAST_PRODOM |
| | | | | | POLYPEPTIDE PRECURSOR | |
| | | | | | OXIDOREDUCTASE | |
| | | | | | MITOCHONDRION TRANSIT | |
| | | | | | PEPTIDE PD013306: | |
| | | | | | E79-K127, Q43-F84 | |
| 11 | 7505843CD1 | 277 | S58 S74 S75 S206 | N186 | signal_cleavage: M1-L23 | SPSCAN |
| | | | T33 T147 T188 | | Oxidoreductase FAD-binding | HMMER_PFAM |
| | | | T271 Y224 | | domain: Y20-Y127 | |
| | | | | | Oxidoreductase NAD-binding | HMMER_PFAM |
| | | | | | domain: V148-P262 | |
| | | | | | Cytochrome b5 family, heme-binding | BLIMPS_BLOCKS |
| | | | | | domain proteins BL00191: | |
| | | | | | R60-S74, G100-P121, G156-E199 | |
| | | | | | Eukaryotic molybdopterin | BLIMPS_BLOCKS |
| | | | | | oxidoreductases proteins | |
| | | | | | BL00559: S30-S58, G100-V151, | |
| | | | | | D173-E199, E242-A259 | |
| | | | | | Flavoprotein pyridine nucleotide | BLIMPS_PRINTS |
| | | | | | cytochrome reductase signature | |
| | | | | | PR00371: R68-S75, G100-S109, | |
| | | | | | G152-M171, T178-Q187, K190-L201, | |
| | | | | | W222-L238, L246-P254 | |
| | | | | | Cytochrome B5 reductase signature | BLIMPS_PRINTS |
| | | | | | PR00406: L47-S58, R68-S75, | |
| | | | | | G113-Y127, G152-M171, | |
| | | | | | K190-L201, L246-P254 | |
| | | | | | PROTEIN ANTIOXIDANT | BLIMPS_PRODOM |
| | | | | | PEROXIDASE | |
| | | | | | PD00210: V12-E27 | |
| | | | | | REDUCTASE NITRATE | BLAST_PRODOM |
| | | | | | OXIDOREDUCTASE FLAVOPROTEIN | |
| | | | | | FAD HEME MOLYBDENUM | |
| | | | | | ASSIMILATION NAD NR PD149632: | |
| | | | | | L23-P121 | |
| | | | | | REDUCTASE OXIDOREDUCTASE | BLAST_PRODOM |
| | | | | | FAD FLAVOPROTEIN NADP | |
| | | | | | NITRATE HEME NAD | |
| | | | | | ELECTRON MEMBRANE | |
| | | | | | PD000183: M153-H266 | |
| | | | | | EUKARYOTIC MOLYBDOPTERIN | BLAST_DOMO |
| | | | | | OXIDOREDUCTASES | |
| | | | | | DM00131\|P07514\|50-274: E27-P252 | |
| | | | | | DM00131\|P39866\|645-864: I29-P253 | |
| | | | | | DM00131\|P23312\|681-900: I29-P253 | |
| | | | | | DM00131\|P39869\|655-874: I29-P253 | |
| 12 | 90001378CD1 | 470 | S325 S340 S344 | N122 N208 | Hexokinase: E16-L463 | HMMER_PFAM |
| | | | S363 S379 S449 | | Hexokinases proteins BL00378: | BLIMPS_BLOCKS |
| | | | T35 T114 T161 | | S445-V459, V22-K49, V61-V97, | |
| | | | T172 T275 T279 | | V207-G250, M255-D266, Y277-G322 | |
| | | | T364 T430 Y27 | | Hexokinases signature: V130-R195 | PROFILESCAN |
| | | | | | Hexokinase family signature | BLIMPS_PRINTS |
| | | | | | PR00475: L81-V97, L150-F175, | |
| | | | | | I203-Y219, I226-E240, Q291-L313, | |
| | | | | | V371-I393, M443-V459 | |
| | | | | | HEXOKINASE TRANSFERASE | BLAST_PRODOM |
| | | | | | KINASE GLYCOLYSIS | |
| | | | | | ATP-BINDING TYPE | |
| | | | | | ALLOSTERIC ENZYME | |
| | | | | | HK DUPLICATION | |
| | | | | | PD001109: E16-D439, E252-A460 | |
| | | | | | HEXOKINASES | BLAST_DOMO |
| | | | | | DM00597\|P52789\|465-915: D17-A466 | |
| | | | | | DM00597\|S48809\|465-915: D17-A466 | |
| | | | | | DM00597\|P27881\|465-915: D17-A466 | |
| | | | | | DM00597\|P27595\|465-915: D17-Q464 | |
| 13 | 7504923CD1 | 298 | S24 S80 S127 S136 | N228 | signal_cleavage: M1-P25 | SPSCAN |
| | | | T159 | | Signal Peptide: M1-P25 | HMMER |
| | | | | | short chain dehydrogenase: P31-N293 | HMMER_PFAM |
| | | | | | Cytosolic domains: | TMHMMER |
| | | | | | M1—M1, R282-A298 | |
| | | | | | Transmembrane domains: | |
| | | | | | L2-S24, I259-V281 | |
| | | | | | Non-cytosolic domain: P25-A258 | |
| | | | | | Short-chain | BLIMPS_BLOCKS |
| | | | | | dehydrogenases/reductases | |
| | | | | | family proteins BL00061: | |
| | | | | | G113-G123, G166-E203 | |
| | | | | | Short-chain | PROFILESCAN |
| | | | | | dehydrogenases/reductases | |
| | | | | | family signature: G166-T219 | |
| | | | | | Alcohol dehydrogenase superfamily | BLIMPS_PRINTS |
| | | | | | signature PR00080: G113-M124, | |
| | | | | | G166-Q174, Y186-K205 | |
| | | | | | Glucose/ribitol dehydrogenase family | BLIMPS_PRINTS |
| | | | | | signature PR00081: H34-E51, G113-M124, | |
| | | | | | M160-G176, Y186-K205 | |
| | | | | | FOLLICULAR VARIANT | BLAST_PRODOM |
| | | | | | TRANSLOCATION PROTEIN 1 | |
| | | | | | PRECURSOR FVT1 | |
| | | | | | PROTOONCOGENE CHROMOSOMAL | |
| | | | | | OXIDOREDUCTASE SIGNAL | |
| | | | | | PD078188: V260-A298, P222-I259 | |
| | | | | | FOLLICULAR VARIANT | BLAST_PRODOM |
| | | | | | TRANSLOCATION PROTEIN | |
| | | | | | 1 PRECURSOR FVT1 | |
| | | | | | PROTOONCOGENE CHROMOSOMAL | |
| | | | | | OXIDOREDUCTASE SIGNAL | |
| | | | | | PD058472: M1-H34 | |
| | | | | | SHORT-CHAIN ALCOHOL | BLAST_DOMO |
| | | | | | DEHYDROGENASE FAMILY | |
| | | | | | DM00034\|Q06136\|27-272: P27-I259 | |
| | | | | | DM00034\|P14802\|1-235: V36-V255 | |
| | | | | | DM00034\|P54554\|1-237: L30-A225 | |
| | | | | | DM00034\|P37694\|1-246: L30-K256 | |
| | | | | | Aminoacyl-transfer RNA synthetases | MOTIFS |
| | | | | | class-II signature 2: V135-I144 | |
| | | | | | Short-chain dehydrogenases/reductases | MOTIFS |
| | | | | | family signature: S173-Q201 | |
| 14 | 7506151CD1 | 331 | S132 S276 T119 | N71 | Fructose-bisphosphate aldolase class-I: | HMMER_PFAM |
| | | | T206 T241 T245 | | E15-A318, A319-Y331 | |
| | | | Y223 | | Fructose-bisphosphate aldolase class-I | BLIMPS_BLOCKS |
| | | | | | BL000158: E11-T52, I74-L128, | |
| | | | | | R173-T227, N283-Y331 | |
| | | | | | Fructose-bisphosphate aldolase class-I | PROFILESCAN |
| | | | | | active site: V205-T253 | |
| | | | | | ALDOLASE FRUCTOSEBISPHOSPHATE | BLAST_PRODOM |
| | | | | | LYASE SCHIFF BASE GLYCOLYSIS | |
| | | | | | MULTIGENE FAMILY B CYTOPLASMIC | |
| | | | | | PD001128: E15-S323 | |
| | | | | | FRUCTOSE-BISPHOSPHATE ALDOLASE | BLAST_DOMO |
| | | | | | DM00718\|S47540\|2-362: | |
| | | | | | A2-F325, K297-T330 | |
| | | | | | DM00718\|S57270\|2-362: | |
| | | | | | H3-F325, A319-Y329 | |
| | | | | | DM00718\|JC4189\|2-362: F5-S320 | |
| | | | | | DM00718\|JC4188\|2-361: F5-K317 | |
| | | | | | Fructose-bisphosphate aldolase | MOTIFS |
| | | | | | class-I active site: V222-N232 | |
| 15 | 7506450CD1 | 102 | S2 S84 T41 | | signal_cleavage: M1-G21 | SPSCAN |
| | | | | | Signal Peptide: M3-G21, M3-C23, | HMMER |
| | | | | | M3-G24, M3-S22, | |
| | | | | | UDP-glucoronosyl and UDP-glucosyl | HMMER_PFAM |
| | | | | | transferas: G24-E52 | |
| | | | | | UDP-GLUCORONOSYL AND UDP-GLUCOSYL | BLAST_DOMO |
| | | | | | TRANSFERASES | |
| | | | | | DM00435\|P36538\|1-185: M1-E87 | |
| | | | | | DM00435\|P06133\|1-185: M1-E87 | |
| | | | | | DM00435\|P54855\|1-186: M1-L93 | |
| | | | | | DM00435\|A48633\|1-186: M1-L93 | |
| 16 | 71380031CD1 | 458 | S4 S132 S136 S153 | N67 N135 N304 | signal_cleavage: M1-G21 | SPSCAN |
| | | | T226 | N325 N410 | Signal Peptide: M1-G21, M1-T23, | HMMER |
| | | | | | M1-A24, M1-L25 | |
| | | | | | GDA1/CD39 (nucleoside phosphatase) | HMMER_PFAM |
| | | | | | family: S34-R424 | |
| | | | | | Cytosolic domains: M1-A12, D458—D458 | TMHMMER |
| | | | | | Transmembrane domains: L13-V35, | |
| | | | | | V435-Q457 | |
| | | | | | Non-cytosolic domain: L36-K434 | |
| | | | | | GDA1/CD39 family of nucleoside | BLIMPS_BLOCKS |
| | | | | | phosphatases proteins BL01238: | |
| | | | | | I45-Y59, P119-R129, L163-V184, | |
| | | | | | G202-F215 | |
| | | | | | HYDROLASE TRANSMEMBRANE | BLAST_PRODOM |
| | | | | | PROTEIN NUCLEOSIDE CD39 | |
| | | | | | NUCLEOSIDETRIPHOSPHATASE | |
| | | | | | TRIPHOSPHATE NTPASE | |
| | | | | | PRECURSOR | |
| | | | | | ATPDIPHOSPHOHYDROLASE | |
| | | | | | PD003822: T33-C329, | |
| | | | | | Q194-Y355, Y355-P420, I45-G68 | |
| | | | | | ACTIVATION; NUCLEOSIDE; | BLAST_DOMO |
| | | | | | ANTIGEN; LYMPHOID; | |
| | | | | | DM02628\|I56242\|40-471: | |
| | | | | | L37-F349, V351-P420 | |
| | | | | | DM02628\|P49961\|40-471: | |
| | | | | | L37-F349, V351-P420 | |
| | | | | | DM02628\|P32621\|84-517: | |
| | | | | | K42-D400, Y365-L411 | |
| | | | | | DM02628\|P52914\|35-454: F43-Y278 | |
| | | | | | GDA1/CD39 family of nucleoside | MOTIFS |
| | | | | | phosphatases signature: L163-Y178 | |
| 17 | 7506054CD1 | 420 | S83 S177 S249 | N70 N102 | Enolase: M1-K418 | HMMER_PFAM |
| | | | S277 S335 S356 | | Enolase proteins BL00164: R32-K54, | BLIMPS_BLOCKS |
| | | | S387 T26 Y57 | | D98-N140, I144-K193, N206-K248, | |
| | | | | | Y273-W287, I299-G334, F366-G404 | |
| | | | | | Enolase signature: Q284-N331 | PROFILESCAN |
| | | | | | Enolase signature PR00148: V35-L49, | BLIMPS_PRINTS |
| | | | | | G107-A123, A164-S177, G303-I314, | |
| | | | | | L326-I340, V355-V372 | |
| | | | | | ENOLASE LYASE | BLAST_PRODOM |
| | | | | | GLYCOLYSIS MAGNESIUM | |
| | | | | | HYDROLYASE | |
| | | | | | 2PHOSPHOGLYCERATE | |
| | | | | | DEHYDRATASE 2PHOSPHOD | |
| | | | | | GLYCERATE | |
| | | | | | 2PHOSPHODGLYCERATE | |
| | | | | | PD000902: M3-A219, D142-N272 | |
| | | | | | PD000948: V371-K418 | |
| | | | | | LYASE MAGNESIUM ENOLASE | BLAST_PRODOM |
| | | | | | GLYCOLYSIS | |
| | | | | | HYDROLYASE DEHYDRATASE | |
| | | | | | 2PHOSPHOGLYCERATE 2PHOSPHOD | |
| | | | | | GLYCERATE 2PHOSPHODGLYCERATE | |
| | | | | | PD003216: D252-E401 | |
| | | | | | ENOLASE | BLAST_DOMO |
| | | | | | DM00487\|P15429\|1-430: A2-K418 | |
| | | | | | DM00487\|JC4187\|1-431: M1-K418 | |
| | | | | | DM00487\|JC1039\|1-431: M1-P417 | |
| | | | | | DM00487\|S52858\|1-431: M1-P417 | |
| | | | | | Enolase signature: L326-S339 | MOTIFS |
| 18 | 7506139CD1 | 512 | S14 S147 S226 | N507 | Dihydroorotase-like: K56-H444 | HMMER_PFAM |
| | | | S322 S372 S405 | | PROTEIN HYDROLASE | BLAST_PRODOM |
| | | | S421 S484 T84 | | PYRIMIDINE BIOSYNTHESIS | |
| | | | T218 T312 | | DIHYDROPYRIMIDINASE | |
| | | | | | DIHYDROOROTASE ZINC | |
| | | | | | RELATED DHOASE | |
| | | | | | ASPARTATE PD001085: I58-E430 | |
| | | | | | DIHYDROPYRIMIDINASE | BLAST_PRODOM |
| | | | | | RELATED PROTEIN | |
| | | | | | COLLAPSIN RESPONSE | |
| | | | | | MEDIATOR PROTEIN2 | |
| | | | | | DRP2 PROTEIN1 DRP1 | |
| | | | | | PD000518: D15-G54 | |
| | | | | | PD149872: P469-S512 | |
| | | | | | DIHYDROOROTASE | BLAST_DOMO |
| | | | | | DM00754\|P47942\|13-429: T13-E430 | |
| | | | | | DM00754\|S55525\|13-429: T13-E430 | |
| | | | | | DM00754\|S58890\|1-366: M64-E430 | |
| | | | | | DM00754\|JC2310\|1-413: L18-E430 | |
| 19 | 7506426CD1 | 327 | S21 S110 S266 T57 | N253 | Adenosine/AMP deaminase: M1-L310 | HMMER_PFAM |
| | | | T125 T197 T206 | | Adenosine and AMP deaminase proteins | BLIMPS_BLOCKS |
| | | | T275 T282 Y97 | | BL00485: A2-R32, D60-A71, S171-L212, | |
| | | | Y254 Y272 Y312 | | A235-L289 | |
| | | | | | Adenosine and AMP deaminase signature: | PROFILESCAN |
| | | | | | A235-F281 | |
| | | | | | DEAMINASE HYDROLASE | BLAST_PRODOM |
| | | | | | NUCLEOTIDE METABOLISM | |
| | | | | | ADENOSINE AMP | |
| | | | | | AMINOHYDROLASE ISOFORM | |
| | | | | | MULTIGENE FAMILY | |
| | | | | | PD005960: V141-L308, F61-H105 | |
| | | | | | ADENOSINE DEAMINASE | BLAST_PRODOM |
| | | | | | AMINOHYDROLASE | |
| | | | | | HYDROLASE NUCLEOTIDE | |
| | | | | | METABOLISM | |
| | | | | | POLYMORPHISM ACETYLATION | |
| | | | | | PHARMACEUTICAL 3DSTRUCTURE | |
| | | | | | PD008716: D8-L58 | |
| | | | | | Adenosine and AMP deaminase signature: | MOTIFS |
| | | | | | S255-P261 | |
| 20 | 7506741CD1 | 421 | S119 S165 S181 | | signal_cleavage: M1-L37 | SPSCAN |
| | | | S221 T300 Y273 | | Cytochrome P450: H267-R410, F51-S203 | HMMER_PFAM |
| | | | | | Cytosolic domain: K33-H117 | TMHMMER |
| | | | | | Transmembrane domains: V15-I32, | |
| | | | | | G118-F140 | |
| | | | | | Non-cytosolic domains: M1-D14, | |
| | | | | | Q141-L421 | |
| | | | | | Cytochrome P450 cysteine heme-iron | BLIMPS_BLOCKS |
| | | | | | ligand proteins BL00086: F349-F380 | |
| | | | | | Cytochrome P450 cysteine heme-iron | PROFILESCAN |
| | | | | | ligand signature: F333-R379 | |
| | | | | | Mitochondrial P450 signature PR00408: | BLIMPS_PRINTS |
| | | | | | L134-R144, D269-P287, L350-C359, | |
| | | | | | C359-K370 | |
| | | | | | E-class P450 group II signature | BLIMPS_PRINTS |
| | | | | | PR00464: G136-K156, L191-Q209, | |
| | | | | | Q270-G290, G310-K325, V326-G341, | |
| | | | | | S346-C359, C359-L382 | |
| | | | | | E-class P450 group IV signature | BLIMPS_PRINTS |
| | | | | | PR00465: M271-P287, H321-A339, | |
| | | | | | A343-C359, C359-L377 | |
| | | | | | Helicase conserved C-terminal domain | BLIMPS_PFAM |
| | | | | | PF00271: Y38-L45 | |
| | | | | | CYTOCHROME P450 ACID | BLAST_PRODOM |
| | | | | | OXIDOREDUCTASE | |
| | | | | | MONOOXYGENASE LAURIC | |
| | | | | | ELECTRON TRANSPORT | |
| | | | | | MEMBRANE HEME PD016961: M1-F51 | |
| | | | | | CYTOCHROME P450 | BLAST_PRODOM |
| | | | | | MONOOXYGENASE | |
| | | | | | OXIDOREDUCTASE HEME | |
| | | | | | ELECTRON TRANSPORT | |
| | | | | | MEMBRANE MICROSOME | |
| | | | | | ENDOPLASMIC PD000021: | |
| | | | | | L268-F338, H321-R409, W89-F202 | |
| | | | | | CYTOCHROME P450 | BLAST_DOMO |
| | | | | | DM00022\|JX0331\|124-501: | |
| | | | | | N266-I404, A124-S214, P384-P414 | |
| | | | | | DM00022\|P14580\|124-501: | |
| | | | | | H267-I404, A124-S214 | |
| | | | | | DM00022\|P14581\|124-502: | |
| | | | | | H267-I404, A124-D211 | |
| | | | | | DM00022\|P10611\|120-497: | |
| | | | | | H267-I404, P125-S214 | |
| | | | | | Cytochrome P450 cysteine heme-iron | MOTIFS |
| | | | | | ligand signature: F352-G361 | |
| 21 | 7506743CD1 | 249 | S119 S165 S181 | | signal_cleavage: M1-L37 | SPSCAN |
| | | | S221 | | Cytochrome P450: F51-S203 | HMMER_PFAM |
| | | | | | Cytosolic domain: K33-H117 | TMHMMER |
| | | | | | Transmembrane domains: V15-I32, | |
| | | | | | G118-F140 | |
| | | | | | Non-cytosolic domains: M1-D14, | |
| | | | | | Q141-V249 | |
| | | | | | E-class P450 group II signature | BLIMPS_PRINTS |
| | | | | | PR00464: G136-K156, L191-Q209 | |
| | | | | | Helicase conserved C-terminal domain | BLIMPS_PFAM |
| | | | | | PF00271: Y38-L45 | |
| | | | | | CYTOCHROME P450 ACID | BLAST_PRODOM |
| | | | | | OXIDOREDUCTASE | |
| | | | | | MONOOXYGENASE LAURIC | |
| | | | | | ELECTRON | |
| | | | | | TRANSPORT MEMBRANE | |
| | | | | | HEME PD016961: M1-F51 | |
| | | | | | CYTOCHROME P450 | BLAST_DOMO |
| | | | | | DM00022\|JX0331\|124-501: A124-S214 | |
| | | | | | DM00022\|P14580\|124-501: A124-S214 | |
| | | | | | DM00022\|P24464\|122-499: A124-R212 | |
| | | | | | DM00022\|P10611\|120-497: P125-S214 | |
| 22 | 7506746CD1 | 311 | S119 S165 S181 | | signal_cleavage: M1-L37 | SPSCAN |
| | | | S221 T300 Y273 | | Cytochrome P450: F51-S203, H267-G310 | HMMER_PFAM |
| | | | | | Cytosolic domain: K33-H117 | TMHMMER |
| | | | | | Transmembrane domains: V15-I32, | |
| | | | | | G118-F140 | |
| | | | | | Non-cytosolic domains: M1-D14, | |
| | | | | | Q141-V311 | |
| | | | | | E-class P450 group II signature | BLIMPS_PRINTS |
| | | | | | PR00464: G136-K156, L191-Q209, | |
| | | | | | Q270-G290 | |
| | | | | | Helicase conserved C-terminal domain | BLIMPS_PFAM |
| | | | | | PF00271: Y38-L45 | |
| | | | | | CYTOCHROME P450 ACID | BLAST_PRODOM |
| | | | | | OXIDOREDUCTASE | |
| | | | | | MONOOXYGENASE LAURIC | |
| | | | | | ELECTRON | |
| | | | | | TRANSPORT MEMBRANE | |
| | | | | | HEME PD016961: M1-F51 | |
| | | | | | CYTOCHROME P450 | BLAST_DOMO |
| | | | | | DM00022\|JX0331\|124-501: | |
| | | | | | A124-S214, N266-V311 | |
| | | | | | DM00022\|P14580\|124-501: | |
| | | | | | A124-S214, H267-V311 | |
| | | | | | DM00022\|P10611\|120-497: | |
| | | | | | P125-S214, H267-V311 | |
| | | | | | DM00022\|P08516\|123-500: | |
| | | | | | A124-V210, N266-V311 | |
| 23 | 7506748CD1 | 152 | S16 S26 S27 S90 | | Glutathione S-transferase, | HMMER_PFAM |
| | | | S107 S120 S134 | | N-terminal domain: P2-R82 | |
| | | | T34 T67 | | Glutathione S-transferas | BLIMPS_PFAM |
| | | | | | PF00043: N59-G88 | |
| | | | | | GLUTATHIONE TRANSFERASE | BLAST_PRODOM |
| | | | | | STRANSFERASE MULTIGENE | |
| | | | | | FAMILY PROTEIN | |
| | | | | | CLASSALPHA SCRYSTALLIN | |
| | | | | | GST LYASE PD000312: | |
| | | | | | M3-D106, E101-P141 | |
| | | | | | GLUTATHIONE TRANSFERASE | BLAST_DOMO |
| | | | | | DM00309\|S17462\|1-38: M3-Y41 | |
| | | | | | DM00309\|P09488\|1-35: P2-D37 | |
| | | | | | DM00309\|S14344\|1-30: P2-K31 | |
| | | | | | DM00127\|S17462\|40-139: D42-K152 | |
| 24 | 1419966CD1 | 453 | S97 S183 S231 | | Hydroxymethylglutaryl-coenzyme A | HMMER_PFAM |
| | | | S343 S367 S378 | | synthase: W50-R451 | |
| | | | S386 S396 S401 | | Hydroxymethylglutaryl-coenzyme A | BLIMPS_BLOCKS |
| | | | T71 T133 T155 | | synthase proteins BL01226: W50-L88, | |
| | | | T236 T293 | | F95-R126, L127-C166, F174-G209, | |
| | | | | | G212-M252, F405-P452, L331-S378 | |
| | | | | | Hydroxymethylglutaryl-coenzyme A | MOTIFS |
| | | | | | synthase active site: N154-G169 | |
| 25 | 7506451CD1 | 285 | S2 S132 S143 S214 | N231 | Signal_cleavage: M1-G21 | SPSCAN |
| | | | T41 T71 T82 T84 | | Signal Peptide: M3-G21, M3-G23, | HMMER |
| | | | T118 T161 T232 | | M3-G24, M1-C23, M1-G24, | |
| | | | | | M3-C23, M3-S22 | |
| | | | | | UDP-glucoronosyl and UDP-glucosyl | HMMER_PFAM |
| | | | | | transferas: G24-G157, R158-L279 | |
| | | | | | UDP-glycosyltransferases BL00375: | BLIMPS_BLOCKS |
| | | | | | S34-L56, I171-C198, F211-P260, | |
| | | | | | N266-L286 | |
| | | | | | TRANSFERASE | BLAST_PRODOM |
| | | | | | GLYCOSYLTRANSFERASE | |
| | | | | | PROTEIN UDP-GLUCURONOSYL- | |
| | | | | | TRANSFERASE PRECURSOR | |
| | | | | | SIGNAL TRANSMEMBRANE | |
| | | | | | UDPGT GLYCOPROTEIN | |
| | | | | | MICROSOMAL | |
| | | | | | PD000190: G24-G157 | |
| | | | | | UDP-GLUCORONOSYL AND | BLAST_DOMO |
| | | | | | UDP-GLUCOSYL | |
| | | | | | TRANSFERASES | |
| | | | | | DM00435\|P06133\|1-185: M1-G157 | |
| | | | | | DM00435\|P36538\|1-185: M1-R175 | |
| | | | | | DM00367\|P36538\|187-461: F145-L279 | |
| | | | | | DM00367\|P06133\|187-461: F145-L279 | |
| 26 | 90015249CD1 | 201 | S60 S188 T78 | | Glutathione S-transferase, | HMMER_PFAM |
| | | | | | N-terminal domain: N9-R61 | |
| | | | | | Glutathione S-transferase | BLIMPS_PFAM |
| | | | | | PF00043: K38-G67 | |
| | | | | | Cell attachment sequence: R162-D164 | MOTIFS |
| 27 | 7487231CD1 | 399 | S107 S221 S279 | | Signal_cleavage: M1-G36 | SPSCAN |
| | | | T112 T115 T272 | | PLAT/LH2 domain: G2-G111 | HMMER_PFAM |
| | | | | | Lipoxygenase: Q121-I371 | HMMER_PFAM |
| | | | | | Lipoxygenases iron-binding region | BLIMPS_BLOCKS |
| | | | | | proteins BL00711: F94-N103, D159-F174, | |
| | | | | | W222-R250, A297-R322 | |
| | | | | | Lipoxygenase signature PR00087: | BLIMPS_PRINTS |
| | | | | | P337-L354, H355-C372 | |
| | | | | | Mammalian lipoxygenase signature | BLIMPS_PRINTS |
| | | | | | PR00467: G11-G28, L57-W76, E133-G147, | |
| | | | | | W197-V218, Q294-P313 | |
| | | | | | OXIDOREDUCTASE | BLAST_PRODOM |
| | | | | | DIOXYGENASE IRON | |
| | | | | | LIPOXYGENASE | |
| | | | | | ARACHIDONATE | |
| | | | | | LEUKOTRIENE | |
| | | | | | BIOSYNTHESIS | |
| | | | | | MULTIGENE FAMILY | |
| | | | | | 12LIPOXYGENASE | |
| | | | | | PD000872: P41-R136, | |
| | | | | | G271-Y385, D199-V288 | |
| | | | | | ARACHIDONATE | BLAST_PRODOM |
| | | | | | OXIDOREDUCTASE | |
| | | | | | DIOXYGENASE IRON | |
| | | | | | LEUKOTRIENE | |
| | | | | | BIOSYNTHESIS | |
| | | | | | 12LIPOXYGENASE 12LOX | |
| | | | | | 15LIPOXYGENASE OMEGA6 | |
| | | | | | PD150166: K137-W197 | |
| | | | | | ARACHIDONATE | BLAST_PRODOM |
| | | | | | 12LIPOXYGENASE | |
| | | | | | PSEUDOGENE | |
| | | | | | EPIDERMISTYPE | |
| | | | | | LIPOXYGENASE | |
| | | | | | 12RLIPOXYGENASE | |
| | | | | | 15SLIPOXYGENASE | |
| | | | | | 8SLIPOXYGENASE | |
| | | | | | 8RLIPOXYGENASEALLENE | |
| | | | | | OXIDE PD150360: M1-F207 | |
| | | | | | LIPOXYGENASES IRON- | BLAST_DOMO |
| | | | | | BINDING REGION | |
| | | | | | DM01661\|P16050\|1-317: G2-Q319 | |
| | | | | | DM01661\|Q02759\|1-318: G2-Q319 | |
| | | | | | DM01661\|B54075\|1-319: M1-Q319 | |
| | | | | | DM01661\|P39654\|1-318: G2-Q319 | |
| | | | | | Lipoxygenases iron-binding region | MOTIFS |
| | | | | | signature 1: H355-E369 | |
| 28 | 7506260CD1 | 364 | S39 S126 S180 | N32 N204 | Acyl CoA binding protein: S39-E123 | HMMER_PFAM |
| | | | S198 S263 S359 | | Enoyl-CoA hydratase/isomerase | HMMER_PFAM |
| | | | T132 T137 T145 | | family: T151-P292 | |
| | | | T277 | | Enoyl-CoA hydratase/isomerase | BLIMPS_BLOCKS |
| | | | | | proteins BL00166: G149-K160, | |
| | | | | | I186-D207, K236-A262 | |
| | | | | | Acyl-CoA-binding protein BL00880: | BLIMPS_BLOCKS |
| | | | | | Y69-L118 | |
| | | | | | Enoyl-CoA hydratase/isomerase | PROFTLESCAN |
| | | | | | signature: L223-A278 | |
| | | | | | Acyl-coA-binding protein signature | BLIMPS_PRINTS |
| | | | | | PR00689: Q40-K55, P57-G75, P80-A95, | |
| | | | | | S101-L118 | |
| | | | | | PROTEIN ACBP/ECHM | BLAST_PRODOM |
| | | | | | DBI-RELATED R06F6.9 | |
| | | | | | CHROMOSOME II | |
| | | | | | PD029846: D293-L364 | |
| | | | | | PROTEIN ACYLCOA- | BLAST_PRODOM |
| | | | | | BINDING ACBP TRANSPORT | |
| | | | | | LIPID-BINDING BINDING | |
| | | | | | DIAZEPAM INHIBITOR DBI | |
| | | | | | ENDOZEPINE | |
| | | | | | PD002965: Q40-L118 | |
| | | | | | PROTEIN HYDRATASE | BLAST_PRODOM |
| | | | | | ENOYLCOA ACID | |
| | | | | | FATTY LYASE ISOMERASE | |
| | | | | | METABOLISM | |
| | | | | | 3HYDROXYACYLCOA | |
| | | | | | DEHYDROGENASE | |
| | | | | | PD000432: I150-A266, R265-P292 | |
| | | | | | ENOYL-COA HYDRATASE/ISOMERASE | BLAST_DOMO |
| | | | | | DM00366\|P41942\|1-254: L142-A383 | |
| | | | | | ENOYL-COA HYDRATASE/ISOMERASE | BLAST_DOMO |
| | | | | | DM00366\|A48956\|4-261: | |
| | | | | | V144-T267, R265-L341 | |
| | | | | | ACYL-COA-BINDING PROTEIN | BLAST_DOMO |
| | | | | | DM01433\|P07106\|40-129: F43-D113 | |
| | | | | | ACYL-COA-BINDING PROTEIN | BLAST_DOMO |
| | | | | | DM01433\|P11030\|1-84: S39-L118 | |
| 29 | 7506270CD1 | 319 | S159 S203 S224 | N201 N264 N308 | Glutamine synthetase: | HMMER_PFAM |
| | | | S268 S289 S296 | | E56-T298, E24-K52 | |
| | | | T62 T139 T247 | | Glutamine synthetase proteins | BLIMPS_BLOCKS |
| | | | T310 Y126 | | BL00180: W76-L85, F93-D114, | |
| | | | | | A128-G179, N192-T204, G281-F295 | |
| | | | | | Glutamine synthetase signatures: | PROFILESCAN |
| | | | | | A167-K225 | |
| | | | | | LIGASE SYNTHETASE GLUTAMINE | BLAST_PRODOM |
| | | | | | GLUTAMATEAMMONIA | |
| | | | | | NITROGEN FIXATION | |
| | | | | | MULTIGENE FAMILY | |
| | | | | | ISOZYME GS PD000349: | |
| | | | | | S101-F295, W32-F100, V26-L47 | |
| | | | | | GLUTAMATE--AMMONIA LIGASE | BLAST_DOMO |
| | | | | | DM00510\|I49706\|26-372: | |
| | | | | | E56-N319, V26-E56 | |
| | | | | | GLUTAMATE--AMMONIA LIGASE | BLAST_DOMO |
| | | | | | DM00510\|P16580\|23-373: | |
| | | | | | E56-N319, G23-E56 | |
| | | | | | GLUTAMATE--AMMONIA LIGASE | BLAST_DOMO |
| | | | | | DM00510\|P51121\|21-391: | |
| | | | | | E56-N319, P21-E56 | |
| | | | | | GLUTAMATE--AMMONIA LIGASE | BLAST_DOMO |
| | | | | | DM00510\|Q04831\|20-361: | |
| | | | | | G35-E309, E24-E56 | |
| | | | | | Glutamine synthetase putative | MOTIFS |
| | | | | | ATP-binding region signature: K187-S203 | |
| 30 | 7506306CD1 | 458 | S4 S132 | N67 N135 N304 | Signal_cleavage: M1-G21 | SPSCAN |
| | | | S136 S153 | N325 N410 | Signal Peptide: M1-G21, M1-T23, M1-A24 | HMMER |
| | | | T226 | | GDA1/CD39 (nucleoside phosphatase) | HMMER_PFAM |
| | | | | | family: S34-R424 | |
| | | | | | Cytosolic domains: M1-A12, D458—D458 | TMHMMER |
| | | | | | Transmembrane domains: L13-V35, | |
| | | | | | V435-Q457 | |
| | | | | | Non-cytosolic domain: L36-K434 | |
| | | | | | GDA1/CD39 family of nucleoside | |
| | | | | | phosphatases proteins BL01238: | |
| | | | | | I45-Y59, P119-R129, L163-V184, | |
| | | | | | G202-F215 | |
| | | | | | HYDROLASE TRANSMEMBRANE | BLAST_PRODOM |
| | | | | | PROTEIN NUCLEOSIDE CD39 | |
| | | | | | NUCLEOSIDETRIPHOSPHATASE | |
| | | | | | TRIPHOSPHATE NTPASE | |
| | | | | | PRECURSOR | |
| | | | | | ATPDIPHOSPHOHYDROLASE | |
| | | | | | PD003822: T33-C329, | |
| | | | | | Q194-Y355, Y355-P420 | |
| | | | | | ACTIVATION; NUCLEOSIDE; | BLAST_DOMO |
| | | | | | ANTIGEN; LYMPHOID; | |
| | | | | | DM02628\|I56242\|40-471: | |
| | | | | | E97-F349, V351-P420 | |
| | | | | | ACTIVATION; NUCLEOSIDE; | BLAST_DOMO |
| | | | | | ANTIGEN; LYMPHOID; | |
| | | | | | DM02628\|P49961\|40-471: | |
| | | | | | L37-F349, V351-P420 | |
| | | | | | ACTIVATION; NUCLEOSIDE; | BLAST_DOMO |
| | | | | | ANTIGEN; LYMPHOID; | |
| | | | | | DM02628\|P32621\|84-517: | |
| | | | | | K42-D400, Y365-L411 | |
| | | | | | ACTIVATION; NUCLEOSIDE; | BLAST_DOMO |
| | | | | | ANTIGEN; LYMPHOID; | |
| | | | | | DM02628\|P52914\|35-454: F43-Y278 | |
| | | | | | GDA1/CD39 family of nucleoside | MOTIFS |
| | | | | | phosphatases signature: L163-Y178 | |
| 31 | 7506428CD1 | 295 | S21 S110 S162 | N221 | Adenosine/AMP deaminase: M1-L278 | HMMER_PFAM |
| | | | S234 T57 T125 | | Adenosine and AMP deaminase proteins | BLIMPS_BLOCKS |
| | | | T174 T243 T250 | | BL00485: A2-R32, D60-A71, A203-L257 | |
| | | | Y97 Y222 Y240 | | Adenosine and AMP deaminase | PROFILESCAN |
| | | | Y280 | | signature: A203-F249 | |
| | | | | | ADENOSINE DEAMINASE | BLAST_PRODOM |
| | | | | | AMINOHYDROLASE HYDROLASE | |
| | | | | | NUCLEOTIDE METABOLISM | |
| | | | | | POLYMORPHISM ACETYLATION | |
| | | | | | PHARMACEUTICAL 3DSTRUCTURE | |
| | | | | | PD008716: D8-L58 | |
| | | | | | DEAMINASE HYDROLASE | BLAST_PRODOM |
| | | | | | NUCLEOTIDE METABOLISM | |
| | | | | | ADENOSINE AMP | |
| | | | | | AMINOHYDROLASE ISOFORM | |
| | | | | | MULTIGENE FAMILY | |
| | | | | | PD005960: F61-H105, | |
| | | | | | L184-L276, Y68-Q119 | |
| | | | | | Adenosine and AMP deaminase | MOTIFS |
| | | | | | signature: S223-P229 | |
| 32 | 7678032CD1 | 472 | S5 S142 S241 S280 | N179 N216 | PWWP domain: S5-R78 | HMMER_PFAM |
| | | | S459 T101 T107 | | 6-phosphogluconate dehydrogenase | BLIMPS_BLOCKS |
| | | | T124 T144 T155 | | proteins BL00461: K188-D223, | |
| | | | T183 T185 T235 | | V250-E289, M339-Q378 | |
| | | | T286 T369 T386 | | 3-hydroxyisobutyrate dehydrogenase | BLIMPS_BLOCKS |
| | | | | | proteins BL00895: I189-G209, | |
| | | | | | G298-F336, G347-Q382 | |
| | | | | | PWWP domain proteins. PF00855: | BLIMPS_PFAM |
| | | | | | D10-I26 | |
| | | | | | DEHYDROGENASE | BLAST_PRODOM |
| | | | | | OXIDOREDUCTASE | |
| | | | | | 6PHOSPHOGLUCONATE | |
| | | | | | PENTOSE SHUNT | |
| | | | | | NADP DECARBOXYLATING | |
| | | | | | GLUCONATE UTILIZATION | |
| | | | | | PROTEIN PD001025: L196-F400 | |
| | | | | | 3-HYDROXYISOBUTYRATE | BLAST_DOMO |
| | | | | | DEHYDROGENASE | |
| | | | | | DM03208\|P23523\|1-298: K188-Y467 | |
| | | | | | DM03208\|P32142\|1-297: I189-V466 | |
| | | | | | DM03208\|P28811\|1-297: I189-Y470 | |
| | | | | | DM03208\|P44979\|1-300: I189-Y470 | |
| 33 | 7508332CD1 | 483 | S142 S170 S177 | N49 N303 | signal_cleavage: M1-G20 | SPSCAN |
| | | | S205 S391 T93 | | Signal Peptide: M1-G20 | HMMER |
| | | | T207 T243 T258 | | UDP-glucoronosyl and UDP-glucosyl | HMMER_PFAM |
| | | | T375 Y234 | | transferase: K288-K481, G21-P287 | |
| | | | | | Cytosolic domain: C470-E483 | TMHMMER |
| | | | | | Transmembrane domain: V447-S469 | |
| | | | | | Non-cytosolic domain: M1-D446 | |
| | | | | | UDP-glycosyltransferases proteins | BLIMPS_BLOCKS |
| | | | | | BL00375: S31-L53, C126-P166, | |
| | | | | | P189-N212, N304-P348, H403-Y442 | |
| | | | | | UDP-glycosyltransferases | PROFILESCAN |
| | | | | | signature: T331-T373 | |
| | | | | | TRANSFERASE | BLAST_PRODOM |
| | | | | | GLYCOSYLTRANSFERASE | |
| | | | | | PROTEIN | |
| | | | | | UDPGLUCURONOSYLTRANSFERASE | |
| | | | | | PRECURSOR SIGNAL | |
| | | | | | TRANSMEMBRANE UDPGT | |
| | | | | | GLYCOPROTEIN MICROSOMAL | |
| | | | | | PD000190: G21-L278, Y392-E483, | |
| | | | | | A283-A396 | |
| | | | | | UDP-GLUCORONOSYL AND | MOTIFS |
| | | | | | UDP-GLUCOSYL TRANSFERASES | |
| | | | | | DM00367\|P36510\|176-459: | |
| | | | | | K288-F416, A176-K288 | |
| | | | | | DM00367\|P54855\|188-462: | |
| | | | | | K288-F416, Y188-K288 | |
| | | | | | DM00367\|A48633\|188-462: | |
| | | | | | K288-F416, Y188-K288 | |
| | | | | | DM00367\|P36538\|187-461: | |
| | | | | | A283-F416, Y188-K288 | |
| | | | | | UDP-glycosyltransferases signature: | MOTIFS |
| | | | | | W310-O353 | |
| 34 | 1288969CD1 | 346 | S36 S69 S127 S211 | N118 N170 N260 | signal_cleavage: M1-I25 | SPSCAN |
| | | | S268 S337 T91 | | Signal Peptide: M1-I25, M1-P27 | HMMER |
| | | | T103 | | Eukaryotic-type carbonic anhydrase: | HMMER_PFAM |
| | | | | | G65-R303 | |
| | | | | | Eukaryotic-type carbonic anhydrases | PROFILESCAN |
| | | | | | signature: S127-A182 | |
| | | | | | Eukaryotic-type carbonic anhydrases | BLIMPS_BLOCKS |
| | | | | | proteins BL00162: R270-R303, N56-P86, | |
| | | | | | H128-N164, L167-A191, G233-Q265 | |
| | | | | | CARBONIC ANHYDRASE | BLAST_PRODOM |
| | | | | | DEHYDRATASE LYASE | |
| | | | | | CARBONATE ZINC PRECURSOR | |
| | | | | | SIGNAL PROTEIN | |
| | | | | | GLYCOPROTEIN PD000865: W35-N302 | |
| | | | | | CARBONIC PROTEIN ANHYDRASE | BLAST_PRODOM |
| | | | | | RELATED ANHYDRASE-LIKE | |
| | | | | | PD084951: M1-W34 | |
| | | | | | CARBONIC ANHYDRASE | BLAST_DOMO |
| | | | | | DM00356\|P00919\|23-258: G65-N302 | |
| | | | | | DM00356\|P00918\|23-258: G65-N302 | |
| | | | | | DM00356\|P00921\|23-258: G65-G301 | |
| | | | | | DM00356\|P23589\|53-290: A63-N302 | |
| 35 | 72069135CD1 | 359 | S6 S37 S158 S174 | N25 | signal_cleavage: M1-S63 | SPSCAN |
| | | | T219 | | Signal Peptide: M1-S27 | HMMER |
| | | | | | ISOMERASE ISOPENTENYL IPP | BLAST_PRODOM |
| | | | | | ISOPENTENYLDIPHOSPHATE | |
| | | | | | DELTAISOMERASE DIPHOSPHATE | |
| | | | | | PYROPHOSPHATE DIPHOSPHATE: | |
| | | | | | DIMETHYLALLYL BIOSYNTHESIS | |
| | | | | | PROTEIN PD004109: L201-D354 | |
| | | | | | ISOMERASE IPP ISOPENTENYL | BLAST_PRODOM |
| | | | | | DIPHOSPHATE: DIMETHYLALLYL | |
| | | | | | DIPHOSPHATE ISOPENTENYL- | |
| | | | | | DIPHOSPHATE DELTA | |
| | | | | | ISOMERASE HOMOLOG OF YEAST | |
| | | | | | PD022398: M185-E218 | |
| | | | | | ISOMERASE; DIPHOSPHATE; | BLAST_DOMO |
| | | | | | DELTA; ISOPENTENYL | |
| | | | | | DM02460\|A53028\|19-176: A202-L338 | |
| | | | | | DM02460\|Q10132\|19-178: E203-L337 | |
| | | | | | DM02460\|P15496\|70-236: E203-L337 | |
| | | | | | DM02460\|P34511\|627-779: E203-L338 | |
| 36 | 7506247CD1 | 275 | S58 S98 S216 S231 | N263 | signal_cleavage: M1-K36 | SPSCAN |
| | | | S265 T33 T235 | | short chain dehydrogenase: Y66-P251 | HMMER_PFAM |
| | | | Y106 | | Pyrokinins proteins BL00539: F52-L56 | BLIMPS_BLOCKS |
| | | | | | Short-chain dehydrogenases/reductases | PROFILESCAN |
| | | | | | family signature: G143-P193 | |
| | | | | | Glucose/ribitol dehydrogenase family | BLIMPS_PRINTS |
| | | | | | signature PR00081: W69-E86, M137-C153, | |
| | | | | | F163-A182, K184-T201 | |
| | | | | | SHORT-CHAIN ALCOHOL | BLAST_DOMO |
| | | | | | DEHYDROGENASE FAMILY | |
| | | | | | DM00034\|P38286\|57-346: | |
| | | | | | F107-K268, G67-V121 | |
| | | | | | DM00034\|Q10245\|52-340: | |
| | | | | | G67-L104, F107-A270 | |
| | | | | | DM00034\|P37058\|43-275: D77-I242 | |
| | | | | | DM00034\|P14802\|1-235: A60-S239 | |
| 37 | 7506363CD1 | 337 | S10 S58 S147 S154 | | GDP-D-MANNOSE DEHYDRATASE RFBD | BLAST_PRODOM |
| | | | S189 S245 T32 T62 | | HYPOTHETICAL PROTEIN PD030255: | |
| | | | T132 T185 T277 | | D209-K275 | |
| | | | T292 Y123 | | UDP GLUCOSE 4-EPIMERASE | BLAST_DOMO |
| | | | | | DM00174\|P55295\|1-329: | |
| | | | | | N71-N182, Y179-E318 | |
| | | | | | DM00174\|P21977\|1-322: | |
| | | | | | L27-G180, V243-A326 | |
| 38 | 7509068CD1 | 445 | S28 S43 S111 S244 | N119 N142 N296 | signal_cleavage: M1-S28 | SPSCAN |
| | | | S441 T85 T144 | N348 | Signal Peptide: M1-A26, M1-S28 | HMMER |
| | | | T200 T420 | | UDP-glucoronosyl and UDP-glucosyl | HMMER_PFAM |
| | | | | | transferase: G29-K435 | |
| | | | | | UDP-glycosyltransferases proteins | BLIMPS_BLOCKS |
| | | | | | BL00375: C128-P168, P190-N213, | |
| | | | | | V254-C281, Y294-P343, N349-P393, | |
| | | | | | S39-L61 | |
| | | | | | UDP-glycosyltransferases signature: | PROFILESCAN |
| | | | | | S376-E418 | |
| | | | | | TRANSFERASE | BLAST_PRODOM |
| | | | | | GLYCOSYLTRANSFERASE | |
| | | | | | PROTEIN | |
| | | | | | UDPGLUCURONOSYLTRANSFERASE | |
| | | | | | PRECURSOR SIGNAL | |
| | | | | | TRANSMEMBRANE UDPGT | |
| | | | | | GLYCOPROTEIN MICROSOMAL | |
| | | | | | PD000190: G29-I279, V245-V431, | |
| | | | | | F291-O444 | |
| | | | | | UDP-GLUCORONOSYL AND | BLAST_DOMO |
| | | | | | UDP-GLUCOSYL TRANSFERASES | |
| | | | | | DM00367\|P22310\|187-460: C187-Q439 | |
| | | | | | DM00367\|P35504\|187-460: C187-Q439 | |
| | | | | | DM00367\|P35503\|187-460: C187-Q439 | |
| | | | | | DM00367\|P22309\|176-459: P177-Q439 | |
| | | | | | Cell attachment sequence: R258-D260 | MOTIFS |
| | | | | | UDP-glycosyltransferases signature: | MOTIFS |
| | | | | | W355-Q398 | |
| 39 | 7505897CD1 | 162 | S111 S123 S145 | N46 | signal_cleavage: M1-A14 | SPSCAN |
| | | | T57 | | Signal Peptide: M1-G20 | HMMER |
| | | | | | Glycosyl hydrolase family: F3-T88, | HMMER_PFAM |
| | | | | | K95-L162 | |
| | | | | | Glycosyl hydrolases family 1 proteins | BLIMPS_BLOCKS |
| | | | | | BL00572: F3-W32, G54-G87 | |
| | | | | | Glycosyl hydrolases family 1 | PROFILESCAN |
| | | | | | signatures: A2-E41 | |
| | | | | | Glycosyl hydrolases family PR00131: | BLIMPS_PRINTS |
| | | | | | K101-W118, R125-A137 | |
| | | | | | HYDROLASE GLYCOSIDASE | BLAST_PRODOM |
| | | | | | BETAGLUCOSIDASE PRECURSOR | |
| | | | | | CELLOBIASE SIGNAL AMYGDALASE | |
| | | | | | GLUCOHYDROLASE GENTIOBIASE | |
| | | | | | CELLULOSE PD000650: F3-Q98, | |
| | | | | | K95-G157 | |
| | | | | | GLYCOSYL HYDROLASES | BLAST_DOMO |
| | | | | | FAMILY 1 N-TERMINAL | |
| | | | | | DM00233\|P09848\|1368-1838: | |
| | | | | | F3-I97, F91-G157 | |
| | | | | | DM00233\|JS0610\|1369-1839: | |
| | | | | | F3-I97, F91-G157 | |
| | | | | | DM00233\|P09848\|899-1366: | |
| | | | | | F3-I97, F91-L158 | |
| | | | | | DM00233\|P26208\|2-446: | |
| | | | | | M1-K101, K95-G157 | |
| | | | | | Glycosyl hydrolases family 1 | MOTIFS |
| | | | | | N-terminal signature: F7-G21 | |
| 40 | 7505898CD1 | 321 | S2 S17 S44 S129 | N248 N290 | AIR synthase related protein, | HMMER_PFAM |
| | | | S271 T117 T133 | | N-terminal do: K32-P188 | |
| | | | T229 T292 | | AIR synthase related protein, | HMMER_PFAM |
| | | | | | C-terminal do: G251-D297, A191-G204 | |
| | | | | | TIGR_selD: selenide, water | HMMER_PFAM |
| | | | | | dikinase: E13-A305 | |
| | | | | | AIR synthase related protein, | BLIMPS_PFAM |
| | | | | | N-terminal domain | |
| | | | | | PF00586: G28-V37, G65-G78 | |
| | | | | | PROTEIN SELENOPHOSPHATE | BLAST_PRODOM |
| | | | | | SYNTHETASE SELD SELENIDE | |
| | | | | | WATER DIKINASE SELENIUM | |
| | | | | | DONOR TRANSFERASE | |
| | | | | | PD022107: R4-L64 | |
| | | | | | ATP NP_BIND | BLAST_DOMO |
| | | | | | DM04434\|P49903\|1-382: | |
| | | | | | M1-L254, T250-A305 | |
| | | | | | DM04434\|P16456\|1-346: D15-V207 | |
| | | | | | DM04434\|P43911\|1-347: L14-G204 | |
| 41 | 7505907CD1 | 468 | S187 S319 S354 | N363 | Mandelate racemase/muconate | HMMER_PFAM |
| | | | S359 S447 T69 T82 | | lactonizing en: R257-S319 | |
| | | | T199 T242 T318 | | Mandelate racemase/muconate | HMMER_PFAM |
| | | | | | lactonizing en: A75-V178 | |
| | | | | | Mandelate racemase/muconate | BLIMPS_BLOCKS |
| | | | | | lactonizing enzyme | |
| | | | | | family signature BL00908: | |
| | | | | | A153-D179, I275-I329 | |
| | | | | | RTSBETA ORF1A | BLAST_PRODOM |
| | | | | | PD041688: L140-L266 | |
| | | | | | PD041686: G383-N468 | |
| | | | | | PD029163: M67-V103 | |
| | | | | | PD135731: V104-Q139 | |
| | | | | | Mandelate racemase/muconate | MOTIFS |
| | | | | | lactonizing enzyme | |
| | | | | | family signature 2: G281-D322 | |
| 42 | 7505925CD1 | 318 | S31 S226 T42 T60 | N85 | signal_cleavage: M1-A28 | SPSCAN |
| | | | T201 T233 T245 | | ilvE_II: branched-chain amino | HMMER_PFAM |
| | | | Y307 | | acid amino: W75-V318 | |
| | | | | | Aminotransferase class IV: Q77-K303 | HMMER_PFAM |
| | | | | | Aminotransferases class-IV proteins | BLIMPS_BLOCKS |
| | | | | | BL00770: L188-F197, L209-Q232, | |
| | | | | | E260-V273, F57-M67, H96-F108 | |
| | | | | | AMINO TRANSFERASE | BLAST_PRODOM |
| | | | | | ACID TRANSFERASE | |
| | | | | | PYRIDOXAL PHOSPHATE | |
| | | | | | BRANCHED CHAIN | |
| | | | | | AMINO BIOSYNTHESIS | |
| | | | | | BCAT PROTEIN | |
| | | | | | PD001961: N176-K300, P82-G175 | |
| | | | | | AMINO TRANSFERASE | BLAST_PRODOM |
| | | | | | BRANCHED CHAIN | |
| | | | | | AMINO ACID MITOCHONDRIAL | |
| | | | | | PRECURSOR BCATM | |
| | | | | | TRANSFERASE BIOSYNTHESIS | |
| | | | | | PYRIDOXAL PD038218: M1-E40 | |
| | | | | | AMINOTRANSFERASE; BRANCHED; | BLAST_DOMO |
| | | | | | ACID; AMINO | |
| | | | | | DM00866\|P54687\|62-365: | |
| | | | | | G164-R297, G74-E177 | |
| | | | | | DM00866\|P54690\|90-392: | |
| | | | | | N176-R297, G74-E177 | |
| | | | | | DM00866\|P54688\|67-377: | |
| | | | | | E177-N290, D72-E177 | |
| | | | | | DM00866\|P47176\|46-351: | |
| | | | | | D72-V191, L179-P292 | |
| | | | | | Aminotransferases class-IV | MOTIFS |
| | | | | | signature: E190-R224 | |

TABLE 4

| Polynucleotide | |
|---|---|
| SEQ ID NO:/ | |
| Incyte ID/Sequence | |
| Length | Sequence Fragments |
| 43/70612021CB1/ | 1-479, 2-479, 69-802, 150-723, 155-801, 156-725, 224-418, 237-725, 243-969, 254-887, 255-915, |
| 1578 | 285-931, 288-879, 293-841, 345-876, 346-987, 355-1062, 370-960, 379-1084, 384-692, 391-1077, |
| | 410-946, 449-710, 458-915, 462-917, 462-955, 462-1042, 490-1113, 502-1012, 529-725, 592-1186, |
| | 595-1247, 624-751, 629-857, 630-1182, 644-1306, 693-1335, 697-943, 717-1323, 726-1130, 727-1283, |
| | 738-1130, 745-1407, 752-915, 828-1516, 831-1451, 831-1515, 847-1067, 916-1084, 930-1558, 996-1564, |
| | 1002-1046, 1002-1076, 1006-1046, 1007-1076, 1008-1036, 1008-1048, 1008-1076, 1038-1092, |
| | 1038-1097, 1038-1106, 1038-1207, 1038-1546, 1050-1388, 1062-1100, 1062-1106, 1066-1091, |
| | 1066-1106, 1067-1106, 1160-1520, 1164-1578, 1220-1564, 1235-1457, 1304-1564 |
| 44/71847235CB1/ | 1-185, 1-201, 1-212, 1-223, 1-452, 1-570, 2-203, 2-210, 3-107, 3-204, 3-209, 3-210, |
| 2770 | 3-212, 3-218, 3-221, 5-194, 5-436, 5-618, 7-200, 7-225, 7-611, 14-221, 14-534, 75-346, |
| | 148-426, 195-464, 245-879, 314-477, 319-830, 339-908, 372-926, 372-993, 379-962, 385-959, |
| | 390-684, 392-1005, 405-621, 407-950, 448-1024, 473-1019, 476-1047, 485-942, 493-1104, 508-1028, |
| | 594-1258, 605-877, 605-1176, 627-1297, 642-1276, 648-1249, 650-1299, 657-1080, 690-872, 698-1265, |
| | 712-1257, 724-1331, 739-1246, 746-955, 746-959, 746-960, 756-1327, 761-1273, 780-1267, 786-1026, |
| | 790-1057, 796-1288, 821-1433, 834-1449, 838-1261, 848-1261, 849-1576, 852-1095, 864-1509, 875-1490, |
| | 895-1484, 897-1485, 902-1374, 905-1535, 908-1328, 910-1118, 922-1433, 935-1485, 936-1550, 945-1523, |
| | 957-1509, 961-1361, 986-1575, 999-1668, 1006-1208, 1007-1579, 1013-1579, 1022-1294, 1027-1658, |
| | 1028-1635, 1028-1687, 1029-1486, 1031-1233, 1041-1553, 1042-1315, 1042-1592, 1083-1378, |
| | 1087-1360, 1107-1360, 1117-1798, 1125-1740, 1137-1357, 1137-1597, 1157-1361, 1177-1469, |
| | 1186-1467, 1188-1452, 1198-1443, 1198-1453, 1209-1404, 1211-1532, 1225-1841, 1253-1836, |
| | 1256-1460, 1256-1465, 1256-1473, 1256-1510, 1259-1747, 1271-1812, 1287-1939, 1289-1925, |
| | 1290-1539, 1299-1571, 1319-1919, 1322-1855, 1333-1599, 1333-1610, 1333-2044, 1347-1773, |
| | 1353-1555, 1353-1557, 1353-1559, 1355-1940, 1360-1842, 1363-1602, 1363-1603, 1367-1576, |
| | 1378-1621, 1380-2050, 1405-1652, 1418-1848, 1418-1973, 1418-2045, 1421-1739, 1430-1634, |
| | 1430-1635, 1430-1639, 1468-1800, 1474-1825, 1474-2010, 1477-1707, 1477-1717, 1478-2031, |
| | 1482-2053, 1482-2073, 1486-2072, 1489-2084, 1497-2062, 1512-1708, 1514-1719, 1515-1749, |
| | 1525-2130, 1534-2039, 1540-1745, 1540-2195, 1551-2037, 1556-1794, 1557-1803, 1557-2217, |
| | 1576-1780, 1577-1771, 1581-2149, 1582-2102, 1585-1792, 1585-2076, 1596-2235, 1603-2159, |
| | 1607-1815, 1607-1932, 1607-1965, 1607-2052, 1607-2054, 1607-2074, 1607-2079, 1607-2084, |
| | 1607-2088, 1607-2092, 1607-2094, 1607-2118, 1607-2121, 1607-2125, 1607-2174, 1607-2179, |
| | 1607-2187, 1607-2188, 1607-2197, 1607-2202, 1607-2326, 1608-2156, 1611-1793, 1618-2216, |
| | 1621-1829, 1623-2072, 1629-2165, 1632-1839, 1639-1859, 1639-2098, 1641-2232, 1656-1867, |
| | 1671-2182, 1676-1814, 1691-2257, 1693-1909, 1698-1871, 1701-2210, 1701-2267, 1706-1928, |
| | 1712-1910, 1712-1914, 1712-1916, 1718-1849, 1723-2061, 1725-2311, 1729-2005, 1730-1926, |
| | 1742-2232, 1744-1957, 1750-2042, 1761-1976, 1761-2108, 1767-2064, 1767-2217, |
| | 1768-2367, 1775-2036, 1782-2026, 1791-2431, 1804-2015, 1810-2102, 1813-2302, 1814-2054, |
| | 1821-2144, 1826-2363, 1828-1953, 1836-2083, 1838-2098, 1846-2398, 1850-2048, 1850-2049, |
| | 1850-2381, 1851-2081, 1852-2287, 1857-2325, 1857-2326, 1865-2138, 1866-2621, 1871-2477, |
| | 1874-2331, 1875-2759, 1877-2097, 1878-2302, 1878-2471, 1879-2154, 1882-2356, 1887-2138, |
| | 1889-2097, 1890-2081, 1890-2136, 1890-2237, 1890-2282, 1890-2428, 1891-2267, 1892-2135, |
| | 1892-2429, 1898-2124, 1898-2388, 1920-2156, 1920-2455, 1921-2516, 1922-2113, 1924-2442, |
| | 1925-2410, 1929-2442, 1936-2471, 1937-2183, 1939-2182, 1944-2429, 1948-2336, 1948-2477, |
| | 1952-2586, 1960-2228, 1962-2049, 1965-2459, 1965-2510, 1966-2408, 1966-2500, 1966-2553, |
| | 1970-2476, 1978-2392, 1979-2599, 1994-2304, 1994-2372, 2013-2315, 2013-2516, 2014-2696, |
| | 2020-2267, 2021-2700, 2027-2695, 2036-2699, 2037-2289, 2040-2269, 2047-2694, 2057-2265, |
| | 2057-2278, 2065-2690, 2069-2696, 2070-2569, 2071-2270, 2071-2331, 2071-2689, 2078-2153, |
| | 2088-2719, 2096-2719, 2100-2355, 2107-2500, 2110-2657, 2112-2552, 2114-2304, 2132-2389, |
| | 2132-2711, 2133-2690, 2135-2414, 2137-2310, 2138-2723, 2141-2381, 2143-2471, |
| | 2146-2346, 2146-2366, 2146-2409, 2146-2482, 2148-2455, 2155-2410, 2160-2701, 2163-2756, |
| | 2166-2692, 2175-2482, 2178-2669, 2184-2443, 2188-2528, 2191-2458, 2198-2708, 2203-2438, |
| | 2203-2481, 2206-2718, 2207-2448, 2207-2470, 2209-2409, 2209-2455, 2209-2463, 2209-2703, |
| | 2209-2726, 2213-2476, 2214-2483, 2220-2633, 2221-2560, 2225-2738, 2227-2734, 2240-2610, |
| | 2241-2726, 2252-2605, 2256-2517, 2258-2723, 2261-2633, 2269-2464, 2269-2726, 2269-2735, |
| | 2269-2737, 2271-2471, 2271-2701, 2271-2740, 2274-2698, 2280-2450, 2282-2515, 2293-2740, |
| | 2296-2720, 2303-2566, 2328-2580, 2339-2759, 2346-2505, 2347-2709, 2365-2630, 2367-2570, |
| | 2367-2762, 2377-2598, 2379-2589, 2379-2590, 2381-2634, 2382-2719, 2383-2666, 2394-2709, |
| | 2399-2637, 2450-2687, 2473-2736, 2480-2723, 2494-2738, 2569-2735, 2588-2770, 2600-2751 |
| 45/7505230CB1/ | 1-244, 1-254, 1-308, 1-324, 1-360, 1-394, 1-427, 1-449, 1-519, 1-544, 1-598, 1-2651, |
| 2651 | 104-362, 144-674, 301-1002, 525-1002, 576-1002, 584-1002, 962-1308, 962-1342, 964-1337, 1047-1404, |
| | 1146-1965, 1184-1417, 1184-1486, 1184-1571, 1184-1630, 1184-1652, 1184-1666, 1184-1716, |
| | 1184-1794, 1184-1831, 1184-1849, 1184-1857, 1184-1869, 1184-1875, 1184-1886, 1184-1909, |
| | 1184-1912, 1184-1913, 1184-1934, 1184-1946, 1184-1980, 1206-1775, 1231-1434, 1231-1439, |
| | 1231-1440, 1285-1723, 1333-1893, 1368-1935, 1411-1888, 1422-1929, 1701-1959, 1900-1992, |
| | 1900-2054, 1900-2175, 1900-2373, 1900-2374, 1900-2488, 1900-2505, 1900-2639, 1903-2169, |
| | 1904-2564, 1949-2651, 1988-2221, 2005-2628, 2078-2143, 2079-2525, 2261-2498, 2354-2651, |
| | 2381-2637 |
| 46/7505235CB1/ | 1-233, 12-2020, 18-783, 18-873, 26-275, 61-350, 89-317, 145-399, 145-468, 254-538, 473-1187, |
| 2133 | 476-1034, 482-690, 487-935, 499-1089, 568-1173, 607-1291, 662-1254, 678-873, 678-1142, 685-1005, |
| | 700-898, 700-1115, 700-1135, 734-1264, 738-1427, 745-984, 745-1252, 767-1403, 767-1737, 769-1039, |
| | 769-1157, 780-1307, 786-1257, 787-1351, 787-1367, 815-1280, 864-1099, 897-1437, 922-1526, 926-1737, |
| | 936-1564, 936-1737, 952-1596, 975-1632, 1006-1605, 1034-1325, 1036-1737, 1045-1737, 1047-1715, |
| | 1055-1540, 1056-1354, 1073-1706, 1153-1708, 1158-1807, 1160-1669, 1165-1656, 1193-1789, |
| | 1194-1562, 1222-1872, 1304-1958, 1306-1576, 1336-1952, 1341-1466, 1341-1467, 1382-1941, |
| | 1390-2031, 1392-1993, 1408-1954, 1409-2010, 1416-1950, 1425-1646, 1427-1646, 1427-2133, |
| | 1437-1995, 1438-1687, 1451-2017, 1457-1757, 1468-1669, 1468-1992, 1468-2014, 1488-1711, |
| | 1500-1787, 1535-1808, 1566-1795, 1627-1902, 1630-1741, 1633-1711, 1655-1957 |
| 47/7505793CB1/ | 1-257, 1-300, 1-313, 30-262, 30-1107, 32-255, 32-282, 32-307, 32-308, 33-312, 34-309, 38-324, |
| 1140 | 40-313, 43-278, 45-287, 51-282, 58-580, 59-490, 64-159, 73-283, 75-330, 86-653, 101-609, |
| | 101-628, 182-330, 256-852, 280-791, 317-538, 330-939, 330-1075, 345-866, 348-649, 368-868, |
| | 369-612, 380-790, 385-652, 388-918, 395-699, 400-626, 400-764, 414-687, 426-540, 475-793, |
| | 481-731, 486-691, 511-1038, 519-1107, 525-1106, 527-1106, 532-1117, 543-788, 551-802, 568-1116, |
| | 573-1136, 574-720, 575-790, 578-1033, 580-1113, 597-972, 598-1107, 598-1136, 615-1023, 646-909, |
| | 646-1045, 646-1116, 655-1122, 680-1124, 686-985, 695-1125, 696-1107, 701-1128, 710-1126, 712-1124, |
| | 716-905, 719-1125, 721-950, 722-1140, 745-1126, 746-977, 752-1021, 758-1126, 771-1126, 804-1120, |
| | 818-1019, 848-1054, 848-1121, 849-1140, 850-1108, 855-1126, 856-1125, 857-1126, 867-1115, 875-1125, |
| | 875-1126, 911-1126, 918-1136, 972-1113 |
| 48/7505861CB1/945 | 1-263, 1-268, 1-307, 1-569, 19-945, 23-286, 28-218, 32-249, 33-218, 39-218, 53-295, 148-775, |
| | 214-612, 214-738, 214-780, 218-613, 232-525, 232-735, 235-541, 251-778, 256-414, 339-613, |
| | 339-768, 339-778, 342-790, 346-644, 347-779, 358-779, 364-644, 369-594, 369-779, 369-783, |
| | 378-779, 384-786, 393-788, 404-683, 408-673, 408-782, 410-786, 416-779, 417-779, 422-789, |
| | 426-789, 438-779, 446-786, 454-589, 454-789, 454-839, 459-787, 461-690, 461-732, 461-751, |
| | 470-790, 530-790, 535-850, 543-787, 544-778, 562-786, 562-788, 572-790, 579-787, 632-874, |
| | 640-778, 657-790, 671-945, 677-788, 680-931 |
| 49/7505864CB1/ | 1-275, 2-1298, 15-202, 27-592, 80-673, 112-412, 135-269, 135-369, 135-383, 148-584, 149-604, |
| 1309 | 154-469, 156-423, 166-427, 166-602, 166-719, 167-448, 168-429, 179-501, 181-295, 181-456, |
| | 192-337, 192-668, 192-725, 200-834, 201-486, 201-804, 205-476, 206-477, 218-530, 225-794, |
| | 229-763, 257-468, 329-450, 365-648, 365-653, 376-866, 405-768, 419-868, 434-714, 438-816, |
| | 444-717, 449-662, 480-735, 487-749, 505-762, 520-718, 534-797, 549-753, 555-807, 572-841, |
| | 616-719, 625-860, 651-920, 655-968, 690-776, 711-1309, 835-1288, 851-1077, 885-1291, 889-1144, |
| | 894-1290, 895-1165, 907-1293, 950-1290, 954-1225, 968-1288, 972-1292, 1029-1295 |
| 50/7506427CB1/ | 1-257, 1-300, 1-313, 1-334, 1-406, 1-407, 1-429, 1-473, 1-489, 1-494, 1-553, 2-479, |
| 1458 | 19-719, 22-653, 30-262, 30-354, 30-1425, 32-255, 32-282, 32-307, 32-308, 33-312, 33-521, 33-532, |
| | 33-616, 34-309, 38-324, 38-627, 40-313, 43-278, 45-287, 51-282, 51-395, 51-414, 64-159, 73-283, |
| | 75-343, 79-713, 83-394, 105-877, 105-929, 105-967, 153-424, 176-666, 182-336, 221-493, |
| | 259-545, 263-1170, 308-1169, 356-1170, 361-592, 361-621, 487-1169, 506-634, 538-1170, 717-1082, |
| | 718-1017, 732-1005, 744-858, 793-1111, 799-1049, 804-1009, 829-1356, 837-1425, 843-1424, 845-1424, |
| | 850-1435, 861-1106, 869-1120, 891-1454, 893-1108, 896-1351, 898-1431, 915-1290, 916-1425, 916-1454, |
| | 933-1341, 964-1227, 964-1363, 964-1434, 973-1440, 998-1442, 1004-1303, 1013-1443, 1014-1425, |
| | 1019-1446, 1028-1444, 1030-1442, 1034-1223, 1037-1443, 1039-1268, 1040-1458, 1063-1444, |
| | 1064-1295, 1070-1339, 1076-1444, 1089-1444, 1122-1438, 1166-1372, 1166-1439, 1167-1458, |
| | 1168-1426, 1173-1444, 1174-1443, 1175-1444, 1185-1433, 1193-1443, 1193-1444, 1229-1444, |
| | 1236-1454, 1290-1431 |
| 51/7506429CB1/ | 1-257, 1-300, 1-313, 1-334, 1-406, 1-407, 1-429, 1-473, 30-262, 30-354, 30-1381, 32-255, |
| 1414 | 32-282, 32-307, 32-308, 33-312, 34-309, 38-324, 40-313, 43-278, 45-287, 51-282, 51-395, 51-414, |
| | 64-159, 73-283, 75-343, 83-394, 105-884, 105-922, 105-1092, 107-470, 153-424, 182-336, |
| | 210-1126, 221-473, 332-1126, 418-1126, 480-1125, 496-1016, 510-1018, 537-750, 537-773, 537-992, |
| | 541-685, 550-851, 552-1064, 552-1142, 561-1142, 569-815, 569-1115, 569-1349, 569-1366, 569-1414, |
| | 594-1213, 619-1140, 622-923, 642-1142, 643-886, 654-1064, 659-926, 662-1192, 669-973, 674-900, |
| | 674-1038, 688-961, 700-814, 749-1067, 755-1005, 760-965, 785-1312, 793-1381, 799-1380, 801-1380, |
| | 806-1391, 817-1062, 825-1076, 847-1410, 848-994, 849-1064, 852-1307, 854-1387, 871-1246, 872-1381, |
| | 872-1410, 889-1297, 920-1183, 920-1319, 920-1390, 929-1396, 954-1398, 960-1259, 969-1399, 970-1381, |
| | 975-1402, 984-1400, 986-1398, 990-1179, 993-1399, 995-1224, 996-1414, 1019-1400, 1026-1295, |
| | 1032-1400, 1045-1400, 1078-1394, 1092-1293, 1122-1328, 1122-1395, 1123-1414, 1124-1382, |
| | 1129-1400, 1130-1399, 1131-1400, 1141-1389, 1149-1399, 1149-1400, 1185-1400, 1192-1410 |
| 52/7505799CB1/526 | 1-176, 1-526, 12-494, 28-232, 28-277, 33-229, 33-251, 34-272, 43-324, 44-324, 45-249, 45-274, |
| | 45-293, 45-324, 46-381, 48-304, 48-512, 53-310, 54-294, 55-318, 356-504 |
| 53/7505843CB1/ | 1-49, 1-1989, 50-393, 50-496, 50-532, 50-564, 50-605, 50-644, 50-693, 50-706, 50-748, 54-676, |
| 1989 | 80-259, 116-342, 116-404, 116-529, 116-666, 126-179, 126-211, 126-258, 131-478, 133-412, |
| | 134-619, 148-431, 149-432, 160-342, 167-689, 170-672, 173-401, 177-731, 179-390, 187-758, |
| | 192-753, 196-690, 197-718, 200-708, 208-469, 209-484, 218-503, 228-438, 229-758, 231-509, |
| | 234-1129, 236-755, 240-896, 242-1134, 244-474, 244-482, 252-454, 256-867, 262-767, 264-551, |
| | 265-507, 265-925, 274-801, 278-736, 282-519, 283-522, 284-890, 286-548, 295-550, 297-580, |
| | 299-949, 308-562, 308-701, 313-446, 313-921, 320-557, 320-678, 325-868, 326-513, 326-571, |
| | 328-516, 328-616, 329-1069, 333-567, 333-584, 333-587, 333-589, 333-599, 335-598, 338-569, |
| | 338-588, 338-611, 338-669, 338-984, 345-599, 346-658, 352-482, 353-600, 357-616, 363-589, |
| | 365-944, 365-959, 367-783, 370-697, 371-578, 371-647, 379-662, 379-677, 379-768, 379-927, |
| | 380-599, 380-699, 380-705, 384-853, 387-1049, 392-615, 394-629, 395-668, 395-871, 396-844, |
| | 403-832, 404-631, 409-1096, 410-801, 411-700, 413-713, 415-615, 419-893, 420-568, 435-1126, |
| | 440-732, |
| | 441-702, 446-728, 449-744, 453-662, 453-679, 453-802, 455-659, 456-1014, 456-1015, 459-716, |
| | 464-692, 464-747, 466-1038, 467-1172, 470-555, 470-751, 472-807, 475-760, 476-767, 483-708, |
| | 486-702, 487-767, 490-682, 492-740, 492-1076, 494-1110, 496-750, 497-694, 501-831, 503-762, |
| | 503-842, 503-879, 505-736, 505-762, 510-759, 512-766, 513-906, 514-979, 515-798, 518-910, |
| | 524-747, 532-660, 533-772, 533-791, 533-1172, 535-782, 539-947, 541-1157, 545-1150, 546-809, |
| | 549-706, 549-785, 552-990, 556-923, 563-706, 563-793, 563-802, 565-1137, 566-1115, 569-846, |
| | 570-838, 572-696, 573-859, 574-739, 575-1092, 576-800, 577-824, 579-1151, 580-871, 581-1186, |
| | 583-878, 584-1176, 585-848, 587-1172, 595-1198, 597-864, 599-864, 599-871, 599-896, 600-1332, |
| | 603-1159, 609-914, 614-829, 617-859, 617-863, 619-1173, 624-871, 626-884, 626-904, 626-1228, |
| | 632-1166, 632-1189, 632-1270, 636-920, 637-880, 637-922, 637-1224, 641-845, 643-838, 643-1164, |
| | 645-804, 650-1171, 651-914, 655-845, 655-849, 659-916, 660-768, 661-1352, 662-863, 663-823, |
| | 664-777, 667-877, 668-1172, 669-927, 673-910, 673-1087, 674-919, |
| | 674-1151, 681-962, 681-1158, 681-1162, 691-955, 691-1282, 692-951, 693-1095, 698-915, 713-1308, |
| | 722-1013, 723-866, 726-1307, 729-994, 735-994, 735-1182, 743-1376, 745-1008, 747-908, 750-1025, |
| | 756-1026, 757-1266, 760-972, 760-1053, 761-991, 761-1286, 763-1060, 768-1158, 771-930, 772-874, |
| | 774-1049, 774-1060, 779-1360, 795-1302, 799-1028, 799-1101, 803-980, 814-1175, 817-1358, 824-1009, |
| | 826-1095, 827-1234, 829-1091, 830-1114, 834-1035, 834-1076, 839-1085, 840-1081, 840-1110, 842-1126, |
| | 847-1054, 849-1118, 852-1494, 858-1504, 859-1080, 861-1332, 863-1112, 868-1037, 871-1131, 871-1159, |
| | 872-1139, 886-1300, 887-1140, 889-1482, 890-1264, 891-1187, 893-1155, 895-1127, 897-1610, 899-1118, |
| | 903-1183, 904-1209, 906-1531, 908-1091, 912-1379, 917-1041, 918-1366, 928-1180, 938-1174, 938-1248, |
| | 938-1623, 939-1200, 947-1149, 948-1160, 949-1224, 949-1356, 953-1222, 954-1630, 955-1082, 956-1236, |
| | 956-1480, 957-1216, 960-1194, 961-1350, 963-1102, 963-1103, 963-1649, 964-1210, 965-1053, 967-1648, |
| | 969-1209, 969-1223, 971-1121, 971-1157, 971-1168, 972-1214, 974-1239, 975-1143, 976-1146, 976-1757, |
| | 976-1820, 978-1332, 980-1247, 981-1223, 981-1244, 981-1264, 985-1610, |
| | 988-1272, 990-1257, 993-1307, 994-1350, 996-1221, 996-1253, 996-1263, 996-1280, 996-1283, 997-1228, |
| | 999-1243, 1001-1239, 1002-1285, 1004-1259, 1004-1260, 1005-1130, 1006-1126, 1009-1747, |
| | 1011-1266, 1011-1332, 1014-1276, 1014-1283, 1014-1313, 1015-1262, 1015-1266, 1015-1276, |
| | 1016-1318, 1017-1293, 1018-1254, 1018-1301, 1022-1264, 1022-1630, 1033-1343, 1042-1616, |
| | 1043-1697, 1045-1231, 1045-1548, 1046-1317, 1047-1282, 1049-1286, 1051-1821, 1053-1581, |
| | 1054-1631, 1055-1334, 1055-1480, 1062-1331, 1062-1341, 1063-1367, 1063-1423, 1067-1517, |
| | 1068-1311, 1072-1712, 1073-1331, 1077-1332, 1077-1333, 1079-1716, 1082-1766, 1087-1642, |
| | 1088-1366, 1094-1273, 1096-1399, 1098-1418, 1105-1785, 1107-1432, 1108-1361, 1108-1388, |
| | 1108-1393, 1110-1665, 1112-1360, 1112-1423, 1112-1707, 1123-1408, 1131-1411, 1131-1439, |
| | 1132-1420, 1133-1272, 1141-1409, 1141-1507, 1142-1404, 1142-1408, 1143-1416, 1146-1436, |
| | 1151-1393, 1154-1388, 1157-1656, 1158-1558, 1161-1417, 1162-1337, 1163-1395, 1164-1787, |
| | 1164-1830, 1167-1610, 1168-1411, 1170-1384, 1170-1386, 1172-1411, 1175-1769, 1180-1425, |
| | 1187-1664, 1188-1664, 1189-1783, 1189-1830, 1190-1779, 1190-1823, 1192-1786, 1193-1457, |
| | 1194-1367, 1195-1411, 1195-1488, 1197-1408, 1197-1458, 1200-1458, 1200-1476, |
| | 1200-1821, 1201-1508, 1202-1830, 1205-1450, 1206-1658, 1210-1783, 1216-1493, 1224-1438, |
| | 1224-1480, 1225-1469, 1227-1820, 1232-1483, 1232-1486, 1235-1461, 1235-1483, 1235-1484, |
| | 1239-1832, 1240-1387, 1240-1513, 1240-1521, 1240-1817, 1240-1822, 1243-1494, 1244-1469, |
| | 1244-1503, 1244-1531, 1249-1824, 1250-1506, 1250-1832, 1251-1387, 1251-1456, 1251-1461, |
| | 1251-1512, 1251-1513, 1251-1544, 1251-1610, 1252-1483, 1257-1537, 1259-1529, 1261-1547, |
| | 1262-1521, 1266-1604, 1266-1609, 1267-1461, 1274-1523, 1275-1487, 1275-1506, 1275-1507, |
| | 1278-1803, 1282-1499, 1282-1583, 1283-1581, 1285-1517, 1289-1529, 1289-1568, 1298-1789, |
| | 1301-1771, 1302-1523, 1303-1441, 1303-1496, 1303-1499, 1304-1823, 1305-1572, 1305-1584, |
| | 1310-1769, 1315-1583, 1315-1592, 1317-1631, 1322-1584, 1329-1571, 1329-1586, 1329-1823, |
| | 1331-1572, 1332-1542, 1332-1773, 1337-1627, 1337-1680, 1338-1628, 1342-1597, 1346-1799, |
| | 1348-1594, 1353-1600, 1353-1797, 1362-1605, 1362-1828, 1366-1637, 1376-1617, 1379-1611, |
| | 1385-1521, 1386-1693, 1387-1545, 1390-1653, 1394-1775, 1395-1578, 1395-1609, 1400-1668, |
| | 1400-1674, 1402-1649, 1402-1695, 1405-1686, 1416-1635, 1416-1652, 1420-1775, 1425-1775, |
| | 1429-1614, 1429-1775, 1432-1775, 1433-1812, 1434-1775, 1435-1775, 1438-1605, |
| | 1439-1775, 1449-1692, 1456-1724, 1459-1710, 1465-1715, 1465-1775, 1466-1823, 1467-1752, |
| | 1469-1764, 1473-1724, 1476-1795, 1476-1810, 1478-1670, 1485-1691, 1488-1782, 1488-1817, |
| | 1492-1775, 1493-1811, 1494-1632, 1495-1798, 1496-1715, 1496-1745, 1497-1775, 1498-1814, |
| | 1499-1775, 1501-1754, 1501-1775, 1502-1775, 1505-1775, 1505-1780, 1506-1775, 1506-1782, |
| | 1509-1775, 1510-1740, 1511-1775, 1519-1823, 1523-1741, 1532-1813, 1535-1798, 1538-1775, |
| | 1538-1812, 1540-1833, 1543-1833, 1545-1805, 1545-1823, 1546-1793, 1549-1775, 1552-1828, |
| | 1560-1775, 1579-1755, 1582-1775, 1583-1775, 1589-1805, 1589-1830, 1591-1833, 1592-1801, |
| | 1595-1823, 1598-1775, 1598-1813, 1620-1830, 1626-1775, 1630-1775, 1631-1823, 1632-1775, |
| | 1668-1775, 1669-1775, 1672-1775, 1680-1775, 1688-1842 |
| 54/90001378CB1/ | 1-488, 1-685, 1-741, 1-757, 2-843, 214-1054, 293-965, 308-978, 331-1257, 404-1456, 412-1315, |
| 1987 | 413-1259, 488-1100, 811-1702, 900-1725, 905-1683, 1054-1907, 1066-1987, 1216-1985, 1238-1987, |
| | 1242-1987, 1331-1987, 1381-1986 |
| 55/7504923CB1/ | 1-337, 28-812, 31-296, 31-590, 31-644, 31-693, 31-741, 31-771, 31-850, 31-865, 31-2338, 72-341, |
| 2338 | 108-1039, 126-369, 158-548, 166-434, 166-561, 166-566, 166-573, 166-574, 166-662, 166-690, |
| | 166-691, 166-696, 166-698, 166-701, 166-789, 192-690, 215-576, 255-562, 265-824, 265-830, |
| | 293-862, 306-860, 345-538, 346-751, 346-839, 400-704, 415-863, 418-690, 425-865, 455-703, |
| | 470-738, 470-1079, 473-763, 513-672, 521-791, 555-751, 566-775, 582-665, 585-840, 588-849, |
| | 637-864, 668-845, 932-1250, 962-1116, 1083-1593, 1169-1624, 1170-1624, 1299-1612, 1308-1591, |
| | 1357-1766, 1403-1616, 1419-1665, 1456-1721, 1465-1680, 1591-2034, 1657-1949, 1732-2007, |
| | 1780-2152, 1797-1997, 1814-2135, 2065-2325, 2083-2338 |
| 56/7506151CB1/ | 1-137, 1-148, 1-160, 1-166, 1-181, 1-187, 1-188, 1-190, 1-197, 1-198, 1-201, 1-205, |
| 1488 | 1-207, 1-211, 1-215, 1-229, 1-230, 1-231, 1-233, 1-237, 1-240, 1-241, 1-242, 1-243, |
| | 1-244, 1-245, 1-247, 1-249, 1-250, 1-252, 1-255, 1-259, 1-260, 1-261, 1-262, 1-263, |
| | 1-265, 1-266, 1-267, 1-268, 1-269, 1-270, 1-271, 1-272, 1-273, 1-274, 1-275, 1-276, |
| | 1-277, 1-278, 1-280, 1-281, 1-282, 1-284, 1-285, 1-286, 1-287, 1-288, 1-289, 1-290, |
| | 1-291, 1-292, 1-293, 1-294, 1-295, 1-296, 1-297, 1-299, 1-302, 1-304, 1-305, 1-307, |
| | 1-308, 1-309, 1-313, 1-327, 1-334, 1-345, 1-347, 1-350, 1-370, 1-379, 1-380, 1-426, |
| | 1-436, 1-458, 1-474, 1-552, 1-553, 1-556, 1-567, 1-569, 1-570, 1-582, 1-585, 1-591, |
| | 1-592, 1-609, 1-610, 1-647, 2-217, 2-235, 2-241, 2-244, 2-248, 2-252, 2-253, 2-257, |
| | 2-259, 2-260, 2-267, 2-268, 2-270, 2-272, 2-273, 2-278, 2-279, 2-281, 2-283, 2-284, |
| | 2-285, 2-289, 2-291, 2-294, 2-295, 2-300, 2-328, 2-338, 2-342, 2-352, 2-360, 2-429, |
| | 2-458, 2-489, 2-556, 2-560, 2-615, 2-623, 2-642, 2-647, 3-194, 3-197, 3-208, 3-222, |
| | 3-230, 3-234, 3-247, 3-251, 3-252, 3-259, 3-261, 3-263, 3-267, 3-269, 3-270, 3-272, |
| | 3-273, 3-275, 3-277, 3-278, 3-279, 3-283, 3-284, 3-286, |
| | 3-293, 3-298, 3-309, 3-317, 3-335, 3-370, 3-392, 3-509, 3-518, 3-539, 3-550, 3-558, |
| | 3-565, 3-574, 3-600, 3-617, 3-623, 3-658, 3-673, 3-688, 3-1488, 4-125, 4-169, 4-172, |
| | 4-195, 4-225, 4-229, 4-236, 4-239, 4-241, 4-249, 4-254, 4-260, 4-264, 4-268, 4-275, |
| | 4-285, 4-287, 4-291, 4-300, 4-306, 4-309, 4-326, 4-336, 4-361, 4-365, 4-370, 4-465, |
| | 4-500, 4-591, 5-111, 5-152, 5-248, 5-279, 5-312, 5-458, 5-630, 5-631, 6-202, 6-248, |
| | 6-267, 6-293, 6-297, 6-304, 6-314, 6-368, 6-404, 6-510, 6-688, 7-282, 7-292, 7-303, |
| | 8-200, 8-210, 8-248, 8-262, 8-264, 8-284, 8-296, 8-298, 8-315, 8-322, 8-527, 8-540, |
| | 8-582, 8-667, 9-329, 9-440, 9-460, 9-745, 10-217, 10-227, 10-294, 10-315, 10-367, 10-510, |
| | 10-636, 10-673, 12-101, 12-253, 12-258, 12-261, 12-283, 13-310, 13-328, 14-151, 14-195, 14-246, |
| | 14-264, 14-407, 14-555, 15-282, 15-366, 15-689, 16-221, 16-252, 16-282, 16-287, 16-294, 16-362, |
| | 16-380, 17-601, 18-272, 18-280, 18-292, 18-297, 18-303, 18-307, 18-552, 19-255, 19-287, 19-292, |
| | 20-280, 20-342, 20-358, 21-799, 24-257, 24-260, 25-574, 27-325, 28-610, 30-174, 30-354, 30-666, |
| | 32-272, 32-453, 32-759, 32-827, 33-567, 34-269, 35-323, 35-327, 36-183, 37-326, |
| | 38-256, 38-344, 38-595, 38-875, 39-602, 39-748, 40-322, 42-159, 42-557, 47-327, 53-354, 54-336, |
| | 57-242, 57-318, 57-431, 63-329, 63-354, 68-417, 71-315, 72-347, 73-649, 74-146, 74-766, 90-334, |
| | 90-340, 90-341, 93-356, 94-285, 97-652, 105-364, 106-345, 106-366, 112-369, 112-398, 112-408, |
| | 112-420, 113-409, 114-552, 128-210, 129-345, 129-396, 136-685, 142-263, 142-425, 143-429, |
| | 144-399, 147-394, 148-527, 150-383, 150-572, 151-709, 157-422, 164-444, 164-453, 166-403, |
| | 170-516, 176-807, 177-388, 181-481, 183-794, 186-484, 189-472, 192-487, 192-504, 193-390, |
| | 194-522, 194-792, 208-838, 209-482, 211-315, 211-484, 212-476, 219-475, 221-343, 222-609, |
| | 225-717, 229-444, 229-574, 233-532, 238-810, 240-517, 241-917, 253-419, 253-444, 260-913, |
| | 265-859, 275-511, 276-535, 277-514, 279-531, 280-551, 285-563, 286-503, 286-523, 290-522, |
| | 293-809, 299-626, 301-823, 309-789, 309-921, 310-1019, 315-774, 318-598, 322-601, 322-610, |
| | 323-762, 324-563, 324-591, 325-599, 326-594, 328-695, 328-699, 330-507, 330-560, 330-577, |
| | 330-624, 330-868, 332-570, 332-616, 334-855, 340-615, 340-617, 340-630, 341-519, |
| | 341-828, 343-595, 343-1006, 348-624, 348-637, 349-693, 350-1015, 351-618, 358-799, 359-645, |
| | 359-647, 361-631, 361-633, 361-646, 362-625, 362-987, 366-623, 366-998, 376-673, 377-499, |
| | 378-619, 380-955, 384-632, 385-545, 388-987, 389-848, 397-982, 397-993, 398-655, 400-647, |
| | 401-683, 401-856, 402-666, 404-669, 406-1020, 407-946, 410-699, 410-812, 414-872, 416-711, |
| | 417-1016, 420-730, 425-718, 425-888, 426-912, 428-734, 429-988, 432-703, 432-900, 433-689, |
| | 436-697, 436-715, 436-888, 437-769, 438-698, 438-954, 440-633, 440-729, 441-887, 446-706, |
| | 451-715, 452-680, 452-697, 453-735, 455-1013, 456-915, 463-710, 464-632, 466-667, 468-959, |
| | 481-695, 482-769, 483-733, 484-755, 484-830, 484-1016, 486-766, 487-739, 489-640, 491-660, |
| | 491-773, 493-748, 493-1020, 496-764, 500-971, 505-785, 508-785, 511-791, 512-1061, 523-731, |
| | 524-742, 531-662, 546-813, 547-847, 547-851, 550-886, 555-1020, 559-663, 559-778, 559-832, |
| | 560-859, 565-800, 565-809, 565-836, 565-840, 570-1203, 571-845, 571-849, 572-870, 575-847, |
| | 582-871, 583-862, 587-822, 589-1007, 601-838, 606-881, 608-839, 611-854, |
| | 611-867, 613-885, 617-888, 620-867, 623-863, 623-879, 623-906, 634-870, 634-888, 634-895, |
| | 634-897, 634-917, 634-927, 635-916, 635-928, 637-848, 637-898, 637-922, 638-911, 638-948, |
| | 640-849, 640-860, 640-905, 642-859, 642-917, 643-889, 644-893, 645-820, 645-1020, 646-908, |
| | 649-1011, 652-895, 654-917, 655-945, 660-875, 667-763, 667-917, 667-938, 669-931, 671-945, |
| | 677-933, 681-949, 683-904, 692-767, 692-939, 692-977, 692-1019, 694-912, 694-966, 694-974, |
| | 695-1019, 699-959, 703-950, 703-965, 704-985, 705-986, 707-1011, 709-790, 710-897, 710-920, |
| | 717-916, 718-998, 722-1011, 727-970, 727-1007, 728-914, 732-983, 735-989, 740-935, 741-972, |
| | 763-1012, 768-1020, 771-980, 773-1007, 798-1014, 803-1016, 825-1015, 854-1014, 889-1383, 985-1281, |
| | 1016-1208, 1016-1264, 1039-1311, 1055-1317, 1072-1204, 1110-1269, 1203-1463 |
| 57/7506450CB1/ | 1-20, 1-752, 1-896, 1-1608, 176-687, 176-739 176-742, 176-748, 201-637, 225-687, 232-748, |
| 1608 | 257-847, 263-705, 273-548, 281-904, 281-1042, 281-1048, 281-1138, 281-1175, 281-1177, 421-1341, |
| | 422-1341, 447-1341, 448-1338, 454-1103, 470-831, 476-1411, 478-1411, 484-988, 486-1341, 487-1411, |
| | 491-1342, 492-1341, 495-945, 495-997, 495-1042, 495-1077, 495-1092, 511-1341, 512-1339, 512-1341, |
| | 515-1171, 527-1411, 531-1411, 546-874, 546-1101, 556-1336, 559-1341, 566-781, 571-1143, 577-1164, |
| | 594-845, 596-1341, 603-1409, 621-1341, 635-1149, 644-1341, 645-1254, 647-1341, 681-1225, 688-1123, |
| | 688-1275, 693-897, 696-1411, 735-1000, 756-1350, 763-1249, 779-1481, 798-1475, 800-1388, 806-1253, |
| | 847-1325, 854-1349, 856-1091, 856-1164, 861-1401, 925-1545, 935-1428, 949-1515, 953-1532, 962-1143, |
| | 1003-1600, 1019-1387, 1057-1515, 1101-1358, 1112-1532, 1116-1506, 1133-1574, 1140-1375, |
| | 1144-1608, 1225-1562, 1563-1608 |
| 58/71380031CB1/ | 1-251, 1-254, 1-480, 1-520, 1-569, 63-608, 97-726, 105-736, 129-265, 132-484, 132-708, |
| 2494 | 254-622, 257-606, 266-888, 313-916, 355-859, 381-541, 401-1088, 454-746, 455-744, 455-746, |
| | 475-848, 507-1090, 513-1183, 567-1137, 568-1157, 580-1168, 587-1129, 599-1112, 607-1119, 611-1085, |
| | 612-1095, 613-1302, 622-1170, 623-1137, 644-1174, 699-1242, 713-1341, 715-1242, 797-1464, 803-1468, |
| | 813-1441, 834-1394, 836-1581, 848-1311, 874-1085, 884-1401, 893-1217, 893-1352, 894-1216, 900-1095, |
| | 901-1149, 915-1429, 919-1415, 934-1174, 953-1456, 955-1576, 957-1327, 981-1555, 987-1456, 1006-1597, |
| | 1022-1396, 1090-1432, 1184-1724, 1248-1505, 1248-1728, 1286-1538, 1298-1667, 1320-1515, |
| | 1327-1732, 1402-1745, 1409-1819, 1415-1641, 1464-2019, 1525-2096, 1528-1793, 1531-2036, |
| | 1587-2107, 1677-2114, 1802-2051, 1802-2082, 1802-2115, 1836-2041, 2019-2118, 2114-2494, |
| | 2122-2157, 2122-2173, 2122-2255, 2122-2257, 2122-2271, 2122-2326, 2122-2347, 2122-2348, |
| | 2122-2357, 2122-2358, 2122-2359, 2122-2363, 2122-2371, 2122-2383, 2122-2390, 2122-2402, |
| | 2122-2403, 2122-2457, 2122-2463, 2122-2494, 2124-2167, 2224-2494, 2226-2494, 2234-2494, |
| | 2244-2494, 2262-2494, 2348-2494, 2362-2494, 2364-2494, 2401-2490, 2429-2494 |
| 59/7506054CB1/ | 1-442, 1-546, 1-570, 1-593, 1-633, 1-648, 1-649, 1-662, 2-259, 10-316, 12-262, 12-269, |
| 1414 | 12-288, 12-293, 12-299, 12-325, 13-284, 14-217, 14-246, 14-276, 14-482, 14-542, 14-634, 15-195, |
| | 15-246, 15-247, 15-255, 15-267, 15-269, 15-272, 15-288, 15-292, 15-299, 15-304, 15-413, 15-483, |
| | 15-497, 15-512, 16-257, 16-269, 16-270, 17-170, 17-250, 17-256, 17-273, 17-284, 17-287, 17-293, |
| | 17-298, 17-301, 17-311, 17-606, 18-306, 18-459, 18-482, 19-272, 19-275, 19-298, 19-482, 19-1414, |
| | 21-268, 21-276, 21-320, 21-335, 21-513, 21-602, 22-266, 22-270, 22-280, 22-297, 22-572, 23-133, |
| | 23-256, 23-276, 23-279, 23-307, 24-304, 25-278, 25-303, 26-270, 27-207, 27-273, 27-275, 27-276, |
| | 27-277, 27-298, 27-306, 27-324, 27-364, 27-660, 28-495, 29-196, 29-283, 29-291, 29-300, 29-320, |
| | 29-626, 30-266, 30-348, 33-267, 33-291, 33-409, 34-113, 35-411, 38-297, 38-304, 38-647, 41-287, |
| | 41-320, 41-357, 50-669, 51-317, 54-258, 63-333, 65-320, 66-319, 66-651, 67-422, 68-702, 69-345, |
| | 71-357, 73-685, 73-711, 75-227, 82-243, 83-490, 93-344, 100-389, 128-692, 147-557, 147-594, |
| | 147-657, 150-608, 163-692, 168-511, 168-594, 168-608, 181-719, |
| | 183-553, 190-650, 194-422, 213-463, 220-480, 227-485, 232-659, 251-399, 261-659, 261-719, |
| | 262-547, 280-659, 290-512, 300-556, 330-588, 331-602, 356-699, 360-628, 362-582, 364-663, |
| | 371-621, 371-719, 380-642, 400-693, 408-681, 419-628, 439-582, 442-581, 449-697, 463-610, |
| | 480-719, 484-593, 496-719, 534-694, 546-713, 568-690, 574-827, 574-1004, 574-1030, 574-1052, |
| | 574-1119, 574-1134, 574-1200, 574-1205, 678-1185, 714-976, 715-901, 715-934, 715-945, 715-955, |
| | 715-971, 715-978, 715-1064, 715-1075, 715-1098, 715-1102, 715-1134, 726-1103, 734-895, 745-1100, |
| | 748-1018, 750-969, 751-1020, 751-1023, 751-1025, 754-1006, 754-1407, 755-996, 755-1100, 757-1251, |
| | 758-906, 758-1276, 761-1203, 766-854, 767-1208, 768-1095, 772-1237, 784-1319, 789-1037, 793-1085, |
| | 794-992, 813-1105, 816-1260, 817-1123, 825-990, 839-1121, 840-1076, 840-1130, 841-1090, 851-1154, |
| | 858-1414, 870-1154, 871-1407, 877-1414, 880-1112, 880-1138, 887-1192, 890-1163, 892-1162, 894-1185, |
| | 894-1188, 895-1205, 901-1177, 901-1180, 901-1189, 901-1210, 907-1414, 911-1414, 912-1414, 916-1203, |
| | 922-1163, 930-1374, 941-1058, 957-1166, 957-1167, 960-1414, 961-1414, 966-1412, 972-1414, 974-1266, |
| | 976-1183, 982-1241, 987-1255, 989-1247, 993-1414, 997-1414, 999-1414, 1020-1413, 1027-1176, |
| | 1035-1242, 1046-1258, 1048-1414, 1051-1413, 1067-1414, 1082-1380, 1083-1387, 1093-1380, |
| | 1100-1242, 1100-1392, 1105-1380, 1115-1399, 1119-1414, 1126-1414, 1128-1414, 1129-1386, |
| | 1136-1372, 1138-1380, 1140-1322, 1145-1414, 1147-1260, 1148-1406, 1149-1408, 1156-1391, |
| | 1159-1414, 1163-1410, 1163-1414, 1166-1406, 1170-1393, 1175-1384, 1181-1414, 1182-1414, |
| | 1185-1366, 1185-1377, 1185-1404, 1186-1414, 1191-1407, 1234-1380, 1241-1410, 1249-1400, |
| | 1259-1414, 1264-1414, 1276-1410, 1280-1412, 1285-1407, 1296-1412, 1310-1413, 1312-1406, |
| | 1345-1410 |
| 60/7506139CB1/ | 1-242, 1-254, 1-527, 1-635, 2-255, 2-273, 4-268, 15-304, 15-379, 15-2352, 54-538, 57-766, |
| 2352 | 131-533, 193-459, 193-462, 237-509, 286-853, 367-1193, 417-967, 421-663, 421-955, 475-970, |
| | 486-888, 490-1028, 521-1110, 585-975, 666-907, 666-912, 666-986, 666-1211, 706-974, 714-1332, |
| | 790-1371, 803-1262, 803-1374, 945-1177, 973-1241, 978-1263, 978-1271, 978-1351, 978-1407, 978-1412, |
| | 978-1458, 978-1498, 978-1568, 978-1584, 978-1585, 978-1601, 978-1605, 989-1202, 997-1279, 999-1600, |
| | 1017-1565, 1041-1600, 1048-1600, 1055-1600, 1061-1205, 1061-1315, 1061-1334, 1093-1600, |
| | 1117-1600, 1157-1497, 1160-1336, 1182-1600, 1184-1600, 1237-1578, 1243-1600, 1244-1600, |
| | 1279-1578, 1313-1600, 1350-1600, 1374-1565, 1601-2008, 1601-2041, 1601-2048, 1601-2094, |
| | 1601-2097, 1601-2101, 1610-2036, 1610-2051, 1624-1940, 1624-2158, 1649-1854, 1651-2123, |
| | 1662-1888, 1840-2141, 1840-2148 |
| 61/7506426CB1/ | 1-257, 1-300, 1-313, 1-334, 1-406, 1-407, 1-429, 1-473, 1-489, 1-494, 2-479, 30-262, |
| 1422 | 30-354, 30-1389, 32-255, 32-282, 32-307, 32-308, 33-312, 33-504, 34-309, 38-324, 40-313, 43-278, |
| | 45-287, 51-282, 51-395, 51-414, 64-159, 73-283, 75-343, 83-394, 105-1068, 107-470, 153-424, |
| | 182-336, 221-493, 343-1134, 504-1024, 518-1026, 545-758, 545-781, 545-1000, 549-693, 558-859, |
| | 560-1072, 560-1150, 569-1150, 577-823, 577-1123, 577-1357, 577-1374, 577-1422, 602-1221, 627-1148, |
| | 650-1150, 651-894, 662-1072, 670-1200, 682-1046, 708-822, 793-1320, 801-1389, 807-1388, 809-1388, |
| | 814-1399, 825-1070, 833-1084, 850-1398, 855-1418, 856-1002, 857-1072, 860-1315, 862-1395, 879-1254, |
| | 880-1389, 880-1418, 897-1305, 928-1191, 928-1327, 928-1398, 937-1404, 962-1406, 968-1267, 977-1407, |
| | 978-1389, 983-1410, 992-1408, 994-1406, 998-1187, 1001-1407, 1003-1232, 1004-1422, 1027-1408, |
| | 1028-1259, 1034-1303, 1040-1408, 1053-1408, 1086-1402, 1100-1301, 1130-1336, 1130-1403, |
| | 1131-1422, 1132-1390, 1137-1408, 1138-1407, 1139-1408, 1149-1397, 1157-1407, 1157-1408, |
| | 1193-1408, 1200-1418, 1254-1395 |
| 62/7506741CB1/ | 1-328, 1-364, 1-514, 1-573, 1-603, 1-1950, 21-602, 22-467, 22-578, 22-595, 22-647, 28-634, |
| 2101 | 33-649, 39-802, 39-810, 39-822, 39-831, 39-833, 39-866, 39-903, 39-932, 39-1003, 42-730, 70-686, |
| | 232-913, 276-628, 352-1333, 373-1333, 386-681, 407-1333, 410-1333, 420-1333, 421-1333, 452-1333, |
| | 457-1333, 499-1333, 509-1333, 569-1213, 703-1333, 878-962, 895-1327, 1063-1608, 1099-1387, 1138-1689, |
| | 1251-1543, 1278-1917, 1298-1518, 1308-1513, 1308-1583, 1330-1852, 1353-2101, 1374-1903, |
| | 1393-1714, 1401-1658 |
| 63/7506743CB1/ | 1-328, 1-364, 1-514, 1-573, 1-603, 1-1698, 21-602, 22-467, 22-578, 22-598, 22-647, 28-634, |
| 1849 | 33-649, 39-757, 39-788, 39-789, 39-794, 39-974, 42-730, 70-686, 276-628, 308-1081, 386-681, |
| | 796-1356, 847-1135, 886-1437, 897-1194, 999-1291, 1026-1665, 1046-1266, 1056-1261, 1056-1331, |
| | 1078-1600, 1101-1849, 1122-1651, 1141-1462, 1149-1406, 1151-1242, 1151-1245 |
| 64/7506746CB1/ | 1-328, 1-514, 1-603, 1-1885, 22-467, 22-578, 414-1268, 1288-2036 |
| 2036 | |
| 65/7506748CB1/ | 1-153, 1-201, 1-202, 1-256, 1-265, 1-271, 1-282, 7-145, 12-289, 17-290, 17-313, 21-264, |
| 1718 | 21-307, 23-261, 23-963, 24-217, 25-227, 25-289, 25-294, 25-316, 26-256, 26-296, 27-305, 27-323, |
| | 35-284, 35-295, 36-280, 36-329, 38-333, 41-296, 43-291, 44-204, 45-201, 45-229, 45-250, 45-264, |
| | 45-281, 45-284, 45-315, 45-323, 45-325, 45-329, 45-340, 45-357, 46-314, 46-335, 46-582, 47-311, |
| | 47-351, 47-389, 48-235, 48-259, 48-266, 48-295, 48-308, 48-315, 48-326, 48-332, 48-346, 48-347, |
| | 49-277, 49-287, 50-234, 50-238, 50-266, 50-276, 50-290, 50-292, 50-313, 50-320, 50-328, 50-332, |
| | 50-341, 50-346, 51-234, 51-307, 52-297, 53-374, 54-304, 54-305, 55-309, 55-336, 55-342, 55-343, |
| | 55-353, 55-387, 56-313, 57-154, 57-228, 57-229, 57-316, 57-354, 57-389, 58-228, 58-237, 58-241, |
| | 58-253, 58-262, 58-266, 58-270, 58-273, 58-276, 58-278, 58-285, 58-286, 58-291, 58-292, 58-296, |
| | 58-308, 58-309, 58-312, 58-315, 58-317, 58-318, 58-328, 58-332, 58-334, 58-341, 58-347, 58-352, |
| | 59-339, 60-296, 60-320, 60-332, 60-345, 60-352, 61-266, 61-275, 61-290, 61-353, 62-220, 62-296, |
| | 62-308, 62-314, 62-355, 63-296, 64-248, 64-292, 64-311, 70-315, |
| | 71-308, 71-341, 73-296, 73-341, 76-311, 79-332, 81-357, 91-365, 94-296, 95-343, 116-356, 137-387, |
| | 153-383, 180-389, 386-533, 386-595, 386-600, 386-609, 386-621, 386-631, 386-661, 386-670, |
| | 386-672, 386-783, 386-962, 388-652, 389-908, 389-1012, 390-636, 390-640, 390-713, 390-966, |
| | 392-968, 394-586, 394-595, 394-619, 394-620, 394-644, 394-660, 394-774, 394-814, 394-865, |
| | 396-889, 397-743, 397-932, 398-653, 399-641, 399-673, 400-611, 402-627, 402-651, 402-837, |
| | 403-672, 404-694, 407-628, 413-606, 413-667, 413-698, 413-708, 413-895, 413-971, 415-666, |
| | 421-869, 423-678, 428-558, 431-874, 432-962, 435-561, 436-582, 439-707, 440-647, 440-727, |
| | 445-656, 445-669, 446-670, 446-774, 450-1012, 451-983, 453-929, 453-944, 453-999, 457-952, |
| | 459-593, 463-729, 463-937, 468-942, 469-971, 473-690, 474-764, 475-727, 475-750, 482-971, |
| | 483-891, 483-923, 486-910, 487-719, 492-767, 492-777, 492-803, 493-970, 500-965, 501-725, |
| | 502-989, 504-645, 510-730, 510-769, 513-960, 514-761, 514-787, 514-899, 520-755, 521-762, |
| | 522-977, 529-973, 530-973, 533-987, 537-978, 537-990, 543-877, 545-989, 552-783, 552-807, |
| | 552-891, 552-906, 554-807, 555-757, 555-832, 555-968, 555-991, 556-993, 558-917, 558-1014, |
| | 560-936, 561-979, 564-813, 564-976, 576-1000, 581-969, 586-824, 592-868, 595-1004, 598-1012, |
| | 599-964, 604-831, 604-870, 604-970, 608-974, 614-971, 618-855, 619-965, 620-912, 620-976, |
| | 623-976, 624-896, 628-798, 628-800, 628-848, 628-974, 628-976, 630-946, 640-802, 641-976, |
| | 642-879, 648-771, 648-915, 652-974, 660-916, 660-932, 667-906, 672-764, 673-887, 674-910, |
| | 674-954, 674-973, 680-914, 684-987, 686-977, 690-981, 697-963, 698-953, 703-976, 705-964, |
| | 707-929, 712-926, 715-973, 718-971, 722-976, 725-1009, 735-981, 735-1003, 749-965, 759-976, |
| | 759-1012, 760-1010, 763-1010, 769-976, 770-966, 779-976, 780-991, 785-976, 791-976, 800-1012, |
| | 801-977, 806-983, 823-947, 823-973, 830-971, 833-1023, 838-1027, 841-971, 851-1697, 852-976, |
| | 853-1012, 1567-1669, 1567-1717, 1567-1718, 1571-1694, 1571-1718, 1572-1718, 1574-1718, |
| | 1575-1718, 1576-1718, 1577-1718, 1581-1718, 1582-1718, 1586-1718, 1587-1718, 1589-1718, |
| | 1593-1718, 1595-1718, 1599-1718, 1604-1716, 1605-1718, 1609-1718, 1610-1718, 1611-1718, |
| | 1618-1718, 1619-1718, 1622-1718, 1623-1718, 1628-1718, 1636-1715, 1637-1716, 1638-1718, |
| | 1640-1718, 1646-1718, 1649-1718, 1650-1718, 1656-1718, 1657-1718, 1658-1718, 1665-1718, |
| | 1666-1718, 1669-1718, 1673-1718, 1679-1718, 1681-1718, 1688-1718, 1690-1718, 1691-1718, |
| | 1692-1718, 1694-1718, 1696-1718, 1697-1718 |
| 66/1419966CB1/ | 1-256, 1-415, 1-491, 1-1845, 317-1237, 353-1179, 793-1612, 914-1591, 1455-1828 |
| 1845 | |
| 67/7506451CB1/ | 1-276, 1-1465, 14-504, 14-782, 442-1177, 506-1175, 526-872, 622-837, 650-859, 666-1177, |
| 1465 | 701-1330, 869-1223, 893-1351, 924-1172, 937-1194, 948-1368, 952-1342, 969-1410, 976-1211, 980-1454, |
| | 1061-1398, 1145-1212, 1399-1453 |
| 68/90015249CB1/ | 1-624, 301-536, 301-603, 301-664, 301-709, 301-824, 342-522, 459-624 |
| 824 | |
| 69/7487231CB1/ | 1-3103, 201-771, 401-642, 401-663, 401-685, 401-768, 401-788, 401-794, 401-844, 401-852, |
| 3103 | 401-863, 401-865, 401-866, 401-867, 401-874, 401-878, 401-885, 401-886, 401-888, 401-895, |
| | 401-896, 401-900, 401-902, 401-907, 401-908, 401-911, 401-918, 401-930, 401-932, 401-933, |
| | 401-936, 401-949, 401-950, 401-952, 401-954, 401-955, 401-959, 401-962, 401-968, 401-975, |
| | 401-978, 401-981, 401-987, 401-993, 401-1017, 401-1018, 401-1022, 401-1027, 401-1028, 401-1036, |
| | 401-1037, 401-1039, 401-1042, 401-1044, 401-1045, 401-1049, 401-1050, 401-1052, 401-1053, 401-1055, |
| | 401-1057, 401-1059, 401-1060, 401-1061, 401-1062, 401-1064, 401-1065, 401-1066, 401-1068, 401-1071, |
| | 401-1073, 401-1077, 401-1082, 401-1088, 401-1091, 401-1095, 401-1097, 401-1098, 401-1100, 401-1101, |
| | 401-1105, 401-1108, 401-1112, 401-1113, 401-1115, 401-1117, 401-1118, 401-1120, 401-1124, 401-1125, |
| | 401-1126, 401-1129, 401-1138, 401-1143, 401-1146, 401-1147, 401-1152, 401-1155, 401-1158, 401-1160, |
| | 401-1163, 401-1167, 401-1175, 401-1176, 401-1183, 401-1186, 401-1191, 401-1194, 401-1195, 401-1198, |
| | 401-1201, 401-1202, 401-1210, 401-1211, 401-1214, 401-1221, 401-1222, 401-1228, 401-1232, 401-1233, |
| | 401-1242, 401-1243, 401-1244, 401-1246, 401-1248, 403-1215, 405-1049, 405-1062, 407-898, 408-1034, |
| | 416-672, 416-1242, 418-736, 421-961, 432-3103, 436-669, 436-1143, 441-688, 452-907, 455-680, 4 |
| | 59-991, 461-1118, 466-1183, 470-1207, 688-1348, 708-1347, 920-1399, 946-1198, 975-1568 |
| 70/7506260CB1/ | 1-247, 1-1324, 1-1333, 16-283, 19-283, 20-284, |
| 1333 | 21-329, 21-360, 23-224, 27-312, 29-332, 31-292, 33-231, 33-289, 42-302, 42-329, 43-311, 43-319, |
| | 44-318, 44-344, 44-460, 44-673, 46-291, 47-215, 48-316, 49-290, 49-329, 50-330, 51-381, 54-271, |
| | 54-274, 55-328, 134-326, 775-1037 |
| 71/7506270CB1/ | 1-237, 1-250, 1-251, 1-252, 1-263, 1-280, 1-436, 1-455, 1-477, 1-478, 1-495, 1-537, |
| 1675 | 1-571, 1-573, 1-586, 1-596, 1-607, 1-611, 2-252, 2-573, 3-183, 3-184, 3-211, 3-232, |
| | 3-247, 3-252, 3-255, 3-257, 3-260, 3-267, 3-270, 3-273, 3-277, 3-424, 4-187, 4-192, |
| | 4-209, 4-221, 4-232, 4-245, 4-249, 4-261, 4-262, 4-263, 4-268, 4-271, 4-281, 4-1675, |
| | 5-245, 5-272, 6-216, 6-238, 6-265, 7-124, 7-150, 7-240, 7-245, 7-246, 7-247, 7-257, |
| | 7-259, 7-260, 7-261, 7-266, 7-274, 7-283, 8-217, 8-259, 8-276, 9-163, 9-181, 9-208, |
| | 9-211, 9-245, 9-250, 9-259, 9-283, 10-202, 10-236, 10-263, 11-155, 11-247, 11-266, 11-267, |
| | 11-269, 12-268, 23-277, 36-245, 55-638, 68-234, 79-233, 82-624, 212-779, 254-827, 276-832, |
| | 280-422, 280-507, 280-545, 280-550, 280-780, 280-796, 280-823, 280-828, 280-831, 280-836, |
| | 280-854, 280-868, 280-978, 280-1122, 281-496, 282-935, 284-516, 285-587, 285-934, 286-396, |
| | 287-501, 287-511, 289-733, 289-876, 291-736, 291-831, 294-572, 294-648, 295-919, 295-979, |
| | 301-805, 301-831, 301-897, 302-900, 305-730, 309-570, 310-792, 314-1137, 316-853, 316-949, |
| | 317-402, 317-596, 317-911, 318-580, 321-909, 324-580, |
| | 325-605, 327-818, 327-963, 328-797, 330-941, 331-609, 331-773, 332-579, 332-634, 333-836, |
| | 334-576, 334-874, 339-639, 344-556, 347-596, 347-616, 349-585, 351-604, 354-627, 356-585, |
| | 356-628, 356-997, 356-1003, 361-603, 361-935, 362-890, 365-1025, 366-636, 366-1003, 367-926, |
| | 367-996, 367-1001, 372-947, 376-697, 376-1021, 377-1070, 378-918, 384-1004, 385-637, 386-628, |
| | 386-942, 389-657, 390-559, 390-569, 390-584, 391-556, 396-932, 398-883, 398-930, 405-831, |
| | 406-886, 410-689, 419-1021, 420-690, 423-1021, 424-753, 424-999, 426-902, 428-726, 428-781, |
| | 429-1056, 430-1061, 437-1093, 440-1037, 443-899, 445-739, 448-982, 449-960, 450-704, 450-1042, |
| | 451-689, 451-694, 451-706, 451-719, 451-1128, 452-717, 452-894, 452-970, 453-709, 453-1109, |
| | 455-1053, 455-1069, 455-1104, 456-652, 458-1079, 459-697, 460-737, 460-767, 461-714, 461-979, |
| | 462-1074, 462-1081, 468-737, 469-733, 470-917, 473-640, 473-752, 476-723, 479-877, 482-1024, |
| | 482-1086, 484-1117, 484-1122, 484-1128, 484-1189, 486-616, 487-856, 491-755, 491-769, 494-746, |
| | 495-1153, 496-1121, 496-1135, 497-700, 497-713, 497-1016, 498-698, 498-753, 498-1016, 501-785, |
| | 503-762, 504-1051, 505-1092, 508-968, 510-1174, 515-748, |
| | 515-757, 515-801, 515-1115, 515-1191, 517-762, 517-787, 517-1061, 518-1023, 521-815, 521-1158, |
| | 522-932, 523-1106, 524-792, 526-831, 527-814, 528-805, 528-1043, 531-1105, 531-1117, 531-1169, |
| | 535-1153, 536-804, 536-1161, 537-1067, 538-1095, 540-773, 542-919, 542-1187, 544-808, 546-1137, |
| | 552-768, 555-1185, 557-824, 558-884, 558-980, 558-1101, 558-1193, 560-1098, 562-1179, 564-794, |
| | 564-797, 564-803, 564-1198, 565-833, 565-864, 565-1231, 567-691, 567-914, 569-785, 571-1168, |
| | 571-1199, 573-1117, 576-804, 576-914, 576-1092, 581-858, 581-1158, 584-1127, 584-1136, 585-835, |
| | 587-838, 588-1116, 589-855, 589-1145, 590-834, 593-1116, 595-847, 595-872, 595-982, 596-1117, |
| | 597-1120, 599-1195, 604-1228, 605-740, 605-836, 609-1212, 610-1082, 611-1192, 611-1194, 613-1069, |
| | 613-1189, 614-1255, 618-871, 618-1064, 618-1071, 620-1236, 621-923, 625-929, 625-936, 626-841, |
| | 626-1191, 626-1194, 627-859, 627-1186, 627-1192, 629-989, 630-811, 633-865, 633-1139, 637-1100, |
| | 637-1262, 639-891, 639-895, 640-910, 641-780, 641-893, 641-899, |
| | 641-925, 641-968, 641-1302, 642-917, 643-880, 643-887, 643-890, 643-896, 643-915, 644-870, |
| | 644-920, 646-875, 646-918, 646-1202, 650-1130, 652-1112, 658-900, 658-927, 659-1186, 660-1144, |
| | 661-1092, 662-1195, 664-901, 666-1195, 670-1054, 671-960, 676-743, 678-917, 678-1028, 678-1054, |
| | 678-1173, 679-912, 683-941, 683-1096, 684-891, 684-912, 685-932, 686-832, 688-943, 691-1415, |
| | 694-938, 694-990, 694-1224, 695-1206, 696-944, 698-973, 699-797, 701-948, 701-983, 702-993, |
| | 702-1189, 704-1192, 706-1326, 710-1114, 712-1195, 712-1200, 715-860, 717-1243, 723-922, |
| | 723-1002, 723-1393, 724-1195, 726-1286, 727-973, 731-1006, 732-972, 734-1206, 735-1195, 736-1004, |
| | 736-1357, 737-1265, 738-992, 740-1195, 741-974, 742-1002, 742-1195, 743-1195, 744-1001, 745-1208, |
| | 746-1195, 750-1002, 750-1029, 750-1195, 751-1267, 751-1270, 752-1195, 754-996, 756-985, 758-1197, |
| | 760-1195, 762-1186, 762-1453, 766-1364, 767-1000, 768-1195, 769-1414, 770-954, 771-1195, 772-1018, |
| | 772-1023, 772-1195, 773-1097, 775-1022, 775-1025, 775-1302, 776-1016, 776-1046, 776-1059, 776-1199, |
| | 777-1062, 778-1056, 778-1194, 779-1053, 787-1195, 787-1200, 793-1123, 796-1015, 797-1095, 797-1199, |
| | 799-1293, 801-1078, 801-1085, 804-1198, 807-1090, 809-1393, |
| | 813-1052, 814-1199, 817-1194, 820-1199, 821-1072, 822-984, 824-1036, 824-1194, 825-1198, 825-1416, |
| | 833-1072, 833-1085, 833-1431, 835-1060, 835-1129, 836-1143, 837-1173, 838-1059, 840-1207, 841-1020, |
| | 843-1067, 843-1098, 845-1475, 847-1082, 849-1091, 849-1392, 850-1199, 852-1274, 855-1102, 855-1189, |
| | 858-1506, 861-1197, 863-1190, 863-1193, 864-1143, 865-1081, 865-1200, 866-1128, 866-1143, 866-1158, |
| | 866-1169, 867-1076, 867-1180, 871-1115, 877-1100, 877-1114, 877-1296, 878-1113, 878-1134, 878-1489, |
| | 880-1123, 880-1159, 880-1185, 880-1186, 880-1198, 881-1145, 881-1438, 883-1033, 883-1200, 883-1434, |
| | 889-1194, 892-1173, 893-1175, 896-1159, 898-1195, 901-1113, 901-1173, 901-1190, 902-1137, 902-1149, |
| | 902-1156, 902-1157, 902-1163, 902-1171, 902-1480, 904-1480, 906-1199, 909-1543, 910-1206, 910-1508, |
| | 913-1158, 913-1187, 913-1200, 913-1508, 919-1200, 920-1173, 921-1171, 921-1491, 921-1504, 923-1176, |
| | 923-1220, 923-1229, 924-1202, 927-1194, 931-1167, 934-1195, 938-1164, 938-1180, 939-1229, 941-1162, |
| | 946-1200, 946-1216, 946-1360, 946-1445, |
| | 947-1186, 950-1229, 951-1154, 951-1500, 955-1227, 956-1245, 957-1482, 961-1194, 961-1248, 961-1251, |
| | 963-1221, 964-1170, 965-1197, 965-1221, 969-1216, 970-1468, 971-1199, 972-1208, 972-1220, 974-1189, |
| | 974-1200, 974-1202, 976-1195, 976-1199, 982-1268, 983-1200, 983-1235, 983-1261, 983-1403, 989-1278, |
| | 990-1200, 996-1285, 997-1195, 997-1200, 1002-1196, 1010-1252, 1011-1228, 1012-1247, 1012-1295, |
| | 1014-1288, 1016-1272, 1016-1273, 1022-1317, 1022-1321, 1038-1195, 1038-1362, 1038-1496, |
| | 1043-1402, 1044-1255, 1046-1293, 1051-1292, 1051-1308, 1051-1346, 1052-1207, 1052-1241, |
| | 1052-1242, 1052-1259, 1052-1261, 1053-1309, 1054-1311, 1054-1312, 1054-1355, 1054-1382, |
| | 1057-1315, 1058-1297, 1062-1293, 1066-1195, 1067-1194, 1072-1391, 1076-1270, 1076-1362, |
| | 1078-1270, 1078-1320, 1078-1574, 1080-1346, 1081-1272, 1081-1525, 1084-1265, 1084-1329, |
| | 1084-1352, 1084-1362, 1084-1364, 1086-1325, 1086-1540, 1088-1374, 1091-1371, 1100-1332, |
| | 1100-1339, 1103-1371, 1108-1398, 1110-1346, 1110-1384, 1111-1194, 1112-1400, 1114-1427, |
| | 1126-1379, 1136-1379, 1140-1263, 1141-1487, 1142-1195, 1142-1278, 1142-1367, 1143-1429, |
| | 1151-1376, 1151-1391, 1153-1383, 1153-1409, 1162-1416, 1162-1435, 1162-1456, 1165-1407, |
| | 1168-1454, 1177-1378, 1177-1395, 1177-1424, |
| | 1177-1433, 1177-1448, 1177-1473, 1179-1500, 1180-1342, 1192-1450, 1195-1474, 1195-1483, |
| | 1197-1400, 1197-1454, 1199-1442, 1201-1475, 1204-1494, 1208-1356, 1208-1425, 1208-1446, |
| | 1208-1469, 1208-1473, 1209-1338, 1209-1463, 1209-1486, 1209-1505, 1215-1437, 1215-1441, |
| | 1215-1464, 1215-1508, 1218-1474, 1218-1480, 1223-1489, 1227-1410, 1232-1495, 1233-1471, |
| | 1233-1476, 1233-1485, 1233-1488, 1233-1497, 1233-1510, 1234-1478, 1234-1510, 1235-1458, |
| | 1237-1500, 1240-1464, 1240-1500, 1241-1488, 1241-1500, 1242-1510, 1243-1378, 1243-1533, |
| | 1244-1320, 1244-1486, 1244-1533, 1245-1490, 1245-1499, 1248-1505, 1250-1540, 1254-1477, |
| | 1275-1495, 1292-1527, 1294-1488, 1296-1500, 1324-1495, 1328-1498 |
| 72/7506306CB1/ | 1-251, 1-254, 1-480, 1-520, 1-569, 1-2122, 63-608, 97-726, 105-736, 129-265, 132-484, |
| 2122 | 132-708, 257-606, 313-916, 355-859, 401-1088, 421-965, 431-945, 438-1023, 454-746, 455-614, |
| | 455-746, 455-752, 455-753, 455-1006, 455-1044, 458-951, 460-746, 498-746, 507-1090, 513-1183, |
| | 555-1137, 567-1137, 568-873, 568-1157, 580-1168, 599-1112, 607-816, 607-1119, 611-1085, 612-1095, |
| | 613-1302, 622-1170, 623-1137, 644-1174, 713-1341, 797-1464, 803-1468, 813-1441, 834-1394, 836-1581, |
| | 848-1311, 874-1085, 874-1146, 874-1583, 884-1401, 893-1217, 893-1352, 894-1216, 900-1095, 901-1149, |
| | 915-1429, 919-1415, 934-1174, 953-1456, 957-1327, 981-1555, 987-1456, 1022-1396, 1039-1231, |
| | 1045-1664, 1051-1712, 1053-1675, 1135-1647, 1172-1690, 1184-1724, 1233-1640, 1240-1817, |
| | 1242-1792, 1248-1505, 1248-1728, 1274-1858, 1286-1538, 1298-1667, 1320-1515, 1367-1463, |
| | 1367-1951, 1402-1745, 1409-1819, 1409-1959, 1415-1641, 1464-2019, 1525-2098, 1528-1793, |
| | 1587-2109, 1658-2116, 1710-2116, 1798-1999, 1802-2052, 1802-2084, 1802-2120, 1836-1939, |
| | 1886-2117 |
| 73/7506428CB1/ | 1-257, 1-300, 1-313, 1-334, 1-406, 1-407, 1-429, 1-473, 1-489, 1-494, 1-553, 1-591, |
| 1326 | 2-479, 6-587, 22-631, 30-262, 30-354, 30-1293, 32-255, 32-282, 32-307, 32-308, 33-312, 33-521, |
| | 33-532, 33-616, 34-309, 38-324, 38-627, 40-313, 43-278, 45-287, 51-282, 51-395, 51-414, 64-159, |
| | 73-283, 75-343, 79-631, 83-394, 105-871, 107-470, 153-424, 182-336, 193-1038, 198-609, |
| | 221-493, 259-545, 311-595, 322-568, 361-592, 361-621, 405-619, 506-631, 627-726, 627-873, |
| | 627-950, 661-979, 667-917, 672-877, 697-1224, 705-1293, 711-1292, 713-1292, 718-1303, 729-974, |
| | 737-988, 759-1322, 760-906, 761-976, 764-1219, 766-1299, 783-1158, 784-1293, 784-1322, 801-1209, |
| | 832-1095, 832-1231, 832-1302, 841-1308, 866-1310, 872-1171, 881-1311, 882-1293, 887-1314, 896-1312, |
| | 898-1310, 902-1091, 905-1311, 907-1136, 908-1326, 931-1312, 932-1163, 938-1207, 944-1312, 957-1312, |
| | 990-1306, 1004-1205, 1034-1240, 1034-1307, 1035-1326, 1036-1294, 1041-1312, 1042-1311, |
| | 1043-1312, 1053-1301, 1061-1311, 1061-1312, 1097-1312, 1104-1322, 1158-1299 |
| 74/7678032CB1/ | 1-205, 1-259, 1-270, 3-241, 3-280, 4-288, 6-278, 7-506, 11-251, 11-266, 11-275, 12-597, |
| 3899 | 17-522, 118-166, 233-814, 375-1127, 522-673, 526-937, 599-1388, 657-1101, 664-1330, 673-1330, |
| | 731-964, 731-1232, 734-977, 734-1263, 740-963, 744-1363, 775-1306, 783-1194, 783-1262, 802-1109, |
| | 870-1086, 890-1325, 911-1430, 942-1293, 958-1330, 974-1509, 981-1266, 1007-1670, 1009-1340, 1035-1456, |
| | 1053-1656, 1091-1696, 1099-1441, 1129-1685, 1139-1411, 1139-1674, 1205-1586, 1205-1802, |
| | 1246-1682, 1280-1431, 1326-1487, 1351-1508, 1404-1699, 1438-1632, 1478-1535, 1535-2156, |
| | 1613-2334, 1661-1891, 1689-1890, 1722-2471, 1726-2354, 1734-2106, 1736-1996, 1736-2032, |
| | 1765-1929, 1777-2368, 1785-2375, 1789-2332, 1834-2091, 1862-2505, 1865-2488, 1879-2304, |
| | 1895-2515, 1921-2354, 1973-2475, 1976-2208, 1976-2224, 1982-2207, 1982-2234, 1982-2236, |
| | 1982-2528, 1995-2418, 2003-2480, 2004-2614, 2033-2643, 2062-2934, 2067-2358, 2070-2324, |
| | 2074-2744, 2079-2746, 2098-2369, 2105-2240, 2105-2244, 2107-2226, 2122-2357, 2146-2763, |
| | 2149-2686, 2165-2511, 2172-2728, 2185-2412, 2206-2763, 2220-2401, 2220-2455, 2242-2739, |
| | 2246-2481, 2247-2525, 2247-2566, 2247-2819, 2247-2893, 2252-2942, 2254-2510, 2254-2795, |
| | 2269-2546, 2269-2578, 2292-2945, |
| | 2304-2439, 2304-2748, 2304-2804, 2306-2573, 2307-2759, 2315-2871, 2316-2672, 2338-2876, |
| | 2340-2635, 2341-2753, 2342-2500, 2342-2763, 2362-3006, 2387-2753, 2396-2955, 2396-2959, |
| | 2400-2943, 2401-2544, 2409-2686, 2415-2617, 2419-2758, 2419-2838, 2420-2814, 2425-2691, |
| | 2436-2636, 2437-2573, 2448-2753, 2464-2758, 2464-2907, 2472-2753, 2481-3029, 2488-2750, |
| | 2492-2997, 2522-2775, 2525-2753, 2532-2763, 2532-3077, 2539-2750, 2552-2847, 2553-3114, |
| | 2554-2823, 2573-2824, 2606-2853, 2621-2851, 2628-2908, 2674-3257, 2690-2995, 2708-2884, |
| | 2719-3033, 2771-3060, 2771-3090, 2774-2839, 2780-3059, 2782-3032, 2782-3282, 2783-3392, |
| | 2784-3449, 2803-3390, 2808-3101, 2819-3466, 2821-3366, 2842-3314, 2843-3419, 2850-3442, |
| | 2852-3272, 2853-3142, 2856-3308, 2860-3383, 2861-3250, 2861-3418, 2861-3468, 2861-3479, |
| | 2864-3127, 2864-3449, 2875-2936, 2875-3099, 2878-3148, 2881-3158, 2881-3172, 2881-3187, |
| | 2883-3159, 2884-3176, 2885-3167, 2888-3138, 2888-3423, 2888-3439, 2888-3469, 2891-3097, |
| | 2902-3147, 2907-3448, 2910-3483, 2916-3493, 2932-3027, 2939-3232, 2951-3194, 2955-3419, |
| | 2958-3193, 2966-3446, 2967-3488, 2971-3052, 2974-3227, 2976-3272, 2977-3455, 2979-3248, |
| | 2979-3443, 2979-3452, 2980-3459, 2988-3461, 2989-3432, 2993-3270, |
| | 2993-3343, 2993-3458, 2996-3271, 3003-3365, 3003-3452, 3008-3460, 3009-3456, 3012-3240, |
| | 3012-3461, 3013-3387, 3014-3462, 3015-3460, 3016-3436, 3016-3459, 3017-3464, 3021-3464, |
| | 3022-3430, 3023-3298, 3024-3457, 3026-3459, 3028-3303, 3028-3456, 3030-3263, 3030-3307, |
| | 3030-3416, 3031-3479, 3034-3456, 3038-3456, 3040-3192, 3040-3457, 3046-3460, 3046-3461, |
| | 3046-3488, 3047-3458, 3047-3459, 3050-3462, 3052-3463, 3056-3461, 3057-3464, 3060-3456, |
| | 3062-3356, 3062-3546, 3065-3455, 3068-3371, 3068-3461, 3068-3480, 3069-3456, 3076-3455, |
| | 3078-3454, 3078-3461, 3086-3456, 3087-3331, 3087-3461, 3091-3476, 3092-3363, 3093-3370, |
| | 3100-3478, 3108-3370, 3108-3372, 3111-3464, 3113-3366, 3113-3458, 3117-3346, 3118-3456, |
| | 3119-3462, 3125-3461, 3126-3458, 3128-3475, 3136-3459, 3141-3453, 3142-3419, 3143-3378, |
| | 3146-3409, 3147-3403, 3161-3446, 3172-3461, 3178-3433, 3180-3553, 3181-3382, 3183-3461, |
| | 3187-3548, 3194-3456, 3196-3460, 3202-3461, 3205-3464, 3207-3455, 3212-3443, 3219-3455, |
| | 3225-3479, 3228-3456, 3253-3457, 3257-3462, 3258-3474, 3264-3461, 3264-3899, 3265-3479, |
| | 3267-3455, 3268-3456, 3280-3468, 3288-3459, 3292-3551, 3302-3459, 3304-3459, 3305-3470, |
| | 3305-3481, 3307-3373, 3308-3461, 3314-3418, 3315-3455, 3320-3471, 3368-3540, 3369-3456, |
| | 3388-3462 |
| 75/7508332CB1/ | 1-561, 1-2491, 161-717, 239-945, 318-977, 329-873, 555-1183, 608-1300, 615-1300, 727-1251, |
| 2491 | 794-1231, 823-1297, 923-1424, 988-1679, 1052-1561, 1110-1545, 1126-1807, 1144-1819, 1164-1775, |
| | 1165-1716, 1225-1736, 1225-1779, 1227-1859, 1262-1879, 1267-1750, 1302-1988, 1343-2012, |
| | 1855-2486 |
| 76/1288969CB1/ | 1-241, 1-275, 1-304, 1-457, 1-466, 1-479, 1-487, 1-509, 1-516, 1-531, 1-536, 1-541, |
| 1196 | 1-562, 1-564, 1-571, 1-576, 1-588, 1-589, 1-596, 1-605, 1-622, 1-630, 1-637, 1-641, |
| | 1-642, 1-643, 1-645, 1-647, 1-661, 1-665, 1-672, 1-677, 1-756, 1-1051, 2-596, 5-593, |
| | 5-646, 5-697, 5-791, 8-628, 14-666, 63-759, 72-641, 93-525, 106-796, 111-980, 151-770, |
| | 235-924, 244-603, 244-924, 247-826, 260-832, 260-979, 264-866, 300-871, 304-869, 312-841, |
| | 318-1015, 335-1065, 343-1032, 399-1092, 418-1096, 428-944, 444-1181, 462-1180, 466-1085, 470-765, |
| | 471-1163, 497-1177, 506-1119, 508-1142, 512-1188, 534-1180, 536-1196, 562-1196, 568-1196, 582-1196, |
| | 585-1139, 597-1193, 604-1189, 606-1175, 608-1165, 615-1192, 615-1193, 622-1193, 631-1196, 643-1196, |
| | 644-1196, 649-1196, 652-1193, 654-1196, 655-1176, 670-1165, 674-1190, 677-1193, 678-1192, 679-1196, |
| | 689-1193, 690-1195, 699-1190, 700-1196, 706-1168, 708-1196, 712-1196, 713-1193, 721-1193, 726-1066, |
| | 757-1175, 940-1145, 954-1193 |
| 77/72069135CB1/ | 1-263, 36-659, 157-436, 172-646, 287-642, 291-646, 313-646, 371-654, 395-508, 398-507, |
| 1730 | 398-952, 398-1064, 398-1173, 400-699, 401-1183, 414-699, 428-670, 453-646, 460-1119, 507-1331, |
| | 649-976, 662-972, 662-1259, 662-1365, 842-932, 842-1045, 843-1154, 844-1097, 847-1097, 847-1124, |
| | 847-1189, 852-1152, 854-1095, 861-1170, 865-1354, 876-1090, 876-1338, 876-1434, 876-1493, 876-1547, |
| | 877-1090, 877-1145, 877-1168, 877-1367, 878-1183, 878-1185, 879-1124, 879-1126, 881-1135, 881-1148, |
| | 881-1168, 881-1183, 881-1200, 881-1299, 882-1120, 882-1141, 882-1145, 882-1157, 882-1168, 882-1169, |
| | 882-1170, 882-1186, 883-1176, 886-1016, 888-1129, 888-1330, 899-1183, 902-1252, 904-1146, 915-1357, |
| | 945-1195, 977-1213, 996-1211, 996-1241, 996-1249, 996-1261, 1060-1362, 1062-1286, 1063-1349, |
| | 1064-1384, 1071-1300, 1078-1321, 1095-1717, 1107-1704, 1146-1423, 1191-1381, 1329-1524, |
| | 1338-1639, 1387-1730, 1393-1667, 1393-1720, 1426-1718, 1565-1730, 1582-1730 |
| 78/7506247CB1/ | 1-167, 1-3322, 36-410, 41-445, 422-630, 422-915, 422-938, 422-951, 458-1050, 706-981, 822-1316, |
| 3322 | 822-1392, 822-1400, 823-1119, 843-1316, 847-1112, 887-1163, 887-1492, 910-1183, 973-1252, 982-1299, |
| | 1010-1276, 1010-1511, 1029-1411, 1054-1248, 1065-1303, 1096-1426, 1107-1515, 1152-1591, |
| | 1152-1688, 1175-1426, 1175-1469, 1175-1624, 1175-1727, 1184-1453, 1192-1472, 1211-1431, |
| | 1228-1833, 1249-1520, 1249-1559, 1273-1495, 1312-1744, 1430-1685, 1479-1672, 1483-1757, |
| | 1486-1985, 1564-2022, 1594-2050, 1616-2335, 1617-2280, 1649-1896, 1649-2099, 1745-1981, |
| | 1824-2059, 1824-2071, 1846-2295, 1905-2229, 1933-2107, 1976-2464, 1998-2275, 2012-2361, |
| | 2014-2255, 2039-2316, 2070-2295, 2079-2944, 2102-2353, 2171-2766, 2192-2847, 2257-2543, |
| | 2272-2434, 2272-2873, 2327-2629, 2340-2546, 2340-2556, 2340-2916, 2346-2592, 2356-2674, |
| | 2384-2662, 2394-2639, 2406-2881, 2406-2927, 2408-2679, 2433-2673, 2446-2752, 2452-2703, |
| | 2464-2881, 2470-2763, 2471-2881, 2484-2881, 2487-2867, 2542-2919, 2555-2880, 2561-3209, |
| | 2569-2877, 2569-2880, 2570-2841, 2570-2852, 2575-2859, 2586-3026, 2590-2785, 2597-2894, |
| | 2601-2943, 2616-2880, 2622-2880, 2638-2917, 2641-3072, 2647-2903, 2647-2911, 2651-2875, |
| | 2678-3303, 2691-3296, 2763-3065, 2783-3043, |
| | 2785-3065, 2785-3303, 2785-3311, 2807-2936, 2815-3312, 2828-2936, 2856-3322, 2861-3322, |
| | 2881-3322, 2935-3322, 2938-3322, 3223-3311, 3235-3311, 3238-3311, 3238-3322 |
| 79/7506363CB1/ | 1-540, 3-496, 7-430, 10-254, 10-1529, 21-572, 41-568, 107-391, 333-593, 353-607, 376-644, |
| 1529 | 583-1157, 626-1157, 679-918, 679-928, 679-950, 729-1024, 822-1071, 860-1117, 872-1082, 879-1081, |
| | 880-1172, 889-1168, 934-1175, 934-1178, 935-1257, 954-1193, 957-1211, 961-1266, 962-1170, 1041-1516, |
| | 1041-1517, 1043-1517, 1044-1516, 1045-1514, 1046-1521, 1048-1518, 1049-1520, 1053-1518, |
| | 1057-1399, 1058-1516, 1060-1512, 1061-1517, 1062-1516, 1065-1520, 1066-1517, 1067-1517, |
| | 1067-1521, 1070-1518, 1070-1529, 1074-1517, 1080-1517, 1089-1234, 1096-1517, 1102-1517, |
| | 1103-1399, 1103-1517, 1106-1514, 1108-1378, 1113-1468, 1116-1517, 1116-1522, 1117-1517, |
| | 1132-1516, 1133-1517, 1136-1517, 1139-1517, 1143-1517, 1147-1517, 1151-1516, 1167-1521, |
| | 1168-1519, 1170-1517, 1170-1518, 1189-1475, 1194-1517, 1197-1516, 1221-1517, 1224-1478, |
| | 1225-1517, 1229-1522, 1238-1529, 1243-1520, 1252-1517, 1282-1484, 1295-1491, 1295-1517, |
| | 1312-1529, 1326-1508, 1326-1529, 1330-1468, 1343-1517, 1345-1516, 1348-1512, 1350-1529, |
| | 1355-1529, 1373-1529, 1385-1529, 1397-1529, 1419-1529, 1445-1516 |
| 80/7509068CB1/ | 1-323, 1-385, 1-519, 1-525, 1-528, 1-575, 1-620, 1-624, 1-677, 1-750, 1-779, 1-804, |
| 2526 | 1-839, 1-2504, 1-2514, 5-712, 12-337, 12-424, 14-645, 18-857, 29-575, 128-382, 164-716, |
| | 199-773, 238-802, 241-477, 307-901, 310-732, 310-876, 316-876, 392-678, 392-900, 392-901, |
| | 415-878, 431-773, 468-900, 509-901, 509-1021, 512-1082, 648-902, 668-1252, 733-1255, 790-982, |
| | 790-1252, 790-1325, 791-901, 873-1007, 993-1687, 1012-1701, 1086-1762, 1099-1470, 1110-1542, |
| | 1128-1689, 1128-1734, 1143-1650, 1223-1780, 1259-1728, 1263-1746, 1271-1699, 1296-1744, |
| | 1305-1934, 1307-2035, 1315-1982, 1341-1586, 1360-1886, 1375-2124, 1377-1937, 1443-1898, |
| | 1451-2010, 1461-1859, 1464-1859, 1496-1994, 1525-2088, 1547-1844, 1565-1740, 1594-2052, |
| | 1594-2101, 1600-1939, 1604-2015, 1646-1879, 1655-2088, 1887-2423, 1935-2521, 1943-2522, |
| | 1956-2522, 1969-2490, 1976-2519, 1977-2524, 1985-2510, 1986-2516, 1999-2493, 2007-2518, |
| | 2008-2521, 2025-2490, 2029-2411, 2031-2493, 2042-2496, 2043-2508, 2046-2506, 2047-2503, |
| | 2048-2496, 2048-2505, 2051-2520, 2059-2499, 2059-2501, 2066-2423, 2066-2472, 2066-2509, |
| | 2069-2510, 2075-2503, 2083-2503, 2087-2508, 2095-2508, 2097-2493, 2097-2503, 2098-2503, |
| | 2103-2508, 2108-2523, 2109-2504, 2115-2505, 2115-2506, 2116-2503, 2119-2503, 2125-2503, |
| | 2126-2494, 2126-2504, 2133-2502, 2133-2503, 2137-2506, 2144-2509, 2146-2504, 2147-2458, |
| | 2158-2503, 2159-2510, 2169-2503, 2178-2443, 2190-2503, 2196-2493, 2222-2504, 2224-2504, |
| | 2226-2503, 2227-2503, 2227-2510, 2229-2503, 2235-2504, 2237-2506, 2247-2504, 2247-2506, |
| | 2259-2524, 2262-2493, 2268-2498, 2269-2504, 2309-2507, 2309-2524, 2330-2526, 2331-2526, |
| | 2334-2462, 2334-2503, 2334-2524 |
| 81/7505897CB1/ | 1-138, 1-230, 1-271, 1-433, 1-454, 1-469, 1-599, 4-299, 5-262, 6-629, 11-149, 11-1225, |
| 1228 | 16-245, 19-109, 33-98, 41-531, 45-727, 48-545, 48-868, 48-891, 48-918, 48-922, 48-923, 48-947, |
| | 49-724, 50-629, 53-643, 71-343, 73-338, 80-344, 138-462, 141-947, 163-945, 167-945, 169-947, |
| | 178-947, 184-947, 194-947, 207-945, 234-947, 240-719, 251-947, 309-947, 316-601, 348-865, |
| | 358-947, 380-581, 399-928, 400-634, 400-920, 410-898, 417-901, 455-901, 487-786, 496-974, |
| | 512-920, 512-942, 519-790, 523-974, 524-974, 537-842, 546-984, 555-1197, 574-965, 574-974, |
| | 574-1016, 602-1045, 635-1114, 643-1201, 660-1083, 666-1202, 679-1200, 681-1201, 688-967, 690-1136, |
| | 691-1050, 720-1046, 724-1176, 728-1210, 733-1114, 735-1199, 739-1198, 743-1209, 751-1213, 752-1210, |
| | 753-1203, 753-1210, 754-1210, 757-1210, 762-1202, 763-1211, 763-1217, 764-1214, 765-1206, 765-1210, |
| | 768-1209, 787-1198, 801-1228, 809-1048, 812-1211, 827-1211, 847-1208, 865-1210, 886-1210, 896-1210, |
| | 957-1210, 974-1195, 974-1228, 981-1205, 1012-1228, 1064-1208 |
| 82/7505898CB1/ | 1-1772, 222-385, 222-432, 222-437, 222-476, 222-478, 222-494, 222-532, 222-623, 222-631, |
| 2034 | 222-665, 222-724, 222-819, 222-835, 222-858, 222-862, 222-863, 222-896, 222-1039, 223-414, |
| | 223-435, 223-600, 223-915, 224-474, 224-517, 225-932, 226-509, 230-659, 231-479, 231-497, |
| | 231-504, 233-481, 238-538, 238-669, 248-845, 258-530, 267-536, 282-532, 290-562, 313-495, |
| | 331-975, 334-526, 335-968, 342-589, 352-589, 361-663, 362-613, 372-589, 414-1038, 417-1050, |
| | 426-819, 447-545, 447-688, 457-1053, 471-927, 484-1053, 488-1053, 489-773, 496-1053, 503-1050, |
| | 509-780, 509-1053, 514-1053, 516-1021, 525-1053, 529-1018, 532-639, 539-1055, 543-1053, 544-1053, |
| | 549-823, 550-1053, 552-1053, 556-1053, 568-1053, 569-1053, 573-1050, 581-811, 586-1055, 588-845, |
| | 588-1010, 590-1049, 595-885, 597-819, 598-806, 602-1053, 605-864, 612-1053, 618-831, 619-956, |
| | 653-897, 658-1050, 667-1057, 676-808, 698-912, 698-977, 720-1067, 752-1052, 764-1067, 772-1027, |
| | 791-1077, 791-1086, 810-1020, 810-1029, 833-1067, 853-1044, 868-1053, 993-1050, 995-1448, 1008-1449, |
| | 1065-1217, 1065-1355, 1065-1358, 1065-1359, 1065-1361, 1065-1367, 1065-1444, 1065-1447, |
| | 1065-1449, 1070-1362, 1072-1450, |
| | 1080-1359, 1091-1434, 1097-1433, 1100-1214, 1105-1362, 1108-1445, 1120-1676, 1121-1361, |
| | 1121-1364, 1122-1361, 1126-1362, 1128-1362, 1151-1419, 1151-1434, 1152-1444, 1153-1733, |
| | 1161-1448, 1187-1362, 1212-1310, 1212-1749, 1220-1359, 1235-1490, 1250-1710, 1255-1364, |
| | 1257-1449, 1258-1353, 1262-1473, 1262-1505, 1277-1510, 1282-1449, 1307-1449, 1320-1450, |
| | 1339-1597, 1340-1588, 1362-1590, 1375-1609, 1395-1662, 1412-1711, 1412-1773, 1412-2034, |
| | 1446-1711, 1449-1674, 1469-1725, 1479-1712, 1485-1712, 1512-1753, 1519-1768, 1549-1773, |
| | 1552-1732, 1552-1773 |
| 83/7505907CB1/ | 1-1854, 36-271, 150-642, 191-652, 204-458, 204-1854, 242-642, 277-525, 305-642, 306-566, |
| 1854 | 307-590, 381-687, 499-1098, 551-818, 567-921, 574-896, 585-874, 612-932, 649-854, 659-944, |
| | 661-906, 667-958, 676-900, 692-972, 692-993, 772-983, 772-993, 995-1588, 1005-1538, 1010-1532, |
| | 1020-1313, 1024-1257, 1030-1154, 1030-1670, 1031-1701, 1037-1696, 1048-1513, 1053-1658, |
| | 1053-1696, 1064-1343, 1072-1323, 1099-1730, 1127-1555, 1159-1525, 1159-1640, 1176-1436, |
| | 1188-1377, 1220-1502, 1234-1529, 1253-1592, 1255-1549, 1281-1514, 1284-1550, 1294-1712, |
| | 1301-1554, 1314-1706, 1336-1705, 1365-1554, 1370-1712, 1370-1717, 1375-1699, 1384-1728, |
| | 1407-1712, 1439-1713, 1454-1666, 1486-1804, 1546-1716, 1548-1712, 1605-1800 |
| 84/7505925CB1/ | 1-238, 10-214, 10-239, 10-252, 10-262, 10-263, 10-382, 10-474, 10-526, 10-1341, 11-128, 11-300, |
| 1346 | 11-389, 11-496, 12-294, 14-546, 15-140, 15-274, 15-282, 16-263, 16-268, 16-442, 18-152, |
| | 18-220, 18-258, 18-261, 18-266, 18-268, 18-276, 18-316, 18-398, 18-402, 18-414, 18-438, 18-516, |
| | 18-546, 19-231, 19-250, 19-545, 21-223, 21-276, 21-403, 22-296, 23-316, 24-289, 26-119, 26-237, |
| | 26-274, 26-282, 26-287, 31-283, 34-293, 37-191, 43-339, 48-352, 48-531, 49-268, 50-304, 53-546, |
| | 54-546, 58-259, 62-372, 97-281, 107-546, 115-373, 154-391, 159-378, 190-443, 234-880, |
| | 248-537, 256-766, 259-533, 315-543, 322-446, 346-546, 360-540, 536-1265, 545-708, 545-760, |
| | 545-777, 545-792, 545-795, 545-801, 545-820, 545-852, 545-870, 545-877, 545-1009, 545-1030, |
| | 545-1056, 545-1071, 545-1178, 560-679, 560-1175, 563-1187, 564-832, 566-812, 570-821, 572-803, |
| | 572-846, 574-1072, 575-816, 575-1028, 577-817, 589-1149, 594-880, 612-897, 619-880, 621-883, |
| | 621-901, 637-919, 647-912, 662-920, 663-1254, 664-934, 664-1253, 678-1317, 682-886, 682-1197, |
| | 686-964, 697-937, 701-1345, 706-968, 718-942, 723-1154, 727-1325, |
| | 736-1346, 743-1255, 756-1293, 771-1318, 774-1324, 775-1296, 777-1024, 777-1027, 780-1346, 788-1339, |
| | 798-1119, 800-1323, 810-1065, 810-1315, 823-1266, 823-1320, 829-1278, 852-1045, 853-1346, 871-1332, |
| | 874-1324, 876-1119, 876-1328, 879-1295, 879-1328, 880-1332, 883-1344, 886-1328, 900-1324, 907-1324, |
| | 911-1346, 920-1346, 922-1346, 923-1179, 928-1324, 929-1330, 930-1325, 935-1238, 949-1224, 951-1212, |
| | 951-1323, 954-1207, 954-1224, 957-1331, 959-1332, 964-1260, 1024-1345, 1026-1346, 1032-1332, |
| | 1058-1332, 1062-1331, 1062-1346, 1064-1332, 1067-1324, 1078-1313, 1079-1271, 1092-1346, |
| | 1093-1344, 1093-1346, 1101-1332, 1117-1346, 1121-1346, 1133-1331, 1136-1331, 1145-1346, |
| | 1172-1346, 1202-1332, 1210-1334, 1236-1340, 1236-1346 |

TABLE 5

| Polynucleotide SEQ | Incyte | Representative |
|---|---|---|
| ID NO: | Project ID: | Library |
| 43 | 70612021CB1 | BRAXNOT03 |
| 44 | 71847235CB1 | UCMCL5T01 |
| 45 | 7505230CB1 | LIVRNON08 |
| 46 | 7505235CB1 | FTUBTUR01 |
| 47 | 7505793CB1 | TLYJNOT01 |
| 48 | 7505861CB1 | THP1AZS08 |
| 49 | 7505864CB1 | BRAINOT09 |
| 50 | 7506427CB1 | TLYJNOT01 |
| 51 | 7506429CB1 | TLYJNOT01 |
| 52 | 7505799CB1 | PROSTUT10 |
| 53 | 7505843CB1 | SMCBUNT01 |
| 55 | 7504923CB1 | LUNGNOT14 |
| 56 | 7506151CB1 | SININOT05 |
| 57 | 7506450CB1 | ADMEDNV17 |
| 58 | 71380031CB1 | COLNFET02 |
| 59 | 7506054CB1 | MUSLTMT01 |
| 60 | 7506139CB1 | FIBPFEN06 |
| 61 | 7506426CB1 | TLYJNOT01 |
| 62 | 7506741CB1 | LIVRTMR01 |
| 63 | 7506743CB1 | LIVRTMR01 |
| 64 | 7506746CB1 | LIVRTUT04 |
| 65 | 7506748CB1 | PROSTUT09 |
| 66 | 1419966CB1 | KIDNNOT09 |
| 67 | 7506451CB1 | LIVRNOT01 |
| 68 | 90015249CB1 | LUNGTUT09 |
| 69 | 7487231CB1 | NOSEDIC02 |
| 70 | 7506260CB1 | LYMBTXT01 |
| 71 | 7506270CB1 | BRAXTDR15 |
| 72 | 7506306CB1 | COLNFET02 |
| 73 | 7506428CB1 | TLYJNOT01 |
| 74 | 7678032CB1 | COLNFET02 |
| 75 | 7508332CB1 | PITUNOT01 |
| 76 | 1288969CB1 | BRAINOT11 |
| 77 | 72069135CB1 | KIDNNOT09 |
| 78 | 7506247CB1 | BRAITUT12 |
| 79 | 7506363CB1 | BRAITUT13 |
| 80 | 7509068CB1 | LIVRTUT04 |
| 81 | 7505897CB1 | SININOT04 |
| 82 | 7505898CB1 | MIXDTME01 |
| 83 | 7505907CB1 | COLNNOT16 |
| 84 | 7505925CB1 | COLNCRT01 |

TABLE 6

| Library | Vector | Library Description |
|---|---|---|
| ADMEDNV17 | PCR2-TOPOTA | Library was constructed using pooled cDNA from different donors. cDNA |
| | | was generated using mRNA isolated from pooled skeletal muscle tissue |
| | | removed from ten 21 to 57-year-old Caucasian male and female donors who |
| | | died from sudden death; from pooled thymus tissue removed from nine 18 |
| | | to 32-year-old Caucasian male and female donors who died from sudden |
| | | death; from pooled liver tissue removed from 32 Caucasian male and |
| | | female fetuses who died at 18-24 weeks gestation due to spontaneous |
| | | abortion; from kidney tissue removed from 59 Caucasian male and female |
| | | fetuses who died at 20-33 weeks gestation due to spontaneous |
| | | abortion; and from brain tissue removed from a Caucasian male fetus who |
| | | died at 23 weeks gestation due to fetal demise. |
| BRAINOT09 | pINCY | Library was constructed using RNA isolated from brain tissue removed |
| | | from a Caucasian male fetus, who died at 23 weeks' gestation. |
| BRAINOT11 | pINCY | Library was constructed using RNA isolated from brain tissue removed |
| | | from the right temporal lobe of a 5-year-old Caucasian male during a |
| | | hemispherectomy. Pathology indicated extensive polymicrogyria and mild |
| | | to moderate gliosis (predominantly subpial and subcortical), consistent |
| | | with chronic seizure disorder. Family history included a cervical neoplasm. |
| BRAITUT12 | pINCY | Library was constructed using RNA isolated from brain tumor tissue |
| | | removed from the left frontal lobe of a 40-year-old Caucasian female |
| | | during excision of a cerebral meningeal lesion. Pathology indicated |
| | | grade 4 gemistocytic astrocytoma. |
| BRAITUT13 | pINCY | Library was constructed using RNA isolated from brain tumor tissue |
| | | removed from the left frontal lobe of a 68-year-old Caucasian male |
| | | during excision of a cerebral meningeal lesion. Pathology indicated |
| | | a meningioma in the left frontal lobe. |
| BRAXNOT03 | pINCY | Library was constructed using RNA isolated from sensory-motor cortex |
| | | tissue obtained from the brain of a 35-year-old Caucasian male who died |
| | | from cardiac failure. Pathology indicated moderate leptomeningeal |
| | | fibrosis and multiple microinfarctions of the cerebral neocortex. |
| | | Patient history included dilated cardiomyopathy, congestive heart |
| | | failure, cardiomegaly and an enlarged spleen and liver. |
| BRAXTDR15 | PCDNA2.1 | This random primed library was constructed using RNA isolated from |
| | | superior parietal neocortex tissue removed from a 55-year-old Caucasian |
| | | female who died from cholangiocarcinoma. Pathology indicated mild |
| | | meningeal fibrosis predominately over the convexities, scattered axonal |
| | | spheroids in the white matter of the cingulate cortex and the thalamus, |
| | | and a few scattered neurofibrillary tangles in the entorhinal cortex |
| | | and the periaqueductal gray region. Pathology for the associated tumor |
| | | tissue indicated well-differentiated cholangiocarcinoma of the liver |
| | | with residual or relapsed tumor. Patient history included |
| | | cholangiocarcinoma, post-operative Budd-Chiari syndrome, biliary |
| | | ascites, hydrothorax, dehydration, malnutrition, oliguria and acute |
| | | renal failure. Previous surgeries included cholecystectomy and |
| | | resection of 85% of the liver. |
| COLNCRT01 | PSPORT1 | Library was constructed using RNA isolated from a diseased section of |
| | | the ascending colon of a 40-year-old Caucasian male during a partial |
| | | colectomy. Pathology indicated Crohn's disease involving the proximal |
| | | colon and including the cecum. The ascending and transverse colon |
| | | displayed linear ulcerations and skip lesions. There was transmural |
| | | inflammation but no fistulas. |
| COLNFET02 | pINCY | Library was constructed using RNA isolated from the colon tissue of a |
| | | Caucasian female fetus, who died at 20 weeks' gestation. |
| COLNNOT16 | pINCY | Library was constructed using RNA isolated from sigmoid colon tissue |
| | | removed from a 62-year-old Caucasian male during a sigmoidectomy and |
| | | permanent colostomy. |
| FIBPFEN06 | pINCY | The normalized prostate stromal fibroblast tissue libraries were |
| | | constructed from 1.56 million independent clones from a prostate |
| | | fibroblast library. Starting RNA was made from fibroblasts of prostate |
| | | stroma removed from a male fetus, who died after 26 weeks' gestation. |
| | | The libraries were normalized in two rounds using conditions adapted |
| | | from Soares et al., PNAS (1994) 91: 9228 and Bonaldo et al., Genome |
| | | Research (1996) 6: 791, except that a significantly longer |
| | | (48-hours/round)reannealing hybridization was used. The library was |
| | | then linearized and recircularized to select for insert containing |
| | | clones as follows: plasmid DNA was prepped from approximately 1 million |
| | | clones from the normalized prostate stromal fibroblast tissue libraries |
| | | following soft agar transformation. |
| FTUBTUR01 | PCDNA2.1 | This random primed library was constructed using RNA isolated from |
| | | fallopian tube tumor tissue removed from an 85-year-old Caucasian |
| | | female during bilateral salpingo-oophorectomy and hysterectomy. |
| | | Pathology indicated poorly differentiated mixed endometrioid (80%) and |
| | | serous (20%) adenocarcinoma, which was confined to the mucosa without |
| | | mural involvement. Endometrioid carcinoma in situ was also present. |
| | | Pathology for the associated uterus tumor indicated focal endometrioid |
| | | adenocarcinoma in situ and moderately differentiated invasive |
| | | adenocarcinoma arising in an endometrial polyp. Metastatic endometrioid |
| | | and serous adenocarcinoma was present at the cul-de-sac tumor. Patient |
| | | history included medullary carcinoma of the thyroid and myocardial |
| | | infarction. |
| KIDNNOT09 | pINCY | Library was constructed using RNA isolated from the kidney tissue of a |
| | | Caucasian male fetus, who died at 23 weeks' gestation. |
| LIVRNON08 | pINCY | This normalized library was constructed from 5.7 million independent |
| | | clones from a pooled liver tissue library. Starting RNA was made from |
| | | pooled liver tissue removed from a 4-year-old Hispanic male who died |
| | | from anoxia and a 16 week female fetus who died after 16-weeks |
| | | gestation from anencephaly. Serologies were positive for |
| | | cytolomegalovirus in the 4-year-old. Patient history included asthma in |
| | | the 4-year-old. Family history included taking daily prenatal vitamins |
| | | and mitral valve prolapse in the mother of the fetus. The library was |
| | | normalized in 2 rounds using conditions adapted from Soares et al., |
| | | PNAS (1994) 91: 9228 and Bonaldo et al., Genome Research 6 (1996): 791, |
| | | except that a significantly longer (48 hours/round) reannealing |
| | | hybridization was used. |
| LIVRNOT01 | PBLUESCRIPT | Library was constructed at Stratagene, using RNA isolated from the |
| | | liver tissue of a 49-year-old male. |
| LIVRTMR01 | PCDNA2.1 | This random primed library was constructed using RNA isolated from |
| | | liver tissue removed from a 62-year-old Caucasian female during partial |
| | | hepatectomy and exploratory laparotomy. Pathology for the matched tumor |
| | | tissue indicated metastatic intermediate grade neuroendocrine carcinoma, |
| | | consistent with islet cell tumor, forming nodules ranging in size, in |
| | | the lateral and medial left liver lobe. The pancreas showed fibrosis, |
| | | chronic inflammation and fat necrosis consistent with pseudocyst. The |
| | | gallbladder showed mild chronic cholecystitis. Patient history included |
| | | malignant neoplasm of the pancreas tail, pulmonary embolism, |
| | | hyperlipidemia, thrombophlebitis, joint pain in multiple joints, type |
| | | II diabetes, benign hypertension, cerebrovascular disease, and normal |
| | | delivery. Previous surgeries included distal pancreatectomy, total |
| | | splenectomy, and partial hepatectomy. Family history included pancreas |
| | | cancer with secondary liver cancer, benign hypertension, and hyperlipidemia. |
| LIVRTUT04 | pINCY | Library was constructed using RNA isolated from liver tumor tissue |
| | | removed from a 50-year-old Caucasian male during a partial hepatectomy. |
| | | Pathology indicated a grade 3-4 hepatoma, forming a mass. Patient |
| | | history included benign hypertension and hepatitis. Hepatitis B core |
| | | antigen and hepatitis B surface antigen was present in the patient. |
| LUNGNOT14 | pINCY | Library was constructed using RNA isolated from lung tissue removed |
| | | from the left lower lobe of a 47-year-old Caucasian male during a |
| | | segmental lung resection. Pathology for the associated tumor tissue |
| | | indicated a grade 4 adenocarcinoma, and the parenchyma showed calcified |
| | | granuloma. Patient history included benign hypertension and chronic |
| | | obstructive pulmonary disease. Family history included type II diabetes |
| | | and acute myocardial infarction. |
| LUNGTUT09 | pINCY | Library was constructed using RNA isolated from lung tumor tissue |
| | | removed from a 68-year-old Caucasian male during segmental lung |
| | | resection. Pathology indicated invasive grade 3 squamous cell carcinoma |
| | | and a metastatic tumor. Patient history included type II diabetes, |
| | | thyroid disorder, depressive disorder, hyperlipidemia, esophageal ulcer, |
| | | and tobacco use. |
| LYMBTXT01 | pINCY | The library was constructed using RNA isolated from a treated K-562 |
| | | cell line, derived from chronic myelogenous leukemia precursor cells |
| | | removed from a 53-year-old female. The cells were treated with 9 cis |
| | | retinoic acid (RA), 1 micromolar, for 13 days. |
| MIXDTME01 | PBK-CMV | This 5′ biased random primed library was constructed using pooled |
| | | cDNA from five donors. cDNA was generated using mRNA isolated from small |
| | | intestine tissue removed from a Caucasian male fetus (donor A), who died |
| | | at 23 weeks' gestation from premature birth; from colon epithelium |
| | | tissue removed from a 13-year-old Caucasian female (donor B) who died |
| | | from a motor vehicle accident; from diseased gallbladder tissue removed |
| | | from a 58-year-old Caucasian female (donor C) during cholecystectomy |
| | | and partial parathyroidectomy; from stomach tissue removed from a |
| | | 68-year-old Caucasian female (donor D) during a partial gastrectomy; |
| | | and from breast skin removed from a 71-year-old Caucasian female (donor |
| | | E) during a unilateral extended simple mastectomy. For donor C, |
| | | pathology indicated chronic cholecystitis and cholelithiasis. The |
| | | patient presented with abdominal pain and benign parathyroid neoplasm. |
| | | Patient medications included Capoten, Catapres, Norvasc, Synthroid, |
| | | and Xanax. For donor D, pathology indicated the uninvolved stomach |
| | | tissue showed mild chronic gastritis. Patient medications included |
| | | Prilosec, zidoxin, Metamucil, calcium, and vitamins. |
| | | Donor E presented with malignant breast neoplasm and induration. |
| | | Patient medications included insulin, aspirin, and beta carotene. |
| MUSLTMT01 | pINCY | Library was constructed using RNA isolated from glossal muscle tissue |
| | | removed from a 41-year-old Caucasian female during partial glossectomy. |
| | | Pathology indicated the excision margins were negative for tumor. |
| | | Pathology for the matched tumor tissue indicated invasive grade 3, |
| | | squamous cell carcinoma forming an ulcerated mass of the tongue. The |
| | | patient presented with a complicated open wound of the tongue. Patient |
| | | history included obesity, an unspecified nasal and sinus disease, and |
| | | normal delivery. Patient medications included Premarin, Hydrocodone, |
| | | vitamins, and Equate nasal spray. Family history included benign |
| | | hypertension, atherosclerotic coronary artery disease, upper lobe lung |
| | | cancer, type II diabetes, hyperlipidemia, and cirrhosis of the liver in |
| | | the father. |
| NOSEDIC02 | PSPORT1 | This large size fractionated library was constructed using RNA isolated |
| | | from nasal polyp tissue. |
| PITUNOT01 | PBLUESCRIPT | Library was constructed using RNA obtained from Clontech (CLON 6584-2, |
| | | lot 35278). The RNA was isolated from the pituitary glands removed from |
| | | a pool of 18 male and female Caucasian donors, 16 to 70 years old, who |
| | | died from trauma. |
| PROSTUT09 | pINCY | Library was constructed using RNA isolated from prostate tumor tissue |
| | | removed from a 66-year-old Caucasian male during a radical |
| | | prostatectomy, radical cystectomy, and urinary diversion. Pathology |
| | | indicated grade 3 transitional cell carcinoma. The patient presented |
| | | with prostatic inflammatory disease. Patient history included lung |
| | | neoplasm, and benign hypertension. Family history included a malignant |
| | | breast neoplasm, tuberculosis, cerebrovascular disease, atherosclerotic |
| | | coronary artery disease and lung cancer. |
| PROSTUT10 | pINCY | Library was constructed using RNA isolated from prostatic tumor tissue |
| | | removed from a 66-year-old Caucasian male during radical prostatectomy |
| | | and regional lymph node excision. Pathology indicated an adenocarcinoma |
| | | (Gleason grade 2 + 3). Adenofibromatous hyperplasia was also present. |
| | | The patient presented with elevated prostate specific antigen (PSA). |
| | | Family history included prostate cancer and secondary bone cancer. |
| SININOT04 | pINCY | Library was constructed using RNA isolated from diseased ileum tissue |
| | | obtained from a 26-year-old Caucasian male during a partial colectomy, |
| | | permanent colostomy, and an incidental appendectomy. Pathology indicated |
| | | moderately to severely active Crohn's disease. Family history included |
| | | enteritis of the small intestine |
| SININOT05 | pINCY | Library was constructed using RNA isolated from ileum tissue obtained |
| | | from a 30-year-old Caucasian female during partial colectomy, open liver |
| | | biopsy, incidental appendectomy, and permanent colostomy. Patient |
| | | history included endometriosis. Family history included hyperlipidemia, |
| | | anxiety, and upper lobe lung cancer, stomach cancer, liver cancer, and cirrhosis. |
| SMCBUNT01 | pINCY | Library was constructed using RNA isolated from untreated bronchial |
| | | smooth muscle cell tissue removed from a 21-year-old Caucasian male. |
| THP1AZS08 | PSPORT1 | This subtracted THP-1 promonocyte cell line library was constructed |
| | | using 5.76 × 1e6 clones from a 5-aza-2′-deoxycytidine |
| | | (AZ) treated THP-1 cell library. Starting RNA was made from THP-1 |
| | | promonocyte cells treated for three days with 0.8 micromolar AZ. The |
| | | donor had acute monocytic leukemia The hybridization probe for |
| | | subtraction was derived from a similarly constructed library, made from |
| | | 1 microgram of polyA RNA isolated from untreated THP-1 cells. 5.76 |
| | | million clones from the AZ-treated THP-1 cell library were then |
| | | subjected to two rounds of subtractive hybridization with 5 million |
| | | clones from the untreated THP-1 cell library. Subtractive hybridization |
| | | conditions were based on the methodologies of Swaroop et al., NAR |
| | | (1991) 19: 1954, and Bonaldo et al., Genome Research (1996) 6: 791. |
| TLYJNOT01 | pINCY | Library was constructed using RNA isolated from an untreated Jurkat |
| | | cell line derived from the T cells of a male. Patient history included |
| | | acute T-cell leukemia. |
| UCMCL5T01 | PBLUESCRIPT | Library was constructed using RNA isolated from mononuclear cells |
| | | obtained from the umbilical cord blood of 12 individuals. The cells |
| | | were cultured for 12 days with IL-5 before RNA was obtained from the |
| | | pooled lysates. |

TABLE 7

| Program | Description | Reference | Parameter Threshold |
|---|---|---|---|
| ABI FACTURA | A program that removes vector | Applied Biosystems, | |
| | sequences and masks ambiguous | Foster City, CA. | |
| | bases in nucleic acid sequences. | | |
| ABI/PARACEL FDF | A Fast Data Finder useful in | Applied Biosystems, | Mismatch <50% |
| | comparing and annotating amino | Foster City, CA; | |
| | acid or nucleic acid sequences. | Paracel Inc., | |
| | | Pasadena, CA. | |
| ABI AutoAssembler | A program that assembles nucleic | Applied Biosystems, | |
| | acid sequences. | Foster City, CA. | |
| BLAST | A Basic Local Alignment Search | Altschul, S. F. et al. | ESTs: Probability value = |
| | Tool useful in sequence similarity | (1990) J. Mol. Biol. | 1.0E−8 or less; Full Length |
| | search for amino acid and nucleic | 215: 403-410; Altschul, | sequences: |
| | acid sequences. BLAST includes five | S. F. et al. (1997) | Probability value = 1.0E−10 |
| | functions: blastp, blastn, blastx, | Nucleic Acids Res. 25: | or less |
| | tblastn, and tblastx. | 3389-3402. | |
| FASTA | A Pearson and Lipman algorithm that | Pearson, W. R. and D. J. | ESTs: fasta E value = 1.06E−6; |
| | searches for similarity between a | Lipman (1988) Proc. | Assembled ESTs: fasta Identity = |
| | query sequence and a group of | Natl. Acad Sci. USA 85: | 95% or greater and Match |
| | sequences of the same type. FASTA | 2444-2448; Pearson, | length = 200 bases or greater; |
| | comprises as least five functions: | W. R. (1990) Methods Enzymol. | fastx E value = 1.0E−8 or less; |
| | fasta, tfasta, fastx, tfastx, and | 183: 63-98; and Smith, | Full Length sequences: fastx |
| | ssearch. | T. F. and M. S. Waterman (1981) | score = 100 or greater |
| | | Adv. Appl. Math. 2: 482-489. | |
| BLIMPS | A BLocks IMProved Searcher that | Henikoff, S. and J. G. Henikoff | Probability value = |
| | matches a sequence against those | (1991) Nucleic Acids Res. 19: | 1.0E−3 or less |
| | in BLOCKS, PRINTS, DOMO, PRODOM, | 6565-6572; Henikoff, J. G. | |
| | and PFAM databases to search | and S. Henikoff (1996) Methods | |
| | for gene families, sequence | Enzymol. 266: 88-105; and | |
| | homology, and structural | Attwood, T. K. et al. (1997) J. | |
| | fingerprint regions. | Chem. Inf. Comput. Sci. 37: | |
| | | 417-424. | |
| HMMER | An algorithm for searching a | Krogh, A. et al. (1994) J. Mol. | PFAM, INCY, SMART or |
| | query sequence against | Biol. 235: 1501-1531; | TIGRFAM hits: Probability |
| | hidden Markov model (HMM)-based | Sonnhammer, E. L. L. et al. | value = 1.0E−3 or less; |
| | databases of protein family | (1988) Nucleic Acids Res. 26: | Signal peptide hits: Score = |
| | consensus sequences, such as | 320-322; Durbin, R. et al. | 0 or greater |
| | PFAM, INCY, SMART and TIGRFAM. | (1998) Our World View, in | |
| | | a Nutshell, Cambridge Univ. | |
| | | Press, pp. 1-350. | |
| ProfileScan | An algorithm that searches for | Gribskov, M. et al. (1988) | Normalized quality score ≧ GCG |
| | structural and sequence motifs | CABIOS 4: 61-66; | specified “HIGH” value for |
| | in protein sequences that match | Gribskov, M. et al. (1989) | that particular Prosite motif. |
| | sequence patterns defined in | Methods Enzymol. 183: | Generally, score = 1.4-2.1. |
| | Prosite. | 146-159; Bairoch, A. et al. | |
| | | (1997) Nucleic Acids Res. | |
| | | 25: 217-221. | |
| Phred | A base-calling algorithm that | Ewing, B. et al. (1998) | |
| | examines automated sequencer | Genome Res. 8: 175-185; | |
| | traces with high sensitivity | Ewing, B. and P. Green (1998) | |
| | and probability. | Genome Res. 8: 186-194. | |
| Phrap | A Phils Revised Assembly Program | Smith, T. F. and M. S. | Score = 120 or greater; Match |
| | including SWAT and CrossMatch, | Waterman (1981) Adv. Appl. | length = 56 or greater |
| | programs based on efficient | Math. 2: 482-489; Smith, | |
| | implementation of the Smith- | T. F. and M. S. Waterman | |
| | Waterman algorithm, useful | (1981) J. Mol. Biol. 147: | |
| | in searching sequence homology | 195-197; and Green, P., | |
| | and assembling DNA sequences. | University of | |
| | | Washington, Seattle, WA. | |
| Consed | A graphical tool for viewing | Gordon, D. et al. (1998) | |
| | and editing Phrap assemblies. | Genome Res. 8: 195-202. | |
| SPScan | A weight matrix analysis program | Nielson, H. et al. (1997) | Score = 3.5 or greater |
| | that scans protein sequences for | Protein Engineering | |
| | the presence of secretory signal | 10: 1-6; Claverie, J. M. | |
| | peptides. | and S. Audic (1997) | |
| | | CABIOS 12: 431-439. | |
| TMAP | A program that uses weight | Persson, B. and P. Argos | |
| | matrices to delineate | (1994) J. Mol. Biol. | |
| | transmembrane segments on | 237: 182-192; Persson, | |
| | protein sequences and | B. and P. Argos. (1996) | |
| | determine orientation. | Protein Sci. 5: 363-371. | |
| TMHMMER | A program that uses a hidden | Sonnhammer, E. L. et al. | |
| | Markov model (HMM) to delineate | (1998) Proc. Sixth | |
| | transmembrane segments on protein | Intl. Conf. On Intelligent | |
| | sequences and determine orientation. | Systems for Mol. Biol., | |
| | | Glasgow et al., eds., The | |
| | | Am. Assoc. for Artificial | |
| | | Intelligence (AAAI) Press, | |
| | | Menlo Park, CA, and MIT | |
| | | Press, Cambridge, | |
| | | MA, pp. 175-182. | |
| Motifs | A program that searches amino acid | Bairoch, A. et al. (1997) | |
| | sequences for patterns that matched | Nucleic Acids Res. | |
| | those defined in Prosite. | 25: 217-221; Wisconsin | |
| | | Package Program | |
| | | Manual, version 9, page | |
| | | M51-59, Genetics | |
| | | Computer Group, Madison, WI. | |

TABLE 8

| | | | | | | | | | | | African | Asian | |
|---|---|---|---|---|---|---|---|---|---|---|---|---|---|
| SEQ | | | | | | EST | | | | Caucasian | Allele 1 | Allele 1 | Hispanic |
| ID | | | | EST | CB1 | Al- | Allele | Allele | Amino | Allele 1 | fre- | fre- | Allele 1 |
| NO: | PID | EST ID | SNP ID | SNP | SNP | lele | 1 | 2 | Acid | frequency | quency | quency | frequency |
| 66 | 1419966 | 1419966H1 | SNP00068260 | 110 | 110 | C | C | T | L23 | n/d | n/d | n/d | n/d |
| 66 | 1419966 | 1579008H1 | SNP00033325 | 157 | 1138 | C | C | T | F365 | n/a | n/a | n/a | n/a |
| 66 | 1419966 | 1689422H1 | SNP00033326 | 19 | 1379 | C | C | T | R446 | n/d | n/a | n/d | n/d |
| 66 | 1419966 | 2744383H1 | SNP00033324 | 221 | 779 | C | C | T | H246 | n/d | n/d | n/d | n/d |
| 67 | 7506451 | 1630029H1 | SNP00003610 | 131 | 841 | G | C | G | L269 | 0.13 | n/a | n/a | n/a |
| 67 | 7506451 | 2864905H1 | SNP00023566 | 127 | 969 | C | C | T | noncoding | n/a | n/a | n/a | n/a |
| 67 | 7506451 | 6804092J1 | SNP00051018 | 108 | 844 | C | C | T | Y270 | n/a | n/a | n/a | n/a |
| 69 | 7487231 | 2442777H1 | SNP00018702 | 67 | 2711 | C | C | T | noncoding | 0.88 | 0.95 | 0.98 | 0.91 |
| 69 | 7487231 | 7266545H2 | SNP00131703 | 349 | 876 | T | T | C | L148 | n/a | n/a | n/a | n/a |
| 69 | 7487231 | 7266545H2 | SNP00131704 | 360 | 887 | T | T | C | N151 | n/a | n/a | n/a | n/a |
| 70 | 7506260 | 1236536H1 | SNP00031875 | 99 | 454 | C | C | T | T137 | n/a | n/a | n/a | n/a |
| 70 | 7506260 | 1237215H1 | SNP00006989 | 95 | 185 | G | G | A | M47 | 0.60 | n/a | 0.98 | 0.80 |
| 70 | 7506260 | 1252384H1 | SNP00006989 | 127 | 177 | G | G | A | D45 | 0.60 | n/a | 0.98 | 0.80 |
| 70 | 7506260 | 1252384H1 | SNP00045634 | 33 | 81 | C | C | T | R13 | n/a | n/a | n/a | n/a |
| 70 | 7506260 | 1817856H1 | SNP00012493 | 246 | 985 | C | T | C | A314 | n/a | n/a | n/a | n/a |
| 72 | 7506306 | 1627842H1 | SNP00113452 | 178 | 2013 | G | G | T | noncoding | n/d | n/a | n/a | n/a |
| 72 | 7506306 | 2062244H1 | SNP00146087 | 60 | 1307 | C | C | T | 1372 | n/a | n/a | n/a | n/a |
| 72 | 7506306 | 3219268H1 | SNP00113453 | 241 | 1669 | G | C | G | noncoding | n/a | n/a | n/a | n/a |
| 73 | 7506428 | 1631117H1 | SNP00040510 | 46 | 806 | C | C | T | 1231 | n/d | n/a | n/a | n/a |
| 73 | 7506428 | 1886692H1 | SNP00112245 | 208 | 352 | G | A | G | R80 | n/a | n/a | n/a | n/a |
| 74 | 7678032 | 1400954H1 | SNP00068536 | 79 | 812 | A | A | G | Q268 | 0.94 | 0.95 | 0.90 | 0.80 |
| 74 | 7678032 | 1555819H1 | SNP00039328 | 35 | 35 | C | C | T | G9 | n/a | n/a | n/a | n/a |
| 74 | 7678032 | 2098357H1 | SNP00003619 | 125 | 1385 | C | C | T | S459 | 0.55 | 0.75 | 0.44 | 0.66 |
| 74 | 7678032 | 2417427H1 | SNP00099273 | 108 | 2292 | G | G | C | noncoding | n/d | n/d | n/d | n/d |
| 74 | 7678032 | 3200727H1 | SNP00154047 | 32 | 2670 | T | C | T | noncoding | n/a | n/a | n/a | n/a |
| 74 | 7678032 | 3676141H1 | SNP00099272 | 20 | 1757 | C | C | T | noncoding | old | n/a | n/a | n/a |
| 74 | 7678032 | 3733796H1 | SNP00024636 | 146 | 3332 | C | C | T | noncoding | n/d | n/a | n/a | n/a |
| 74 | 7678032 | 6083427H1 | SNP00155088 | 14 | 1739 | G | G | C | noncoding | n/a | n/a | n/a | n/a |
| 74 | 7678032 | 6421967H1 | SNP00039327 | 11 | 22 | G | G | C | S5 | n/d | n/a | n/a | n/a |
| 74 | 7678032 | 7013228H1 | SNP00012521 | 370 | 2850 | C | T | C | noncoding | 0.28 | 0.38 | 0.20 | 0.39 |
| 74 | 7678032 | 7044055H1 | SNP00118575 | 80 | 1349 | T | T | C | N447 | n/a | n/a | n/a | n/a |
| 74 | 7678032 | 7044055H1 | SNP00118576 | 179 | 1250 | A | G | A | K414 | n/d | n/a | n/a | n/a |
| 74 | 7678032 | 7047391H1 | SNP00099270 | 398 | 1030 | G | A | G | R341 | n/d | n/a | n/a | n/a |
| 74 | 7678032 | 7221767H1 | SNP00095551 | 358 | 386 | C | C | A | 5126 | 0.92 | n/a | n/a | n/a |
| 74 | 7678032 | 7637218H1 | SNP00068536 | 162 | 798 | A | A | G | S264 | 0.94 | 0.95 | 0.90 | 0.80 |
| 76 | 1288969 | 1787369H1 | SNF00053600 | 259 | 379 | C | C | A | P115 | n/a | n/a | n/a | n/a |
| 76 | 1288969 | 2760479H1 | SNP00128679 | 83 | 961 | G | G | C | E309 | n/d | n/a | n/a | n/a |
| 76 | 1288969 | 6147071H1 | SNP00128679 | 483 | 1011 | G | G | C | S325 | n/d | n/a | n/a | n/a |
| 76 | 1288969 | 8615852H1 | SNP00153352 | 80 | 1171 | C | A | C | noncoding | n/a | n/a | n/a | n/a |
| 77 | 72069135 | 1418077H1 | SNP00139576 | 172 | 1167 | G | G | A | T266 | n/a | n/a | n/a | n/a |
| 77 | 72069135 | 2412258H1 | SNP00046339 | 6 | 1570 | G | G | A | noncoding | n/a | n/a | n/a | n/a |
| 78 | 7506247 | 2158676H1 | SNP00024506 | 207 | 2639 | T | T | G | noncoding | n/a | n/a | n/a | n/a |
| 78 | 7506247 | 2279409H1 | SNP00154639 | 46 | 2453 | C | C | T | noncoding | n/a | n/a | n/a | n/a |
| 78 | 7506247 | 261068H1 | SNP00024502 | 62 | 1592 | A | G | A | noncoding | 0.76 | n/a | n/a | n/a |
| 78 | 7506247 | 3333887H1 | SNP00062425 | 63 | 952 | C | G | C | S272 | 0.23 | n/a | n/a | n/a |
| 78 | 7506247 | 488020H1 | SNP00024505 | 219 | 2564 | G | A | G | noncoding | n/a | n/a | n/a | n/a |
| 78 | 7506247 | 6450195H1 | SNP00149736 | 73 | 386 | C | C | T | Y83 | n/a | n/a | n/a | n/a |
| 80 | 7509068 | 2512889H1 | SNP00039813 | 21 | 32 | G | G | C | R3 | n/a | n/a | n/a | n/a |
| 80 | 7509068 | 3700531H1 | SNP00039813 | 61 | 29 | C | G | C | A2 | n/a | n/a | n/a | n/a |
| 81 | 7505897 | 2099024H1 | SNP00014945 | 85 | 893 | C | C | T | noncoding | 0.01 | n/a | n/a | n/a |
| 81 | 7505897 | 2618646H1 | SNP00014944 | 207 | 211 | G | T | G | D46 | n/d | n/a | n/a | n/a |
| 81 | 7505897 | 2924229H1 | SNP00014944 | 145 | 216 | T | T | G | H47 | n/d | n/a | n/a | n/a |
| 81 | 7505897 | 3700347H1 | SNP00014945 | 204 | 891 | C | C | T | noncoding | 0.01 | n/a | n/a | n/a |
| 81 | 7505897 | 7093916H1 | SNP00014944 | 180 | 219 | T | T | G | T48 | n/d | n/a | n/a | n/a |
| 82 | 7505898 | 021795H1 | SNP00004602 | 99 | 787 | A | G | A | T157 | 0.64 | n/a | n/a | n/a |
| 82 | 7505898 | 1274130H1 | SNP00025427 | 108 | 1369 | C | C | T | noncoding | n/a | n/a | n/a | n/a |
| 82 | 7505898 | 1621864H1 | SNP00025428 | 126 | 1574 | C | C | T | noncoding | n/a | n/a | n/a | n/a |
| 82 | 7505898 | 2209415H1 | SNP00025429 | 29 | 1726 | G | G | A | noncoding | n/a | n/a | n/a | n/a |
| 82 | 7505898 | 2259911H1 | SNP00025429 | 177 | 1725 | G | G | A | noncoding | n/a | n/a | n/a | n/a |
| 82 | 7505898 | 263670H1 | SNP00004602 | 170 | 786 | A | G | A | K157 | 0.64 | n/a | n/a | n/a |
| 82 | 7505898 | 3165455H1 | SNP00004602 | 190 | 784 | A | G | A | G156 | 0.64 | n/a | n/a | n/a |
| 82 | 7505898 | 3233533H1 | SNP00025427 | 91 | 1368 | C | C | T | noncoding | n/a | n/a | n/a | n/a |
| 82 | 7505898 | 3486645H1 | SNP00004602 | 88 | 785 | G | G | A | A157 | 0.64 | n/a | n/a | n/a |
| 82 | 7505898 | 4111006H1 | SNP00025428 | 54 | 1572 | C | C | T | noncoding | n/a | n/a | n/a | n/a |
| 82 | 7505898 | 4111006H1 | SNP00025429 | 204 | 1723 | G | G | A | noncoding | n/a | n/a | n/a | n/a |
| 82 | 7505898 | 5568111H1 | SNP00025427 | 53 | 1359 | C | C | T | noncoding | n/a | n/a | n/a | n/a |
| 82 | 7505898 | 6273644H1 | SNP00025428 | 125 | 1568 | C | C | T | noncoding | n/a | n/a | n/a | n/a |
| 82 | 7505898 | 6273644H1 | SNP00025429 | 275 | 1719 | G | G | A | noncoding | n/a | n/a | n/a | n/a |
| 83 | 7505907 | 077146H1 | SNP00036633 | 137 | 554 | C | T | C | T184 | n/a | n/a | n/a | n/a |
| 83 | 7505907 | 1674178H1 | SNP00036632 | 224 | 212 | A | G | A | D70 | n/a | n/a | n/a | n/a |
| 83 | 7505907 | 2193971H1 | SNP00053195 | 115 | 1395 | T | T | C | P464 | n/d | n/a | n/a | n/a |
| 83 | 7505907 | 2581095H1 | SNP00036632 | 190 | 210 | G | C | A | T69 | n/a | n/a | n/a | n/a |
| 83 | 7505907 | 367107H1 | SNP00053195 | 84 | 1372 | T | T | C | stop457 | n/d | n/a | n/a | n/a |
| 83 | 7505907 | 3804436H1 | SNP00053195 | 139 | 1393 | T | T | C | S464 | n/d | n/a | n/a | n/a |
| 83 | 7505907 | 3805960H1 | SNP00053195 | 162 | 1394 | T | T | C | L464 | n/d | n/a | n/a | n/a |
| 83 | 7505907 | 7391942H1 | SNP00096172 | 158 | 545 | C | T | C | A181 | n/a | n/a | n/a | n/a |
| 84 | 7505925 | 3102607H1 | SNP00004256 | 270 | 573 | C | G | C | H186 | 0.02 | n/a | n/a | n/a |
| 84 | 7505925 | 3556214H1 | SNP00004256 | 240 | 572 | C | G | C | P186 | 0.02 | n/a | n/a | n/a |
| 84 | 7505925 | 3558094H1 | SNP00053544 | 151 | 537 | T | T | G | I174 | n/a | n/a | n/a | n/a |
| 84 | 7505925 | 5034018H1 | SNP00053544 | 241 | 535 | T | T | G | F174 | n/a | n/a | n/a | n/a |

1. An isolated polypeptide selected from the group consisting of:
a) a polypeptide comprising an amino acid sequence selected from the group consisting of SEQ ID NO:1-42,
b) a polypeptide comprising a naturally occurring amino acid sequence at least 90% identical to an amino acid sequence selected from the group consisting of SEQ ID NO:6, SEQ ID NO:12, SEQ ID NO:15-16, SEQ ID NO:20-22, SEQ ID NO:26, SEQ ID NO:30, SEQ ID NO:35, and SEQ ID NO₄₁,
c) a polypeptide comprising a naturally occurring amino acid sequence at least 94% identical to an amino acid sequence selected from the group consisting of SEQ ID NO:9 and SEQ ID NO:27,
d) a polypeptide comprising a naturally occurring amino acid sequence at least 99% identical to the amino acid sequence of SEQ ID NO:25,
e) a polypeptide comprising a naturally occurring amino acid sequence at least 93% identical to an amino acid sequence selected from the group consisting of SEQ ID NO:34 and SEQ ID NO:36,
f) a polypeptide comprising a naturally occurring amino acid sequence at least 98% identical to the amino acid sequence of SEQ ID NO:38,
g) a polypeptide consisting essentially of a naturally occurring amino acid sequence at least 90% identical to an amino acid sequence selected from the group consisting of SEQ ID NO:1, SEQ ID NO:3-5, SEQ ID NO:7-8, SEQ ID NO:10-11, SEQ ID NO: 13-14, SEQ ID NO: 17-19, SEQ ID NO:23-24, SEQ ID NO:28-29, SEQ ID NO:31-33, SEQ ID NO:37, SEQ ID NO:39-40, and SEQ ID NO:42,
h) a biologically active fragment of a polypeptide having an amino acid sequence selected from the group consisting of SEQ ID NO:1-42, and
i) an immunogenic fragment of a polypeptide having an amino acid sequence selected from the group consisting of SEQ ID NO: 1-42.

2. An isolated polypeptide of claim 1 comprising an amino acid sequence selected from the group consisting of SEQ ID NO:1-42.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:43-84.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-42.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:43-84,
b) a polynucleotide comprising a naturally occurring polynucleotide sequence at least 90% identical to a polynucleotide sequence selected from the group consisting of SEQ ID NO:44-75, SEQ ID NO:78, and SEQ ID NO:81-83,
c) a polynucleotide comprising a naturally occurring polynucleotide sequence at least 92% identical to the polynucleotide sequence of SEQ ID NO:43,
d) a polynucleotide comprising a naturally occurring polynucleotide sequence at least 98% identical to the polynucleotide sequence of SEQ ID NO:76,
e) a polynucleotide comprising a naturally occurring polynucleotide sequence at least 95% identical to the polynucleotide sequence of SEQ ID NO:80,
f) a polynucleotide consisting essentially of a naturally occurring polynucleotide sequence at least 90% identical to a polynucleotide sequence selected from the group consisting of SEQ ID NO:77, SEQ ID NO:79, and SEQ ID NO:84,
g) a polynucleotide complementary to a polynucleotide of a),
h) a polynucleotide complementary to a polynucleotide of b),
i) a polynucleotide complementary to a polynucleotide of c),
j) a polynucleotide complementary to a polynucleotide of d),
k) a polynucleotide complementary to a polynucleotide of e),
l) a polynucleotide complementary to a polynucleotide of f), and
m) an RNA equivalent of a)-l).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-42.

19. A method for treating a disease or condition associated with decreased expression of functional ENZM, comprising administering to a patient in need of such treatment the composition of claim 17.

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional ENZM, comprising administering to a patient in need of such treatment a composition of claim 21.

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional ENZM, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A method for a diagnostic test for a condition or disease associated with the expression of ENZM in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of ENZM in a subject, comprising administering to said subject an effective amount of the composition of claim 32.

34. A composition of claim 32, further comprising a label.

35. A method of diagnosing a condition or disease associated with the expression of ENZM in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of an amino acid sequence selected from the group consisting of SEQ ID NO:1-42, or an immunogenic fragment thereof, under conditions to elicit an antibody response,
b) isolating antibodies from the animal, and
c) screening the isolated antibodies with the polypeptide, thereby identifying a polyclonal antibody which specifically binds to a polypeptide comprising an amino acid sequence selected from the group consisting of SEQ ID NO:1-42.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of an amino acid sequence selected from the group consisting of SEQ ID NO:1-42, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which specifically binds to a polypeptide comprising an amino acid sequence selected from the group consisting of SEQ ID NO:1-42.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide comprising an amino acid sequence selected from the group consisting of SEQ ID NO:1-42 in a sample, the method comprising:
a) incubating the antibody of claim 11 with the sample under conditions to allow specific binding of the antibody and the polypeptide, and
b) detecting specific binding, wherein specific binding indicates the presence of a polypeptide comprising an amino acid sequence selected from the group consisting of SEQ ID NO:1-42 in the sample.

45. A method of purifying a polypeptide comprising an amino acid sequence selected from the group consisting of SEQ ID NO:1-42 from a sample, the method comprising:
a) incubating the antibody of claim 11 with the sample under conditions to allow specific binding of the antibody and the polypeptide, and
b) separating the antibody from the sample and obtaining the purified polypeptide comprising an amino acid sequence selected from the group consisting of SEQ ID NO:1-42.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising an amino acid sequence selected from the group consisting of:
a) the amino acid sequence of SEQ ID NO:1;
b) the amino acid sequence of SEQ ID NO:2;
c) the amino acid sequence of SEQ ID NO:3;
d) the amino acid sequence of SEQ ID NO:4;
e) the amino acid sequence of SEQ ID NO:5;
f) the amino acid sequence of SEQ ID NO:6;
g) the amino acid sequence of SEQ ID NO:7;
h) the amino acid sequence of SEQ ID NO:8;
i) the amino acid sequence of SEQ ID NO:9;
i) the amino acid sequence of SEQ ID NO:10;
k) the amino acid sequence of SEQ ID NO:11;
l) the amino acid sequence of SEQ ID NO:12;
m) the amino acid sequence of SEQ ID NO:13;
n) the amino acid sequence of SEQ ID NO:14;
o) the amino acid sequence of SEQ ID NO:15;
p) the amino acid sequence of SEQ ID NO:16;
q) the amino acid sequence of SEQ ID NO:17;
r) the amino acid sequence of SEQ ID NO:18;
s) the amino acid sequence of SEQ ID NO:19;
t) the amino acid sequence of SEQ ID NO:20;
u) the amino acid sequence of SEQ ID NO:21;
v) the amino acid sequence of SEQ ID NO:22;
w) the amino acid sequence of SEQ ID NO:23;
x) the amino acid sequence of SEQ ID NO:24;
y) the amino acid sequence of SEQ ID NO:25;
z) the amino acid sequence of SEQ ID NO:26;
aa) the amino acid sequence of SEQ ID NO:27;
bb) the amino acid sequence of SEQ ID NO:28;
cc) the amino acid sequence of SEQ ID NO:29;.
dd) the amino acid sequence of SEQ ID NO:30;
ee) the amino acid sequence of SEQ ID NO:31;
ff) the amino acid sequence of SEQ ID NO:32;
gg) the amino acid sequence of SEQ ID NO:33;
hh) the amino acid sequence of SEQ ID NO:34;
ii) the amino acid sequence of SEQ ID NO:35;
jj) the amino acid sequence of SEQ ID NO:36;
kk) the amino acid sequence of SEQ ID NO:37;
ll) the amino acid sequence of SEQ ID NO:38;
mm) the amino acid sequence of SEQ ID NO:39;
nn) the amino acid sequence of SEQ ID NO:40;
oo) the amino acid sequence of SEQ ID NO:41; and
pp) the amino acid sequence of SEQ ID NO:42.

57-97. (canceled)

98. A polynucleotide of claim 12, comprising a polynucleotide sequence selected from the group consisting of:
a) the polynucleotide sequence of SEQ ID NO:43;
b) the polynucleotide sequence of SEQ ID NO:44;
c) the polynucleotide sequence of SEQ ID NO:45;
d) the polynucleotide sequence of SEQ ID NO:46;
e) the polynucleotide sequence of SEQ ID NO:47;
f) the polynucleotide sequence of SEQ ID NO:48;
g) the polynucleotide sequence of SEQ ID NO:49;
h) the polynucleotide sequence of SEQ ID NO:50;
i) the polynucleotide sequence of SEQ ID NO:51;
i) the polynucleotide sequence of SEQ ID NO:52;
k) the polynucleotide sequence of SEQ ID NO:53;
l) the polynucleotide sequence of SEQ ID NO:54;
m) the polynucleotide sequence of SEQ ID NO:55;
n) the polynucleotide sequence of SEQ ID NO:56;
o) the polynucleotide sequence of SEQ ID NO:57;
p) the polynucleotide sequence of SEQ ID NO:58;
q) the polynucleotide sequence of SEQ ID NO:59;
r) the polynucleotide sequence of SEQ ID NO:60;
s) the polynucleotide sequence of SEQ ID NO:61;
t) the polynucleotide sequence of SEQ ID NO:62;
u) the polynucleotide sequence of SEQ ID NO:63;
v) the polynucleotide sequence of SEQ ID NO:64;
w) the polynucleotide sequence of SEQ ID NO:65;
x) the polynucleotide sequence of SEQ ID NO:66;
y) the polynucleotide sequence of SEQ ID NO:67;
z) the polynucleotide sequence of SEQ ID NO:68;
aa) the polynucleotide sequence of SEQ ID NO:69;
bb) the polynucleotide sequence of SEQ ID NO:70;
cc) the polynucleotide sequence of SEQ ID NO:71;
dd) the polynucleotide sequence of SEQ ID NO:72;.
ee) the polynucleotide sequence of SEQ ID NO:73;.
ff) the polynucleotide sequence of SEQ ID NO:74;.
gg) the polynucleotide sequence of SEQ ID NO:75;.
hh) the polynucleotide sequence of SEQ ID NO:76;.
ii) the polynucleotide sequence of SEQ ID NO:77;.
jj) the polynucleotide sequence of SEQ ID NO:78;.
kk) the polynucleotide sequence of SEQ ID NO:79;.
ll) the polynucleotide sequence of SEQ ID NO:80;.
mm) the polynucleotide sequence of SEQ ID NO:81;.
nn) the polynucleotide sequence of SEQ ID NO:82;.
oo) the polynucleotide sequence of SEQ ID NO:83; and.
pp) the polynucleotide sequence of SEQ ID NO:84.

99-139. (canceled)


SEQUENCE LISTING (84 sequences)

SEQ ID NO: 1 - protein, 378 aa, Homo sapiens
  misc_feature: Incyte ID No 70612021CD1
MKTGHFEIVTMLLATMILVDIFQVKAEVLDMADNAFDDEYLKCTDRMEIKYVPQLLKEEKASHQQLDTVWENAKAKWAARKTQIFLPMNFKDNHGIALMAYISEAQEQTPFYHLFSEAVKMAGQSREDYIYGFQFKAFHFYLTRALQLLRKPCEASSKTVVYRTSQGTSFTFGGLNQARFGHFTLAYSAKPQAANDQLTVLSIYTCLGVDIENFLDKESERITLIPLNEVFQVSQEGAGNNLILQSINKTCSHYECAFLGGLKTENCIENLEYFQPIYVYNPGEKNQKLEDHSEKNWKLEDHGEKNQKLEDHGVKILEPTQIPGMKIPEPFPLPAPGPVPVPGPKSHPSASLGKLLLPQFGMVIILISVSAINLFVAL

SEQ ID NO: 2 - protein, 715 aa, Homo sapiens
  misc_feature: Incyte ID No 71847235CD1
MHLLPALAGVLATLVLAQPCEGTDPASPGAVETSVLRDCIAEAKLLVDAAYNWTQKSIKQRLRSGSASPMDLLSYFKQPVAATRTVVRAADYMHVALGLLEEKLQPQRSGPFNVTDVLTEPHLRLLSQASGCALRDQAERCSDKYRTITGRCNNKRRPLLGASNQALARWLPAEYEDGLSLPFGWTPSRRRNGFLLPLVRAVSNQIVRFPNERLTSDRGRALMFMQWGQFIDHDLDFSPESPARVAFTAGVDCERTCAQLPPCFPIKIPPNDPRIKNQRDCIPFFRSAPSCPQNKNRVRNQINALTSFVDASMVYGSEVSLSLRLRNRTNYLGLLAINQRFQDNGRALLPFDNLHDDPCLLTNRSARIPCFLAGDTRSTETPKLAAMHTLFMREHNRLATELRRLNPRWNGDKLYNEARKIMGAMVQIITYRDFLPLVLGKARARRTLGHYRGYCSNVDPRVANVFTLAFRFGHTMLQPFMFRLDSQYRASAPNSHVPLSSAFFASWRIVYEGGIDPILRGLMATPAKLNRQDAMLVDELRDRLFRQVRRIGLDLAALNMQRSRDHGLPGYNAWRRFCGLSQPRNLAQLSRVLKNQDLARKFLNLYGTPDNIDIWIGAIAEPLLPGARVGPLLACLFENQFRRARDGDRFWWQKRGVFTKRQRKALSRISLSRIICDNTGITTVSRDIFRANIYPRGFVNCSRIPRLNLSAWRGT

SEQ ID NO: 3 - protein, 811 aa, Homo sapiens
  misc_feature: Incyte ID No 7505230CD1
MSAFRLWPGLLIMLGSLCHRGSPCGLSTHIEIGHRALEFLQLHNGRVNYRELLLEHQDAYQAGIVFPDCFYPSICKGGKFHDVSESTHWTPFLNASVHYIRENYPLPWEKDTEKLVAFLFGITSHMAADVSWHSLGLEQGFLRTMGAIDFHGSYSEAHSAGDFGGDVLSQFEFNFNYLARRWYVPVKDLLGIYEKLYGRKVITENVIVDCSHIQFLEMYGEMLAVSKLYPTYSTKSPFLVEQFQEYFLGGLDDMAFWSTNIYHLTSFMLENGTSDCNLPENPLFIACGGQQNHTQGSKMQKNDFHRNLTTSLTESVDRNINYTERGVFFSVNSWTPDSMSFIYKALERNIRTMFIGGSQLSQKHVSSPLASYFLSFPYARLGWAMTSADLNQDGHGDLVVGAPGYSRPGHIHIGRVYLIYGNDLGLPPVDLDLDKEAHRILEGFQPSGRFGSALAVLDFNVDGVPDLAVGAPSVGSEQLTYKGAVYVYFGSKQGGMSSSPNITISCQDIYCNLGWTLLAADVNGDSEPDLVIGSPFAPGGGKQKGIVAAFYSGPSLSDKEKLNVEAANWTVRGEEDFSWFGYSLHGVTVDNRTLLLVGSPTWKNASRLGHLLHIRDEKKSLGRVYGYFPPNGQSWFTISGAPTYDDVSKVAFLTVTLHQGGATRMYALISDAQPLLLSTFSGDRRFSRFGGVLHLSDLDDDGLDEIIMAAPLRIADVTSGLIGGEDGRVYVYNGKETTLGDMTGKCKSWITPCPEEKAQYVLISPEASSRFGSSLITVRSKAKNQVVIAAGRSSLGARLSGALHVYSLGSD

SEQ ID NO: 4 - protein, 564 aa, Homo sapiens
  misc_feature: Incyte ID No 7505235CD1
MAPKKLSCLRSLLLPLSLTLLLPQADTRSFVVDRGHDRFLLDGAPFRYVSGSLHYFRVPRVLWADRLLKMRWSGLNAIQFYVPWNYHEPQPGVYNFNGSRDLIAFLNEAALANLLVILRPGPYICAEWEMGGLPSWLLRKPEIHLRTSDPADNMTKIFTLLRKYEPHGPLVNSEYYTGWLDYWGQNHSTRSVSAVTKGLENMLKLGASVNMYMFHGGTNFGYWNGADKKGRFLPITTSYDYDAPISEAGDPTPKLFALRDVISKFQEVPLGPLPPPSPKMMLGPVTLHLVGHLLAFLDLLCPRGPIHSILPMTFEAVKQDHGFMLYRTYMTHTIFEPTPFWVPNNGVHDRAYVMVDGVFQGVVERNMRDKLFLTGKLGSKLDILVENMGRLSFGSNSSDFKGLLKPPILGQTILTQWMMFPLKIDNLVKWWFPLQLPKWPYPQAPSGPTFYSKTFPILGSVGDTFLYLPGWTKGQVWINGFNLGRYWTKQGPQQTLYVPRFLLFPRGALNKITLLELEDVPLQPQVQFLDKPILNSTSTLHRTHINSLSADTLSASEPMELSGH

SEQ ID NO: 5 - protein, 233 aa, Homo sapiens
  misc_feature: Incyte ID No 7505793CD1
MAQTPAFDKPKVELHVHLDGSIKPETILYYGRRRGIALPANTAEGLLNVIGMDKPLTLPDFLAKFDYYMPAIEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 6 - protein, 221 aa, Homo sapiens
  misc_feature: Incyte ID No 7505861CD1
MAAASGSVLQRCIVSPAGRHSASLIFLHGSGDSGQGLRMWIKQVLNQDLTFQHIKIIYPTAPPRFKITNDCPEHLESIDVMCQVLTDLIDEEVKSGIKKNRILIGGFSMGGCMAMHLAYRNHQDVAGVFALSSFLNKASAVYQALQKSNGVLPELFQCHGTADELVLHSWAEETNSMLKSLGVTTKFHSFPNVYHELSKTELDILKLWILTKLPGEMEKQK

SEQ ID NO: 7 - protein, 283 aa, Homo sapiens
  misc_feature: Incyte ID No 7505864CD1
METGPEDPSSMPEESSPRRTPQSIPYQDLPHLVNADGQYLFCRYWKPTGTPKALIFVSHGAGEHSGRYEELARMLMGLDLLVFAHDHVGHGQSEGERMVVSDFHVFVRDVLQHVDSMQKDYPGLPVFLLGHSMGGAIAILTAAERPGHFAGMVLISPLVLANPESATTFKVDIYNSDPLICRAGLKVCFGIQLLNAVSRVERALPKLTVPFLLLQGSADRLCDSKGAYLLMELAKSQDKTLKIYEGAYHVLHKELPEVTNSVFHEINMWVSQRTATAGTASPP

SEQ ID NO: 8 - protein, 339 aa, Homo sapiens
  misc_feature: Incyte ID No 7506427CD1
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 9 - protein, 130 aa, Homo sapiens
  misc_feature: Incyte ID No 7506429CD1
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAELVPQGGGAV

SEQ ID NO: 10 - protein, 127 aa, Homo sapiens
  misc_feature: Incyte ID No 7505799CD1
MLPRAAWSLVLRKGGGGRRGMHSSEGTTRGGGKMSPYTNCYAQRYYPMPEEPFCTELNAEEQALKEKEKGSWTQLTHAEKVALFPPKPITLTDERKAQQLQRMLDMKVNPVQGLASHWDYEKKQWKK

SEQ ID NO: 11 - protein, 277 aa, Homo sapiens
  misc_feature: Incyte ID No 7505843CD1
MGAQLSTLGHMVLFPVWFLYSLLIDREIISHDTRRFRFALPSPQHILGLPVGQHIYLSARIDGNLVVRPYTPISSDDDKGFVDLVIKVYFKDTHPKFPAGGKMSQYLESMQIGDTIEFRGPSGLLVYQGKGKFAIRPDKKSNPIIRTVKSVGMIAGGTGITPMLQVIRAIMKDPDDHTVCHLLFANQTEKDILLRPELEELRNKHSARFKLWYTLDRAPEAWDYGQGFVNEEMIRDHLPPPEEEPLVLMCGPPPMIQYACLPNLDHVGHPTERCFVF

SEQ ID NO: 12 - protein, 470 aa, Homo sapiens
  misc_feature: Incyte ID No 90001378CD1
MFAVHLMAFYFSKLKEDQIKKVDRFLYHMRLSDDTLLDIMRRFRAEMEKGLAKDTNPTAAVKMLPTFVRAIPDGSENGEFLSLDLGGSKFRVLKVQVAEEGKRHVQMESQFYPTPNEIIRGNGTELFEYVADCLADFMKTKDLKHKKLPLGLTFSFPCRQTKLEEGVLLSWTKKFKARGVQDTDVVSRLTKAMRRHKDMDVDILALVNDTVGTMMTCGYEDPNCEIGLIAGTGSNMCYMEDMRNIEMVEGGEGKMCINTEWGGFGDNGCIDDIRTRYDTEVDEGSLNPGKQRYEKMTSGMYLGEIVRQILIDLTKQGLLFRGQISERLRTRGIFETKFLSQIESDRLALLQVRRILQQLGLDSTCEDSIVVKEVCGAVSRRAAQLCGAGLAAIVEKRREDQGLEHLRITVGVDGTLYKLHPHFSRILQETVKELAPRCDVTFMLSEDGSGKGAALITAVAKRLQQAQKEN

SEQ ID NO: 13 - protein, 298 aa, Homo sapiens
  misc_feature: Incyte ID No 7504923CD1
MLLLAAAFLVAFVLLLYMVSPLISPKPLALPGAHVVVTGGSSGIGKCIAIECYKQGAFITLVARNEDKLLQAKKEIEMHSINDKQVVLCISVDVSQDYNQVENVIKQAQEKLGPVDMLVNCAGMAVSGKFEDLEVSTFERLMSINYLGSVYPSRAVITTMKERRVGRIVFVSSQAGQLGLFGFTAYSASKFAIRGLAEALQMEVKPYNVYITVAYPPDTDTPGFAEENRTKPLETRLISETTSVCKPEQVAKQIVKDAIVVTMGLFRTIALFYLGSFDSIVRRCMMQREKSENADKTA

SEQ ID NO: 14 - protein, 331 aa, Homo sapiens
  misc_feature: Incyte ID No 7506151CD1
MAHRFPALTQEQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAASTQSLFTACYTY

SEQ ID NO: 15 - protein, 102 aa, Homo sapiens
  misc_feature: Incyte ID No 7506450CD1
MSMKWTSALLLIQLSCYFSSGSCGKVLVWPTEFSHWMNIKTILDELVQRGHEMLFSPLVSCWPSYLKYPLSTASASLLATQLKSIVEDFCSLLPMCLLLCQN

SEQ ID NO: 16 - protein, 458 aa, Homo sapiens
  misc_feature: Incyte ID No 71380031CD1
MGLSRKEQVFLALLGASGVSGLTALILLLVEATSVLLPTDIKFGIVFDAGSSHTSLFLYQWPANKENGTGVVSQALACQVEGPGISSYTSNAAQAGESLQGCLEEALVLIPEAQHRKTPTFLGATAGMRLLSRKNSSQARDIFAAVTQVLGRSPVDFWGAELLAGQAEGAFGWITVNYGLGTLVKYSFTGEWIQPPEEMLVGALDMGGASTQITFVPGGPILDKSTQADFRLYGSDYSVYTHSYLCFGRDQMLSRLLVGLVQSRPAALLRHPCYLSGYQTTLALGPLYESPCVHATPPLSLPQNLTVEGTGNPGACVSAIRELFNFSSCQGQEDCAFDGVYQPPLRGQFYVEASYPGQDRWLRDYCASGLYILTLLHEGYGFSEETWPSLEFRKQAGGVDIGWTLGYMLNLTGMIPADAPAQWRAESYGVWVAKVVFMVLALVAVVGAALVQLFWLQD

SEQ ID NO: 17 - protein, 420 aa, Homo sapiens
  misc_feature: Incyte ID No 7506054CD1
MAMQKIFAREILDSRGNPTVEVDLHTAKGRFRAAVPSGASTGIYEALELRDGDKGRYLGKGVLKAVENINSTLGPALLQKKLSVADQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 18 - protein, 512 aa, Homo sapiens
  misc_feature: Incyte ID No 7506139CD1
MSFQGKKSIPRITSDRLLIRGGRIVNDDQSFYADVHVEDGLIKQIGENLIVPGGIKTIDAHGLMVLPGGVDVHTRLQMPVLGMTPADDFCQGTKAALAGGTTMILDHVFPDTGVSLLAAYEQWRERADSAACCDYSLHVDITRWHESIKEELEALVKEKGVNSFLVFMAYKDRCQCSDSQMYEIFSIIRDLGALAQVHAENGDIVEEEQKRLLELGITGPEGHVLSHPEEVEAEAVYRAVTIAKQANCPLYVTKVMSKGAADAIAQAKRRGVVVFGEPITASLGTDGSHYWSKNWAKAAAFVTSPPVNPDPTTADHLTCLLSSGDLQVTGSAHCTFTTAQKAVGKDNFALIPEGTNGIEERMSMVWEKCVASGKMDENEFVAVTSTNAAKIFNFYPRKGRVAVGSDADLVIWNPKATKIISAKTHNLLAEIHGVPRGLYDGPVHEVMVPAKPGSGAPARASCPGKISVPPVRNLHQSGFSLSGSQADDHIARRTAQKIMAPPGGRSNITSLS

SEQ ID NO: 19 - protein, 327 aa, Homo sapiens
  misc_feature: Incyte ID No 7506426CD1
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 20 - protein, 421 aa, Homo sapiens
  misc_feature: Incyte ID No 7506741CD1
MSVSVLSPSRLLGDVSGILQAASLLILLLLLIKAVQLYLHRQWLLKALQQFPCPPSHWLFGHIQELQQDQELQRIQKWVETFPSACPHWLWGGKVRVQLYDPDYMKVILGRSDPKSHGSYRFLAPWIGYGLLLLNGQTWFQHRRMLTPAFHYDILKPYVGLMADSVRVMLDKWEELLGQDSPLEVFQHVSLMTLDTIMKCAFSHQGSIQVDRPSDPTEEGSTTEGGGAGEDQEEEAFGFSGYPPLGQRAPGLPRPCWCSGWNCFRNHLDQMPYTTMCIKEALRLYPPVPGIGRELSTPVTFPDGRSLPKGIMVLLSIYGLHHNPKVWPNPEVFDPFRFAPGSAQHSHAFLPFSGGSRNCIGKQFAMNELKVATALTLLRFELLPDPTRIPIPIARLVLKSKNGIHLRLRRLPNPCEDKDQL

SEQ ID NO: 21 - protein, 249 aa, Homo sapiens
  misc_feature: Incyte ID No 7506743CD1
MSVSVLSPSRLLGDVSGILQAASLLILLLLLIKAVQLYLHRQWLLKALQQFPCPPSHWLFGHIQELQQDQELQRIQKWVETFPSACPHWLWGGKVRVQLYDPDYMKVILGRSDPKSHGSYRFLAPWIGYGLLLLNGQTWFQHRRMLTPAFHYDILKPYVGLMADSVRVMLDKWEELLGQDSPLEVFQHVSLMTLDTIMKCAFSHQGSIQVDRPSDPTEEGSTTEGGGAGEDQEEEAFGFSGYPPLGQSV

SEQ ID NO: 22 - protein, 311 aa, Homo sapiens
  misc_feature: Incyte ID No 7506746CD1
MSVSVLSPSRLLGDVSGILQAASLLILLLLLIKAVQLYLHRQWLLKALQQFPCPPSHWLFGHIQELQQDQELQRIQKWVETFPSACPHWLWGGKVRVQLYDPDYMKVILGRSDPKSHGSYRFLAPWIGYGLLLLNGQTWFQHRRMLTPAFHYDILKPYVGLMADSVRVMLDKWEELLGQDSPLEVFQHVSLMTLDTIMKCAFSHQGSIQVGRPSDPTEEGSTTEGGGAGEDQEEEAFGFSGYPPLGQRAPGLPRPCWCSGWNCFRNHLDQMPYTTMCIKEALRLYPPVPGIGRELSTPVTFPDGRSLPKGV

SEQ ID NO: 23 - protein, 152 aa, Homo sapiens
  misc_feature: Incyte ID No 7506748CD1
MPMTLGYWNIRGLAHSIRLLLEYTDSSYEEKKYTMGDAPDYDRSQWLNEKFKLGLDFPNLPYLIDGTHKITQSNAILRYIARKHNLCGESEKEQIREDILENQFMDSCLDAFPNLKDFISRFEGLEKISAYMKSSRFLPRPVFSKMAVWGNK

SEQ ID NO: 24 - protein, 453 aa, Homo sapiens
  misc_feature: Incyte ID No 1419966CD1
MQRLLTPVKRILQLTRAVQETSLTPARLLPVAHQRFSTASAVPLAKTDTWPKDVGILALEVYFPAQYVDQTDLEKYNNVEAGKYTVGLGQTRMGFCSVQEDINSLCLTVVQRLMERIQLPWDSVGRLEVGTETIIDKSKAVKTVLMELFQDSGNTDIEGIDTTNACYGGTASLFNAANWMESSSWDGRYAMVVCGDIAVYPSGNARPTGGAGAVAMLIGPKAPLALERAGSDRPFTLDDLQYMIFHTPFCKMVQKSLARLMFNDFLSASSDTQTSLYKGLEAFGGLKLEDTYTNKDLDKALLKASQDMFDKKTKASLYLSTHNGNMYTSSLYGCLASLLSHHSAQELAGSRIGAFSYGSGLAASFFSFRVSQDAAPGSPLDKLVSSTSDLPKRLASRKCVSPEEFTEIMNQREQFYHKVNFSPPGDTNSLFPGTWYLERVDEQHRRKYARRPV

SEQ ID NO: 25 - protein, 285 aa, Homo sapiens
  misc_feature: Incyte ID No 7506451CD1
MSMKWTSALLLIQLSCYFSSGSCGKVLVWPTEFSHWMNIKTILDELVQRGHEVTVLASSASISFDPNSPSTLKFEVYPVSLTKTEFEDIIKQLVKRWAELPKDTFWSYFSQVQEIMWTFNDILRKFCKDIVSNKKLMKKLQESRFDVVLADAVFPFGRPTTLSETMAKADIWLIRNYWDFQFPHPLLPNVEFVGGLHCKPAKPLPKEMEEFVQSSGENGVVVFSLGSMVSNTSEERANVIASALAKIPQKVLWRFDGNKPDTLGLNTRLYKWIPQNDLLDIKRML

SEQ ID NO: 26 - protein, 201 aa, Homo sapiens
  misc_feature: Incyte ID No 90015249CD1
MLSTFARQNDIPFQLQTVELAWGEHLKPEFLKVNPLGKVPALRDGDFLLAESMVIVLYLSRKYQIRGHWYPPELQACTCVDEYLAWKHVTIQLPATNVYLCKTACRCCTAGAAVGEADASPAAPGWGGPGGQALPGNGADLPGRLGADGGDAGEAFLPTCPRGDSGHGAEPQPQLPCLPSPLPLAATSSKTGPGWQCDRPI

SEQ ID NO: 27 - protein, 399 aa, Homo sapiens
  misc_feature: Incyte ID No 7487231CD1
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVICCGHHEVPAVDTSYLQAYNSPPAIHPGN

SEQ ID NO: 28 - protein, 364 aa, Homo sapiens
  misc_feature: Incyte ID No 7506260CD1
MAMAYLAWRLARRSCPSSLQVTSFPVVQLHMNRTAMRASQKDFENSMNQVKLLKKDPGNEVKLKLYALYKQATEGPCNMPKPGVFDLINKAKWDAWNALGSLPKEAARQNYVDLVSSLSPSLESSSQVEPGTDRKSTGFETLVVTSEDGITKIMFNRPKKKNAINTEMYHEIMRALKAASKDDSIITVLTGNGDYYSSGNDLTNFTDIPPGGVEEKAKNNAVLLREFVGCFIDFPKPLIAVVNGPAVGISVTLLGLFDAVYASDRATEMLIFGKKLTAGEACAQGLVTEVFPDSTFQKEVWTRLKAFAKLPPNVLRISKEVIRKREREKLHAVNAEECNVLQGRWLSDECTNAVVNFLSRKSKL

SEQ ID NO: 29 - protein, 319 aa, Homo sapiens
  misc_feature: Incyte ID No 7506270CD1
MTTSASSHLNKGIKQVYMSLPQGEKVQAMYIWIDGTGEGLRCKTRTLDSEPKCVEETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 30 - protein, 458 aa, Homo sapiens; identical to SEQ ID NO: 16
  misc_feature: Incyte ID No 7506306CD1

SEQ ID NO: 31 - protein, 295 aa, Homo sapiens
  misc_feature: Incyte ID No 7506428CD1
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 32 - protein, 472 aa, Homo sapiens
  misc_feature: Incyte ID No 7678032CD1
MAAVSLRLGDLVWGKLGRYPPWPGKIVNPPKDLKKPRGKKCFFVKFFGTEDHAWIKVEQLKPYHAHKEEMIKINKGKRFQQAVDAVEEFLRRAKGKDQDLTIPESSTVKGMMAGPMAAFKWQPTASEPVKDADPHFHHFLLSQTEKPAVCYQAITKKLKICEEETGSTSIQAADSTAVNGSITPTDKKIGFLGLGLMGSGIVSNLLKMGHTVTVWNRTAEKCDLFIQEGARLGRTPAEVVSTCDITFACVSDPKAAKDLVLGPSGVLQGIRPGKCYVDMSTVDADTVTELAQVIVSRGGRFLEAPVSGNQQLSNDGMLVILAAGDRGLYEDCSSCFQAMGKTSFFLGEVGNAAKMMLIVNMVQGSFMATIAEGLTLAQVTGQSQQTLLDILNQGQLASIFLDQKCQNILQGNFKPDFYLKYIQKDLRLAIALGDAVNHPTPMAAAANEVYKRAKALDQSDNDMSAVYRAYIH

SEQ ID NO: 33 - protein, 483 aa, Homo sapiens
  misc_feature: Incyte ID No 7508332CD1
MLNNLLLFSLQISLIGTTLGGNVLIWPMEGSHWLNVKIIIDELIKKEHNVTVLVASGALFITPTSNPSLTFEIYKVPFGKERIEGVIKDFVLTWLENRPSPSTIWRFYQEMAKVIKDFHMVSQEICDGVLKNQQLMAKLKKSKFEVLVSDPVFPCGDIVALKLGIPFMYSLRFSPASTVEKHCGKVPYPPSYVPAVLSELTDQMSFTDRIRNFISYHLQDYMFETLWKSWDSYYSKALGRPTTLCETMGKAEIWLIRTYWDFEFPRPYLPNFEFVGGLHCKPAKPLPKVLWRYKGKKPATLGNNTQLFDWIPQNDLLGHPKTKAFITHGGTNGIYEAIYHGVPMVGVPMFADQPDNIAHMKAKGAAVEVNLNTMTSVDLLSALRTVINEPSYKENAMRLSRIHHDQPVKPLDRAVFWIEFVMRHKGAKHLRVAAHDLTWFQYHSLDVIGFLLVCVTTAIFLVIQCCLFSCQKFGKIGKKKKRE

SEQ ID NO: 34 - protein, 346 aa, Homo sapiens
  misc_feature: Incyte ID No 1288969CD1
MGAAARLSAPRALVLWAALGAAAHIGPAPDPEDWWSYKDNLQGNFVPGPPFWGLVNAAWSLCAVGKRQSPVDVELKRVLYDPFLPPLRLSTGGEKLRGTLYNTGRHVSFLPAPRPVVNVSGGPLLYSHRLSELRLLFGARDGAGSEHQINHQGFSAEVQLIHFNQELYGNFSAASRGPNGLAILSLFVNVASTSNPFLSRLLNRDTITRISYKNDAYFLQDLSLELLFPESFGFITYQGSLSTPPCSETVTWILIDRALNITSLQMHSLRLLSQNPPSQIFQSLSGNSRPLQPLAHRALRGNRDPRHPERRCRGPNYRLHAQCFSTWYILVPRSQTSDVDGVPHGR

SEQ ID NO: 35 - protein, 359 aa, Homo sapiens
  misc_feature: Incyte ID No 72069135CD1
MGRRASGCRAWRPGAFGLGGLPPWNASLETSDVRHRSQRLGLARQDRKRDWLAASVVSGTAQSRDAASAELGVGRVDPLKGARARGHGSGSRCFARLPLSQAREVAPPGEAWRDCVVKRVRAPIGRAMWRGLALARAIGCAARGRGQWAVRAADCAQSGRHPGPAVVCGRRLISVLEQIRHFVMMPEINTNHLDKQQVQLLAEMCILIDENDNKIGAETKKNCHLNENIEKGLLHRAFSVFLFNTENKLLLQQRSDAKITFPGCFTNTCCSHPLSNPAELEESDALGVRRAAQRRLKAELGIPLEEVPPEEINYLTRIHYKAQSDGIWGEHEIDYILLDGEQRPSHVTPTVMPDVARCP

SEQ ID NO: 36 - protein, 275 aa, Homo sapiens
  misc_feature: Incyte ID No 7506247CD1
MAAVDSFYLLYREIARSCNCYMEALALVGAWYTARKSITVICDFYSLIRLHFIPRLGSRADLIKQYGRWAVVSGATDGIGKAYAEELASRGLNIILISRNEEKLQYFTQLSEDKLWDIINVNIAAASLMVHVVLPGMVERKKGAIVTISSGSCCKPTPQLAAFSASKAYLDHFSRALQYEYASKGIFVQSLIPFYVATSMTAPSNFLHRCSWLVPSPKVYAHHAVSTLGISKRTTGYWSHSIQFLFAQYMPEWLWVWGANILNRSLRKEALSCTA

SEQ ID NO: 37 - protein, 337 aa, Homo sapiens
  misc_feature: Incyte ID No 7506363CD1
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 38 - protein, 445 aa, Homo sapiens
  misc_feature: Incyte ID No 7509068CD1
MARGLQVPLPRLATGLLLLLSVQPWAESGKVLVVPTDGSPWLSMREALRELHARGHQAVVLTPEVNMHIKEEKFFTLTAYAVPWTQKEFDRVTLGYTQGFFETEHLLKRYSRSMAIMNNVSLALHRCCVELLHNEALIRHLNATSFDVVLTDPVNLCGAVLAKYLSIPAVFFWRYIPCDLDFKGTQCPNPSSYIPKLLTTNSDHMTFLQRVKNMLYPLALSYICHTFSAPYASLASELFQREVSVVDLVSYASVWLFRGDFVMDYPRPIMPNMVFIGGINCANGKPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKRKKQQSGRQM

SEQ ID NO: 39 - protein, 162 aa, Homo sapiens
  misc_feature: Incyte ID No 7505897CD1
MAFPAGFGWAAATAAYQVEGGWDADGKGPCVWDTFTHQGGERVFKNQTGDVACGSYTLWEEDLKCIKQLGLTHYRFSLSWSRLLPDGTTGFINQKAIQLDKVNLQVYCAWSLLDNFEWNQGYSSRFGLFHVDFEDPARPRVPYTSAKEYAKIIRNNGLEAHL

SEQ ID NO: 40 - protein, 321 aa, Homo sapiens
  misc_feature: Incyte ID No 7505898CD1
MSTRESFNPESYELDKSFRLTRFTELKGTGCKVPQDVLQKLLESLQENHFQEDEQFLGAVMPRLGIGMDTCVIPLRHGGLSLVQTTDYIYPIVDDPYMMGRIACANVLSDLYAMGVTECDNMLMLLGVSNKMTDRERDKVMPLIIQGFKDAAEEAGTSVTGGQTVLNPWIVLGGVATTVCQPNEFIMPDNAVPGDVLVLTKPLGTQVAVAVHQWLDIPEKWNKIKLVVTQEDVELAYQEAMMNMARLNRTGGLLICLPREQAARFCAEIKSPKYGEGHQAWIIGIVEKGNRTARIIDKPRIIEVAPQVATQNVNPTPGATS

SEQ ID NO: 41 - protein, 468 aa, Homo sapiens
  misc_feature: Incyte ID No 7505907CD1
MVSADAMVSADAMVSADAMVSADAMVSADAMVSADAMVSADAMVSADAMVSADAMVSADAMVSADAMHTDPDYSAAYVVIETDAEDGIKGCGITFTLGKGTEVVVCAVNALAHHVLNKDLKDIVGDFRGFYRQLTSDGQLRWIGPEKGVVHLATAAVLNAVWDLWAKQEGKPVWKLLVDMDPRMLVSCIDFRYITDVLTEEDALEILQKGQIGKKEREKQMLAQGYPAYTTSCAWLGYSDDTLKQLCAQALKDGWTRFKVKVGADLQDDMRRCQIIRDMIGPEKTLMMDANQRWDVPEAVEWMSKLAKFKPLWIEEPTSPDDILGHATISKCHNRVIFKQLLQAKALQFLQIDSCRLGSVNENLSVLLMAKKFEIPVCPHAGGVGLCELVQHLIIFDYISVSASLENRVCEYVDHLHEHFKYPVMIQRASYMPPKDPGYSTEMKEESVKKHQYPDGEVWKKLLPAQEN

SEQ ID NO: 42 - protein, 318 aa, Homo sapiens
  misc_feature: Incyte ID No 7505925CD1
MAAAALGQIWARKLLSVPWLLCGPRRYASSSFKAADLQLEMTQKPHKKPGPGEPLVFGKTFTDHMLMVEWNDKGWGQPRIQPFQNLTLHPASSSLHYSLQLFEGMKAFKGKDQQVRLFRPWLNMDRMLRSAMRLCLPSFDKLELLECIRRLIEVDKDWVPDAAGTSLYVRPVLIGNEVLWLYGPDHQLTEVGTMNIFVYWTHEDGVLELVTPPLNGVILPGVVRQSLLDMAQTWGEFRVVERTITMKQLLRALEEGRVREVFGSGTACQVCPVHRILYKDRNLHIPTMENGPELILRFQKELKEIQYGIRAHEWMFPV

SEQ ID NO: 43 - DNA, 1578 bp, Homo sapiens
  misc_feature: Incyte ID No 70612021CB1
ttcggctcgaggtcttccagaaccttcttcagtgaggaggttatacccggaggtctcgacgctcgctcgtgctgctcgtgttgccgctgggtacgtttgctgccagtgctgatccagccggaggcgaagagaagcactttgggggcaaaagaagagaaaaatgaagacgggacattttgaaatagtcaccatgctgctggcaaccatgattctagtggacattttccaggtgaaggctgaagtgttagacatggcagataatgcatttgatgatgaatacctgaaatgtacggacaggatggaaattaaatacgttccccaactgctaaaggaggaaaaagcaagccaccagcaattagatactgtgtgggaaaatgcaaaagccaaatgggcagcccgaaagactcaaatctttctccctatgaattttaaggataaccatggaatagccctgatggcatatatttccgaagctcaagagcaaactcccttttaccatctgttcagtgaagctgtgaagatggctggccaatctcgagaagattatatctatggcttccagttcaaagctttccacttttacctcacaagagccctgcagttgctgagaaaaccttgtgaggccagttccaaaactgtggtatatagaacaagccagggcacttcatttacatttggagggctaaaccaagccaggtttggccattttaccttggcatattcagccaaacctcaggctgctaatgaccagctcactgtgttatccatctacacatgccttggagttgacattgaaaattttcttgataaagaaagtgaaagaattactttaatacctctgaatgaggtttttcaagtgtcacaggagggggctggcaataaccttatccttcaaagcataaacaagacctgcagccattatgagtgtgcatttctaggtggactaaaaaccgaaaactgtattgagaacctagaatattttcaacccatctatgtctacaaccctggtgagaaaaaccagaagcttgaagaccatagtgagaaaaactggaagcttgaagaccatggtgagaaaaaccagaagcttgaagaccatggtgtgaaaatccttgaacccacccaaatacctggaatgaaaattccagaaccttttccactacctgctccaggtccagttcctgttccaggtcccaaaagccatccttctgcatccttgggcaaactgctgcttccacagtttgggatggtcatcattttaatcagtgtttctgctataaatctctttgttgctctgtagtttgatgcattgtttatctttcttattctttacttgaaataactatagggatacacaggagatcaaaaggaatgatgtattttttacgtgttggccaaagtcactggataaaatgagaattgtatatttgttattcattttgcaaattcagaaagttggtccagatatatgtcacagaacttttcacttgtatactactcttacaatggaaaaaaatcccgaaaactgtatacttctgattaaattcaataaaagattttgattagaaaaaaaaaaaaaaa

SEQ ID NO: 44 - DNA, 2770 bp, Homo sapiens
  misc_feature: Incyte ID No 71847235CB1
gaggaagtgagaggtcggctgggggtcctcaaagtgagaggggagcagaggatcctcccgtgcaggctgtggatgtcactcacttcccagctggtgaagcctcgctgcagagatgcatctgctcccagccctggcaggggtcctggccacactcgtcctcgcccagccctgtgagggcactgacccagcctcccctggggcagtggagacctcggtcctgcgagactgcatagcagaggccaagttgctggtggatgctgcctacaattggacccagaagagcatcaagcagcggcttcgcagcggttcagccagccccatggacctcctgtcctacttcaaacaaccggtagcagccaccaggacagttgttcgggccgcagattatatgcatgtggctttggggctgcttgaagagaagttacaaccccagcggtccggacccttcaatgtcactgatgtgctaacagaaccacacctgcggctgctgtcccaggccagtggctgtgctctccgggaccaggccgagcgctgcagcgacaagtaccgcaccatcactggacggtgcaacaacaagaggagacccttgctaggggcctccaaccaggctctggctcgctggctgcccgccgagtatgaggatgggctgtcgctccccttcggctggacccccagcaggaggcgcaatggcttccttctccctcttgtccgggctgtctccaaccagattgtgcgcttccccaatgagagactgacctccgaccgtggccgggccctcatgttcatgcagtggggccagttcattgaccatgacctggacttctccccggagtccccggccagagtggccttcactgcaggcgttgactgtgagaggacctgcgcccagctgcccccctgctttcccatcaagatcccacccaatgacccccgcatcaagaaccagcgtgactgcatccctttcttccgctcggcaccctcatgcccccaaaacaagaacagagtccgcaaccagatcaacgcgctcacctcctttgtggacgccagcatggtgtatggcagtgaggtctccctctcgctgcggctccgcaaccggaccaactacctggggctgctggccatcaaccagcgctttcaagacaacggccgggccctgctgcccttcgacaacctgcacgatgacccctgtctcctcaccaaccgctcggcgcgcatcccctgcttcctggcaggtgacacccgatcaacggaaacccccaaactggcagccatgcacaccctctttatgcgagagcacaaccggctggccaccgagctgagacgcctgaatccccggtggaatggagacaaactgtacaatgaggctcggaagatcatgggggccatggtccagatcatcacctaccgagactttctgcccctggttctgggcaaggcccgggccaggagaaccctggggcactacagggggtactgctccaatgtggacccacgggtggccaatgtcttcaccctggccttccgctttggccacacaatgctccagcccttcatgttccgcttggacagtcagtaccgggcctccgcacccaactcgcatgtcccacttagctctgccttctttgccagctggcggatcgtgtatgaagggggcatcgaccccatcctccggggcctcatggccacccctgccaagctgaaccgtcaggatgccatgttagtggatgagctccgggaccggctgtttcggcaagtgaggaggattgggctggacctggcagctctcaacatgcaacgaagccgggaccacggccttccagggtacaatgcttggaggcgcttctgtgggctctcccagccccggaatttggcacagcttagccgggtgctgaaaaaccaggacttggcaaggaagttcctgaatttgtatggaacacctgacaacattgacatctggattggggccatcgctgagcctcttttgccgggggctcgagtggggcctcttctggcttgtctgttcgagaaccagttcagaagagcccgagacggagacaggttctggtggcagaaacgaggtgttttcaccaaaagacagcgcaaggccctgagcagaatttccttgtctcgaattatatgtgacaataccggtatcaccacggtttcaagggacatcttcagagccaacatctaccctcggggctttgtgaactgcagccgtatccccaggttgaacctatcagcctggcgagggacatgaggcttctgcaggagtctatcccaagtctccaacttttggagacaaggggaaggggaggaccatgaggctgccttgtctccctggagcaagtgcaggctgctgacgcttctgctggctacagctcagagctgggttccccagccaggagtgaaggctgggggctcctatcagcaatggaccttcccgccttgggagcctcttaggtattaggctatgaatcagcgccacgtgcaaaggcttgggagccaagccatgtggtcttgcaccccaggcaagaaaagtcagctggagggtttacagcactttctactgtttcccagccctccctcccctccctcaccatgactaagagaccactcggtcctagcctccagacaccccacaatactcctctgagcctgaggccaggcagcatgctctgcttctaccaataaagcactgctaagggcaaaaaaaaaaaaaaaaaaaaaaaaaaaaaaaaaaaaaaaaaaaaaaaaaaaaagggggggg

SEQ ID NO: 45 - DNA, 2651 bp, Homo sapiens
  misc_feature: Incyte ID No 7505230CB1
tgctgctgcagctctgagcattcccacgtcaccagagaagccggtgggcaatgagagcatgtctgctttcaggttgtggcctggcctgctgatcatgttgggttctctctgccatagaggttcaccgtgtggcctttcaacacacatagaaataggacacagagctctggagtttcttcagcttcacaatgggcgtgttaactacagagagctgttactagaacaccaggatgcgtatcaggctggaatcgtgtttcctgattgtttttaccctagcatctgcaaaggaggaaaattccatgatgtgtctgagagcactcactggactccgtttcttaatgcaagcgttcattatatccgagagaactatccccttccctgggagaaggacacagagaaactggtagctttcttgtttggaattacttctcacatggcggcagatgtcagctggcatagtctgggccttgaacaaggattccttaggaccatgggagctattgattttcacggctcctattcagaggctcattcggctggtgattttggaggagatgtgttgagccagtttgaatttaattttaattaccttgcacgacgctggtatgtgccagtcaaagatctactgggaatttatgagaaactgtatggtcgaaaagtcatcaccgaaaatgtaatcgttgattgttcacatatccagttcttagaaatgtatggtgagatgctagctgtttccaagttatatcccacttactctacaaagtccccgtttttggtggaacaattccaagagtattttcttggaggactggatgatatggcattttggtccactaatatttaccatctaacaagcttcatgttggagaatgggaccagtgactgcaacctgcctgagaaccctctgttcattgcatgtggcggccagcaaaaccacacccagggctcaaaaatgcagaaaaatgattttcacagaaatttgactacatccctaactgaaagtgttgacaggaatataaactatactgaaagaggagtgttctttagtgtaaattcctggaccccggattccatgtcctttatctacaaggctttggaaaggaacataaggacaatgttcataggtggctctcagttgtcacaaaagcacgtctccagccccttagcatcttacttcttgtcatttccttatgcgaggcttggctgggcaatgacctcagctgacctcaaccaggatgggcacggtgacctcgtggtgggcgcaccaggctacagccgccccggccacatccacatcgggcgcgtgtacctcatctacggcaatgacctgggcctgccacctgttgacctggacctggacaaggaggcccacaggatccttgaaggcttccagccctcaggtcggtttggctcggccttggctgtgttggactttaacgtggacggcgtgcctgacctggccgtgggagctccctcggtgggctccgagcagctcacctacaaaggtgccgtgtatgtctactttggttccaaacaaggaggaatgtcttcttcccctaacatcaccatttcttgccaggacatctactgtaacttgggctggactctcttggctgcagatgtgaatggagacagtgaacccgatctggtcatcggctccccttttgcaccaggtggagggaagcagaagggaattgtggctgcgttttattctggccccagcctgagcgacaaagaaaaactgaacgtggaggcagccaactggacggtgagaggcgaggaagacttctcctggtttggatattcccttcacggtgtcactgtggacaacagaaccttgctgttggttgggagcccgacctggaagaatgccagcaggctgggccatttgttacacatccgagatgagaaaaagagccttgggagggtgtatggctacttcccaccaaacggccaaagctggtttaccatttctggagcccctacgtacgatgacgtgtctaaggtggcattcctgaccgtgaccctacaccaaggcggagccactcgcatgtacgcactcatatctgacgcgcagcctctgctgctcagcaccttcagcggagaccgccgcttctcccgatttggtggcgttctgcacttgagtgacctggatgatgatggcttagatgaaatcatcatggcagcccccctgaggatagcagatgtaacctctggactgattgggggagaagacggccgagtatatgtatataatggcaaagagaccacccttggtgacatgactggcaaatgcaaatcatggataactccatgtccagaagaaaaggcccaatatgtattgatttctcctgaagccagctcaaggtttgggagctccctcatcaccgtgaggtccaaggcaaagaaccaagtcgtcattgctgctggaaggagttctttgggagcccgactctccggggcacttcacgtctatagccttggctcagattgaagatttcactgcatttccccactctgcccacctctctcatgctgaatcacatccatggtgagcattttgatggacaaagtggcacatccagtggagcggtggtagatcctgatagacatggggctcctgggagtagagagacacactaacagccaca

SEQ ID NO: 46 - DNA, 2133 bp, Homo sapiens
  misc_feature: Incyte ID No 7505235CB1
ccacgcgtccgaacgccatggctcccaagaagctgtcctgccttcgttccctgctgctgccgctcagcctgacgctactgctgccccaggcagacactcggtcgttcgtagtggataggggtcatgaccggtttctcctagacggggccccgttccgctatgtgtctggcagcctgcactactttcgggtaccgcgggtgctttgggccgaccggcttttgaagatgcgatggagcggcctcaacgccatacagttttatgtgccctggaactaccacgagccacagcctggggtctataactttaatggcagccgggacctcattgcctttctgaatgaggcagctctagcgaacctgttggtcatactgagaccaggaccttacatctgtgcagagtgggagatggggggtctcccatcctggttgcttcgaaaacctgaaattcatctaagaacctcagatccagctgacaacatgaccaaaatctttaccctgcttcggaagtatgaaccccatgggccattggtaaactctgagtactacacaggctggctggattactggggccagaatcactccacacggtctgtgtcagctgtaaccaaaggactagagaacatgctcaagttgggagccagtgtgaacatgtacatgttccatggaggtaccaactttggatattggaatggtgccgataagaagggacgcttccttccgattactaccagctatgactatgatgcacctatatctgaagcaggggaccccacacctaagctttttgctcttcgagatgtcatcagcaagttccaggaagttcctttgggacctttacctcccccgagccccaagatgatgcttggacctgtgactctgcacctggttgggcatttactggctttcctagacttgctttgcccccgtgggcccattcattcaatcttgccaatgacctttgaggctgtcaagcaggaccatggcttcatgttgtaccgaacctatatgacccataccatttttgagccaacaccattctgggtgccaaataatggagtccatgaccgtgcctatgtgatggtggatggggtgttccagggtgttgtggagcgaaatatgagagacaaactatttttgacggggaaactggggtccaaactggatatcttggtggagaacatggggaggctcagctttgggtctaacagcagtgacttcaagggcctgttgaagccaccaattctggggcaaacaatccttacccagtggatgatgttccctctgaaaattgataaccttgtgaagtggtggtttcccctccagttgccaaaatggccatatcctcaagctccttctggccccacattctactccaaaacatttccaattttaggctcagttggggacacatttctatatctacctggatggaccaagggccaagtctggatcaatgggtttaacttgggccggtactggacaaagcaggggccacaacagaccctctacgtgccaagattcctgctgtttcctaggggagccctcaacaaaattacattgctggaactagaagatgtacctctccagccccaagtccaatttttggataagcctatcctcaatagcactagtactttgcacaggacacatatcaattccctttcagctgatacactgagtgcctctgaaccaatggagttaagtgggcactgaaaggtaggccgggcatggtggctcatgcctgtaatcccagcactttgggaggctgagacgggtggattacctgaggtcaggacttcaagaccagcctggccaacatggtgaaaccccgtctccactaaaaatacaaaaattagccgggcgtgatggtgggcacctctaatcccagctacttgggaggctgagggcaggagaattgcttgaatccaggaggcagaggttgcagtgagtggaggttgtaccactgcactccagcctggctgacagtgagacactccatctcaaaaaaaaaaaaaaaaaaaagtttccccttgggcccgtggacatggggtggcggaatccctgtgggggctgccacgtggcccttagggactaaagggcccgggccctctaagttttcccttacctttcttgc

SEQ ID NO: 47 - DNA, 1140 bp, Homo sapiens
  misc_feature: Incyte ID No 7505793CB1
gcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaa

SEQ ID NO: 48 - DNA, 945 bp, Homo sapiens
  misc_feature: Incyte ID No 7505861CB1
cgcatgactggcagtggcatcagcgatggcggctgcgtcggggtcggttctgcagcgctgtatcgtgtcgccggcagggaggcatagcgcctctctgatcttcctgcatggctcaggtgattctggacaaggattaagaatgtggatcaagcaggttttaaatcaagatttaacattccaacacataaaaattatttatccaacagctcctcccagatttaaaataaccaatgactgcccagaacaccttgaatcaattgatgtcatgtgtcaagtgcttactgatttgattgatgaagaagtaaaaagtggcatcaagaagaacaggatattaataggaggattctctatgggaggatgcatggcaatgcatttagcatatagaaatcatcaagatgtggcaggagtatttgctctttctagttttctgaataaagcatctgctgtttaccaggctcttcagaagagtaatggtgtacttcctgaattatttcagtgtcatggtactgcagatgagttagttcttcattcttgggcagaagagacaaactcaatgttaaaatctctaggagtgaccacgaagtttcatagttttccaaatgtttaccatgagctaagcaaaactgagttagacatattgaagttatggattcttacaaagctgccaggagaaatggaaaaacaaaaatgaatgaatcaagagtgatttgttaatgtaagtgtaatgtctttgtgaaaagtgatttttactgccaaattataatgataattaaaatattaagaaataacactttcctgacttttttattattaaaatgcttatcactgtagacagtagctaatcttattaatgaaaaacaatagacaaacatctgtgcataatttttcagacacaattctgtaaatatttggaaaccttttaagtatttaaacttttaaattttt

SEQ ID NO: 49 - DNA, 1309 bp, Homo sapiens
  misc_feature: Incyte ID No 7505864CB1
agcgaggtgcctcggtgtgcgcggagctagtttcccagtttcccgggcccctcccttctccgagcccctctagcgatttgtttaggaaaagtgatgacatgaactagtagtggagaatcgcagcgccgctccccgccctggggagggaggggagccccggagagcctgccggtgggagctggaagcaggctcccggctgagcgccccagcccgaaaggcagggtctgggtgcgggaagagggctcggagctgccttcctgctgccttggggccgcccagatgagggaacagcccgatttgcctggttctgattctccaggctgtcgtggttgtggaatgcaaacgccagcacataatggaaacaggacctgaagacccttccagcatgccagaggaaagttcccccaggcggaccccgcagagcattccctaccaggacctccctcacctggtcaatgcagacggacagtacctcttctgcaggtactggaaacccacaggcacacccaaggccctcatctttgtgtcccatggagccggagagcacagtggccgctatgaagagctggctcggatgctgatggggctggacctgctggtgttcgcccacgaccatgttggccacggacagagcgaaggggagaggatggtagtgtctgacttccacgttttcgtcagggatgtgttgcagcatgtggattccatgcagaaagactaccctgggcttcctgtcttccttctgggccactccatgggaggcgccatcgccatcctcacggccgcagagaggccgggccacttcgccggcatggtactcatttcgcctctggttcttgccaatcctgaatctgcaacaactttcaaggtcgacatttataactcagaccccctgatctgccgggcagggctgaaggtgtgcttcggcatccaactgctgaatgccgtctcacgggtggagcgcgccctccccaagctgactgtgcccttcctgctgctccagggctctgccgatcgcctatgtgacagcaaaggggcctacctgctcatggagttagccaagagccaggacaagactctcaagatttatgaaggtgcctaccatgttctccacaaggagcttcctgaagtcaccaactccgtcttccatgaaataaacatgtgggtctctcaaaggacagccacggcaggaactgcgtccccaccctgaatgcattggccggtgcccggctcatggtctgggggatgcaggcaggggaagggcagagatggcttctcagatatggcttgcaaaaaaaaaaaaaaaaaaaaa

SEQ ID NO: 50 - DNA, 1458 bp, Homo sapiens
  misc_feature: Incyte ID No 7506427CB1
gcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaa

SEQ ID NO: 51 - DNA, 1414 bp, Homo sapiens
  misc_feature: Incyte ID No 7506429CB1
gcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaa

SEQ ID NO: 52 - DNA, 526 bp, Homo sapiens
  misc_feature: Incyte ID No 7505799CB1
cggacggtggtccgcagcgggttctcattgctcgctgggcagacccaggtcgcgctcccactgccgagcccgcgagatgctccccagagctgcctggagcttggtgctgaggaaaggtggaggtggaagacgagggatgcacagctcagaaggcaccacccgtggtggggggaagatgtccccctacaccaactgctatgcccagcgctactaccccatgccagaagagcccttctgcacagaactcaacgctgaggagcaggccctgaaggagaaggagaagggaagctggacccagctgacccacgccgaaaaggtggccttatttcctccaaagccgatcaccttgacggacgagcggaaagcccagcagctgcagcgcatgctggacatgaaggtgaatcctgtgcagggcctggcctcccactgggactatgagaagaagcagtggaagaagtgacttgcatccccagctgtctccctgaggctccgccctggctgggagcctctggcggcccctcccctc

SEQ ID NO: 53 - DNA, 1989 bp, Homo sapiens
  misc_feature: Incyte ID No 7505843CB1
tctcggcggcggcggcggcggcgacagagcgagcgcggcgcggggccaccatgggggcccagctcagcacgttgggccatatggtgctcttcccagtctggttcctgtacagtctgctcatcgaccgggagatcatcagccatgacacccggcgcttccgctttgccctgccgtcaccccagcacatcctgggcctccctgtcggccagcacatctacctctcggctcgaattgatggaaacctggtcgtccggccctatacacccatctccagcgatgatgacaagggcttcgtggacctggtcatcaaggtttacttcaaggacacccatcccaagtttcccgctggagggaagatgtctcagtacctggagagcatgcagattggagacaccattgagttccggggccccagtgggctgctggtctaccagggcaaagggaagttcgccatccgacctgacaaaaagtccaaccctatcatcaggacagtgaagtctgtgggcatgatcgcgggagggacaggcatcaccccgatgctgcaggtgatccgcgccatcatgaaggaccctgatgaccacactgtgtgccacctgctctttgccaaccagaccgagaaggacatcctgctgcgacctgagctggaggaactcaggaacaaacattctgcacgcttcaagctctggtacacgctggacagagcccctgaagcctgggactacggccagggcttcgtgaatgaggagatgatccgggaccaccttccacccccagaggaggagccgctggtgctgatgtgtggccccccacccatgatccagtacgcctgccttcccaacctggaccacgtgggccaccccacggagcgctgcttcgtcttctgagggccgggcacggtcacacggccacccgccccgcgcaccccacgccctgttcacgctcacccagtcacctccccacatcgcacactggggccccgggttcagcctggcctgcccgtgccctggtgaatcacctggctgagcagttcccctggagccccttcgggagcagggctgtgtcccagatgggccacggctgagccttcagagtacgtcctgcctggcacttactggtccttaccagagacgcccagccccatccctgtcctcatgacccctcgtccaccccccacacacactataaggctgagggctgccagcagccccgtctgcccaccattcccggccgtggaccatagtcgggatgtcagcagacacacatgggcagcccaaagctgcaggtgccagggcccaccccagcctcgcctgtcacccccactcccgcctcagggccaggcccaggcctcaccacctgacgctgcatgagacattgacaccagaaagccctcttgggggcactgctccctaccccagggccctggccagccgggagcttggctctcctctggctagagtgggaagagggggctggccatggggccctcccagaacctcagcatttccttccagcccatccaaacactgaggcagccttggggaaccccgagctggggggttggcagcccactgcaccgcctcagggttttggggtcctgggctggggccaccatccctgatggcagaactcccacaaccacatgtatttattcctctgtcctaaaccgtcccctccttccctcacccccagcacagggggattctgagcagtgcctcttgtctgagggacatatcagtgacctcgacgttgcctttagactacagttgtgttagcctcttgcgtattggctttttcagagtcatttatgagcagaaaaaaaaaaagtaaaactttttggaaatattaaaaagccattaaaaaagggccggccgttttagagggtttccaagtctttacggtaagcgttgaatggggacgtccatagggtttgcctagtaagggcccaccgaaagttgaaattccatgggcccggccgttttgaaaaacgtt

SEQ ID NO: 54 - DNA, 1987 bp, Homo sapiens
  misc_feature: Incyte ID No 90001378CB1
gtaggagtgaacactgcacaggaatctctgcccatctcaggagaaaccaaacttggggaaaatgtttgcggtccacttgatggcattttacttcagcaagctgaaggaggaccagatcaagaaggtggacaggttcctgtatcacatgcggctctccgatgacacccttttggacatcatgaggcggttccgggctgagatggagaagggcctggcaaaggacaccaaccccacggctgcagtgaagatgttgcccaccttcgtcagggccattcccgatggttccgaaaatggggagttcctttccctggatctcggagggtccaagttccgagtgctgaaggtgcaagtcgctgaagaggggaagcgacacgtgcagatggagagtcagttctacccaacgcccaatgaaatcatccgcgggaacggcacagagctgtttgaatatgtagctgactgtctggcagatttcatgaagaccaaagatttaaagcataagaaattgccccttggcctaactttttctttcccctgtcgacagactaaactggaagagggtgtcctactttcgtggacaaaaaagtttaaggcacgaggagttcaggacacggatgtggtgagccgtctgaccaaagccatgagaagacacaaggacatggacgtggacatcctggccctggtcaatgacaccgtggggaccatgatgacctgtggctatgaagatcctaattgtgagattggcctgattgcaggaacaggcagcaacatgtgctacatggaggacatgaggaacatcgagatggtggaggggggtgaagggaagatgtgcatcaatacagagtggggaggatttggagacaatggctgcatagatgacatccggacccgatacgacacggaggtggatgaggggtccttgaatcctggcaagcagagatacgagaaaatgaccagtgggatgtacttgggggagattgtgcggcagatcctgatcgacctgaccaagcagggtctcctcttccgagggcagatttcagagcgtctccggaccaggggcatcttcgaaaccaagttcctgtcccagatcgaaagcgatcggctggcccttctccaggtcaggaggattctgcagcagctgggcctggacagcacgtgtgaggacagcatcgtggtgaaggaggtgtgcggagccgtgtcccggcgggcggcccagctctgcggtgctggcctggccgctatagtggaaaaaaggagagaagaccaggggctagagcacctgaggatcactgtgggtgtggacggcaccctgtacaagctgcaccctcacttttctagaatattgcaggaaactgtgaaggaactagcccctcgatgtgatgtgacattcatgctgtcagaagatggcagtggaaaaggggcagcactgatcactgctgtggccaagaggttacagcaggcacagaaggagaactaggaacccctgggattggacctgatgcatcttggatactgaacagcttttcctctggcagatcagttggtcagagaccaatgggcaccctcctggctgacctcaccttctggatggccgaaagagaaccccaggttctcgggtactcttagtatcttgtactggatttgcagtgacattacatgacatctctatttggtatatttgggccaaaatgggccaacttatgaaatcaaagtgtctgtcctgagagatcccctttcaacacattgttcaggtgaggcttgagctgtcaattctctatggctttcagtcttgtggctgcgggacttggaaatatatagaatctgcccatgtggctggcaggctgtttccccattgggatgcttaagccatctcttataggggattggaccctgtacttgtggatgaacattggagagcaagaggaactcacgttatgaactagggggatctcatctaacttgtccttaacttgccatgttgacttcaaac

SEQ ID NO: 55 - DNA, 2338 bp, Homo sapiens
  misc_feature: Incyte ID No 7504923CB1
gtgattctcgcctcgccgcagcccagccctgcgcgccttgcccggcggcccccgcccggccgctccgggcccctggccccgcggagcgatgctgctgctggctgccgccttcctcgtggccttcgtgctgctgctgtacatggtgtctccgctcatcagccccaagcccctcgccctgcccggggcgcatgtggtggttacaggaggttccagtggcatcgggaagtgcattgctatcgagtgctataaacaaggagcttttataactctggttgcacgaaatgaggataagctgctgcaggcaaagaaagaaattgaaatgcactctattaatgacaaacaggtggtgctttgcatatcagttgatgtatctcaagactataaccaagtagagaatgtcataaaacaagcacaggagaaactgggtccagtggacatgctggtaaattgtgcaggaatggcagtgtcaggaaaatttgaagatcttgaagttagtacctttgaaaggttaatgagcatcaattacctgggcagcgtgtaccccagccgggccgtgatcaccaccatgaaggagcgccgggtgggcaggatcgtgtttgtgtcctcccaggcaggacagttgggattattcggtttcacagcctactctgcatccaagtttgccataaggggattggcagaagctttgcagatggaggtgaagccatataatgtctacatcacagttgcttacccaccagacacagacacacctggctttgccgaagaaaacagaacaaagcctttggagactcgacttatttcagagaccacatctgtgtgcaaaccagaacaggtggccaaacaaattgttaaagatgccatagtggtcaccatgggccttttccgcactattgctttgttttacctcggaagttttgacagcatagttcgtcgctgcatgatgcagagagaaaaatctgaaaatgcagacaaaactgcctaatcttcttaccccttggaagaagactgtttccaaataatttgaacagcttgctgctaaatgggacccaatttttggcctatagacacttatgtattgttttcgaatacgtcagattggaccagtgctcttcaggaatgtggctgcaagcaaggggctagaagttcacctcctgacagtattattaatactatgcaaatatggaataggagaccatttgattttctaggctttgtggtagagaggtgaaggtatgagaattaatagcgtgtgaacaaagtaaagaacaggattccagaatgatcacttaaatttgtttctatttattcttttttgcccccctagagattaagtccagaaatgtactttctggcacataaagaaatcttgaggactttgtttaaaccttccataaaaaaacaattttcggtttctcgggttctctctctctctctctctgtctctctgtctctctgtctctctgtctctctgtctctctctctctctctctttctttctttgtgtattttattcaagatgagttggacccattgccagtgagtctgaatgtcactgacagccctgtgttgtgctcaggactcactctgctgctggtggaaactcatggcttctctctctctttgatcccataaagctacgagggggacgggagagggcagtgcaatgggaagtaaagagatattttccagtaggaaaagcaatgctttcttgtctttagactcaaatgcttagggaacgtttcatttctcattcatggggaaaggcagcctccttaaatgttttctgaagagcggtaaaatctagaagcttaagaatttacagttccttcaataaccatgatgacctgaagttcacctatcccattttagcatctacttgtttttcccatctcttcctttccaattttgcttatactgctgtaatatttttgtaaaaaaaaaaaaaaaggaaaaaaaagaccagctaaaattttcgacttgactttttaacttaactcatgaattaattaaagcaaatgaaaaaattaaaaagtgtgactttttctcggagcatatatgtagcttttaggaaaggctgatgatggtataaagtttgctcattaagaaaaaaagacaaggctgattttgaagagagttgcttttgaaataaaatgatcacctgttctttatgtgactctcccactgaacctgcagacattatttttataccacgtgctaaggaagcccactcatctaactctgtagccctggaaaccctcttggcccctgaatgttgtttccaggttataggatctgtgttcattagtggattttgacagcaagtgcctctgatgag

SEQ ID NO: 56 - DNA, 1488 bp, Homo sapiens
  misc_feature: Incyte ID No 7506151CB1
ctgctgcctcacccacagcttttgatatctaggaggactcttctctcccaaactacctgtcaccatggcccaccgatttccagccctcacccaggagcagaagaaggagctctcagaaattgcccagagcattgttgccaatggaaaggggatcctggctgcagatgaatctgtaggtaccatggggaaccgcctgcagaggatcaaggtggaaaacactgaagagaaccgccggcagttccgagaaatcctcttctctgtggacagttccatcaaccagagcatcgggggtgtgatccttttccacgagaccctctaccagaaggacagccagggaaagctgttcagaaacatcctcaaggaaaaggggatcgtggtgggaatcaagttagaccaaggaggtgctcctcttgcaggaacaaacaaagaaaccaccattcaagggcttgatggcctctcagagcgctgtgctcagtacaagaaagatggtgttgactttgggaagtggcgtgctgtgctgaggattgccgaccagtgtccatccagcctcgctatccaggaaaacgccaacgccctggctcgctacgccagcatctgtcagcagaatggactggtacctattgttgaaccagaggtaattcctgatggagaccatgacctggaacactgccagtatgttactgagaaggtcctggctgctgtctacaaggccctgaatgaccatcatgtttacctggagggcaccctgctaaagcccaacatggtgactgctggacatgcctgcaccaagaagtatactccagaacaagtagctatggccaccgtaacagctctccaccgtactgttcctgcagctgttcctggcatctgctttttgtctggtggcatgagtgaagaggatgccactctcaacctcaatgctatcaacctttgccctctaccaaagccctggaaactaagtttctcttatggacgggccctgcaggccagtgcactggctgcctggggtggcaaggctgcttccacccagtcgctcttcacagcctgctatacctactagggtccaatgcccgccagcctagctccagtgcttctagtaggagggctgaaagggagcaacttttcctccaatcctggaaattcgacacaattagatttgaactgctggaaatacaacacatgttaaatcttaagtacaagggggaaaaaataaatcagttattgaaacataaaaatgaataccaaggacctgatcaaatttcacacagcagtttccttgcaacactttcagctccccatgctccagaatacccacccaagaaaataataggctttaaaacaatatcggctcctcatccaaagaacaactgctgattgaaacacctcattagctgagtgtagagaagtgcatcttatgaaacagtcttagcagtggtaggttgggaaggagatagctgcaaccgaagaaagaaataaatagtctcagcc

SEQ ID NO: 57 - DNA, 1608 bp, Homo sapiens
  misc_feature: Incyte ID No 7506450CB1
caagcattgcattgcatcaggatgtctatgaaatggacttcagctcttctgctgatacagctgagctgttactttagctctgggagttgtggaaaggtgctggtgtggcccacagaattcagccactggatgaatataaagacaatcctggatgaacttgtccagagaggtcatgagatgctgttttcccctttggtgagctgctggccgagttacttaaaataccctttgtctacagcctccgcttctctcctggctacgcaattgaaaagcatagtggaggacttctgttccctccttcctatgtgcctgttgttatgtcagaactaagtgaccaaatgactttcatagagagggtaaaaaatatgatctatgtgctttattttgaattttggttccaaatatttgacatgaagaagtgggatcagttctacagtgaagttctaggaagacccactacgttatctgagacaatggcaaaagctgacatatggcttattcgaaactactgggattttcaatttcctcacccactcttaccaaatgttgagttcgttggaggactccactgcaaacctgccaaacccctaccgaaggaaatggaagagtttgtccagagctctggagaaaatggtgttgtggtgttttctctggggtcgatggtcagtaacacgtcagaagaaagggccaatgtaattgcatcagcccttgccaagatcccacaaaaggttctgtggagatttgatgggaataaaccagatactttaggactcaatactcggctgtacaagtggataccccagaatgatcttcttggtcacccaaaaaccagagcttttataactcatggtggagccaatggcatctatgaggcaatctaccatggaatccctatggtgggcgttccattgtttgcagatcaacctgataacattgcacacatgaaggccaagggagcagctgttagtttggacttccacacaatgtcgagtacagacttactcaatgcactgaagacagtaattaatgatcctttatataaagagaatgctatgaaattatcaagaattcatcatgatcaaccagtgaagccccttgatcgagcagtcttctggattgaatttgtcatgcgccataaaggagccaagcaccttcgggttgcagcccacgacctcacctggttccagtaccactctttggatgtgactgggttcctgctggcctgtgtggcaactgtgatattcatcatcacaaaatgtctgttttgtgtctggaagtttgttagaacaggaaagaaggggaaaagagattaattacgtctgaggctggaagctgggaaacccaataaatgaactcctttagtttattacaacaagaagacgttgtgatacaagagattcctttcttcttgtgacaaaacatctttcaaaacttaccttgtcaagtcaaaatttgttttagtacctgtttaaccattagaaatatttcatgtcaaggaggaaaacattagggaaaacaaaaatgatataaagccatatgaggttatattgaaatgtattgagcttatattgaaatttattgttccaattcacaggttacatgaaaaaa

SEQ ID NO: 58 - DNA, 2494 bp, Homo sapiens
  misc_feature: Incyte ID No 71380031CB1
cctggagccaggtgcacagcgcatcgcccgaggctgtcaccgccctgccccgcccaccccagctgtcctggacccaggggcagggagaggctggacgccaggtgcgcggacacagaagcgtctaagcacagcttcctccttgccgctccgggaagtgggcagccagcccaggaaccagtaccacctgcaccatggggctgtcccggaaggagcaggtcttcttggccctgctgggggcctcgggggtctcaggcctcacggcactcattctcctcctggtggaggccaccagcgtgctcctgcccacagacatcaagtttgggatcgtgtttgatgcgggctcctcccacacgtccctcttcctgtatcagtggccggcgaacaaggagaatggcacgggtgtggtcagccaggccctggcctgccaggtggaagggcctggaatctcctcctacacttctaatgctgcacaggctggtgagagcctgcagggctgcttggaggaggcgctggtgctgatcccagaggcccagcatcggaaaacacccacgttcctgggggccacggctggcatgaggttgctcagccggaagaacagctctcaggccagggacatctttgcagcagtcacccaggtcctgggccggtctcccgtggacttttggggtgccgagctcctggccgggcaggccgaaggtgcctttggttggatcactgtcaactacggcttggggacgctggtcaagtactccttcactggagaatggatccagcctccggaggagatgctggtgggtgccctggacatgggaggggcctccacccagatcacgttcgtgcctgggggccccatcttggacaagagcacccaggccgattttcgcctctacggctccgactacagcgtctacactcacagctacctgtgctttggacgggaccagatgctgagcaggctcctcgtggggctggtgcagagccgcccggctgccctgctccgtcacccgtgctacctcagcggctaccagaccacactggccctgggcccgctgtatgagtcaccctgtgtccacgccacgcccccgctgagcctcccccagaacctcacagttgaagggacaggcaaccctggagcctgcgtctcagccatccgggaacttttcaacttctccagctgccagggccaggaggactgcgcctttgacggggtctaccagcccccgctgcggggccagttctatgtggaggccagctaccctgggcaggaccgctggctgcgggactactgtgcctcaggcctgtacatcctcaccctcctgcacgagggctacgggttcagcgaggagacctggcccagcctcgagttccgaaagcaggcgggcggtgtggacattggctggacactgggctacatgctgaacctgaccgggatgatcccggccgatgcgccggctcagtggcgggcagagagctacggcgtctgggtggccaaagtggtgttcatggtgctggccctggtggcggtggtgggggctgccttggtccagctcttctggttgcaggactagtgggaaggcggaggtgggcccccacagagcccacaggcagctgcgtcccggatgctggaggcttcctgagccctgagcgccgtggggccttgctctgtggctctgcccacggtcaggtgacagccacctccagggcaccgtcagggtggtgctggccacagaggctgcatgacctcccctcccggcgtccctcccccaacctccttccgcaactgggcttccagggccgtaggtgcctttctgcacacaggccgccaggactcgtggtgtctccaggctgtgtgactgcagggccacatgctgcctgcaaacagggcaagaccacggaggcacaggggtcctgctcctgatggggcctcaggaggggcggagaggggtggaagggagggagctgccccacctggacccccgctctccctgctgttgtctgagcagatggatggagtccaggcctgggggcttctgctgggccagcccggcctcccacacccacttggagggtgagactgcagtgggggttgtttttattaaaagcatcatggacacagcagaagcccccaggcacgaacgtgatctgggtggaggcccctcccatgtccagggcacccaccagcatctcctccggaggctggatccattctccagtgaaggagtacttgaccagcgtccccaagccgtagttgacagtgatccaaccaaaggcaccttcggcctgcccggccaggagctcggcaccccaaaagtccacgggagaccggcccaggacctgggtgactgctgcaaagatgtccctggcctgagagtagttcttccggctgagcaacctcatgccagccgtggcccccaggaacgtgggtgttttccgatgctgggcctctgggatcagcaccagcgcctcctccaaggaccctgcaggctctcaccag

SEQ ID NO: 59 - DNA, 1414 bp, Homo sapiens
  misc_feature: Incyte ID No 7506054CB1
ggacactgtcccagctgccacctagactcggagctccatccaaacctccagcgaagacatcccagccatggccatgcagaaaatctttgcccgggaaatcttggactccaggggcaaccccacggtggaggtggacctgcacacggccaagggccgattccgagcagctgtgcccagtggggcttccacgggtatctatgaggctctggaactaagagacggagacaaaggccgctacctggggaaaggagtcctgaaggctgtggagaacatcaacagtactctgggccctgctctgctgcaaaagaaactaagcgttgcggatcaagaaaaagttgacaaatttatgattgagctagatgggaccgagaataagtccaagtttggggccaatgccatcctgggcgtgtccttggccgtgtgtaaggcgggagcagctgagaagggggtccccctgtaccgccacatcgcagatctcgctgggaaccctgacctcatactcccagtgccagccttcaatgtgatcaacgggggctcccatgctggaaacaagctggccatgcaggagttcatgattctgcctgtgggagccagctccttcaaggaagccatgcgcattggcgccgaggtctaccaccacctcaagggggtcatcaaggccaagtatgggaaggatgccaccaatgtgggtgatgaaggtggcttcgcacccaacatccaggcggctggttacccagacaaggtggtgatcggcatggatgtggcagcatctgagttctatcgcaatgggaagtacgatcttgacttcaagtcgcctgatgatcccgcacggcacatcactggggagaagctcggagagctgtataagagctttatcaagaactatcctgtggtctccatcgaagacccctttgaccaggatgactgggccacttggacctccttcctctcgggggtgaacatccagattgtgggggatgacttgacagtcaccaaccccaagaggattgcccaggccgttgagaagaaggcctgcaactgtctgctgctgaaggtcaaccagatcggctcggtgaccgaatcgatccaggcgtgcaaactggctcagtctaatggctggggggtgatggtgagccaccgctctggggagactgaggacacattcattgctgaccttgtggtggggctctgcacaggacagatcaagactggcgccccctgccgctcggagcgtctggccaaatacaaccaactcatgaggatcgaggaggctcttggggacaaggcaatctttgctggacgcaagttccgtaacccgaaggccaagtgagaagctggaggctccaggactccactggacagacccaggtcttccagacctgcttcctgaaataaacactggtgccaaccaaaa

SEQ ID NO: 60 - DNA, 2352 bp, Homo sapiens
  misc_feature: Incyte ID No 7506139CB1
cccgccgtccccacgcacgcgtcccggctcacgcgtccgcccgcccgcccgcccccgcttgtgccgcccctaccagagacccccaggagcaggatgtccttccagggcaagaaaagcatcccccggatcacgagtgaccgccttctgatcagaggtgggaggatcgtgaatgacgaccagtccttttacgctgatgtgcacgtggaagatggcttgataaaacaaatcggagaaaacctcatcgtccctgggggcatcaagaccattgacgcccacggcctgatggtccttcctggtggcgttgacgtccacacaaggctgcagatgcctgtcctgggcatgacaccggctgacgacttctgtcagggcaccaaggcagcgctagcaggaggaaccaccatgatcttggaccacgtcttccccgacacgggtgtgagcctgctggcggcctacgagcagtggcgggagcgggcggacagcgcggcctgctgcgactactccctgcacgtggacatcacccgatggcatgagagcatcaaggaggagctggaggccctggtcaaggagaagggtgtgaactccttcctggtcttcatggcatacaaggaccggtgccagtgcagcgacagccagatgtacgagatcttcagcatcatccgggacctgggggccttggcccaggtgcacgctgagaacggggacatcgtggaggaggagcagaagcggttgctggagctcggcatcactggccccgagggccacgtgctcagccaccccgaggaggtggaggctgaggcggtgtaccgagctgtcaccatcgccaagcaggcaaactgcccgctgtacgtcaccaaggtgatgagcaagggggcggccgacgccatcgctcaggccaagcgcagaggggtggtcgtgtttggggagcccatcaccgccagcctgggcaccgacggttcacactactggagcaagaactgggccaaggccgcagccttcgtcacatcaccccctgtcaacccagaccccaccacggcagaccacctcacctgcttgctgtccagcggggacctccaggtgacaggcagcgcccactgcaccttcaccactgcccagaaggctgtgggcaaggacaacttcgcgctgatccccgagggcaccaacggcattgaggagcgcatgtcgatggtctgggagaaatgtgtggcctctgggaagatggacgagaatgagttcgtcgcggtgaccagtacaaatgctgccaaaatcttcaatttttacccaaggaaggggcgagtggctgtgggctctgacgctgacctggtcatatggaaccccaaggccaccaagatcatctctgccaagacccacaatctgctggcggagatccacggtgtgccccgtgggctgtatgacgggcccgtccacgaggtgatggtgcctgccaagccagggagtggcgctccggcccgcgcgtcctgcccaggcaagatctccgtgcctcctgtgcgcaacctacatcagtcggggttcagcctatctgggtctcaggctgatgaccacatcgcccgacgcacagcacagaagatcatggcaccacctggcggccgctccaacatcacctctctctcctagacgcccaggaccggccgccctgtgagccgtgctggccccacccgaggccgcgggggccccagggcactcgcccccctccttagcattttcttttgtagaagtttctcgaaggtgcttggcggtcttgccttccccctccccacaggctctccttgtggggtcccaggtcctgctgccaagagcccctcaagagaagggctgaacctggggagatgtcactgccagggtgaggtggagccacatggcagggacaatgccggcagcctgagcccaggcaccccagtgcccgctgggcccagcctggggacagggaacctgccgggctcacagtgtgggagcagctggacaccaggcttcttggtgaaccggcgaggggccgagtcccgcctggtgggcatttgccgccgcctccccaccaccagtcactgcctcgcagagccctacactcccgcagccgctcctcagaggcctgtgcccatcgcaggcctgggaggaaagtgtggcgcagagcccttccttgctcacaacagcttgctgaagactttcaggggaacccattcagaaactttgggttgcaagcaacaaggcccccggccccgtgggaaggggtcccctttttttacggcaaccccaaaagggcccaacagtcctaaaagcctttcccaatgttaagcccccttcaattcccaaggggaaagatttttggttggaatccccctt

SEQ ID NO: 61 - DNA, 1422 bp, Homo sapiens
  misc_feature: Incyte ID No 7506426CB1
gcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaa

SEQ ID NO: 62 - DNA, 2101 bp, Homo sapiens
  misc_feature: Incyte ID No 7506741CB1
gggtggtcagagagaggtaggggcactcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagctccaacaggaccaggagctacaacggattcagaaatgggtggagacattcccaagtgcctgtcctcattggctatggggaggcaaagttcgtgtccagctctatgaccctgactatatgaaggtgattctggggagatcagacccgaaatcccatggttcctacagattcctggctccatggattgggtacggcttgctcctgttgaatgggcagacatggttccagcatcgacggatgctgaccccagccttccactatgacatcctgaagccctatgtggggctcatggcagactctgtacgagtgatgctggacaaatgggaagagctccttggccaggattcccctctggaggtctttcagcacgtctccttgatgaccctggacaccatcatgaagtgtgccttcagccatcagggcagcatccaggtggacagaccaagtgatccaactgaggaaggctcaactacagaaggagggggagctggagaagatcaagaggaagaggcatttggattttctggatatcctcctcttggccaaagggcccctggtctgcccaggccttgctggtgttcaggatggaattgtttcaggaaccacctggaccagatgccctacaccaccatgtgcattaaggaggcactgaggctctacccaccggtgccaggcattggcagagagctcagcactcccgtcaccttccctgatgggcgctccttgcccaaaggtatcatggtcctcctctccatttatggccttcaccacaacccaaaagtgtggcccaacccagaggtgtttgaccctttccgttttgcaccgggttctgctcaacacagccacgctttcctgcccttctcaggaggatcaaggaactgcattgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggtctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtggcttggatagaatccagggctcgtggctctggaatgtgtctgaacttgctgcctcctcgctccttgctctcccaggatcaattgtatcttgagttaaaagaacctgctctccattatctcaagtagcagagcagatgctaaaccgtcacagctgtaaatcatgtgcttaatgcaacatgccctttcgacccccacattctcaccacctgtttctttgtttgatcaccacataaataatctgcacttccagagctcggggcttcacagtctccatccttagcttggcgccctggacccactttctctctcaaactgtcttttctcactgctttgactctgacagactttgtcacccccaacgactggtgttgggtctgaacaccccat

SEQ ID NO: 63 - DNA, 1849 bp, Homo sapiens
  misc_feature: Incyte ID No 7506743CB1
gggtggtcagagagaggtaggggcactcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagctccaacaggaccaggagctacaacggattcagaaatgggtggagacattcccaagtgcctgtcctcattggctatggggaggcaaagttcgtgtccagctctatgaccctgactatatgaaggtgattctggggagatcagacccgaaatcccatggttcctacagattcctggctccatggattgggtacggcttgctcctgttgaatgggcagacatggttccagcatcgacggatgctgaccccagccttccactatgacatcctgaagccctatgtggggctcatggcagactctgtacgagtgatgctggacaaatgggaagagctccttggccaggattcccctctggaggtctttcagcacgtctccttgatgaccctggacaccatcatgaagtgtgccttcagccatcagggcagcatccaggtggacagaccaagtgatccaactgaggaaggctcaactacagaaggagggggagctggagaagatcaagaggaagaggcatttggattttctggatatcctcctcttggccaaagtgtttgacctttccgttttgcaccgggttctgctcaacacagccacgctttcctgcccttctcaggaggatcaaggaactgcattgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggcctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtggcttggatagaatccagggctcgtggctctggaatgtgtctgaacttgctgcctcctcgctccttgctctcccaggatcaattgtatcttgagttaaaagaacctgctctccattatctcaagtagcagagcagatgctaaaccgtcacagctgtaaatcatgtgcttaatgcaacatgccctttcgacccccacattctcaccacctgtttctttgtttgatcaccacataaataatctgcacttccagagctcggggcttcacagtctccatccttagcttggcgccctggacccactttctctctcaaactgtcttttctcactgctttgactctgacagactttgtcacccccaacgactggtgttgggtctgaacaccccat

SEQ ID NO: 64 - DNA, 2036 bp, Homo sapiens
  misc_feature: Incyte ID No 7506746CB1
gggtggtcagagagaggtaggggcactcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagctccaacaggaccaggagctacaacggattcagaaatgggtggagacattcccaagtgcctgtcctcattggctatggggaggcaaagttcgtgtccagctctatgaccctgactatatgaaggtgattctggggagatcagacccgaaatcccatggttcctacagattcctggctccatggattgggtacggcttgctcctgttgaatgggcagacatggttccagcatcgacggatgctgaccccagccttccactatgacatcctgaagccctatgtggggctcatggcagactctgtacgagtgatgctggacaaatgggaagagctccttggccaggattcccctctggaggtctttcagcacgtctccttgatgaccctggacaccatcatgaagtgtgccttcagccatcagggcagcatccaggtgggcagaccaagtgatccaactgaggaaggctcaactacagaaggagggggagctggagaagatcaagaggaagaggcatttggattttctggatatcctcctcttggccaaagggcccctggtctgcccaggccttgctggtgttcaggatggaattgtttcaggaaccacctggaccagatgccctacaccaccatgtgcattaaggaggcactgaggctctacccaccggtgccaggcattggcagagagctcagcactcccgtcaccttccctgatgggcgctccttgcccaaaggtgtttgacccttcccgttttgcaccgggttctgctcaacacagccacgctttcctgcccttctcaggaggatcaaggaactgcatcgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggtctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtggcttggatagaatccagggctcgtggctctggaatgtgtctgaacttgctgcctcctcgctccttgctctcccaggatcaattgtatcttgagttaaaagaacctgctctccattatctcaagtagcagagcagatgctaaaccgtcacagctgtaaatcatgtgcttaatgcaacatgccctttcgacccccacattctcaccacctgtttctttgtttgatcaccacataaataatctgcacttccagagctcggggcttcacagtctccatccttagcttggcgccctggacccactttctctctcaaactgtcttttctcactgctttgactctgacagactttgtcacccccaacgactggtgttgggtctgaacaccccat

SEQ ID NO: 65 - DNA, 1718 bp, Homo sapiens
  misc_feature: Incyte ID No 7506748CB1
gggcctctcacaaacgctgagccccgccccgctgaggcctgtctgcagaatccacagcaaccagcaccatgcccatgacactggggtactggaacatccgcgggctggcccattccatccgcctgctcctggaatacacagactcaagctacgaggaaaagaagtacacgatgggggacgctcctgactatgacagaagccagtggctgaatgaaaaattcaagctgggcctggactttcccaatctgccctacttgattgatgggactcacaagatcacccagagcaacgccatcctgcggtacattgcccgcaagcacaacctgtgcggggaatcagaaaaggagcagattcgcgaagacattttggagaaccagtttatggacagctgcctggatgccttcccaaatctgaaggacttcatctcccgctttgagggcttggagaagatctctgcctacatgaagtccagccgcttcctcccaagacctgtgttctcaaagatggctgtctggggcaacaagtagggccttgaaggccaggaggtgggagtgaggagcccatactcagcctgctgcccaggctgtgcagcgcagctggactctgcatcccagcacctgcctcctcgttcctttctcctgtttattcccatctttactcccaagacttcattgtccctcttcactccccctaaacccctgtcccatgcaggccctttgaagcctcagctacccactatccttcgtgaacatcccctcccatcattacccttccctgcactaaagccagcctgaccttccttcctgttagtggttgtgtctgctttaaagggcctgcctggcccctcgcctgtggagctcagccccgagctgtccccgtgttgcatgaaggagcagcattgactggtttacaggccctgctcctgcagcatggtccctgccttaggcctacctgatggaagtaaagcctcaaccacaaaaaaaaaaaaaaaaaaaaaaaaaaaaaaaaaaaagggggggcggcggaataaggggcctcgtacccggggaattaattgcgggacgggaaccgggggggttacattttccttttggggggtcgttataagagctggggcggtaatccagggggccaataccgggttcccggggtggtgcaatggggtctactggggctccagaaatcccaccaccaatcttccgaacaagtgggaccaggtaggagagaatcgagggagcacagtcgcgcgagaccgagtaatatggaccagcgcatgcaaacggagaagagtcgatcaacgagcgagagtccggcgcaccacgcaagccagcacgtaccatgaggcactacccgagaaaactgcctgacaggcgtcacaaagaacacaacaaatgaccgcgggacgcccgcgcctgggaatagcaacgcagagaagcaagcaaccacgctcacgacgggatcgactcgctcaccgaggcaccttggacaagcaacgcacaggcaatcggagcaccaacctggcgggatggcagtcccagaaaacccgaagggacaacatccaccagaattcgagggcttggagaagatctctgcctacatgaagtccagccgcttcctcccaagacctgtgttcacaaagatggctgtctggggcaacaagtagggccttgaaggccaggaggtgggagtgaggagcccatactcagcctgctgcccag

SEQ ID NO: 66 - DNA, 1845 bp, Homo sapiens
  misc_feature: Incyte ID No 1419966CB1
gctgggtttctgaactgctgggtttctgcttgctcctctggagatgcagcgtctgttgactccagtgaagcgcattctgcaactgacaagagcggtgcaggaaacctccctcacacctgctcgcctgctcccagtagcccaccaaaggttttctacagcctctgctgtccccctggccaaaacagatacttggccaaaggacgtgggcatcctggccctggaggtctacttcccagcccaatatgtggaccaaactgacctggagaagtataacaatgtggaagcaggaaagtatacagtgggcttgggccagacccgtatgggcttctgctcagtccaagaggacatcaactccctgtgcctgacggtggtgcaacggctgatggagcgcatacagctcccatgggactctgtgggcaggctggaagtaggcactgagaccatcattgacaagtccaaagctgtcaaaacagtgctcatggaactcttccaggattcaggcaatactgatattgagggcatagataccaccaatgcctgctacggtggtactgcctccctcttcaatgctgccaactggatggagtccagttcctgggatggtcgttatgccatggtggtctgtggagacattgccgtctatcccagtggtaatgctcgtcccacaggtggggccggagctgtggctatgctgattgggcccaaggcccctctggccctggagcgagctggcagcgatcgacccttcacccttgacgatttacagtacatgatctttcatacacccttttgcaagatggtccagaagtctctggctcgcctgatgttcaatgacttcctgtcagccagcagtgacacacaaaccagcttatataaggggctggaggctttcggggggctaaagctggaagacacctacaccaacaaggacctggataaagcacttctaaaggcctctcaggacatgttcgacaagaaaaccaaggcttccctttacctctccactcacaatgggaacatgtacacctcatccctgtacgggtgcctggcctcgcttctgtcccaccactctgcccaagaactggctggctccaggattggtgccttctcttatggctctggtttagcagcaagtttcttttcatttcgagtatcccaggatgctgctccaggctctcccctggacaagttggtgtccagcacatcagacctgccaaaacgcctagcctcccgaaagtgtgtgtctcctgaggagttcacagaaataatgaaccaaagagagcaattctaccataaggtgaatttctccccacctggtgacacaaacagccttttcccaggtacttggtacctggagcgagtggacgagcagcatcgccgaaagtatgcccggcgtcccgtctaaaggtgttctgcagatccatggaaagcttcctgggaaacgtatgctagcagagcttctccccgtgaatcatatttttaagatcccactcttagctggtaaatgaatttgaatcgacatagtagccccataagcatcagccctgtagagtgaggagccatctctagcgggcccttcattcctctccatgctgcaatcactgtcctgggcttatggtgctatggactaggggtcctttgtgaaagagcaagatggagcaatggagagaagacctcttcctgaatcactggactccagaaatgtgcatgcagatcagctgttgccttcaagatccagataaactttcctgtcatgtgttagaactttattattattaatattgttaaacttctgtgctgttcctgtgaatctcctaattttgtaccttgttctaagctaatata

SEQ ID NO: 67 - DNA, 1465 bp, Homo sapiens
  misc_feature: Incyte ID No 7506451CB1
agcaactggaaaacaagcattgcattgcatcaggatgtctatgaaatggacttcagctcttctgctgatacagctgagctgttactttagctctgggagttgtggaaaggtgctggtgtggcccacagaattcagccactggatgaatataaagacaatcctggatgaacttgtccagagaggtcatgaggtgactgtattggcatcttcagcttccatttctttcgatcccaacagcccatctactcttaaatttgaagtttatcctgtatctttaactaaaactgagtttgaggatattatcaagcagctggttaagagatgggcagaacttccaaaagacacattttggtcatatttttcacaagtacaagaaatcatgtggacatttaatgacatacttagaaagttctgtaaggatatagtttcaaataagaaacttatgaagaaactacaggagtcaagatttgatgttgttcttgcagatgctgttttcccctttggaagacccactacgttatctgagacaatggcaaaagctgacatatggcttattcgaaactactgggattttcaatttcctcacccactcttaccaaatgttgagttcgttggaggactccactgcaaacctgccaaacccctaccgaaggaaatggaagagtttgtccagagctctggagaaaatggtgttgtggtgttttctctggggtcgatggtcagtaacacgtcagaagaaagggccaatgtaattgcatcagcccttgccaagatcccacaaaaggttctgtggagatttgatgggaataaaccagatactttaggactcaatactcggctgtacaagtggataccccagaatgatcttcttgatataaagagaatgctatgaaattatcaagaattcatcatgatcaaccagtgaagccccttgatcgagcagtcttctggattgaatttgtcatgcgccataaaggagccaagcaccttcgggttgcagcccacgacctcacctggttccagtaccactctttggatgtgactgggttcctgctggcctgtgtggcaactgtgatattcatcatcacaaaatgtctgttttgtgtctggaagtttgttagaacaggaaagaaggggaaaagagattaattacgtctgaggctggaagctgggaaacccaataaatgaactcctttagtttattacaacaagaagacgttgtgatacaagagattcctttcttcttgtgacaaaacatctttcaaaacttaccttgtcaagtcaaaatttgttttagtacctgtttaaccattagaaatatttcatgtcaaggaggaaaacattagggaaaacaaaaatgatataaagccatatgaggttatattgaaatgtattgagcttatattgaaatttattgttccaattcacaggttacatgaaaaaaaatttactaggggtacgagct

SEQ ID NO: 68 - DNA, 824 bp, Homo sapiens
  misc_feature: Incyte ID No 90015249CB1
atgctatccacttttgccaggcagaatgacatcccttttcagctgcagacagtggagttggcttggggggagcacctgaagcctgagttcctgaaggtgaaccccctggggaaggtgcctgccctcagagatggcgacttcctactagcagagagcatggtcatcgttttatacctgagtcgaaagtaccagatacggggacactggtacccacctgagctgcaagcctgcacctgcgtggatgagtacttggcgtggaagcatgtcaccatccagctgcctgccaccaatgtctacctgtgcaagacagcctgcagatgctgcacagctggagcggctgttggggaggctgacgccagccctgcagcacctggatgggggggtcctggtggccaggcccttcctggcaatggagcagatctccctggaagacttggtgctgacggaggtgatgcaggtgaagctttcctacccacctgccctcggggggactctgggcatggggctgagccccaaccccagctgccctgtcttcccagcccactgccgttggctgcgacctcttccaagactggccctggctggcagtgtgacaggcccatatgaaggctgcccttggtactgagctcttcctggaggcccacaagctcatactccagcctctgaaccacagtgaggctcggcaggacccccagctggcccagaagcaggtgcagttgctgcaggagtggctccactgagtgggtacagcccactgcactgcaccctgctgtctgcaataaacacactgcatgctcttcaaaaaaaaaaaaaaaaaaaaaaaa

SEQ ID NO: 69 - DNA, 3103 bp, Homo sapiens
  misc_feature: Incyte ID No 7487231CB1
tccttgcgtgtgtgtgtgtgtgcacgcgtgcgtttagatggaggttggcttcccagccacccaggcttggcccaaagtccgtggtacacacgtgcataactcctacccccacctcgcctgcctgctgtaccaggcgttgattccctcttcctgcgtgttttcggtccaaatccttttctttttctccctcccgtcaagatagtggtttccactccctgctctcgccaggacaccgccttttggactggggctgaattctgccccttgaagctctgctccttggagctgggggccccagcggtaggcggagttgattggagacctgccacccacattccgaccccaagcgacctccgagagggcggggtctcaggctgggttatttagctcgtccacccttctccaccagaaggagcgaaacatctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcatttgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaag

SEQ ID NO: 70 - DNA, 1333 bp, Homo sapiens
  misc_feature: Incyte ID No 7506260CB1
cggccccacccccgagcccccgcagccctagagccgcccaagggatggcgatggcgtacttggcttggagactggcgcggcgttcgtgtccgagttctctgcaggtcactagtttcccggtagttcagctgcacatgaatagaacagcaatgagagccagtcagaaggactttgaaaattcaatgaatcaagtgaaactcttgaaaaaggatccaggaaacgaagtgaagctaaaactctacgcgctatataagcaggccactgaaggaccttgtaacatgcccaaaccaggtgtatttgacttgatcaacaaggccaaatgggacgcatggaatgcccttggcagcctgcccaaggaagctgccaggcagaactatgtggatttggtgtccagtttgagtccttcattggaatcctctagtcaggtggagcctggaacagacaggaaatcaactgggtttgaaactctggtggtgacctccgaagatggcatcacaaagatcatgttcaaccggcccaaaaagaaaaatgccataaacactgagatgtatcatgaaattatgcgtgcacttaaagctgccagcaaggatgactcaatcatcactgttttaacaggaaatggtgactattacagtagtgggaatgatctgactaacttcactgatattccccctggtggagtagaggagaaagctaaaaataatgccgttttactgagggaatttgtgggctgttttatagattttcctaagcctctgattgcagtggtcaatggtccagctgtgggcatctccgtcaccctccttgggctattcgatgccgtgtatgcatctgacagggcaacagagatgcttatttttggaaagaagttaacagcgggagaggcatgtgctcaaggacttgttactgaagttttccctgatagcacttttcagaaagaagtctggaccaggctgaaggcatttgcaaagcttcccccaaatgtcttgagaatttcaaaagaggtaatcaggaaaagagagagagaaaaactacacgctgttaatgctgaagaatgcaatgtccttcagggaagatggctatcagatgaatgcacaaatgctgtggtgaacttcttatccagaaaatcaaaactgtgatgaccactacagcagagtaaagcatgtccaaggaaggatgtgctgttacctctgatttccagtactggaactaaataagcttcattgtgccttttgtagtgctagaatatcaattacaatgatgatatttcactacagctctgatgaataaaaagttttgtaaaacaagcttaagaattcacacaaaaaaaaaa

SEQ ID NO: 71 - DNA, 1675 bp, Homo sapiens
  misc_feature: Incyte ID No 7506270CB1
gtgggagaagagcggagcgtgtgagcagtactgcggcctcctctcctctcctaacctcgctctcgcggcctacctttacccgcccgcctgctcggcgaccagaacaccttccaccatgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaattaagtggactagacctccagctgttgagcccctcctagttcttcatcccactccaactcttccccctctcccagttgtcccgattgtaactcaaagggtggaatatcaaggtcgtttttttcattccatgtgcccagttaatcttgctttctttgtttggctgggatagaggggtcaagttattaatttcttcacacctaccctcctttttttccctatcactgaagctttttagtgcattagtggggaggagggtggggagacataaccactgcttccatttaatggggtgcacctgtccaataggcgtagctatccggacagagcacgtttgcagaagggggactcttcttccaggtagctgaaaggggaagacctgacgtactctggttaggttaggacttgccctcgtggtggaaacttttctttaaaaagttaataccccnacttttctaattaaaaagtggggnaatttaggggagagaagggtaggggggtttggganatcaagaggaggaatggnctttgggggctctttgcctttttgggggactaggccttnggcttttgggacctaaaatggccccctggttctggaaacaa

SEQ ID NO: 72 - DNA, 2122 bp, Homo sapiens
  misc_feature: Incyte ID No 7506306CB1
cctggagccaggtgcacagcgcatcgcccgaggctgtcaccgccctgccccgcccaccccagctgtcctggacccaggggcagggagaggctggacgccaggtgcgcggacacagaagcgtctaagcacagcttcctccttgccgctccgggaagtgggcagccagcccaggaaccagtaccacctgcaccatggggctgtcccggaaggagcaggtcttcttggccctgctgggggcctcgggggtctcaggcctcacggcactcattctcctcctggtggaggccaccagcgtgctcctgcccacagacatcaagtttgggatcgtgtttgatgcgggctcctcccacacgtccctcttcctgtatcagtggccggcgaacaaggagaatggcacgggtgtggtcagccaggccctggcctgccaggtggaagggcctggaatctcctcctacacttctaatgctgcacaggctggtgagagcctgcagggctgcttggaggaggcgctggtgctgatcccagaggcccagcatcggaaaacacccacgttcctgggggccacggctggcatgaggttgctcagccggaagaacagctctcaggccagggacatctttgcagcagtcacccaggtcctgggccggtctcccgtggacttttggggtgccgagctcctggccgggcaggccgaaggtgcctttggttggatcactgtcaactacggcttggggacgctggtcaagtactccttcactggagaatggatccagcctccggaggagatgctggtgggtgccctggacatgggaggggcctccacccagatcacgttcgtgcctgggggccccatcttggacaagagcacccaggccgattttcgcctctacggctccgactacagcgtctacactcacagctacctgtgctttggacgggaccagatgctgagcaggctcctcgtggggctggtgcagagccgcccggctgccctgctccgtcacccgtgctacctcagcggctaccagaccacactggccctgggcccgctgtatgagtcaccctgtgtccacgccacgcccccgctgagcctcccccagaacctcacagttgaagggacaggcaaccctggagcctgcgtctcagccatccgggaacttttcaacttctccagctgccagggccaggaggactgcgcctttgacggggtctaccagcccccgctgcggggccagttctatgtggaggccagctaccctgggcaggaccgctggctgcgggactactgtgcctcaggcctgtacatcctcaccctcctgcacgagggctacgggttcagcgaggagacctggcccagcctcgagttccgaaagcaggcgggcggtgtggacattggctggacactgggctacatgctgaacctgaccgggatgatcccggccgatgcgccggctcagtggcgggcagagagctacggcgtctgggtggccaaagtggtgttcatggtgctggccctggtggcggtggtgggggctgccttggtccagctcttctggttgcaggactagtgggaaggcggaggtgggcccccacagagcccacaggcagctgcgtcccggatgctggaggcttcctgagccctgagcgccgtggggccttgctctgtgggtctgcccacggtcaggtgacagccacctccagggcaccgtcagggtggtgctggccacagaggctgcatgacctcccctcccggcgtccctcccccaacctccttccgcaactgggcttccagggccgtaggtgcctttctgcacacaggccgccaggactcgtggtgtctccaggctgtgtgactgcagggccacatgctgcctgcaaacagggcaagaccacggaggcacaggggtcctgctcctgatggggcctcaggaggggcggagaggggtggaagggagggagctgccccacctggacccccgctctccctgctgttgtctgagcagatggatggagtccaggcctgggggcttctgctgggccagcccggcctcccacacccacttggagggtgagactgcagtgggggttgtttttattaaaagcatcatggacacagcaaaaa

SEQ ID NO: 73 - DNA, 1326 bp, Homo sapiens
  misc_feature: Incyte ID No 7506428CB1
gcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaa

SEQ ID NO: 74 - DNA, 3899 bp, Homo sapiens
  misc_feature: Incyte ID No 7678032CB1
gtggtaagatggcggctgtgagtctgcggctcggcgacttggtgtgggggaaactcggccgatatcctccttggccaggaaagattgttaatccaccaaaggacttgaagaaacctcgcggaaagaaatgcttctttgtgaaattttttggaacagaagatcatgcctggatcaaagtggaacagctgaagccatatcatgctcataaagaggaaatgataaaaattaacaagggtaaacgattccagcaagcggtagatgctgtcgaagagttcctcaggagagccaaagggaaagaccaggatctcaccatcccggagtctagtaccgtgaaggggatgatggccggaccgatggccgcgtttaaatggcagccaaccgcaagcgagcctgttaaagatgcagatcctcatttccatcatttcctgctaagccaaacagagaagccagctgtctgttaccaggcaatcacgaagaagttgaaaatatgtgaagaggaaactggctccacctccatccaggcagctgacagcacagccgtgaatggcagcatcacacccacagacaaaaagataggatttttgggccttggtctcatgggaagtggaatcgtctccaacttgctaaaaatgggtcacacagtgactgtctggaaccgcactgcagagaaatgtgatttgttcatccaggagggggcccgtctgggaagaacccccgctgaagtcgtctcaacctgcgacatcactttcgcctgcgtgtcggatcccaaggcggccaaggacctggtgctgggccccagtggtgtgctgcaagggatccgccctgggaagtgctacgtggacatgtcaacagtggacgctgacaccgtcactgagctggcccaggtgattgtgtccaggggggggcgctttctggaagcccccgtctcagggaatcagcagctgtctaatgacgggatgttggtgatcttagcggctggagacaggggcttatatgaggactgcagcagctgcttccaggcgatggggaagacctccttcttcctaggtgaagtgggcaatgcagccaagatgatgctgatcgtgaacatggtccaagggagcttcatggccactattgccgaggggctgaccctggcccaggtgacaggccagtcccagcagacactcttggacatcctcaatcagggacagttggccagcatcttcctggaccagaagtgccaaaatatcctgcaaggaaactttaagcctgatttctacctgaaatacattcagaaggatctccgcttagccattgcgctgggtgatgcggtcaaccatccgactcccatggcagctgcagcaaatgaggtgtacaaaagagccaaggcgctggaccagtctgacaacgatatgtccgccgtgtaccgagcctacatacactaagctgtcgacaccccgccctcacccctccaatcccccctctgaccccctcttcctcacatggggtcgggggcctgggagttcattctggaccagcccacctatctccatttccttttatacagactttgagacttgccatcagcacagcacacagcagcacccttcccctgaggccggtggggaggggacaagtgtcagcaggattggcgtgtgggaaagctcttgagctgggcactggccccccggacgaggtggctgtgtgttcacacacacacacacacacacacacacacacacacacacaggctctcgccccaggatagaagctgcccagaaactgctgcctggctttttttcttccgagcttgtcttatctcaaaccccttccagtcaaggaactagaatcagcaacgagagttggaagccttcccacagcttcccccagagcgaagaggctgtagtcatgtccccatcccccactggattccctacaaggagaggccttgggcccagatgagccagtacagactccagacagaggggcccttggggccctccaacctcaggtgatgagctgagaaagatgttcacgtctaagcgtccagtgtgcacccagcgctccatagacgcctttgtgaactgaaaagagactggcagagtcccgagaagatggggccctggctttccagggagtgcagcaagcagccggcctgcaggtgagcatggaggcccggccctcaccgcctcgaagccatgccccagatgccactgccacagcgggcgctcgctcctccctaggctgttttagtatttggatttgcattccatcccttgggagggagtcctcagggccactagtgatgagccaagaggagtgggggttgggggcgctcctttctgtttccgttaggccacagactcttcacctggctctgaagagccactcttacctcggtcccctcccagtggtcccaccttctccaccctgccctgccaagtcccctgcatgcccaccgctctccatcctccctcctctccctcttcctcccgtggagacagtatttctttctgtctgtccctttggcccagacccagcctgaccaacgatgagcatttcttaggctcagctcttgatacggaaacgagtgtcttcactccagccagcatcatggtcttcggtgcttcccgggcccggggtctgtcgggagggaagagaactgggcctgacctacctgaactgactggccctccgaggtgggtctgggacatcctagaggccctacatttgtccttggataggggaccggggggggcttggaatgttgcaaaaaaaaaagttacccaagggatgtcagttttttatccctctgcatgggttggattttccaaaatcataatttgcagaaggaaggccagcatttacgatgcaatatgtaattatatatagggtggccacactagggcggggtccttcccccctcacagctttggcccctttcagagattagaaactgggttagaggattgcagaagacgagtggggggagggcagggaagatgcctgtcgggtttttagcacagttcatttcactgggattttgaagcatttctgtctgaacacaaagcctgttctagtcctggcggaacacactgggggtgggggcgggggaagatgcggtaatgaaaccggttagtcaattttgtcttaatattgttgacaattctgtaaagttcctttttatgaatatttctgtttaagctatttcacctttcttttgaaatccttcccttttaaggagaaaatgtgacacttgtgaaaaagcttgtaagaaagcccctcccttttttctttaaacctttaaatgacaaatctaggtaattaaggttgtgaatttttatttttgctttgtttttaatgaacatttgtctttcagaataggattgtgtgataatgtttaaatggcaaaaacaaaacatgattttgtgcaattaacaaagctactgcaagaaaaataaaacacttcttggtaacacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagccaacaaaaaaaagcaaaatatacacaacaagtagcacatacaataaagataacaataaaatcagaagagcagatagtaaatacagatgcagacagatacaaggcgaccacagatccccacgtacattacgtgcgataatccaggctataagctcaccagatactacgccgcgcaaccatacacgacataactgcatacgacagcaagagcacggcttaaaaaaaaacacaatacaagtatttcaaataaacaataaacagaacaaagaataatgaaccactaagaaaatctcaaacaacaatagtatacaacactctacactaaaaaccatagaaacaacat

SEQ ID NO: 75 - DNA, 2491 bp, Homo sapiens
  misc_feature: Incyte ID No 7508332CB1
caaatgagtgctgttaaagttcctccaggaaacttcagcagagaaaaacatttgcttcacatctcatcaaatcttctgcatcaagccacatcatgttaaacaaccttctgctgttctcccttcagataagtctcataggaaccactcttggtgggaatgttttgatttggccaatggaaggtagtcattggctaaatgttaagataattatagatgagctcattaaaaaggagcataatgtgactgtcctagttgcctctggtgcacttttcatcacaccaacctctaacccatctctgacatttgaaatatataaggtgccctttggcaaagaaagaatagaaggagtaattaaggacttcgttttgacatggctggaaaatagaccatctccttcaaccatttggagattctatcaggagatggccaaagtaatcaaggacttccacatggtgtctcaggagatctgtgatggcgttcttaaaaaccaacagctgatggcaaagctaaagaaaagcaagtttgaagtcctggtgtctgatccagtatttccttgtggcgatatagtagctttaaaacttggaattccatttatgtactccttgaggttttctccagcctcaacagtggaaaagcactgtgggaaggtaccataccctccttcctatgttcctgctgttttatcagaactcaccgaccaaatgtctttcactgacagaataagaaatttcatctcctaccacctacaggactacatgtttgaaactctttggaaatcatgggattcatactatagtaaagctttaggaagacccactacgttatgtgagactatggggaaagctgaaatttggttaatccgaacatattgggattttgaatttcctcgtccatacttacctaattttgagtttgttggaggattgcactgcaaacctgccaaacctttacctaaggttttatggagatacaaaggaaagaaaccagccacattaggaaacaatactcagctctttgattggataccccagaatgatcttcttggacatcccaaaaccaaagcttttatcactcatggtggaactaatgggatctacgaagctatttaccacggagtccctatggtgggagttcccatgtttgctgatcagcctgataacattgctcacatgaaggccaaaggagcagctgtggaagtgaacctaaacacaatgacaagtgtggatttgcttagcgctttgagaacagtcattaatgaaccttcttataaagagaatgctatgaggttatcaagaattcaccatgatcaacctgtaaagcccctggatcgagcagtcttctggatcgagtttgtcatgcgccacaaaggagccaagcaccttcgggttgcagcccatgacctcacctggttccagtaccactctttggatgtaattgggttcttgctggtctgtgtgacaacggctatatttttggtcatacaatgttgtttgttttcctgtcaaaaatttggtaagataggaaagaagaaaaaaagagaataggtcaagaaaaagaggaaatatatatatttttaagtttggcaaaatcctgagtagtgtagtcctattaattccagacaaaaggagtttaacaaaaacacgtctcccatcctgtttccaaattttctatttctctacctgcgataagcctactgataaagcctagattttggcatgattattattaacttgtgagttatagtcttctatttttcctttgtctctccctgctgactataccctcttcctgtcacttttctgacacaaggatactacctaattttaaatatgttctattcatagtatcaaattattttatcgttaaccttaattaatgattaacaacatgctgaatcctggtaatgcatacagtgtagatggaatttgataggtgtaaggaagagtcaaattcacaaatttccatataccaaacaaatcagggagccaccgtaggagagtagtgtgttatgagaaaggtaatgatttccttttttaataaaaacaaactcttctgcttgctcaatgtttcaggagttagagaatgaattttaagtgtgacgtgcgtccctattaaatgtctacaaaattttcattaagcatatctagaaaatcacggcataacttgcctgcctttcttcaacatatattcttatataacctgtagtggaagatttgggtactgtctttaataaatcaatcaatcgactcttttatttcaaggagaaagttctatgttatatgttgaaggtgaacagatcatatttagaggatataacaattagaaatctagaaaataattatcatttttataaaatttttagtcaactgtacaaataattacataaaacatcaattaattatgcttaaaaatcactaatgttcataatatataatcactatttgtaatcaaaagtttaattttatgccaaaaaataaaaaatgcttacttg

SEQ ID NO: 76 - DNA, 1196 bp, Homo sapiens
  misc_feature: Incyte ID No 1288969CB1
ggcgttgggcaggggtccctcggaggcctcctggggatgggggctgcagctcgtctgagcgcccctcgagcgctggtactctgggctgcactgggggcagcagctcacatcggaccagcacctgaccccgaggactggtggagctacaaggataatctccagggaaacttcgtgccagggcctcctttctggggcctggtgaatgcagcgtggagtctgtgtgctgtggggaagcggcagagccccgtggatgtggagctgaagagggttctttatgacccctttctgcccccattaaggctcagcactggaggagagaagctccggggaaccttgtacaacaccggccgacatgtctccttcctgcctgcaccccgacctgtggtcaatgtgtctggaggtcccctcctttacagccaccgactcagtgaactgcggctgctgtttggagctcgcgacggagccggctcggaacatcagatcaaccaccagggcttctctgctgaggtgcagctcattcacttcaaccaggaactctacgggaatttcagcgctgcctcccgcggccccaatggcctggccattctcagcctctttgtcaacgttgccagtacctctaacccattcctcagtcgcctccttaaccgcgacaccatcactcgcatctcctacaagaatgatgcctactttcttcaagacctgagcctggagctcctgttccctgaatccttcggcttcatcacctatcagggctctctcagcaccccgccctgctccgagactgtcacctggatcctcattgaccgggccctcaatatcacctcccttcagatgcactccctgagactcctgagccagaatcctccatctcagatcttccagagcctcagcggtaacagccggcccctgcagcccttggcccacagggcactgaggggcaacagggacccccggcaccccgagaggcgctgccgaggccccaactaccgcctgcatgcccagtgcttctcgacctggtacattctggttccccgctcgcagacttccgacgtggatggtgtcccccatggtcgctgagactccccttcgaggattgcacccgcccgtcctaagcctccccacaaggcgaggggagttacccctaaaacaaagctattaaagggacagaatacttaaaaaaaaaaaaaaaaaaaggg

SEQ ID NO: 77 - DNA, 1730 bp, Homo sapiens
  misc_feature: Incyte ID No 72069135CB1
ctgaacccgggcggcggaggttgctgtgagcggagactgcaccattgcactcaagcctgggcaacaagaatgaaactgtctcaaaaaaaaaaatttagaacgtagtcatgtacctcgaaagcgaacactcccaacatagcgtggaacaccccacacccccggcgactttgctccgccttggtcccggtcgcagacaccgcagaaaggccatcgttctagacatacgagaactccccgtgcagccaaacgctgtagtcccagcggcggaactcgggggggggaggggggggagtgcccaaccagccagtcagcagtgacgtcccgcctttcctccgtttccattggtccagccatgcggaggtcgctcccatgggacgccgagcttccggctgcagggcttggcgcccgggcgctttcggattgggagggcttcctccatggaacgcgagcctcgagacgtctgacgttaggcaccgttcgcagcgcctcgggctcgcacggcaggatcgaaagcgtgattggctggcggcgtctgtggtctcgggcaccgcccagtcgcgggacgctgcctctgcggaactgggggtggggcgtgttgacccccttaaaggcgccagagcccgcggtcacggctcaggttcccggtgcttcgcgcgtctgccgttgtcacaggccagggaggtggcaccaccaggcgaagcttggcgagattgtgtcgtcaagcgcgtacgggcgccaattggccgggcgatgtggcgtggactggcgctggcgcgagcgattggctgcgcggcccgggggcggggccagtgggcggtgcgcgccgcagactgtgctcaaagcgggcgccatccgggaccggcggttgtctgtggccggaggctgatcagtgttctagaacagatcagacattttgtaatgatgcctgaaataaacactaaccacctcgacaagcaacaggttcaactcctggcagagatgtgtatccttattgatgaaaatgacaataaaattggagctgagaccaagaagaattgtcacctgaacgagaacattgagaaaggattattgcatcgagcttttagtgtcttcttattcaacaccgaaaataagcttctgctacagcaaagatcagatgctaagattacctttccaggttgttttacgaatacgtgttgtagtcatccattaagcaatccagccgagcttgaggaaagtgacgcccttggagtgaggcgagcagcacagagacggctgaaagctgagctaggaattcccttggaagaggttcctccagaagaaattaattatttaacacgaattcactacaaagctcagtctgatggtatctggggtgaacatgaaattgattacattttgttggatggtgagcagcgtccctctcatgtgacacccacagttatgccggatgttgccagatgcccctaggggacagagtcaacccccaactgaggaccactgtcctacagagtcaggaaatattgtagggagaaaaaaataacaacaacaaaggcctgtgttaatgttaaatagatgagattatggaatatgtatattaatgttaaaaattgtaccttgatcaatgtactttttataaacttgccatagatatctcagatttgaaacctcaagacagatttattattcttaaatgctgtatgataatgaagaaaaataaaaatttatttcttgcaaagttaaaaaaaaaa

SEQ ID NO: 78 - DNA, 3322 bp, Homo sapiens
  misc_feature: Incyte ID No 7506247CB1
gcggccgagacgggggcggcgccgcgcgggtctggcgggaccggtttggaagactttgccggcctgcagattggccttaagagaaggacggagccacatactgctgacggcccagaactggcagagagaaggttgccatggctgctgttgacagtttctacctcttgtacagggaaatcgccaggtcttgcaattgctatatggaagctctagctttggttggagcctggtatacggccagaaaaagcatcactgtcatctgtgacttttacagcctgatcaggctgcattttatcccccgcctggggagcagagcagacttgatcaagcagtatggaagatgggccgttgtcagcggtgcaacagatgggattggaaaagcctacgctgaagagttagcaagccgaggtctcaatataatcctgattagtcggaacgaggagaagttgcagtatttcactcagctgtccgaggacaagctctgggacatcataaatgtgaacattgccgccgctagtttgatggtccatgttgtgttaccgggaatggtggagagaaagaaaggtgccatcgtcacgatctcttctggctcctgctgcaaacccactcctcagctggctgcattttctgcttctaaggcttatttagaccacttcagcagagccttgcaatatgaatatgcctctaaaggaatctttgtacagagtctaatccctttctatgtagccaccagcatgacagcacccagcaactttctgcacaggtgctcgtggttggtgccttcgccaaaagtctatgcacatcatgctgtttctactcttgggatttccaaaaggaccacaggatattggtcccattctattcagtttctttttgcacagtatatgcctgaatggctctgggtgtggggagcaaatattctcaaccgttcactacgtaaggaagccttatcctgcacagcctgagtctggatggccacttgagaagttttgccaactcctgggaacctcgatattctgacatttggaaaaacacatttaatttatctcctgtgtttcattgctgattattcagcatactgttgattcgtcatttgcaaaacacacataataccgtcagagtgctgtgaaaaaccttaagggtgtgtggatggcacaggatcaataatgcctgaggctgattgacgacatctacatttcagtgctttttccctaagctgtttgaaagttacgcttttctgttgttctagagccacagcagtctaatattgaaatataatatgatttgtcaggtcttataatttcagatgttgttttttaagggaaattgaccatttcactagaggagttgtgctggtttttaaatgtgcatcaagaaagactactgaaaagtattattttgtaactaagattgctggtactattaggaaaaatctgtgtgtattgtatagctctagctgtttgactatctgtaatgaaaatgctgcacttcaactggtatttcattagagaaccgtgtgtgtgcgtgtgtgtggtgcctttgagcaactttatttatggttaccatatttttaaaaagattttttgtcagggtgacttaacatggactcttatagggtattaaaacaatctagattattccttttcatcctaaataagcctaccaaatttcatgctgttggtttgccatgaatgatattacttcctacattatatttgtgttttttcaaatctgctatggaatgaacttattcctagatttggatatgtaagagaaacctgcagtcatcttttgatttataaggcaattcttgtggataaatagtgatttctcagcctctgacccattttataactgaaatttagccctttagagcttgttatatctggttttcctacgtttttctatgtaatattattccattccagtagcattattgatagaaatagtaagtatttatggaatagtaaaatatggacaaattacgtgtgtgacatatctgtcaatataagttagaagcttattcttggtttgtgtaatgaatttatgtattgtagtgaatacctttactggtgtgaagataattatgcacaaaccctcacaatacgcgttaacattgaaacctgtgaaatgtccttagtttgggtcatataaagccaaccatttttgaggaccatgtacctagtgctttgaaaactctaagtcactatatgaatatgacaatatgtgcacatttaaaattcagagctcggcattgtgatactgatgcagaagctagtagattggttaaaagtctggacttctgtggcatttttttcgtgacgtgataatctatcataagcagacctaagcacagttttatgaacacaattttgcccatgacattgcctacaggatttccagatgtgacttgcactcagaagatcagtggtcaacttcagaagttcttccacgcttagatcatgtcttcagaacttagatgtgaaaatctacacactgggagatgctgtgagccccaaggttttgatggagtttgcttggaatcctcttgacttcatgccacattgacgtgaactttgatgtataataagcagcagcaacttcatgtgaaaatatggtcaggtagttatatgtaaggttacgtggtccagtaatgtcttagattgataaattaggtatggaatccatcagtgttacgtgatgagaataggtgaacacaccttgtcagtgatgatgtaaacttctctccttggcaggacatgggcaaacatgctgattggtgcaaatgtggtgccgagctgtccatagctgcagtgaaagatgaagagcaagaccttctctaggttttctagctttcattaaatgtatttttttccccagagctaatttgaaagttgattggaccactgtggatggggtgtcattaagaatgtgggaaataggggccgagtgcggtggctcacacctgtaatcccagcagtttggaaggccagggcaggtggatcgcttgatcccaggaggtcgagaccagcctggggaacacatcctgtctctacaaaaaatacaaaaattagccaggcagggtggtgcatgcctgtagtcccagctacttgggaggctgaggcaggagaattttttgagcccaggatgcagaggttgaagtgagccaagatcgtgccactgcactccagccttgagacagagcgagaccctgtctcaaaaaaaaaaaaagaacgtgggaaatatgaacctttgaaagttaatctgtgaattgaaagtttaacaataaaagtagttgtttgtttcctttgaaaaaaaaaaa

SEQ ID NO: 79 - DNA, 1529 bp, Homo sapiens
  misc_feature: Incyte ID No 7506363CB1
gccggtcgcgcggtgcgctctccctccctgcccgcagcctggagaggcgcttcgtgctgcacacccccgcgttcctgccggcaccgcgcctgccctctgccgcgctccgccctgccgccgaccgcacgcccgccgcgggacatggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctgagcagcgcctcggagcccggcccgccctccggctacaatccccgcagagtctccggtgcagacgcgctgcggggatggggagcggcgtgccaatctgcgggtcccctgcggcccctgctgccgctgcgctgtcccggccgcaagagcggggccgccccgccgaggtttgtagcagccgggatgtgaccctccagggtttgggtcgctttgcgtttgtcgaagcctcctctgaatggctttgtgaaatcaagatgttttaatcacattcactttacttgaaattatgttgttacacaacaaattgtggggccttcaaattgtttttctcttttcatattaaaaatggtctttctgtgaactagcaaaaaaaaaaaa

SEQ ID NO: 80 - DNA, 2526 bp, Homo sapiens
  misc_feature: Incyte ID No 7509068CB1
cagctgtcggtggcttctgctgagatggccagaggactccaggttcccctgccgcggctggccacaggactgctgctcctcctcagtgtccagccctgggctgagagtggaaaggtgttggtggtgcccactgatggcagcccctggctcagcatgcgggaggccttgcgggagctccatgccagaggccaccaggcggtggtcctcaccccagaggtgaatatgcacatcaaagaagagaaatttttcaccctgacagcctatgctgttccatggacccagaaggaatttgatcgcgttacgctgggctacactcaagggttctttgaaacagaacatcttctgaagagatattctagaagtatggcaattatgaacaatgtatctttggcccttcataggtgttgtgtggagctactgcataatgaggccctgatcaggcacctgaatgctacttcctttgatgtggttttaacagaccccgttaacctctgcggggcggtgctggctaagtacctgtcgattcctgctgtgtttttttggaggtacattccatgtgacttagactttaagggcacacagtgtccaaatccttcctcctatattcctaagttactaacgaccaattcagaccacatgacattcctgcaaagggtcaagaacatgctctaccctctggccctgtcctacatttgccatactttttctgccccttatgcaagtcttgcctctgagctttttcagagagaggtgtcagtggtggatcttgtcagctatgcatccgtgtggctgttccgaggggactttgtgatggactaccccaggccgatcatgcccaacatggtcttcattgggggcatcaactgtgccaacgggaagccactatctcaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagaaagaagcagcagtcaggaagacagatgtgaagagctggagcatgttcagatgagaggagacggaacacggggacacaccagcttgagcaagggacaacaggggaggactgatgactgacttcccacctttgagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctaaaaaaaaaaaaaaaaaaaaagg

SEQ ID NO: 81 - DNA, 1228 bp, Homo sapiens
  misc_feature: Incyte ID No 7505897CB1
aattgcctggctcctctggccaaagggtgctctgcttctggcagctgaagatcccagtagacagcttcttaaaccatggctttccctgcaggatttggatgggcggcagccactgcagcttatcaagtagaaggaggctgggatgcagatggaaaaggcccttgtgtctgggacacatttactcatcagggaggagagagagttttcaagaaccagactggcgatgtagcttgtggcagctacactctgtgggaggaagatttgaaatgtatcaaacagcttggattgactcattaccgcttctctctttcctggtcacgtctgttacctgatgggacgacaggtttcatcaaccagaaagctatccaacttgataaagtcaatcttcaagtatattgtgcatggtctcttctggataactttgagtggaaccagggatacagcagccggtttggtctcttccacgttgattttgaagacccagctagaccccgagtcccttacacatcggccaaggaatatgccaagatcatccgaaacaatggccttgaagcacatctgtaggcaagatggctgagaaatacaggagaggcgtctgcttttggaaaggaaatctgctttggtgatgatctttcaggcaatctcaacttacttctttaatcaacatttaatatcaatggatctgtgattaaaaggtctgaatatgtaatgcctcgtgaagtatttaataatggcctttatttgtatttggatcaatgaggtttttaaaaaaaatggaagagaaaaccactaaccttgatttttgtattgcaaaatcagatagacctggaaacataaatttaaatccttagacatttttctagaaaaaaatgcaaagtttataaagatgatacaaccatgatttgcaactgtaacaggagaccatttattataagcgtacctgtttgtgaacttaattattctgattccataagctgtttttgcttaggtgatccactgccatgtgatccataatttttctacataaaaaatcaaagttaaaagtcacattatacagttatgcattcatttcaacaaaatagtgaattgataatctacttgttaatatattcggcccatattttgtgtgtttggacaagtacatctcccttttgcctaatgaacttttgaaaaataataaaataatagaataaattagactttgaatggcaaaaaaaaaaaaaaaaaa

SEQ ID NO: 82 - DNA, 2034 bp, Homo sapiens
  misc_feature: Incyte ID No 7505898CB1
cccttttttaacacatgagtttctcaatcaatatatttattttcctaaactaaatgttgtatagatacacaatggaatactattcagccttcaaaggtggtaagtttcatcatttgaatcaatatagataaatttagaagacagcatactagatggaataatcctgacatagaaagacaaatactacatgatctcatatatatagaaaaaagtttaactcggggcgcaaagcgagccggtggatccataaagaacccagccaacccgcagagggaggggaggggctgagctgtgaggagagcggggcccaagaaccatgtctacgcgggagtcctttaacccggaaagttacgaattggacaaaagcttccggctaaccagattcactgaactgaagggcacaggctgcaaagtgccccaagatgtcctgcaaaaattgctggaatctttacaggagaaccacttccaagaagatgagcagtttctgggagccgttatgccaaggcttggcattggaatggatacttgtgtcattcctttgaggcacggtgggctttccttggttcaaaccacagattacatttacccgatcgtagacgacccttacatgatgggcaggatagcgtgtgccaatgtcctcagtgacctctatgcaatgggggtcacggaatgtgacaatatgctgatgctccttggagtcagtaataaaatgaccgacagggaaagggataaagtgatgcctctgattatccaaggttttaaagacgcagctgaggaagcaggaacgtctgtaacaggcggccaaacagtactaaacccctggattgtcctgggaggagtggctaccactgtctgccaacccaatgaatttatcatgccagacaatgcagtgccaggggacgtgctggtgctgacaaaacccctggggacacaggtggcagtggctgtgcaccagtggctggatatccctgagaaatggaataagattaaactagtggtcacccaagaagatgtagagctggcctaccaggaggcgatgatgaacatggcgaggctcaacaggacaggcggccttctgatctgtttaccacgtgagcaagcagctcggttctgtgcagagataaagtcccccaaatatggtgaaggccaccaagcatggattattgggattgtagagaagggcaaccgcacagccagaatcatagacaaaccccggatcatcgaggtcgcaccacaagtggccactcaaaatgtgaatcccacacccggggccacctcttaatctagacagaaatagctgtttggttttgtttttaaatagatctatttcccttatcacttcaattaaagactataaacaacaaaaatctcattgtgtctacacatcggggtgaccttaggtcggtttgtaagtggatacaattaataaaataaaatccattgcctttttttcctgttacattaactgaagatgcacctaatcttgaggcagcttctgagttgagaattatattgttatccaatactgttgattcattttgaatctttagacacttatctcttgccgcataggctttttaaaggtgctttcacatagcacaggcattacccgtagtcgtgtcaaatagcagttggtgtcttcattttatgtatatttatcatataagtctgattttttttttttaagcgtcttgaatggttttctggagagacagcattggtaagtggcacatgacggtatcccagtcataagagggttgcatgattcctttgggggtgcacgagtgggttacatcgaactggatctcaacagcggtaagatccttgagagttttcgccccgaagaacgttttccaatgatgagcacttttaaagttctgctatgtggcgcggtattatcccgtatggacgccgggcaagagcaactcggtcgccgcatacactattctcagaatgacttggttgagtactcaccagtcacagaaaagcatcttacggatggcatgacagtagaaattttttgacacctcgtac

SEQ ID NO: 83 - DNA, 1854 bp, Homo sapiens
  misc_feature: Incyte ID No 7505907CB1
gccatggtaagcgcggacgccatggtaagcgcggacgccatggtaagcgcggacgccatggtaagcgcggacgccatggtaagcgcggacgccatggtaagcgcggacgccatggtaagcgcggacgccatggtaagcgcggacgccatggtaagcgcggacgccatggtaagcgcggacgccatggtaagcgcggacgccatgcacacggaccctgactactcggctgcctatgtcgtcatagaaactgatgcagaagatggaatcaaggggtgtggaattaccttcactctgggaaaaggcactgaagttgttgtctgtgctgtgaatgccctcgcccaccatgtgctcaacaaggacctcaaggacattgttggtgacttcagaggcttctataggcagctcacaagtgatgggcagctcagatggattggtccagaaaagggcgtggtgcacctggcgacagcggccgtcctaaacgcggtgtgggacttgtgggccaagcaggagggaaagcctgtctggaagttacttgtggacatggatcccaggatgctggtatcctgcatagatttcaggtacatcactgatgtcctgactgaggaggatgccctagaaatactgcagaaaggtcaaattggtaaaaaagaaagagagaagcaaatgctggcacaaggataccctgcttacacgacatcgtgcgcctggctggggtactcagatgacacgttgaagcagctctgtgcccaggcgctgaaggatggctggaccaggtttaaagtaaaggtgggtgctgatctccaggatgacatgcgaagatgccaaatcatccgagacatgattggaccggaaaagactttgatgatggatgccaaccagcgctgggatgtgcctgaggcggtggagtggatgtccaagctggccaagttcaagccattgtggattgaggagccaacctcccctgatgacattctggggcacgccaccatttccaagtgccacaatagagtgatatttaagcaactcctacaggcgaaggccctgcagttcctccagattgacagttgcagactgggcagtgtcaatgagaacctctcagtattgctgatggccaaaaagtttgaaattcctgtttgcccccatgctggtggagttggcctctgtgaactggtgcagcacctgattatatttgactacatatcagtttctgcaagccttgaaaatagggtgtgtgagtatgttgaccacctgcatgagcatttcaagtatcccgtgatgatccagcgggcttcctacatgcctcccaaggatcccggctactcaacagaaatgaaggaggaatctgtaaagaaacaccagtatccagatggtgaagtttggaagaaactccttcctgctcaagaaaattaagtgctcagccccaacaacttttttctttctgaagtgaaagggcttaaaatttcttggaaatagttttacaaaaatggatttaaaaaatcctaccgatcaagatgagttcagctagaagtcataccaccctcaggaatcagctaagtaattattacttgattcttttagcaaatcaatgcacgttatcctacttaatccttaaataagtttagatttaactaacccaaagtccaggaggatgttcttacaaaaatagctatatcaagggctggcacctagacattaaactgtactttgaaaataagcaacgtgttgcataacttgttgtgggaataattccttgttctgtttaacacttgtcataaattagcagaataaaaatagtcgtgcaacaccgggggtatctggtatgcaacgaagggaaaaatatttcactgattaacc

SEQ ID NO: 84 - DNA, 1346 bp, Homo sapiens
  misc_feature: Incyte ID No 7505925CB1
cggctcgagcggatcatggccgcagccgctctggggcagatctgggcacgaaagcttctctctgtcccttggcttctgtgtggtcccagaagatatgcctcctccagtttcaaggctgcagacctgcagctggaaatgacacagaagcctcataagaagcctggccccggcgagcccctggtgtttgggaagacatttaccgaccacatgctgatggtggaatggaatgacaagggctggggccagccccgaatccagcccttccagaacctcacgctgcacccagcctcctccagcctccactactccctgcagctgtttgagggcatgaaggcgttcaaaggcaaagaccagcaggtgcgcctcttccgcccctggctcaacatggaccggatgctgcgctcagccatgcgcctgtgcctgccgagtttcgacaagctggagttgctggagtgcatccgccggctcatcgaagtggacaaggactgggtccccgatgccgccggcaccagcctctatgtgcggcctgtgctcattgggaacgaggtcctctggctgtatgggcccgaccaccagctcaccgaggtgggaaccatgaacatctttgtctactggacccacgaagatggggtgctggagctggtgacgcccccgctgaatggtgttatcctgcctggagtggtcagacagagtctactggacatggctcagacctggggtgagttccgggtggtggagcgcacgatcaccatgaagcagttgctgcgggccctggaggagggccgcgtgcgggaagtctttggctcgggcaccgcttgccaggtctgcccagtgcaccgaatcctgtacaaagacaggaacctccacattcccaccatggaaaatgggcctgagctgatcctccgcttccagaaggagctgaaggagatccagtacggaatcagagcccacgagtggatgttcccggtgtgaagctgcaggctgtgctccagatccaccgacccgtagcatctcgtcacgccagcactcgcctccctaccaatgactcacctgaagtgcaatacgaaataaaaggccagcgggcggcgtctgggtctctggcgcccccatgtggttgcgacactcccaaagccgtaagggccgacccaggcatcttggcccccagcccctcgtcgcgggttcaggtccgcccattactccttgtcgtgcggtcaaggatacaccttggccccgattccggatctctccgttctcaggccagacccctggtgctgccgttgatttttttttctctgtctttgctgcaattttgaaataaaatgccaaagaacaaaaaaaaaaaaaaa